(19) United States
(12) Patent Application Publication
(10) Pub. No.: US 2025/0074957 A1
(43) Pub. Date: Mar. 6, 2025

(54) MOLECULAR SWITCH-MEDIATED CONTROL OF ENGINEERED CELLS

(71) Applicants: THE GENERAL HOSPITAL CORPORATION, Boston, MA (US); THE BRIGHAM & WOMEN'S HOSPITAL, INC., Boston, MA (US); PRESIDENT AND FELLOWS OF HARVARD COLLEGE, Cambridge, MA (US)

(72) Inventors: Max Jan, Boston, MA (US); Quinlan L. Sievers, Boston, MA (US); Benjamin Ebert, Boston, MA (US); Marcela Maus, Boston, MA (US)

(73) Assignees: THE GENERAL HOSPITAL CORPORATION, Boston, MA (US); THE BRIGHAM & WOMEN'S HOSPITAL, INC., Boston, MA (US); PRESIDENT AND FELLOWS OF HARVARD COLLEGE, Cambridge, MA (US)

(21) Appl. No.: 18/784,373

(22) Filed: Jul. 25, 2024

Related U.S. Application Data
(63) Continuation of application No. 16/759,490, filed on Apr. 27, 2020, now Pat. No. 12,049,482.
(60) Provisional application No. 62/579,454, filed on Oct. 31, 2017.
(60) Provisional application No. 62/633,725, filed on Feb. 22, 2018.

(51) Int. Cl. C07K 14/47; A61K 39/00; C07K 14/705; C07K 14/725; C07K 16/00; C12N 5/0783

(52) U.S. Cl. CPC C07K 14/4702; A61K 39/4611; A61K 39/4631; A61K 39/464412; C07K 14/7051; C07K 14/70521; C07K 14/70578; C07K 16/00; C12N 5/0638; A61K 2239/23; C07K 2317/622; C07K 2319/02; C07K 2319/03

(57) ABSTRACT

The present disclosure relates to therapeutic methods and clinically useful molecular switches, for which activity or degradation of a switch-presenting polypeptide can be precisely induced via administration or withdrawal of an FDA-approved drug. Certain aspects of the disclosure relate to an engineered drug-inducible heterodimeric system including a first polypeptide presenting a CRBN polypeptide disrupted for or lacking a DDB1-interacting domain and a second polypeptide presenting a CRBN polypeptide substrate, where binding between the CRBN polypeptide and the CRBN polypeptide substrate are inducible via administration of an FDA-approved thalidomide analog immunomodulatory drug (IMiD). Another aspect of the disclosure relates to a chimeric antigen receptor (CAR) that presents a minimal fragment of the CRBN polypeptide substrate IKZF3 capable of triggering proteasomal degradation of CAR upon administration of an FDA-approved IMiD.

### CROSS-REFERENCE TO RELATED APPLICATIONS

The present application is a continuation of U.S. application Ser. No. 16/759,490, filed Apr. 27, 2020, which is a national stage application, filed under 35 U.S.C. § 371, of International Application No. of PCT/2018/058210, filed Oct. 30, 2018, which is related to and claims priority under 35 U.S.C. § 119(e) to U.S. provisional patent application No. 62/579,454, entitled “Molecular Switch-Mediated Control of Engineered Cells,” filed Oct. 31, 2017, and to U.S. provisional patent application No. 62/633,725, entitled “Molecular Switch-Mediated Control of Engineered Cells,” filed Feb. 22, 2018. The entire content of the aforementioned patent applications is incorporated herein by this reference.

### STATEMENT REGARDING FEDERALLY SPONSORED RESEARCH

This invention was made with government support under Grant No. CA066996 awarded by the National Institutes of Health. The government has certain rights in the invention.

### FIELD OF THE INVENTION

The invention relates generally to small molecule-responsive molecular switches.

### SEQUENCE LISTING

The instant application contains a Sequence Listing which has been submitted electronically as an XML file entitled “BN00007.0105.xml” created on Nov. 1, 2024, and having a size of 272,258 bytes. The content of the Sequence Listing is hereby incorporated by reference in its entirety.

### BACKGROUND OF THE INVENTION

With recent FDA approval of the first chimeric antigen receptor (CAR) T-cell therapy (Wall Street Journal, Aug. 30, 2017), certain cell-based immunotherapeutics have begun to realize their clinical potential. However, current cellular therapies are constitutively active and lack user-control, meaning that if/when a cell-based immunotherapy elicits a negative reaction in a subject, little can be done (at least with any molecular precision) to halt such a negative reaction. A need therefore exists for clinically applicable systems that enable precise and ideally small molecule-mediated modulation of molecular pathways within engineered cells, particularly CAR-T cells.

### BRIEF SUMMARY OF THE INVENTION

The current disclosure relates, at least in part, to discovery and development of engineered, clinically useful control systems gated by FDA-approved small molecules, that can be employed in engineered cells, particularly therapeutic engineered cells including, e.g., chimeric antigen receptor (CAR) T-cells.

In one aspect, the disclosure provides a method for treating a subject with a chimeric antigen receptor (CAR) cellular therapy, the method including administering to the subject a mammalian cell harboring a drug-responsive CAR that includes: an extracellular antigen-binding domain; a transmembrane domain (TMD); a co-stimulatory domain; a signaling domain; and a CRBN polypeptide substrate domain capable of binding CRBN in response to drug, thereby promoting ubiquitin pathway-mediated degradation of the drug-responsive CAR, where administration of the CAR cellular therapy thereby treats the subject.

In some embodiments, the mammalian cell overexpresses a CRBN polypeptide. Optionally, the overexpressed CRBN polypeptide is targeted to the plasma membrane with a targeting sequence derived from LAT, PAG, LCK, FYN, LAX, CD2, CD3, CD4, CD5, CD7, CD8a, PD1, SRC, or LYN. Optionally, the local concentration of the ubiquitin ligase CRL4CRBN is increased at the plasma membrane via inclusion of such targeting sequence, as compared to an appropriate control polypeptide.

In one embodiment, the method further includes administering the drug.

In another embodiment, the method further includes identifying a CAR cellular therapy-related side effect in the subject. In a related embodiment, the method further includes administering the drug to the subject after the CAR cellular therapy-related side effect is identified in the subject.

In certain embodiments, the subject has or is at risk of developing cancer.

In certain embodiments, the CRBN polypeptide substrate is IKZF1, IKZF3, CK1α, ZFP91, GSPT1, MEIS2, GSS, E4F1, ZN276, ZN517, ZN582, ZN653, ZN654, ZN692, ZN787, ZN827 or a fragment thereof that is capable of drug-inducible binding the CRBN polypeptide disrupted for or lacking a DDB1-interacting domain, or where the CRBN polypeptide substrate is a chimeric fusion product of native CRBN polypeptide substrate sequences, optionally the IKZF3/ZFP91/IKZF3 polypeptide SEQ ID NO: 95.

In some embodiments, the CRBN polypeptide substrate domain includes a hybrid fusion polypeptide that includes ten or more residues of a non-IKZF3 C2H2 zinc finger degron sequence that are flanked by an N-terminal IKZF3 degron sequence and a C-terminal IKZF3 degron sequence. Optionally, the N-terminal IKZF3 degron sequence includes or is amino acids 130-145 (SEQ ID NO: 97) of IKZF3 or a K0 from thereof (SEQ ID NO: 100) and/or the C-terminal IKZF3 degron sequence includes or is amino acids 169-189 (SEQ ID NO: 102) of IKZF3 or a K0 form thereof (SEQ ID NO: 103). Optionally, the non-IKZF3 C2H2 zinc finger degron sequence is a ZFP91 sequence.

Another aspect of the disclosure provides a drug-responsive chimeric antigen receptor (CAR) that includes: an extracellular antigen-binding domain; a transmembrane domain (TMD); a co-stimulatory domain; a signaling domain; and a CRBN polypeptide substrate domain capable of binding CRBN in response to drug, thereby promoting ubiquitin pathway-mediated degradation of the drug-responsive CAR.

In an additional aspect, the disclosure provides a mammalian cell harboring a drug-responsive CAR of the instant disclosure.

In one embodiment, the mammalian cell is a T cell.

In another embodiment, the mammalian cell is a B cell, a plasma cell, a NK cell, a NKT cell, an innate lymphoid cell, a macrophage, a dendritic cell, a monocyte, a neutrophil, a basophil, an eosinophil, a mast cell, a hematopoietic progenitor cell, a hematopoietic stem cell, or another adult stem cell such as neural, cornea, muscle, skin, small intestine, colon, bone, mesenchyme, embryonic stem cell or an induced pluripotent stem cell.

Another aspect of the disclosure provides a polypeptide that includes SEQ ID NO: 95.

A further aspect of the disclosure provides a nucleic acid sequence that includes SEQ ID NO: 84, SEQ ID NO: 85, SEQ ID NO: 86, SEQ ID NO: 87 or SEQ ID NO: 88.

In one aspect, the disclosure provides a method for treating a subject with a chimeric antigen receptor (CAR) cellular therapy, the method involving administering to the subject a mammalian cell having a split chimeric antigen receptor (CAR) system suitable for clinical application that includes a drug-inducible heterodimer, where the split CAR system includes a first polypeptide and a second polypeptide having the following: the first polypeptide includes an extracellular antigen-binding domain, a transmembrane domain (TMD), a co-stimulatory domain and a first domain of a drug-inducible heterodimer; and the second polypeptide includes a second domain of the drug-inducible heterodimer and a signaling domain, where administration of the mammalian cell/CAR cellular therapy thereby treats the subject.

In one embodiment, the first and second domains of the drug-inducible heterodimer bind one another in the presence of the drug. In a related embodiment, the drug-inducible heterodimer is an IMiD-inducible CRBN/CRBN polypeptide substrate heterodimer. In certain embodiments, the IMiD-inducible CRBN/CRBN polypeptide substrate heterodimer includes a CRBN polypeptide disrupted for or lacking a DDB1-interacting domain, optionally the CRBN polypeptide is selected from SEQ ID NOs: 1-4.

In certain embodiments, the IMiD-inducible CRBN/CRBN polypeptide substrate heterodimer includes a CRBN polypeptide disrupted for or lacking a DDB1-interacting domain further including a residue substitution at one or more of positions 371 and 388, optionally where the CRBN polypeptide disrupted for or lacking a DDB1-interacting domain includes a residue substitution selected from I371A, I371G, V388A, and V388G.

In one embodiment, the IMiD-inducible CRBN/CRBN polypeptide substrate heterodimer includes a CRBN polypeptide substrate that is IKZF3, IKZF1, ZFP91, GSPT1, GSS, or a fragment thereof that is capable of drug-inducible binding to CRBN polypeptide, optionally where the CRBN polypeptide substrate is SEQ ID NO: 5.

In another embodiment, the IMiD-inducible CRBN/CRBN polypeptide substrate heterodimer includes a CRBN polypeptide substrate including a substituted form of IKZF3 aa130-189 that includes K→R residue substitutions at positions 137, 158, 166, 172 and 175 of the IKZF3 aa130-189 polypeptide sequence.

In one embodiment, the IMiD-inducible CRBN/CRBN polypeptide substrate heterodimer includes a CRBN polypeptide substrate having a substituted form of IKZF3 aa130-189 that includes a residue substitution at position 153, optionally where the residue substitution at position 153 is selected from A153I, A153M, A153T, A153N, A153Q, A153R, A153H, A153K, A153D, A153E and A153C.

In another embodiment, the method further includes administering the drug to the subject. In a related embodiment, induction of the CAR cellular therapy occurs upon administration of the drug to the subject.

In one embodiment, the drug is an FDA-approved drug, optionally an FDA-approved small molecule drug. Optionally, the drug is a thalidomide analog immunomodulatory drug (IMiD).

In a related embodiment, the drug is thalidomide, lenalidomide or pomalidomide.

In one embodiment, the signaling domain is selected from a CD3ζ domain, a CD3 gamma domain, a CD3 delta domain, a CD3 epsilon domain, a FcR gamma domain, a FcR beta domain, a CD5 domain, a CD79a domain, a CD79b domain, a CD66d domain, a CD4 domain, a CD8 domain, a Dap10 domain and a Dap-12 domain.

In another embodiment, the second polypeptide having the signaling domain further includes one or more domains that is a transmembrane domain (TMD) and/or a co-stimulatory domain.

In one embodiment, the co-stimulatory domain is selected from a CD28 co-stimulatory domain, a 4-1BB co-stimulatory domain, or additional co-stimulatory domains from CD27, OX40, CD30, CD40, ICOS, LFA-1, CD2, CD7, NKG2C, or B7-H3, optionally where the co-stimulatory domain includes K→R residue substitutions at positions 182 and 204 of a CD28 co-stimulatory domain sequence, or where the co-stimulatory domain includes K->R residue substitutions at positions 214, 218, 219, and 225 of a 4-1BB co-stimulatory domain sequence, or where the co-stimulatory domain includes K->R residue substitutions as shown in any of SEQ ID NOs: 46-69.

In another embodiment, the extracellular antigen-binding domain includes a scFv.

Optionally, the extracellular antigen-binding domain includes an anti-CD19/BCMA scFv or a scFv targeting CD19, CD20, CD22, BCMA, CD138, CD38, SLAMF7, kappa light chain, lambda light chain, CD123, CD33, CD45, CD30, CD40, CD70, ErbB2, EGFR, EpCAM, EGFRvIII, mesothelin, ROR1, LeY, PSMA, PSCA, CD34, CD90, TIM3, CD99, CD3, CD4, CD8, CD52 or TCR recognizing WT1.

In one embodiment, the method further includes identifying a CAR cellular therapy-related side effect in the subject. In a related embodiment, the method further includes halting administration of the drug after the CAR cellular therapy-related side effect is identified in the subject.

In another embodiment, the heterodimer is constitutively paired in the absence of the drug. In a related embodiment, the heterodimer is capable of being destabilized by administration of the drug (e.g., a small molecule). Optionally, the drug-destabilized heterodimer is an IMiD-destabilized CRBN/CRBN polypeptide substrate heterodimer, optionally where the heterodimer is CRBN/MEIS2, a MDM2/P53 polypeptide heterodimer inhibited by RG7112, a VHL/HIF-1α or VHL/HIF-2α polypeptide heterodimer inhibited by VH298, or a cIAP/SMAC heterodimer inhibited by birinapant.

Another aspect of the disclosure provides a method for treating a subject with a cellular therapy, the method involving administering to the subject a mammalian cell harboring a drug-inducible heterodimer composition that includes: (i) a first polypeptide having an N-terminus and a C-terminus and including a CRBN polypeptide disrupted for or lacking a DDB1-interacting domain and (ii) a second polypeptide having an N-terminus and a C-terminus and including a CRBN polypeptide substrate, where the CRBN polypeptide and the CRBN polypeptide substrate associate with one another upon administration of the drug, where administering the mammalian cell/cellular therapy thereby treats the subject.

In one embodiment, the cellular therapy is a CAR T cellular therapy.

In certain embodiments, the drug is a small molecule.

In one embodiment, the drug can be administered to a human subject in a clinical setting.

Optionally, the drug is an IMiD, e.g., thalidomide, lenalidomide or pomalidomide.

In one embodiment, the CRBN polypeptide disrupted for or lacking a DDB1-interacting domain is one of SEQ ID NOs: 1-4.

In one embodiment, the CRBN polypeptide substrate is IKZF3 or a fragment thereof that is capable of drug-inducible binding of the CRBN polypeptide disrupted for or lacking a DDB1-interacting domain. Optionally, the CRBN polypeptide substrate is SEQ ID NO: 5.

In certain embodiments, the first and second polypeptides form a system that is a drug-gated split chimeric antigen receptor (CAR) system or a drug-gated heterodimeric cytokine receptor, including class I cytokine receptors, class II cytokine receptors, TNF receptors, IL-1 receptors, tyrosine kinase receptors, and chemokine receptors, drug-gated heterodimeric TGF-beta receptors, drug-gated split genome editing proteins such as CAS9, drug-gated split transcription factors, optionally where a first component (“component A”) encodes a DNA binding motif and a second component (“component B”) encodes an effector motif such as transactivation, repression, or recruitment of an epigenetic reader, writer, or eraser protein.

In one embodiment, the drug-inducible heterodimer incorporates any of the components recited immediately above (receptors, kinases, transcription factors, epigenetic modifiers, genome editing proteins), where the second component serves as a tether to a particular subcellular localization, such that the drug-dependent heterodimerization serves as a location-based gain-, loss- or change-of function switch.

In another embodiment, the first or second polypeptide includes one or more domains that are an extracellular antigen-binding domain, a transmembrane domain (TMD) or a co-stimulatory domain.

In one embodiment, the first or second polypeptide includes a signaling domain. In a related embodiment, the signaling domain is a CD3ζ domain, a CD3 gamma domain, a CD3 delta domain, a CD3 epsilon domain, a FcR gamma domain, a FcR beta domain, a CD5 domain, a CD79a domain, a CD79b domain, a CD66d domain, a CD4 domain, a CD8 domain, a Dap10 domain or a Dap-12 domain. Optionally, the first or second polypeptide including the signaling domain further includes one or more of a transmembrane domain (TMD) and/or a co-stimulatory domain.

In certain embodiments, the co-stimulatory domain is a CD28 co-stimulatory domain, a 4-1BB co-stimulatory domain, and/or includes a co-stimulatory domains from CD27, OX40, CD30, CD40, ICOS, LFA-1, CD2, CD7, NKG2C and/or B7-H3.

In one embodiment, the co-stimulatory domain includes K→R residue substitutions at positions 182 and 204 of a CD28 (Homo sapiens CD28 isoform 1 Uniprot identifier P10747-1) co-stimulatory domain sequence.

In another embodiment, the CRBN polypeptide substrate includes a substituted form of IKZF3 aa130-189 that includes a residue substitution at position 153, optionally where the residue substitution at position 153 is selected from among A153I, A153M, A153T, A153N, A153Q, A153R, A153H, A153K, A153D, A153E and A153C.

In one embodiment, the CRBN polypeptide disrupted for or lacking a DDB1-interacting domain, such as the minCRBN variants 1-4 described in SEQ ID NOs: 1-4, additionally includes a residue substitution at one or more of positions 371 and 388, optionally where the CRBN polypeptide disrupted for or lacking a DDB1-interacting domain includes a residue substitution selected from I371A, I371G, V388A and V388G.

In another embodiment, the CRBN polypeptide substrate includes a substituted form of IKZF3 aa130-189 that includes K→R residue substitutions at positions 137, 158, 166, 172 and 175 of the IKZF3 aa130-189 polypeptide sequence.

In one embodiment, the subject has or is at risk of developing cancer.

In another embodiment, the cellular therapy is administered in a therapeutically effective amount.

In an additional embodiment, the method further includes identifying a cellular therapy-related side effect in the subject. In a related embodiment, the method additionally includes halting administration of the drug after the cellular therapy-related side effect is identified in the subject.

Another aspect of the disclosure provides a drug-inducible heterodimer composition that includes: (i) a first polypeptide having an N-terminus and a C-terminus and including a CRBN polypeptide disrupted for or lacking a DDB1-interacting domain and (ii) a second polypeptide having an N-terminus and a C-terminus and including a CRBN polypeptide substrate, where the CRBN polypeptide and the CRBN polypeptide substrate associate upon administration of the drug.

In one embodiment, the drug-inducible heterodimer composition further includes the drug.

In certain embodiments, the drug is a small molecule. In a related embodiment, the drug is an FDA-approved drug. Optionally, the drug can be administered to a human subject in a clinical setting.

In some embodiments, the drug is an IMiD. Optionally, the drug is thalidomide, lenalidomide or pomalidomide.

In one embodiment, the CRBN polypeptide disrupted for or lacking a DDB1-interacting domain is selected from SEQ ID NOs: 1-4.

In certain embodiments, the extracellular antigen-binding domain includes a scFv. In a related embodiment, the extracellular antigen-binding domain includes a scFv targeting CD19, CD20, CD22, BCMA, CD138, CD38, SLAMF7, kappa light chain, lambda light chain, CD123, CD33, CD45, CD30, CD40, CD70, ErbB2, EGFR, EpCAM, EGFRvIII, mesothelin, ROR1, LeY, PSMA, PSCA, CD34, CD90, TIM3, CD99, CD3, CD4, CD8, CD52, or TCR recognizing WT1.

In another aspect, the disclosure provides a mammalian cell harboring a drug-inducible heterodimer composition of the instant disclosure.

In one embodiment, the mammalian cell is a T cell. In another embodiment, the mammalian cell is a B cell, plasma cell, NK cell, NKT cell, innate lymphoid cell, macrophage, dendritic cell, monocyte, neutrophil, basophil, eosinophil, mast cell, hematopoietic progenitor cell, hematopoietic stem cell, other adult stem cell such as neural, cornea, muscle, skin, small intestine, colon, bone, mesenchyme, embryonic stem cell, or induced pluripotent stem cell.

In certain embodiments, the mammalian cell includes a genomic disruption of native CRBN, optionally a biallelic disruption of native CRBN. In a related embodiment, the mammalian cell includes a genomic disruption of CRBN exon 5, optionally a CRBNΔe5 disruption.

Another aspect of the disclosure provides a split chimeric antigen receptor (CAR) system suitable for clinical application including a drug-inducible heterodimer, where the split CAR system includes a first polypeptide and a second polypeptide, where: the first polypeptide includes an extracellular antigen-binding domain, a transmembrane domain (TMD), a co-stimulatory domain and a first domain of a drug-inducible heterodimer; and the second polypeptide includes a second domain of the drug-inducible heterodimer and a signaling domain, where the first and second domains of the drug-inducible heterodimer bind one another in the presence of the drug.

In one embodiment, the drug-inducible heterodimer is an IMiD-inducible CRBN/CRBN polypeptide substrate heterodimer.

In another embodiment, the heterodimer is constitutively paired. In a related embodiment, the heterodimer is capable of being destabilized with the addition of a small molecule, optionally where the drug-destabilized heterodimer is an IMiD-destabilized CRBN/CRBN polypeptide substrate heterodimer such as CRBN/MEIS2, a MDM2/P53 polypeptide heterodimer inhibited by RG7112, a VHL/HIF-1α or VHL/HIF-2α polypeptide heterodimer inhibited by VH298, or a cIAP/SMAC heterodimer inhibited by birinapant.

In certain embodiments, the IMiD-inducible CRBN/CRBN polypeptide substrate heterodimer includes a CRBN polypeptide disrupted for or lacking a DDB1-interacting domain, optionally where the CRBN polypeptide is selected from SEQ ID NOs: 1-4.

In one embodiment, the IMiD-inducible CRBN/CRBN polypeptide substrate heterodimer includes a CRBN polypeptide disrupted for or lacking a DDB1-interacting domain further including a residue substitution at one or more of positions 371 and 388, optionally where the CRBN polypeptide disrupted for or lacking a DDB1-interacting domain includes a residue substitution selected from I371A, I371G, V388A and V388G.

In another embodiment, the IMiD-inducible CRBN/CRBN polypeptide substrate heterodimer includes a CRBN polypeptide substrate selected from IKZF3, IKZF1, ZFP91, GSPT1, GSS, or a fragment thereof that is capable of drug-inducible binding to CRBN polypeptide, optionally where the CRBN polypeptide substrate is SEQ ID NO: 5.

In certain embodiments, the IMiD-inducible CRBN/CRBN polypeptide substrate heterodimer includes a CRBN polypeptide substrate including a substituted form of IKZF3 aa130-189 that includes K→R residue substitutions at positions 137, 158, 166, 172 and 175 of the IKZF3 aa130-189 polypeptide sequence.

In another embodiment, the IMiD-inducible CRBN/CRBN polypeptide substrate heterodimer includes a CRBN polypeptide substrate including a substituted form of IKZF3 aa130-189 that includes a residue substitution at position 153, optionally where the residue substitution at position 153 is selected from A153I, A153M, A153T, A153N, A153Q, A153R, A153H, A153K, A153D, A153E and A153C.

Another aspect of the disclosure provides a mammalian cell including a split CAR system of the instant disclosure.

A further aspect of the disclosure provides a CRBN polypeptide disrupted for or lacking a DDB1-interacting domain and including one or more domains selected from a CRBN thalidomide binding domain (TBD), a CRBN LLP1-C domain, a CRBN LLP1-N domain and a CRBN N-terminal domain, where the CRBN polypeptide is not SEQ ID NO: 1 or SEQ ID NO: 4.

Another aspect of the disclosure provides a polypeptide including SEQ ID NO: 3.

An additional aspect of the disclosure provides polypeptide including SEQ ID NO: 2 in the absence of any other CRBN sequence.

An additional aspect of the disclosure provides a drug-responsive polypeptide that includes an inhibitor of CAR signaling and a CRBN polypeptide substrate domain capable of binding CRBN in response to drug, and which thereby promotes ubiquitin pathway-mediated degradation of the drug-responsive polypeptide, thereby activating CAR signaling.

In one embodiment, the inhibitor of CAR signaling is a proximal, pan-CAR/TCR signal transduction inhibitor, optionally the inhibitor of CAR signaling is Carboxy-terminal Src Kinase (CSK).

In another embodiment, the inhibitor of CAR signaling selectively abrogates a CAR signal transduction pathway and/or a CAR effector function, optionally the inhibitor of CAR signaling selectively abrogates a pathway or function that is Ras signaling, PKC, calcium-dependent signaling, NF-kappaB, NFAT, actin and cytoskeletal responses, cytokine secretion, cell proliferation, degranulation, and/or tumor cell killing, differentiation, or exhaustion.

In some embodiments, the inhibitor of CAR signaling is a ubiquitin ligase involved in TCR/CAR signal transduction. Optionally, the inhibitor of CAR signaling is c-CBL, CBL-B, ITCH, RNF125, RNF128 or WWP2.

In other embodiments, the inhibitor of CAR signaling is a TCR/CAR negative regulatory enzyme. Optionally, the inhibitor of CAR signaling is SHP1, SHP2, SHIP1, SHIP2, CD45, CSK, CD148, PTPN22, DGKalpha, DGKzeta, DRAK2, HPK1, HPK1, STS1, STS2 or SLAT.

In certain embodiments, the inhibitor of CAR signaling is a TCR/CAR negative regulatory scaffold/adapter protein. Optionally, the inhibitor of CAR signaling is PAG, LIME, NTAL, LAX31, SIT, GAB2, GRAP, ALX, SLAP, SLAP2, DOK1 or DOK2.

In another embodiment, the inhibitor of CAR signaling is a dominant negative version of an activating TCR signaling component. Optionally, the inhibitor of CAR signaling is ZAP70, LCK, FYN, NCK, VAV1, SLP76, ITK, ADAP, GADS, PLCgamma1, LAT, p85, SOS, GRB2, NFAT, p50, p65, AP1, RAP1, CRKII, C3G, WAVE2, ARP2/3, ABL, ADAP, RIAM or SKAP55.

In other embodiments, the inhibitor of CAR signaling includes the cytoplasmic tail of a TCR/CAR negative co-regulatory receptor. Optionally, the inhibitor of CAR signaling includes the cytoplasmic tail of a CD5, PD1, CTLA4, BTLA, LAG3, B7-H1, B7-1, CD160, TIM3, 2B4 or TIGIT TCR/CAR negative co-regulatory receptor.

In certain embodiments, the inhibitor of CAR signaling is targeted to the plasma membrane. Optionally, the inhibitor of CAR signaling possesses a targeting sequence derived from LAT, PAG, LCK, FYN, LAX, CD2, CD3, CD4, CD5, CD7, CD8a, PD1, SRC, or LYN.

In one embodiment, the drug-responsive polypeptide is cytosolic.

In another embodiment, the drug-responsive polypeptide possesses a membrane tether and/or transmembrane domain.

Another aspect of the disclosure provides a method for treating a subject with a chimeric antigen receptor (CAR) cellular therapy, the method involving administering to the subject a mammalian cell harboring a CAR and a drug-responsive polypeptide that includes an inhibitor of CAR signaling and a CRBN polypeptide substrate domain capable of binding CRBN in response to drug, the CRBN polypeptide substrate domain thereby promoting ubiquitin pathway-mediated degradation of the drug-responsive polypeptide, which activates CAR signaling (as the inhibitory element is released/degraded).

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Unless otherwise clear from context, all numerical values provided herein are modified by the term “about.” “Activation”, as used herein, refers to the state of a T-cell that has been sufficiently stimulated to induce detectable cellular proliferation. Activation can also be associated with induced cytokine production, and detectable effector functions. The term “activated T-cells” refers to, among other things, T-cells that are undergoing cell division.

The term “administration” refers to introducing a substance into a subject. In general, any route of administration may be utilized including, for example, parenteral (e.g., intravenous), oral, topical, subcutaneous, peritoneal, intra-arterial, inhalation, vaginal, rectal, nasal, introduction into the cerebrospinal fluid, or instillation into body compartments. In some embodiments, administration is oral. Additionally or alternatively, in some embodiments, administration is parenteral. In some embodiments, administration is intravenous.

By “agent” is meant any small compound (e.g., small molecule), antibody, nucleic acid molecule, or polypeptide, or fragments thereof.

The term “antigen” or “Ag” as used herein is defined as a molecule that can be targeted by an antibody or antibody fragment thereof.

As used herein, a “tumor antigen” means a biological molecule having antigenicity, expression of which is associated with a neoplastic cell. The tumor antigens targeted in the present disclosure include a tumor specific antigen (an antigen which is present only in tumor cells and is not found in other normal cells), and a tumor-associated antigen (an antigen which is also present in other organs and tissues or heterogeneous and allogeneic normal cells, or an antigen which is expressed on the way of development and differentiation).

The terms “antibodies” and “immunoglobulin” include antibodies or immunoglobulins of any isotype, fragments of antibodies that retain specific binding to antigen, including, but not limited to, Fab, Fv, scFv, and Fd fragments, chimeric antibodies, humanized antibodies, single-chain antibodies (scAb), single domain antibodies (dAb), single domain heavy chain antibodies, a single domain light chain antibodies, bi-specific antibodies, multi-specific antibodies, and fusion proteins comprising an antigen-binding (also referred to herein as antigen binding) portion of an antibody and a non-antibody protein. The antibodies can be detectably labeled, e.g., with a radioisotope, an enzyme that generates a detectable product, a fluorescent protein, and the like. The antibodies can be further conjugated to other moieties, such as members of specific binding pairs, e.g., biotin (member of biotin-avidin specific binding pair), and the like. The antibodies can also be bound to a solid support, including, but not limited to, polystyrene plates or beads, and the like. Also encompassed by the term are Fab′, Fv, F(ab′)₂, and or other antibody fragments that retain specific binding to antigen, and monoclonal antibodies. As used herein, a monoclonal antibody is an antibody produced by a group of identical cells, all of which were produced from a single cell by repetitive cellular replication. That is, the clone of cells only produces a single antibody species. While a monoclonal antibody can be produced using hybridoma production technology, other production methods known to those skilled in the art can also be used (e.g., antibodies derived from antibody phage display libraries). An antibody can be monovalent or bivalent. An antibody can be an Ig monomer, which is a “Y-shaped” molecule that consists of four polypeptide chains: two heavy chains and two light chains connected by disulfide bonds.

The term “humanized immunoglobulin” as used herein refers to an immunoglobulin comprising portions of immunoglobulins of different origin, wherein at least one portion comprises amino acid sequences of human origin. For example, the humanized antibody can comprise portions derived from an immunoglobulin of nonhuman origin with the requisite specificity, such as a mouse, and from immunoglobulin sequences of human origin (e.g., chimeric immunoglobulin), joined together chemically by conventional techniques (e.g., synthetic) or prepared as a contiguous polypeptide using genetic engineering techniques (e.g., DNA encoding the protein portions of the chimeric antibody can be expressed to produce a contiguous polypeptide chain). Another example of a humanized immunoglobulin is an immunoglobulin containing one or more immunoglobulin chains comprising a CDR derived from an antibody of nonhuman origin and a framework region derived from a light and/or heavy chain of human origin (e.g., CDR-grafted antibodies with or without framework changes).

Chimeric or CDR-grafted single chain antibodies are also encompassed by the term humanized immunoglobulin. See, e.g., Cabilly et al., U.S. Pat. No. 4,816,567; Cabilly et al., European Patent No. 0,125,023 B 1; Boss et al., U.S. Pat. No. 4,816,397; Boss et al., European Patent No. 0,120,694 B 1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Padlan, E. A. et al., European Patent Application No. 0,519,596 A1. See also, Ladner et al., U.S. Pat. No. 4,946,778; Huston, U.S. Pat. No. 5,476,786; and Bird, R. E. et al., Science, 242: 423-426 (1988)), regarding single chain antibodies.

The term “nanobody” (Nb), as used herein, refers to the smallest antigen binding fragment or single variable domain (VHH) derived from naturally occurring heavy chain antibody and is known to the person skilled in the art. They are derived from heavy chain only antibodies, seen in camelids (Hamers-Casterman et al., 1993; Desmyter et al., 1996). In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a VHH antibody.

“Antibody fragments” comprise a portion of an intact antibody, for example, the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 (1995)); domain antibodies (dAb; Holt et al. (2003) Trends Biotechnol. 21:484); single-chain antibody molecules; and multi-specific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

“Fv” is the minimum antibody fragment that contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CHj) of the heavy chain. Fab fragments differ from Fab′ fragments by the addition of a few residues at the carboxyl terminus of the heavy chain CHj domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Single-chain Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

As used herein, the term “autologous” is meant to refer to any material derived from the same individual to which it is later to be re-introduced into the individual.

As used herein “endogenous” refers to any material from or produced inside an organism, cell, tissue or system.

As used herein, the term “exogenous” refers to any material introduced from or produced outside an organism, cell, tissue, or system.

A “co-stimulatory molecule” refers to the cognate binding partner on a T-cell that specifically binds with a co-stimulatory ligand, thereby mediating a co-stimulatory response by the T-cell, such as, but not limited to, proliferation. Co-stimulatory molecules include, but are not limited to an MHC class I molecule, BTLA and a Toll ligand receptor.

A “co-stimulatory signal”, as used herein, refers to a signal, which in combination with a primary signal, such as TCR/CD3 ligation, leads to T-cell proliferation, activation, and/or upregulation or downregulation of key molecules.

By “control” or “reference” is meant a standard of comparison. In one aspect, as used herein, “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. Determination of statistical significance is within the ability of those skilled in the art, e.g., the number of standard deviations from the mean that constitute a positive result.

The term “cancer” refers to a malignant neoplasm (Stedman's Medical Dictionary, 25th ed.; Hensyl ed.; Williams & Wilkins: Philadelphia, 1990). Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. The term “CAR” is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled. CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. 5c/Transl Med (2013); 5(215):215ra172; Glienke et al. Front Pharmacol (2015) 6:21; Kakarla & Gottschalk 52 Cancer J (2014) 20(2): 151-5; Riddell et al. Cancer J (2014) 20(2): 141-4; Pegram et al. Cancer J (2014) 20(2): 127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety.

The terms “domain” and “motif, used interchangeably herein, refer to both structured domains having one or more particular functions and unstructured segments of a polypeptide that, although unstructured, retain one or more particular functions. For example, a structured domain may encompass but is not limited to a continuous or discontinuous plurality of amino acids, or portions thereof, in a folded polypeptide that comprise a three-dimensional structure which contributes to a particular function of the polypeptide. In other instances, a domain may include an unstructured segment of a polypeptide comprising a plurality of two or more amino acids, or portions thereof, that maintains a particular function of the polypeptide unfolded or disordered. Also encompassed within this definition are domains that may be disordered or unstructured but become structured or ordered upon association with a target or binding partner. Non-limiting examples of intrinsically unstructured domains and domains of intrinsically unstructured proteins are described, e.g., in Dyson & Wright. Nature Reviews Molecular Cell Biology 6: 191-208.

By “fragment” is meant a portion, e.g., a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. For example, a fragment may contain 10, 20, 30, 40, 50, 60, 70, 80, 90, or 100, 200, 300, 400, 500, 600, 700, 800, 900, or 1000 nucleotides or amino acids. However, the disclosure also comprises polypeptides and nucleic acid fragments, so long as they exhibit the desired/indicated biological activity/activities of the full length polypeptides and nucleic acid, respectively. A nucleic acid fragment of almost any length is employed. For example, illustrative polynucleotide segments with total lengths of about 10,000, about 5000, about 3000, about 2,000, about 1,000, about 500, about 200, about 100, about 50 base pairs in length (including all intermediate lengths) are included in many implementations of this disclosure. Similarly, a polypeptide fragment of almost any length is employed. For example, illustrative polypeptide segments with total lengths of about 10,000, about 5,000, about 3,000, about 2,000, about 1,000, about 5,000, about 1,000, about 500, about 200, about 100, or about 50 amino acids in length (including all intermediate lengths) are included in many implementations of this disclosure.

“Heterologous,” as used herein, means a nucleotide or polypeptide sequence that is not found in the native (e.g., naturally-occurring) nucleic acid or protein, respectively.

As used herein, the term “immune cells” generally includes white blood cells (leukocytes) which are derived from hematopoietic stem cells (HSC) produced in the bone marrow. “Immune cells” includes, e.g., lymphocytes (T cells, B cells, natural killer (NK) cells) and myeloid-derived cells (neutrophil, eosinophil, basophil, monocyte, macrophage, dendritic cells).

“T cell” includes all types of immune cells expressing CD3 including T-helper cells (CD4⁺ cells), cytotoxic T-cells (CD8⁺ cells), T-regulatory cells (Treg) and gamma-delta T cells.

A “cytotoxic cell” includes CD8⁺ T cells, natural-killer (NK) cells, and neutrophils, which cells are capable of mediating cytotoxicity responses.

As used herein, the term “stem cell” generally includes pluripotent or multipotent stem cells. “Stem cells” includes, e.g., embryonic stem cells (ES); mesenchymal stem cells (MSC); induced-pluripotent stem cells (iPS); and committed progenitor cells (hematopoietic stem cells (HSC); bone marrow derived cells, neural progenitor cells, etc.).

As used herein, the term “heteromeric” refers to a polypeptide or protein that contains more than one kind of subunit. Such heteromeric polypeptides may, in some instances, be referred to as “a heteromer”. Heteromeric polypeptides may contain two or more different polypeptides, wherein different polypeptides are defined at least as two polypeptides that are not identical, however, such different polypeptides may or may not include one or more portions of similar and/or identical amino acid sequence. In some instances, the two or more polypeptides of a heteromer share no identical amino acid sequence or share no identical domains. A heteromer may, in some instances, consist of two different polypeptides or two different types of polypeptides and may be referred to as a heterodimer. In some instances, a heteromer may consist of three different polypeptides or three different types of polypeptides and may be referred to as a heterotrimer. In some instances, a heteromer may consist of two or more different polypeptides or two or more different types of polypeptides, including but not limited to, e.g., three or more different polypeptides, four or more different polypeptides, five or more different polypeptides, six or more different polypeptides, seven or more different polypeptides, eight or more different polypeptides, etc.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation.

By “marker” is meant any protein or polynucleotide having an alteration in expression level or activity that is associated with a disease or disorder.

As used herein, “neoplasia” means a disease state of a human or an animal in which there are cells and/or tissues which proliferate abnormally. Neoplastic conditions include, but are not limited to, cancers, sarcomas, tumors, leukemias, lymphomas, and the like. A neoplastic condition refers to the disease state associated with the neoplasia. Hepatocellular carcinoma, colon cancer (e.g., colorectal cancer), lung cancer and ovarian cancer are examples (non-limiting) of a neoplastic condition. A “cancer” in a subject refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within a subject, or may be a non-tumorigenic cancer cell, such as a leukemia cell. Examples of cancer include but are not limited to hepatic carcinoma, colon cancer, colorectal cancer, breast cancer, a melanoma, adrenal gland cancer, biliary tract cancer, bladder cancer, brain or central nervous system cancer, bronchus cancer, blastoma, carcinoma, a chondrosarcoma, cancer of the oral cavity or pharynx, cervical cancer, esophageal cancer, gastrointestinal cancer, glioblastoma, hepatoma, kidney cancer, leukemia, liver cancer, lung cancer, lymphoma, non-small cell lung cancer, osteosarcoma, ovarian cancer, pancreas cancer, peripheral nervous system cancer, prostate cancer, sarcoma, salivary gland cancer, small bowel or appendix cancer, small-cell lung cancer, squamous cell cancer, stomach cancer, testis cancer, thyroid cancer, urinary bladder cancer, uterine or endometrial cancer, and vulval cancer.

“Operably linked” refers to a juxtaposition wherein the components so described are in a relationship permitting them to function in their intended manner. For instance, a promoter is operably linked to a coding sequence if the promoter affects its transcription or expression.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

An “isolated” polypeptide is one that has been identified and separated and/or recovered from a component of its natural environment. Contaminant components of its natural environment are materials that would interfere with diagnostic or therapeutic uses for the polypeptide, and may include enzymes, hormones, and other proteinaceous or nonproteinaceous solutes. In some embodiments, the polypeptide will be purified (1) to greater than 90%, greater than 95%, or greater than 98%, by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or nonreducing conditions using Coomassie blue or silver stain. Isolated polypeptide includes the polypeptide in situ within recombinant cells since at least one component of the polypeptide's natural environment will not be present. In some instances, isolated polypeptide will be prepared by at least one purification step.

As used herein, the term “subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

As used herein, the term “tumor” means a mass of transformed cells that are characterized by neoplastic uncontrolled cell multiplication and at least in part, by containing angiogenic vasculature. The abnormal neoplastic cell growth is rapid and continues even after the stimuli that initiated the new growth has ceased. The term “tumor” is used broadly to include the tumor parenchymal cells as well as the supporting stroma, including the angiogenic blood vessels that infiltrate the tumor parenchymal cell mass. Although a tumor generally is a malignant tumor, i.e., a cancer having the ability to metastasize (i.e., a metastatic tumor), a tumor also can be nonmalignant (i.e., non-metastatic tumor). Tumors are hallmarks of cancer, a neoplastic disease the natural course of which is fatal. Cancer cells exhibit the properties of invasion and metastasis and are highly anaplastic.

As used herein, the terms “treatment,” “treating,” “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease or condition in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

A “vector” or “expression vector” is a replicon, such as plasmid, phage, virus, or cosmid, to which another DNA segment, i.e. an “insert”, may be attached so as to bring about the replication of the attached segment in a cell.

Unless specifically stated or obvious from context, as used herein, the term “or” is understood to be inclusive. Unless specifically stated or obvious from context, as used herein, the terms “a”, “an”, and “the” are understood to be singular or plural.

The phrase “pharmaceutically acceptable carrier” is art recognized and includes a pharmaceutically acceptable material, composition or vehicle, suitable for administering compounds of the present disclosure to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

A “therapeutically effective amount” of an agent described herein is an amount sufficient to provide a therapeutic benefit in the treatment of a condition or to delay or minimize one or more symptoms associated with the condition. A therapeutically effective amount of an agent means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

The following detailed description, given by way of example, but not intended to limit the disclosure solely to the specific embodiments described, may best be understood in conjunction with the accompanying drawings, in which:

FIG. 1 shows a structural schematic that displays the binding of an IMiD glutarimide ring to the tri-TRP binding pocket of CRBN, further shows a schematic of a split-receptor design, and additionally documents the design and demonstrated efficacy of certain IMiD-responsive ON switches. Particularly shown is an IMiD-responsive (here, pomalidomide) ON-switch that brings together split receptor elements upon IMiD-induced binding between IKZF3 degron and minimal CRBN polypeptide. Specifically, CRBN deletion variants were engineered to retain binding to Pom/IKZF3 but not DDB1/CUL4. Four deletion variants were generated in CRBN that removed the DDB1-binding domain, as shown in the middle image (NTD is the N-terminal domain; LLP1 is Lon-like protease domain 1 (split into LLP1-N and LLP1-C regions); TBD is the Thalidomide binding domain). At middle, BRET assays were performed upon 293T cells harboring bialleleic CRISPR/Cas9 disruption of endogenous CRBN (293T-CRBNΔe5), to assess dimerization between IKZF3aa130-189 and CRBN or minCRBN variants (dose-response data shown are CRBN, minCRBN1, minCRBN2, minCRBN3 and minCRBN4). Effective concentrations for dimerization were identified in various cellular assays—apparent Kd values for CRBN and minCRBN3 were 30 nM, whereas Kd values were not determined for minCRBN1 and minCRBN2. Pomalidomide-dependent dimerization was observed between IKZF3aa130-189-HaloTag and CRBN-Nanoluciferase (NLuc) or minCRBN2-4-NLuc. The bottom plot shows the results of co-transfection of minCRBN1-4 and IKZF3 degron-GFP-IRES-mCherry expression vectors in 293T-CRBNΔe5 cells. 1 day after transfection, cells were exposed to 1 μM pomalidomide for the number of hours indicated and GFP and mCherry fluorescence was measured by flow cytometry (eGFPmCherry levels were detected as 0.5 or less for the CRBN trace). (Kd values for various split receptor designs were further determined to be around 100 nM, with a much higher saturation value; using different transformation conditions, Kd eff for iK0 split receptor was observed to be 50 nM, while Kd eff for a K+ split receptor was observed to be 67 nM.)

FIG. 2 shows that cell surface localization in a split receptor configuration augmented heterodimerization of IKZF3 degron and minCRBN (minCRBN3, aka CRBNΔ3). Dimerization of indicated pairs of molecules (CD19scFv-CD28-IKZF3 respectively paired with CD8ht-CD28c-CRBNΔ3, PD1htc-CRBNΔ3, myrCD28c-CRBNΔ3 or CRBNΔ3; or degron paired with CRBNΔ3) was assessed by BRET in 293T cells with 10 μM MG132 (a proteasome inhibitor) after 2 hours of drug treatment.

FIG. 3 shows that an IKZF3 degron engineered to remove ubiquitination sites (“iK0”) produced enhanced IKZF3 degron-minCRBN3 split receptor dimerization, in the presence of endogenous CRBN. Specifically, intracellular lysine-to-arginine substitutions enhanced the duration and amplitude of IKZF3 degron—minCRBN3 split receptor dimerization in the presence of endogenous CRBN. From the position of the lysines in the IKZF3 zinc finger degron in an IKZF3 ZF2-CRBN crystal structure (not shown), it was hypothesized that substituting arginines for all lysines would not disrupt protein-protein binding between the IKZF3 degron and CRBN. Because the CD28 intracellular domain contains three signaling adapter motifs that do not contain conserved lysine residues, it was further hypothesized that it would be possible to substitute arginines for both lysines in the CD28 intracellular tail without altering the functional properties of this domain. With these seven lysine-to-arginine substitutions, a split receptor was generated that contained no lysine residues in the intracellular compartment (iK0). It was further hypothesized that extracellular lysines would not need to be modified to avoid ubiquitination by CRL4CRBN, because these residues would be separated from the ubiquitin ligase by the plasma membrane. At left, dimerization was assessed in 293T cells after varying hours of exposure to 1 μM pomalidomide. At right, dimerization between the specified anti-CD19-CD28-IKZF3 protein and myrCD28-minCRBN3 (CRBNΔ3) was assessed in 293T cells 2 hours after addition of various concentrations of pomalidomide and 10 mM MG132. Wild type (wt) and iK0 Kd (SE) values were 53.4 (6.7) and 46.3 (6.3), respectively. Bmax 17.5 (0.5) and 22.5 (0.7).

FIG. 4 shows that strategic residue substitutions, made respectively within the IMiD (pomalidomide) binding pocket of the IKZF3 degron and the CRBN polypeptide could be used (a) to make the IKZF3 degron less responsive/effectively non-responsive to pomalidomide-dependent degradation by native forms of CRBN polypeptide, while (b) “offsetting” substituted forms of CRBN polypeptide could also be identified that restored pomalidomide-dependent association to the substituted IKZF3 degron-substituted CRBN interaction (thereby effectively freeing this substituted IKZF3 degron-substituted CRBN dimerization from the potential degradative impacts of wild-type CRBN, where present, thus reducing or eliminating the need for genomic disruption of native CRBN in candidate therapeutic cells that employ such a substituted IKZF3 degron-substituted CRBN dimer system). In particular, the top plot demonstrates that substitutions at IKZF3 residue A153 increased the observed EC50 of pomalidomide-dependent degradation. Notably, the IKZF3 A153M substitution disrupted binding and degradation by CRBN (among the various substituted forms tested—in the topplot, wild-type is the left-hand trace, while various substituted forms as indicated provide the traces to the right of the wild-type trace). As shown in the bottom row of plots, a CRBN V388A substitution restored binding affinity with IKZF3 A153M, thereby generating a preferential knob-in-hole binding pair (see structure schematic at left). The bottom row of charts shows that in 293T cells exposed for 2 hours to 10 μM MG132 and either 1 μM Pomalidomide or DMSO, the mBU fold change at 1 μM Pomalidomide/DMSO treatment was calculated for IKZF3 A153 substitutions and CRBN, CRBN V388A, or CRBN I371A. mBU fold change was plotted for mutant versus wild type CRBN. The CRBN V388A mutation increased the mBU fold change versus wild type CRBN (FIG. 4 at bottom right).

FIG. 5 demonstrates that the “ON”-switch was further improved using a “d913” degron for dimerization. Specifically, an engineered hybrid degron, d913, enabled more robust control than the endogenous IKZF3 degron for tumor antigen-dependent activation marker expression and TL2 release. As for the IKZF3 degron described above, a “K0” form of the “d913” degron was produced, and the chemically-induced dimerization system employing a “K0” d913 super-degron-derived dimerization domain (sCARAB 913) was identified to enable more robust drug-dependent activation than the corresponding IKZF3-CRBN dimerization system (sCARAB). To generate figure plots, T cells were transduced with lentivirus generated with the lentivectors BigSur and Eureka driving the expression of the indicated split CAR constructs (sCARAB, sCARAB 913 and a CAR control). 100,000 Jurkat cells were co-cultured with 20,000 K562 or K562-CD19 cells for 18 hours. Via flow cytometry analysis, cell surface split CAR expression (myc tag) and activation marker expression (CD69) were assessed in eGFP+/mCherry+ Jurkat cells transduced with both components of the split receptor. At left, observed cell surface CAR expression (as assessed by flow cytometry using anti-Myc-tag antibody) is shown. At right, activation (% CD69+) by flow cytometry of Jurkat CAR-T cells is shown. IL2 secretion from Jurkat CAR-T cells was also assessed (data not shown), with IL2 ELISA performed upon cell culture supernatant.

FIG. 6 shows primary T cell activation data obtained for the split CAR system. In particular, lenalidomide-dependent activation of the split CAR was successfully observed in primary T cells. In such experiments, co-culture of NALM6 B cells with primary human T cells transduced with BigSur_sCARA and Eureka_sCARB or BigSur_1928z and subsequently expanded in vitro. mCherry+(CAR) or mCherry+/eGFP+(split CAR) cells were sorted by FACS and subsequently co-cultured for 24 hours with NALM6-CBG-eGFP target cells. CD69 expressed was assessed by flow cytometry. UTD=untransduced. CAR=BigSur_19-28z. “split CAR”=cells transduced both with BigSur_sCARA and Eureka_sCARB, as demonstrated by co-expression of mCherry and eGFP.

FIG. 7 demonstrates the effect of IKZF3 degron inclusion within a GFP-tagged CD28 fusion protein. Specifically, a minimal IKZF3 degron polypeptide conferred IMiD-dependent internalization and degradation to a CAR-like transmembrane protein.

FIG. 8 shows that the IKZF3 degron (comprising amino acids 130-189 of IKZF3) functioned as a highly pomalidomide-responsive OFF-switch when integrated into the context of a CAR construct. In particular, CAR-like CRL4CRBN degron-tagged transmembrane proteins were confirmed as degraded with pomalidomide. The instant CD28-CD3ζ-degron protein differed from an anti-CD19 CAR sequence published previously (PMID 19561539) in two ways. First, the FMC63 anti-CD19 scFv was exchanged for the CD28 Ig-like V-type extracellular domain. Second, the protein was fused in-frame at its C-terminus with the degron IKZF3aa130-189, which was previously shown to mediate lenalidomide-dependent degradation by CRL4^{CRBN} (PMID 24292625). Jurkat T cells were transduced with lentivirus encoding the three degron-eGFP fusion proteins (pSFFV-insert-linker-eGFP-IRES-mCherry). mCherry expression served as an internal control for transgene expression. Degradation of the degron-GFP fusion protein at varying concentrations of pomalidomide was quantified as the eGFP/mCherry ratio normalized to the DMSO treatment control. Experiments were performed in triplicate. EC50 and standard error values for degron, CD28-degron, and CD28-CD3ζ-degron, respectively were 0.42+/−0.03, 23.5+/−2.7, and 8.2+/−0.9. Anti-BCMA-CD28-CD3ζ-degron CAR is also assessed for tumor killing, cytokine release, and cell surface co-receptor expression in primary human T cells, both in vitro and in vivo.

FIG. 9 shows a plasmid map for the “Artichoke” expression plasmid of the instant disclosure, where the “+1” sites mark the BsmBI restriction endonuclease cloning sites that were used to insert genes of interest.

FIG. 10 shows that a ZFP91/IKZF3 hybrid degron polypeptide was a degron polypeptide that was more sensitive than either the ZFP91 degron sequence alone or the IKZF3 degron sequence alone (sequences from which the hybrid degron derives).

FIG. 11 shows a plot demonstrating the dynamics of transmembrane protein internalization upon addition of pomalidomide for the OFF-switch CAR design.

FIG. 12 shows a plot demonstrating the dynamics of re-synthesis of transmembrane degron-tagged protein after washout of pomalidomide.

FIG. 13 depicts a degron-tagged CAR (dCAR) reversible drug-OFF switch design. At left, a CRBN substrate degron has been appended to the cytoplasmic C-terminus of a second generation CAR. At right, a schematic of the drug-OFF switch design is shown: dCAR-T cells are able to respond to antigen-positive tumor cells. With the addition of pomalidomide, lenalidomide, or another small molecule controller, the dCAR-T cell degrades the dCAR, thereby reducing or eliminating the capacity of these cells to respond to tumor cells. With cessation of the controller drug, the dCAR-T cell can reactivate as the protein concentration of the dCAR increases.

FIG. 14 shows results of experiments that demonstrated degron position, length, and linker sequences to impact CAR protein abundance and chemical control. In particular, a relationship was discovered between degron position, length, and linker for CAR expression and functional control. The left panel shows observed mean fluorescence intensity (MFI) of Jurkat CAR-T cells incubated with lenalidomide for 18 hours, with cells stained with anti-Myc-tag antibody that detected cell surface CAR expression. The right panel shows observed levels of activation (% CD69+) as assessed by flow cytometry of Jurkat T cells transduced with the indicated degron-tagged CAR constructs and incubated with K562-CD19 cells for 18 hours. To produce these data, Jurkat T cells were transduced with lentivirus generated with the lentivector BigSur driving the expression of the indicated CAR construct. mCherry+ cells were then analyzed for surface Myc-tag and CD69 expression. In the panels, 19=anti-CD19 scFv FMC63; 28z=CD28 hinge, transmembrane, and costimulatory domain and CD3zeta intracellular signaling domain; G4S2=polypeptide linker with the sequence GGGGSGGGGS (SEQ ID NO: 93); EAAAK3=polypeptide linker with the sequence AEAAAKEAAAKEAAAKA (SEQ ID NO: 94); dm913=seq. 32; d913=IKZF3 aa 130-145+ZFP91 aa 400-410+IKZF3 aa 157-189 (FNVLMVHKRSHTGERPLQCEICGFTCRQKGNLLRHIKLHTGEKPFKCHLCNYACQRRDA L; SEQ ID NO: 95).

FIG. 15 demonstrates that a degron tag (here, the d913 degron tag) reduced tonic cell surface CAR expression without altering the ability of CARs to express activation markers in response to target antigen presentation. The left panel shows observed expression of cell surface CAR (anti-Myc tag) on Jurkat T cells. To obtain these results, Jurkat T cells were transduced with lentivirus generated with the lentivector BigSur driving the expression of the indicated CAR construct. mCherry+ cells were analyzed for surface Myc-tag and CD69 expression. Addition of the degron (d913) resulted in lower tonic CAR cell surface protein abundance versus the control CAR. The right panel demonstrates the activation observed for Jurkat T cells transduced with the indicated CAR, or untransduced, after 18 hour co-culture with K562-CD19 cells. Although the degron-tagged CAR was expressed at lower protein abundance, activation marker expression was indistinguishable between it and the non-degron-tagged form.

FIG. 16 demonstrates that degron sequence determined the efficacy of the drug OFF-switch. In particular, the engineered hybrid degron (d913) was observed to enable more robust control over CAR protein expression, activation, and cytokine secretion than the endogenous IKZF3 degron for tumor antigen-dependent activation marker expression and IL2 release. To obtain the displayed results, T cells were transduced with lentivirus generated with the lentivector BigSur driving the expression of the indicated CAR construct. mCherry+ cells were purified by fluorescence-activated cell sorting. 100,000 CAR-T Jurkat cells were co-cultured with 20,000 K562 or K562-CD19 cells for 18 hours. The top left panel shows observed cell surface CAR expression (as assessed by flow cytometry using anti-Myc-tag antibody). The top right panel shows observed activation (% CD69+) by flow cytometry of Jurkat CAR-T cells. The bottom panel shows IL2 secretion results obtained from Jurkat CAR-T cells—IL2 ELISA was performed on cell culture supernatant for each of the indicated constructs.

FIG. 17 demonstrates degron-tagged construct-mediated control of U87-CD19 tumor cell killing in primary T cells. In particular, control of target cell killing (U87 human cell line engineered to express CD19) with lenalidomide was observed in primary human degron-tagged CAR-T cells. Displayed images are the result of co-culture of U87-CD19-eGFP cells with primary human T cells transduced with lentivirus generated with the lentivector BigSur driving the expression of the indicated CAR construct and subsequently expanded in vitro. Equal numbers of U87-CD19-eGFP cells and the indicated CAR-T cells were co-cultured with 1000 nM lenalidomide or DMSO control for 24 hours. Fluorescence live cell microscopy was then performed throughout the 24 hour timecourse, yielding the displayed images.

FIG. 18 shows plots that further demonstrate degron-tagged construct-mediated control of U87-CD19 tumor cell killing in primary T cells. In particular, control of target cell killing (U87 human cell line engineered to express CD19) with lenalidomide was observed in primary human degron-tagged CAR-T cells, as above. Plots were generated following co-culture of U87-CD19-eGFP cells with primary human T cells transduced with lentivirus generated with the lentivector BigSur driving the expression of the indicated CAR construct and subsequently expanded in vitro. Equal numbers of U87-CD19-eGFP cells and the indicated CAR-T cells were co-cultured with 1000 nM or 100 nM lenalidomide or DMSO control for 24 hours. Fluorescence live cell microscopy was performed throughout the 24 hour timecourse. GFP (left plot) or mCherry (right plot) fluorescence intensity was depicted for each condition, normalized to the fluorescence at T=0 hours for each well.

FIG. 19 demonstrates partial control of CAR-T cell killing of NALM6 target cells (human B-ALL cell line; ND33) in primary T cells, with lenalidomide in primary human degron-tagged CAR-T cells. The displayed graph shows observed levels of killing of NALM6-CBG-GFP cells after 18 hour co-culture at the specified ratios. To generate the displayed results, primary human T cells were transduced with lentivirus generated with the lentivector BigSur driving the expression of the indicated CAR construct and subsequently expanded in vitro. NALM6-CBG-GFP cells and CAR-T cells were mixed at the indicated ratios and incubated in culture for 18 hours with DMSO or 1000 nM lenalidomide. Experiments were performed in triplicate from a single normal donor (ND33). Killing from 1928z-d913 without lenalidomide approximated killing from 1928z (with or without lenalidomide). 1928z-d913 killing was blunted with 1000 nM but not 100 nM lenalidomide.

FIG. 20 further demonstrates partial control of CAR-T cell killing of NALM6 target cells (human B-ALL cell line; ND34) in primary T cells, with lenalidomide in primary human degron-tagged CAR-T cells. Plots show killing of NALM6-CBG-GFP cells after 18 hour co-culture at the specified ratios. Primary human T cells were transduced with lentivirus generated with the lentivector BigSur driving the expression of the indicated CAR construct and subsequently expanded in vitro. NALM6-CBG-GFP cells and CAR-T cells were mixed at the indicated ratios and incubated in culture for 18 hours with DMSO, 100 nM lenalidomide, or 1000 nM lenalidomide. Experiments were performed in triplicate from a single normal donor (ND34). As above, killing from 1928z-d913 without lenalidomide approximated killing from 1928z (with or without lenalidomide). Meanwhile 1928z-d913 killing was blunted with 1000 nM but not 100 nM lenalidomide.

FIG. 21 exemplifies a split CAR reversible drug-ON switch design. Top left: components A (antigen-binding, co-stimulatory domain, and IKZF3-derived dimerization domain) and B (transmembrane domain, co-stimulatory domain, CD3z intracellular domain, and CRBN-derived dimerization domain) are split and inactive without drug. Bottom left: upon addition of the controller drug, e.g. lenalidomide or pomalidomide, the split components dimerize and are licensed to activate in the presence of the target antigen. Top right: the split CAR is designed to be inactive in the absence of drug. Bottom right: upon addition of the controller drug, the CAR-T cell can activate when interacting with a cell expressing the target antigen. In Multiple Myeloma, Non-Hodgkin's Lymphoma, and other indications, synergy occurs between the on-target effect of the controller drug on the tumor cells, activation of the split CAR, and derepression of IL2 via degradation of IKZF1/3 in the CAR-T cells.

FIG. 22 demonstrates the increased protein concentration with increasing drug concentration of the intracellular K0 versus the unmodified variant of the split CAR component A. Split CAR component A variants, either (A) FMC63-CD28-IKZF3 iK0 or (A[K+]) FMC63-CD28-IKZF3, were delivered via the lentiviral expression vector Jenner and bear a N-terminal myc tag and a C-terminal 2A sequence followed by the coding sequence for mCherry. Component B1 (CD8-CD28-mCRBN3-IKZF3) of the split CAR were delivered via the lentiviral expression vector Eureka and bear a C-terminal eGFP tag. Cells were analyzed by flow cytometry after co-culture for 24 hours with K562 or K562-CD19 target cells and the indicated concentration of lenalidomide, pomalidomide, or DMSO control.

FIG. 23 demonstrates the increased drug-dependent T cell activation, as assessed by CD69 marker expression, of the intracellular K0 versus the unmodified variant of the split CAR component A. Split CAR component A variants, either (A) FMC63-CD28-IKZF3 iK0 or (A[K+]) FMC63-CD28-IKZF3, were delivered via the lentiviral expression vector Jenner. Component B1 (CD8-CD28-mCRBN3-IKZF3) of the split CAR were delivered via the lentiviral expression vector Eureka. Cells were analyzed by flow cytometry after co-culture for 24 hours with K562 or K562-CD19 target cells and the indicated concentration of lenalidomide, pomalidomide, or DMSO control.

FIG. 24 demonstrates drug- and antigen-dependent activation, as assessed by CD69 marker expression, for Jurkat cells expressing the split CAR A/B1 versus the split CAR A/B1a and a control CAR (FMC63-CD28-CD3z). Cells were analyzed by flow cytometry after co-culture for 24 hours with K562 or K562-CD19 target cells and the indicated concentration of lenalidomide, pomalidomide, or DMSO control. Split CAR A=FMC63-CD28-IKZF3 iK0. Split CAR B1=CD8-CD28-mCRBN3-CD3z. Split CAR B1a=CD8-CD28-mCRBN2-CD3z.

FIG. 25 shows the requirement of antigen, drug, and both split CAR components A and B1 for Jurkat T cell activation, as assessed by CD69 marker expression. Jurkat cells expressing single split CAR components A (FMC63-CD28-IKZF3 iK0) or B1 (CD8-CD28-mCRBN3-IKZF3), both split CAR components A and B1, a control CAR (FMC63-CD28-CD3z), or untransduced cells were analyzed. Cells were analyzed by flow cytometry after co-culture for 24 hours with K562 or K562-CD19 target cells and the indicated concentration of lenalidomide, pomalidomide, or DMSO control.

FIG. 26 shows the requirement of antigen, drug, and both split CAR components A and B1 for Jurkat cell IL2 secretion, as assessed by IL2 ELISA. Jurkat cells expressing single split CAR components A (FMC63-CD28-IKZF3 iK0) or B1 (CD8-CD28-mCRBN3-IKZF3), both split CAR components A and B1, a control CAR (FMC63-CD28-CD3z), or untransduced cells were analyzed. Cell culture supernatant was collected after 24 hours of co-culture with K562 or K562-CD19 target cells and the indicated concentration of lenalidomide, pomalidomide, or DMSO control.

FIG. 27 shows that the expression of eGFP-tagged split CAR component B1 (CD8-CD28-mCRBN3-IKZF3) was stable across concentrations of pomalidomide and with either K562 or K562-CD19 target cells. Cells were analyzed by flow cytometry after co-culture for 24 hours with K562 or K562-CD19 target cells and the indicated concentration of lenalidomide, pomalidomide, or DMSO control.

FIG. 28 demonstrates the drug-ON switch design combining a CAR and a Chimeric Degradable Inhibitor (CDI). Top left: The CDI tonically inhibits some or all effects of CAR signal transduction. Bottom left: with addition of the controller drug, degradation of the CDI results in unopposed CAR signaling, allowing for full activation. Top right: the CAR+CDI system is designed to be inactive in the absence of drug. Bottom right: upon addition of the controller drug, the CAR+CDI system is unmasked and licensed for activation. In Multiple Myeloma, Non-Hodgkin's Lymphoma, and other indications, synergy between the on-target effect of the controller drug on the tumor cells, de-repression of the CAR, and de-repression of IL2 via degradation of IKZF1/3 in the CAR-T cells is envisioned.

FIG. 29 demonstrates that various CDIs are able to inhibit antigen-dependent Jurkat T cell activation, as assessed by CD69 marker expression, with de-repression of antigen-independent activation with 1 uM pomalidomide. Jurkat cells were transduced with Bolinas lentiviral expression vector encoding a CAR (FMC63-CD28-CD3z) and also transduced with Eureka lentiviral expression vector encoding a CDI targeted to the plasma membrane with a N-terminal LAT transmembrane anchor and C-terminal tagged with the IKZF3 degron. The CDI constructs tested here are CSK, CSK E154A, CSK W47A/R107K/E154A and SHP1 (amino acids 203-595). Cells were analyzed by flow cytometry after co-culture for 24 hours with K562 or K562-CD19 target cells and 1 uM pomalidomide or DMSO control.

FIG. 30 demonstrates that various CDIs are able to inhibit antigen-dependent Jurkat T cell activation, as assessed by CD69 marker expression, and lenalidomide or pomalidomide results in full activation in the presence of antigen and partial antigen-independent activation. Jurkat cells were transduced with Bolinas lentiviral expression vector encoding a CAR (FMC63-CD28-CD3z) and also transduced with Eureka lentiviral expression vector encoding a CDI targeted to the plasma membrane with a N-terminal LAT transmembrane anchor and C-terminal tagged with the IKZF3 degron. The CDI constructs tested here are CSK and CSK E154A. Cells were analyzed by flow cytometry after co-culture for 24 hours with K562 or K562-CD19 target cells and the indicated concentration of lenalidomide, pomalidomide, or DMSO control.

FIG. 31 demonstrates that various CDIs are able to inhibit Jurkat IL2 secretion, and lenalidomide or pomalidomide licenses antigen-dependent activation. Jurkat cells were transduced with Bolinas lentiviral expression vector encoding a CAR (FMC63-CD28-CD3z) and also transduced with Eureka lentiviral expression vector encoding a Chimeric Degradable Inhibitor (CDI) targeted to the plasma membrane with a N-terminal LAT transmembrane anchor and C-terminal tagged with the IKZF3 degron. The CDI constructs tested here are CSK and CSK E154A. Cell culture supernatant was collected after co-culture for 24 hours with K562 or K562-CD19 target cells and the indicated concentration of lenalidomide, pomalidomide, or DMSO control.

FIG. 32 shows a plasmid map for the “Elk” expression plasmid of the instant disclosure, where the BsmBI restriction endonuclease cloning sites were used to insert genes of interest.

FIG. 33 shows a plasmid map for the “Jenner” expression plasmid of the instant disclosure, where the BsmBI restriction endonuclease cloning sites were used to insert genes of interest.

FIG. 34 shows a plasmid map for the “Bolinas” expression plasmid of the instant disclosure, where the BsmBI restriction endonuclease cloning sites were used to insert genes of interest.

FIG. 35 shows a plasmid map for the “Eureka” expression plasmid of the instant disclosure, where the BsmBI restriction endonuclease cloning sites were used to insert genes of interest.

FIG. 36 shows schematic diagrams of additional CAR degron constructs that were made in the current study, including 1928z-dIKZF3 aka dCAR IKZF3 and 1928z-d913 aka dCAR 913. In the schematics, CD28 H/TM/CO=hinge/transmembrane/costimulatory domain. 1928z-d913=DCAR 913. d913=degron derived from the fusion of ZFP91 and IKZF3 zinc finger degrons. FT2A=furin cleavage site+T2A.

### DETAILED DESCRIPTION OF THE INVENTION

The present disclosure is directed, at least in part, to discovery and engineering of switch systems that are responsive at the molecular level to small molecules approved for human use, which renders such engineered switch systems applicable for a variety of clinical uses (e.g., CAR T-cell use, precision control of gene therapies for other immune and stem cell therapies, etc.), in contrast to previously described molecular switch systems.

Negative reactions to cell-based immunotherapies can be fatal, as seen in certain CAR-T therapy clinical trials (“In staggering setback, toxic reaction kills Cellectis' first CAR-T patient, forcing trial halt,” Endpoints News, Sep. 5, 2017; see also Juno Therapeutics, in which an altered preconditioning regimen was used to enhance efficacy and persistence of CAR-T cell product, but five deaths attributable to cerebral edema occurred, as well as high rates of grade 3-4 cytokine release syndrome (71%) and grade 3 neurotoxicity (25%) occurred—the JCAR015 phase II Rocket clinical trial was therefore placed on clinical hold (Turtle et al. “Biomarkers of Cytokine Release Syndrome and Neurotoxicity after CD19 CAR-T Cells and Mitigation of Toxicity By Cell Dose” ASH Annual Meeting Abstract (2016) 1852)).

Cytokine release syndrome (CRS) is an inflammatory response clinically manifesting with fever, nausea, headache, tachycardia, hypotension, hypoxia, as well as cardiac and/or neurologic manifestations. Severe cytokine release syndrome is described as a cytokine storm, and can be fatal. CRS is believed to be a result of the sustained activation of a variety of cell types such as monocytes and macrophages, T-cells and B cells, and is generally characterized by an increase in levels of TNFα and IFNγ within 1 to 2 hours of stimulus exposure, followed by increases in interleukin (IL)-6 and IL-10 and, in some cases, IL-2 and IL-8 (Doessegger et al., “Clinical development methodology for infusion-related reactions with monoclonal antibodies.” Nat. Clin. Transl. Immuno. 4 (2015): e39).

Tumor lysis syndrome (TLS) is a metabolic syndrome that is caused by the sudden killing of tumor cells with chemotherapy, and subsequent release of cellular contents with the release of large amounts of potassium, phosphate, and nucleic acids into the systemic circulation. Catabolism of the nucleic acids to uric acid leads to hyperuricemia; the marked increase in uric acid excretion can result in the precipitation of uric acid in the renal tubules and renal vasoconstriction, impaired autoregulation, decreased renal flow, oxidation, and inflammation, resulting in acute kidney injury. Hyperphosphatemia with calcium phosphate deposition in the renal tubules can also cause acute kidney injury. High concentrations of both uric acid and phosphate potentiate the risk of acute kidney injury because uric acid precipitates more readily in the presence of calcium phosphate and vice versa that results in hyperkalemia, hyperphosphatemia, hypocalcemia, remia, and acute renal failure. It usually occurs in patients with bulky, rapidly proliferating, treatment-responsive tumors (Wintrobe M M, et al., “Complications of hematopoietic neoplasms.” Wintrobe's Clinical Hematology, 11th ed. Philadelphia, Pa: Lippincott Williams & Wilkins; Vol II (2003):1919-1944).

The dramatic clinical activity of CAR T-cell therapy has provoked design of additional “safety” strategies to rapidly reverse or abort the T-cell responses in patients that are undergoing CRS or associated adverse events. Metabolic approaches including co-expression of Herpes simplex virus-thymidine kinase (HSV-TK) induce apoptosis of CAR T-cells upon treatment with ganciclovir. This approach is limited by the delayed kinetics of response and the potential for immunogenic reaction to HSV. Apoptosis promoting strategies have been developed in which a drug binding domain is expressed in frame with components of the apoptotic machinery, including Caspase 9 and FAS. This system allows for conditional activation of apoptosis upon administration of a small molecule inducer of dimerization. The effect is rapid, non-immunogenic, and reduces payload of transduced cells by 90%. Both approaches are currently being evaluated in clinical trials. While expression of “suicide” genes provides a mechanism to reverse the unwanted toxicities, both approaches are considered irreversible, effectively limiting any further therapeutic benefit to the patient.

Other strategies for controlling CAR T-cell activation include certain systems of separating dual costimulatory domains from the antigen-recognition domain, wherein stimulation of the CAR T-cell is controlled by the small-molecule drug rimiducid. These T-cells, known as GoCAR-Ts, can only be fully activated when they are exposed to both cancer cells and the drug. In addition, strategies incorporating bispecific CARs which includes a second binding domain on the CAR T-cell that can lead to either an inhibitory or amplifying signal, allows for decreased off-target effects, wherein the presence of one target protein leads to activation of the CAR T-cell while the presence of a second protein leads to inhibition.

WO2016/115177 to Juno Therapeutics, Inc. titled “Modified Hepatitis Post-Transcriptional Regulatory Elements” describes the inclusion of post-transcriptional regulatory elements (PREs) in administered proteins to hasten degradation by encouraging natural ubiquitination of the protein and shorten half-life, including for example chimeric antigen receptors. The employed strategy, however, is not regulatable.

True precision control of CAR-T cells (including effective and clinically useful reversible treatments for modulating the activity of CAR T-cells) is therefore and unmet need, at least in view of the fact that CAR-T cell hyperactivation is common, life-threatening and impedes clinical development. Reversible user control of gene and cellular therapies, such as disclosed herein, can therefore increase safety and unlock new therapeutic opportunities. Precision control of gene therapies can therefore open new designs for immune and stem cell therapies. In exemplified embodiments, precision control of CAR-T cell activation is a pivotal unmet need for safer clinical development and use of CAR-T cell therapies.

Three distinct systems for IMiD-gated control of CARs and other proteins have been described herein: (1) an ON-switch IMiD-dependent heterodimer forming system, (2) an OFF-switch that employs a CRBN substrate (“degron”) as an IMiD-responsive target that induces degradation, and (3) an ON-switch that employs a fusion polypeptide having a degron joined to a CAR inhibitor, where the drug-responsive fusion polypeptide is degraded upon drug administration, thereby releasing the CAR from inhibition. Structure-guided engineering of CRBN and the CRL4^{CRBN} substrate IKZF3 was initially performed to generate minimal protein domains that functioned as drug-inducible ON- and OFF-switch peptide logic gates. For the OFF-switch, such studies were further extended to incorporate sequences of additional CRBN substrate ZFP91 (an E3 ubiquitin-protein ligase) into hybrid degron sequences (in various configurations as described herein), some of which demonstrated improved functionality as an OFF-switch. As shown in the below Examples, these control systems were functionally tested in human cell lines.

### ON-Switch—Chemically Inducible Dimerization (CID)-Mediated

Chemically inducible dimerization (CID) is an ideal molecular control system. However, no current CID system is broadly clinically tractable. Conserved design features of CID systems include: (1) specific drug-dependent interaction; (2) modular domain structures; (3) minimal dimerization domains; and (4) minimal interaction with endogenous proteins. Requirements for clinical use include: (1) non-toxicity, including non-immunosuppressive and non-immunogenic; (2) availability, including both pharmacodynamics and pharmacokinetic availability; and (3) free of regulatory impediment, i.e., modulatory agent(s) must be FDA-approved. Rapamycin-induced dimerization of FRB and FKBP is the canonical example of chemically-induced dimerization. However, potent immunosuppression severely limits the clinical scenarios where rapamycin or related compounds may be beneficial (Hubbard, Paul A., Colleen L. Moody, and Ramachandran Murali. “Allosteric modulation of Ras and the PI3K/AKT/mTOR pathway: emerging therapeutic opportunities.” Frontiers in physiology 5 (2014)).

### CRBN-CRBN Substrate Dimerization

In certain aspects, the instant disclosure relates to repurposing the thalidomide analog immunomodulatory drug (IMiD)-dependent heterodimer that forms between cereblon and a cereblon substrate (Krönke et al. Oncoimmunology 3: e941742), such heterodimer which in native forms promotes E3 ubiquitin-mediated degradation, into a drug-dependent heterodimer capable of activating heterodimerized polypeptides when brought together, while removing E3 ubiquitin-mediated degradation from the engineered system (see also Krönke et al. Science 343.6168: 301-305; Krönke et al. Nature 523.7559: 183-188; Petzold et al. Nature 532: 127-130). CRBN and CRBN substrates have therefore been reengineered to arrive at the ON-switch of the instant disclosure. Individual components of such heterodimers are considered in greater detail below.

An exemplified IMiD-responsive ON-switch of the disclosure brings together the following split receptor elements upon IMiD-induced binding between IKZF3 degron and minimal CRBN polypeptide (in certain embodiments described herein, minimal CRBN is a CRBN deleted for DDB1/CUL4 interaction domain but retaining IMiD/IKZF3 interacting domains): an antigen-responsive scFV, CD28 co-stimulatory domain(s) and a CD3ζ domain. As described herein, multiple protein engineering steps were undertaken to turn the IKZF3-CRBN protein-protein interaction into a chemically-inducible dimerization system as exemplified herein.

### Cereblon (CRBN)

Cereblon (CRBN) is a 442 amino acid protein that forms an E3 ubiquitin ligase complex with damaged DNA binding protein 1 (DDB1), Cullin-4A (CUL4A) and regulator of cullins 1 (ROC1; Angers et al. Nature 443: 590-593). This complex ubiquitinates a number of other proteins. Preclinical studies identified CRBN as a direct molecular target for the teratogenecity of thalidomide. CRBN binds directly to thalidomide analog affinity beads and is linked to the teratogenic effects of thalidomide in zebrafish and chicks (Ito et al. Science 327: 1345-1350). It was also shown that thalidomide, lenalidomide and pomalidomide each binds to CRBN in vitro (Lopez-Girona et al. Leukemia 26: 2326-2335).

Native Homo sapiens CRBN polypeptide has the
following sequence (GenBank Accession
No. AAH17419.1):
(SEQ ID NO: 6)
MAGEGDQQDAAHNMGNHLPLLPAESEEEDEMEVEDQDSKEAKKPNIINFD
TSLPTSHTYLGADMEEFHGRTLHDDDSCQVIPVLPQVMMILIPGQTLPLQ
LFHPQEVSMVRNLIQKDRTFAVLAYSNVQEREAQFGTTAEIYAYREEQDF
GIEIVKVKAIGRQRFKVLELRTQSDGIQQAKVQILPECVLPSTMSAVQLE
SLNKCQIFPSKPVSREDQCSYKWWQKYQKRKFHCANLTSWPRWLYSLYDA
ETLMDRIKKQLREWDENLKDDSLPSNPIDFSYRVAACLPIDDVLRIQLLK
IGSAIQRLRCELDIMNKCTSLCCKQCQETEITTKNEIFSLSLCGPMAAYV
NPHGYVHETLTVYKACNLNLIGRPSTEHSWFPGYAWTVAQCKICASHIGW
KFTATKKDMSPQKFWGLTRSALLPTIPDTEDEISPDKVILCL

Minimal forms of CRBN described herein have the following sequences:

minCRBN1:
(SEQ ID NO: 1)
SLSLCGPMAAYVNPHGYVHETLTVYKACNLNLIGRPSTEHSWFPGYAWTV
AQCKICASHIGWKFTATKKDMSPQKFWGLTRSALLPTIPDTEDEISPDKV
ILCL
minCRBN2:
(SEQ ID NO: 2)
CTSLCCKQCQETEITTKNEIFSLSLCGPMAAYVNPHGYVHETLTVYKACN
LNLIGRPSTEHSWFPGYAWTVAQCKICASHIGWKFTATKKDMSPQKFWGL
TRSALLPTIPDTEDEISPDKVILCL
minCRBN3:
(SEQ ID NO: 3)
AGEGDQQDAAHNMGNHLPLLPESEEEDEMEVEDQDSKEAKKPNIINFDTS
LPTSHTYLGADMEEFHGRTLHDDDSCQVIPVLPQVMMILIPGQTLPLQLF
HPQEVSMVRNLIQKDRTFAVLAYSNVQEREAQFGTTAEIYAYREEQDFGI
EIVKVKAIGRQRFKVLELRTQSDGIQQAKVQILPECVLPSTYDAETLMDR
IKKQLREWDENLKDDSLPSNPIDFSYRVAACLPIDDVLRIQLLKIGSAIQ
RLRCELDIMNKCTSLCCKQCQETEITTKNEIFSLSLCGPMAAYVNPHGYV
HETLTVYKACNLNLIGRPSTEHSWFPGYAWTVAQCKICASHIGWKFTATK
KDMSPQKFWGLTRSALLPTIPDTEDEISPDKVILCL
minCRBN4:
(SEQ ID NO: 4)
MAGEGDQQDAAHNMGNHLPLLPESEEEDEMEVEDQDSKEAKKPNIINFDT
SLPTSHTYLGADMEEFHGRTLHDDDSCQVIPVLPQVMMILIPGQTLPLQL
FHPQEVSMVRNLIQKDRTFAVLAYSNVQEREAQFGTTAEIYAYREEQDFG
IEIVKVKAIGRQRFKVLELRTQSDGIQQAKVQILPLREWDENLKDDSLPS
NPIDFSYRVAACLPIDDVLRIQLLKIGSAIQRLRCELDIMNKCTSLCCKQ
CQETEITTKNEIFSLSLCGPMAAYVNPHGYVHETLTVYKACNLNLIGRPS
TEHSWFPGYAWTVAQCKICASHIGWKFTATKKDMSPQKFWGLTRSALLPT
IPDTEDEISPDKVILCL

Variant/residue-substituted forms of CRBN as described herein have the following sequences:

minCRBN3 I371A:
(SEQ ID NO: 7)
AGEGDQQDAAHNMGNHLPLLPESEEEDEMEVEDQDSKEAKKPNIINFDTS
LPTSHTYLGADMEEFHGRTLHDDDSCQVIPVLPQVMMILIPGQTLPLQLF
HPQEVSMVRNLIQKDRTFAVLAYSNVQEREAQFGTTAEIYAYREEQDFGI
EIVKVKAIGRQRFKVLELRTQSDGIQQAKVQILPECVLPSTYDAETLMDR
IKKQLREWDENLKDDSLPSNPIDFSYRVAACLPIDDVLRIQLLKIGSAIQ
RLRCELDIMNKCTSLCCKQCQETEITTKNEIFSLSLCGPMAAYVNPHGYV
HETLTVYKACNLNLAGRPSTEHSWFPGYAWTVAQCKICASHIGWKFTATK
KDMSPQKFWGLTRSALLPTIPDTEDEISPDKVILCL
minCRBN3 I371G:
(SEQ ID NO: 8)
AGEGDQQDAAHNMGNHLPLLPESEEEDEMEVEDQDSKEAKKPNIINFDTS
LPTSHTYLGADMEEFHGRTLHDDDSCQVIPVLPQVMMILIPGQTLPLQLF
HPQEVSMVRNLIQKDRTFAVLAYSNVQEREAQFGTTAEIYAYREEQDFGI
EIVKVKAIGRQRFKVLELRTQSDGIQQAKVQILPECVLPSTYDAETLMDR
IKKQLREWDENLKDDSLPSNPIDFSYRVAACLPIDDVLRIQLLKIGSAIQ
RLRCELDIMNKCTSLCCKQCQETEITTKNEIFSLSLCGPMAAYVNPHGYV
HETLTVYKACNLNLGGRPSTEHSWFPGYAWTVAQCKICASHIGWKFTATK
KDMSPQKFWGLTRSALLPTIPDTEDEISPDKVILCL
minCRBN3 V388A:
(SEQ ID NO: 9)
AGEGDQQDAAHNMGNHLPLLPESEEEDEMEVEDQDSKEAKKPNIINFDTS
LPTSHTYLGADMEEFHGRTLHDDDSCQVIPVLPQVMMILIPGQTLPLQLF
HPQEVSMVRNLIQKDRTFAVLAYSNVQEREAQFGTTAEIYAYREEQDFGI
EIVKVKAIGRQRFKVLELRTQSDGIQQAKVQILPECVLPSTYDAETLMDR
IKKQLREWDENLKDDSLPSNPIDFSYRVAACLPIDDVLRIQLLKIGSAIQ
RLRCELDIMNKCTSLCCKQCQETEITTKNEIFSLSLCGPMAAYVNPHGYV
HETLTVYKACNLNLIGRPSTEHSWFPGYAWTAAQCKICASHIGWKFTATK
KDMSPQKFWGLTRSALLPTIPDTEDEISPDKVILCL
minCRBN3 V388G:
(SEQ ID NO: 10)
AGEGDQQDAAHNMGNHLPLLPESEEEDEMEVEDQDSKEAKKPNIINFDTS
LPTSHTYLGADMEEFHGRTLHDDDSCQVIPVLPQVMMILIPGQTLPLQLF
HPQEVSMVRNLIQKDRTFAVLAYSNVQEREAQFGTTAEIYAYREEQDFGI
EIVKVKAIGRQRFKVLELRTQSDGIQQAKVQILPECVLPSTYDAETLMDR
IKKQLREWDENLKDDSLPSNPIDFSYRVAACLPIDDVLRIQLLKIGSAIQ
RLRCELDIMNKCTSLCCKQCQETEITTKNEIFSLSLCGPMAAYVNPHGYV
HETLTVYKACNLNLIGRPSTEHSWFPGYAWTGAQCKICASHIGWKFTATK
KDMSPQKFWGLTRSALLPTIPDTEDEISPDKVILCL

### CRBN Substrates

Known CRBN substrates include: Ikaros (IKZF1), Aiolos (IKZF3; Krönke et al. Science 343.6168: 301-305; Lu et al. Science 343: 305-309) casein kinase 1α (Ck1α; Krönke et al. Nature 523: 183-188), Homeobox protein Meis2 (MEIS2; Fischer et al. Nature 512: 49-53), E3 ubiquitin-protein ligase (ZFP91; 28530236), Eukaryotic peptide chain release factor GTP-binding subunit ERF3A (GSPT1; 27338790), and Glutathione synthetase (GSS; 26990986).

Native Homo sapiens IKZF1 polypeptide has
the following sequence (GenBank Accession
No. AAH18349.1):
(SEQ ID NO: 11)
MDADEGQDMSQVSGKESPPVSDTPDEGDEPMPIPEDLSTTSGGQQSSKSD
RVVASNVKVETQSDEENGRACEMNGEECAEDLRMLDASGEKMNGSHRDQG
SSALSGVGGIRLPNGKLKCDICGIICIGPNVLMVHKRSHTGERPFQCNQC
GASFTQKGNLLRHIKLHSGEKPFKCHLCNYACRRRDALTGHLRTHSVIKE
ETNHSEMAEDLCKIGSERSLVLDRLASNVAKRKSSMPQKFLGDKGLSDTP
YDSSASYEKENEMMKSHVMDQAINNAINYLGAESLRPLVQTPPGGSEVVP
VISPMYQLHKPLAEGTPRSNHSAQDSAVENLLLLSKAKLVPSEREASPSN
SCQDSTDTESNNEEQRSGLIYLTNHIAPHARNGLSLKEEHRAYDLLRAAS
ENSQDALRVVSTSGEQMKVYKCEHCRVLFLDHVMYTIHMGCHGFRDPFEC
NMCGYHSQDRYEFSSHITRGEHRFHMS
Native Homo sapiens IKZF3 polypeptide has
the following sequence (GenBank Accession
No. NP_001271445.1):
(SEQ ID NO: 12)
MGSERALVLDRLASNVAKRKSSMPQKFIGEKRHCFDVNYNSSYMYEKESE
LIQTRMMDQAINNAISYLGAEALRPLVQTPPAPTSEMVPVISSMYPIALT
RAEMSNGAPQELEKKSIHLPEKSVPSERGLSPNNSGHDSTDTDSNHEERQ
NHIYQQNHMVLSRARNGMPLLKEVPRSYELLKPPPICPRDSVKVINKEGE
VMDVYRCDHCRVLFLDYVMFTIHMGCHGFRDPFECNMCGYRSHDRYEFSS
HIARGEHRALLK
Native Homo sapiens CK1α polypeptide has
the following sequence (GenBank Accession
No. NP_001020276.1):
(SEQ ID NO: 13)
MASSSGSKAEFIVGGKYKLVRKIGSGSFGDIYLAINITNGEEVAVKLESQ
KARHPQLLYESKLYKILQGGVGIPHIRWYGQEKDYNVLVMDLLGPSLEDL
FNFCSRRFTMKTVLMLADQMISRIEYVHTKNFIHRDIKPDNFLMGIGRHC
NKLFLIDFGLAKKYRDNRTRQHIPYREDKNLTGTARYASINAHLGIEQSR
RDDMESLGYVLMYFNRTSLPWQGLKAATKKQKYEKISEKKMSTPVEVLCK
GFPAEFAMYLNYCRGLRFEEAPDYMYLRQLFRILFRTLNHQYDYTFDWTM
LKQKAAQQAASSSGQGQQAQTPTGKQTDKTKSNMKGF
Native Homo sapiens ZFP91 polypeptide has
the following sequence (GenBank Accession
No. NP_444251.1):
(SEQ ID NO: 14)
MPGETEEPRPPEQQDQEGGEAAKAAPEEPQQRPPEAVAAAPAGTTSSRVL
RGGRDRGRAAAAAAAAAVSRRRKAEYPRRRRSSPSARPPDVPGQQPQAAK
SPSPVQGKKSPRLLCIEKVTTDKDPKEEKEEEDDSALPQEVSIAASRPSR
GWRSSRTSVSRHRDTENTRSSRSKTGSLQLICKSEPNTDQLDYDVGEEHQ
SPGGISSEEEEEEEEEMLISEEEIPFKDDPRDETYKPHLERETPKPRRKS
GKVKEEKEKKEIKVEVEVEVKEEENEIREDEEPPRKRGRRRKDDKSPRLP
KRRKKPPIQYVRCEMEGCGTVLAHPRYLQHHIKYQHLLKKKYVCPHPSCG
RLFRLQKQLLRHAKHHTDQRDYICEYCARAFKSSHNLAVHRMIHTGEKPL
QCEICGFTCRQKASLNWHMKKHDADSFYQFSCNICGKKFEKKDSVVAHKA
KSHPEVLIAEALAANAGALITSTDILGTNPESLTQPSDGQGLPLLPEPLG
NSTSGECLLLEAEGMSKSYCSGTERVSLMADGKIFVGSGSSGGTEGLVMN
SDILGATTEVLIEDSDSAGP
Native Homo sapiens GSPT1 polypeptide has
the following sequence (GenBank Accession
No. AAH09503.2):
(SEQ ID NO: 15)
MDPGSGGGGGGGGSSSGSSSSDSAPDCWDQADMEAPGPGPCGGGGSLAAA
AEAQRENLSAAFSRQLNVNAKPFVPNVHAAEFVPSFLRCPAAPPPPAGGA
ANNHGAGSGAGGRAAPVESSQEEQSLCEGSNSAVSMELSEPIENGETEMS
PEESWEHKEEISEAEPGGGSLGDGRPPEESAHEMMEEEEEIPKPKSVVAP
PGAPKKEHVNVVFIGHVDAGKSTIGGQIMYLTGMVDKRTLEKYEREAKEK
NRETWYLSWALDTNQEERDKGKTVEVGRAYFETEKKHFTILDAPGHKSFV
PNMIGGASQADLAVLVISARKGEFETGFEKGGQTREHAMLAKTAGVKHLI
VLINKMDDPTVNWSNERYEECKEKLVPFLKKVGFNPKKDIHFMPCSGLTG
ANLKEQSDFCPWYIGLPFIPYLDNLPNFNRSVDGPIRLPIVDKYKDMGTV
VLGKLESGSICKGQQLVMMPNKHNVEVLGILSDDVETDTVAPGENLKIRL
KGIEEEEILPGFILCDPNNLCHSGRTFDAQIVIIEHKSIICPGYNAVLHI
HTCIEEVEITALICLVDKKSGEKSKTRPRFVKQDQVCIARLRTAGTICLE
TFKDFPQMGRFTLRDEGKTIAIGKVLKLVPEKD
Native Homo sapiens Glutamine Synthetase
polypeptide has the following sequence
(GenBank Accession No. NP_001309423.1):
(SEQ ID NO: 16)
MATNWGSLLQDKQQLEELARQAVDRALAEGVLLRTSQEPTSSEVVSYAPF
TLFPSLVPSALLEQAYAVQMDFNLLVDAVSQNAAFLEQTLSSTIKQDDFT
ARLFDIHKQVLKEGIAQTVFLGLNRSDYMFQRSADGSPALKQIEINTISA
SFGGLASRTPAVHRHVLSVLSKTKEAGKILSNNPSKGLALGIAKAWELYG
SPNALVLLIAQEKERNIFDQRAIENELLARNIHVIRRTFEDISEKGSLDQ
DRRLFVDGQEIAVVYFRDGYMPRQYSLQNWEARLLLERSHAAKCPDIATQ
LAGTKKVQQELSRPGMLEMLLPGQPEAVARLRATFAGLYSLDVGEEGDQA
IAEALAAPSRFVLKPQREGGGNNLYGEEMVQALKQLKDSEERASYILMEK
IEPEPFENCLLRPGSPARVVQCISELGIFGVYVRQEKTLVMNKHVGHLLR
TKAIEHADGGVAAGVAVLDNPYPV
Native Homo sapiens MEIS2 polypeptide has
the following sequence (GenBank Accession
No. NP_733777.1):
(SEQ ID NO: 17)
MAQRYDELPHYGGMDGVGVPASMYGDPHAPRPIPPVHHLNHGPPLHATQH
YGAHAPHPNVMPASMGSAVNDALKRDKDAIYGHPLFPLLALVFEKCELAT
CTPREPGVAGGDVCSSDSFNEDIAVFAKQVRAEKPLFSSNPELDNLMIQA
IQVLRFHLLELEKVHELCDNFCHRYISCLKGKMPIDLVIDERDGSSKSDH
EELSGSSTNLADHNPSSWRDHDDATSTHSAGTPGPSSGGHASQSGDNSSE
QGDGLDNSVASPGTGDDDDPDKDKKRQKKRGIFPKVATNIMRAWLFQHLT
HPYPSEEQKKQLAQDTGLTILQVNNWFINARRRIVQPMIDQSNRAGFLLD
PSVSQGAAYSPEGQPMGSFVLDGQQHMGIRPAGPMSGMGMNMGMDGQWHY
M

Meis2 is noted as an endogenous substrate of CRBN (PMID: 25043012). IMiDs and IMiD-dependent substrates compete with Meis2. Therefore, it is contemplated herein that Meis2 can be stabilized by the addition of IMiDs, generating the inverse switch-behavior when using Meis2 as the degron or dimerization partner.

Other IMiD-dependent CRBN targets include E4F1, ZN276, ZN517, ZN582, ZN653, ZN654, ZN692, ZN787 and ZN827.

Native Homo sapiens ZNF692 polypeptide has the
following sequence (GenBank Accession No.
NP_001129508.1):
(SEQ ID NO: 18)
MPLVHMASSPAVDVSCRRREKRRQLDARRSKCRIRLGGHMEQWCLLKERL
GFSLHSQLAKFLLDRYTSSGCVLCAGPEPLPPKGLQYLVLLSHAHSRECS
LVPGLRGPGGQDGGLVWECSAGHTFSWGPSLSPTPSEAPKPASLPHTTRR
SWCSEATSGQELADLESEHDERTQEARLPRRVGPPPETFPPPGEEEGEEE
EDNDEDEEEMLSDASLWTYSSSPDDSEPDAPRLLPSPVTCTPKEGETPPA
PAALSSPLAVPALSASSLSSRAPPPAEVRVQPQLSRTPQAAQQTEALAST
GSQAQSAPTPAWDEDTAQIGPKRIRKAAKRELMPCDFPGCGRIFSNRQYL
NHHKKYQHIHQKSFSCPEPACGKSFNFKKHLKEHMKLHSDTRDYICEFCA
RSFRTSSNLVIHRRIHTGEKPLQCEICGFTCRQKASLNWHQRKHAETVAA
LRFPCEFCGKRFEKPDSVAAHRSKSHPALLLAPQESPSGPLEPCPSISAP
GPLGSSEGSRPSASPQAPTLLPQQ
IKZF3 degron polypeptide, amino acids 130-189:
(SEQ ID NO: 5)
FNVLMVHKRSHTGERPFQCNQCGASFTQKGNLLRHIKLHTGEKPFKCHLC
NYACQRRDAL
IKZF3 degron polypeptide, amino acids 146-168:
(SEQ ID NO: 19)
FQCNQCGASFTQKGNLLRHIKLH

In certain embodiments, a C2H2 zinc finger degron can be produced by adding IKZF3 flanking sequences to core sequences of a non-IKZF3 C2H2 degron. For example, as specifically described below for the IKZF3/ZFP91/IKZF3 “d913” hybrid degron polypeptide, a C2H2 zinc finger degron that includes IKZF3 amino acids 130-145 (FNVLMVHKRSHTGERP; SEQ ID NO: 97) positioned N-terminal to amino acids 400-410 of ZFP91 (LQCEICGFTCR; SEQ ID NO: 98), and IKZF3 amino acids 157-189 (QKGNLLRHIKLHTGEKPFKCHLCNYACQRRDAL; SEQ ID NO: 99) positioned C-terminal to the ZFP91 sequence has been demonstrated herein to be a particularly effective degron sequence. Corresponding “K0” forms of such sequences can also be employed, as demonstrated elsewhere herein—e.g., “K0” forms of IKZF3 amino acids 130-145 (FNVLMVHRRSHTGERP; SEQ ID NO: 100) and 157-189 (QRGNLLRHIRLHTGERPFRCHLCNYACQRRDAL; SEQ ID NO: 101) can also be used to flank a non-IKZF3-C2H2 zinc finger degron sequence.

More generally, in certain embodiments, improved degron performance can be imparted by modifying a given non-IKZF3 C2H2 zinc finger degron via addition of IKZF3 amino acids 130-145 (FNVLMVHKRSHTGERP; SEQ ID NO: 97) to the N-terminus and IKZF3 amino acids 169-189 (TGEKPFKCHLCNYACQRRDAL; SEQ ID NO: 102) to the C-terminus of the non-IKZF3 C2H2 zinc finger degron sequence, in order to generate an approximately 60 amino acid hybrid zinc finger.

Experiments described elsewhere herein have demonstrated that such longer hybrid degrons result in lower protein abundance, and more sensitive/deep drug-induced protein degradation. Optionally, “K0” forms of such flanking sequences (e.g., SEQ ID NO: 100 and TGERPFRCHLCNYACQRRDAL; SEQ ID NO: 103) can be used, as described above. Exemplary such embodiments include IKZF3 sequence-flanked forms of the E4F1 amino acids 220-242 sequence (HECKLCGASFRTKGSLIRHHRRH; SEQ ID NO: 104); ZN276 amino acids 524-546 sequence (LQCEVCGFQCRQRASLKYHMTKH; SEQ ID NO: 105); ZN517 amino acids 452-474 (YRCRACGRACSRLSTLIQHQKVH; SEQ ID NO: 106); ZN582 amino acids 395-417 (YQCKVCGRAFKRVSHLTVHYRIH; SEQ ID NO: 107); ZN653 amino acids 556-578 (LQCEICGYQCRQRASLNWHMKKH; SEQ ID NO: 108); ZN654 amino acids 25-47 (FACVICGRKFRNRGLMQKHLKNH; SEQ ID NO: 109); ZN692 amino acids 417-439 (LQCEICGFTCRQKASLNWHQRKH; SEQ ID NO: 110); ZN787 amino acids 178-200 (FVCPRCGRGFSQPKSLARHLRLH; SEQ ID NO: 111); ZN827 amino acids 374-396 (FQCPICGLVIKRKSYWKRHMVIH; SEQ ID NO: 112); and ZFP91 amino acids 400-422 (LQCEICGFTCRQKASLNWHMKKH; SEQ ID NO: 113). The following table (Table 1) presents these exemplary non-IKZF3 C2H2 zinc finger degrons modified via addition of IKZF3 amino acids 130-145 (FNVLMVHKRSHTGERP; SEQ ID NO: 97) to the N-terminus and IKZF3 amino acids 169-189 (TGEKPFKCHLCNYACQRRDAL; SEQ ID NO: 102) to the C-terminus of the non-IKZF3 C2H2 zinc finger degron sequence:

TABLE 1

| Component 1 | X | Component 2 | X | Component 3 |
|---|---|---|---|---|
| FNVLMVHKRSHTGERP | | HECKLCGASFRTKGSLIRHHRRH | | TGEKPFKCHLCNYACQRRDAL |
| (IKZF3 aa130-145; | | (SEQ ID NO: 104) | | (IKZF3 aa169-189; |
| SEQ ID NO: 97) | | | | SEQ ID NO: 102) |
| SEQ ID NO: 97 | | LQCEVCGFQCRQRASLKYHMTKH | | SEQ ID NO: 102 |
| | | (SEQ ID NO: 105) | | |
| SEQ ID NO: 97 | | YRCRACGRACSRLSTLIQHQKVH | | SEQ ID NO: 102 |
| | | (SEQ ID NO: 106) | | |
| SEQ ID NO: 97 | | YQCKVCGRAFKRVSHLTVHYRIH | | SEQ ID NO: 102 |
| | | (SEQ ID NO: 107) | | |
| SEQ ID NO: 97 | | LQCEICGYQCRQRASLNWHMKKH | | SEQ ID NO: 102 |
| | | (SEQ ID NO: 108) | | |
| SEQ ID NO: 97 | | FACVICGRKFRNRGLMQKHLKNH | | SEQ ID NO: 102 |
| | | (SEQ ID NO: 109) | | |
| SEQ ID NO: 97 | | LQCEICGFTCRQKASLNWHQRKH | | SEQ ID NO: 102 |
| | | (SEQ ID NO: 110) | | |
| SEQ ID NO: 97 | | FVCPRCGRGFSQPKSLARHLRLH | | SEQ ID NO: 102 |
| | | (SEQ ID NO: 111) | | |
| SEQ ID NO: 97 | | FQCPICGLVIKRKSYWKRHMVIH | | SEQ ID NO: 102 |
| | | (SEQ ID NO: 112) | | |
| SEQ ID NO: 97 | | LQCEICGFTCRQKASLNWHMKKH | | SEQ ID NO: 102 |
| | | (SEQ ID NO: 113) | | |

ZFP91 degron polypeptide, amino acids 400-422:
(SEQ ID NO: 20)
LQCEICGFTCRQKASLNWHMKKH
IKZF1 degron polypeptide, amino acids 145-167
(which are identical to IKZF3 amino acids
146-168):
(SEQ ID NO: 21)
FQCNQCGASFTQKGNLLRHIKLH
IKZF1 degron polypeptide, amino acids 129-188:
(SEQ ID NO: 22)
PNVLMVHKRSHTGERPFQCNQCGASFTQKGNLLRHIKLHSGEKPFKCHLC
NYACRRRDAL

The following specific zinc finger sequences of Table 2 have also been identified as IMiD-dependent CRBN targets.

TABLE 2

| | AA. | AA. | AA. | AA. |
|---|---|---|---|---|
| Gene | Start | Stop | Length | Sequence |
| E4F1 | 220 | 242 | 23 | HECKLCGASFRTKGS |
| | | | | LIRHHRRH |
| | | | | (SEQ ID NO: 23) |
| ZN276 | 524 | 546 | 23 | LQCEVCGFQCRQRAS |
| | | | | LKYHMTKH |
| | | | | (SEQ ID NO: 24) |
| ZN517 | 452 | 474 | 23 | YRCRACGRACSRLST |
| | | | | LIQHQKVH |
| | | | | (SEQ ID NO: 25) |
| ZN582 | 395 | 417 | 23 | YQCKVCGRAFKRVSH |
| | | | | LTVHYRIH |
| | | | | (SEQ ID NO: 26) |
| ZN653 | 556 | 578 | 23 | LQCEICGYQCRQRAS |
| | | | | LNWHMKKH |
| | | | | (SEQ ID NO: 27) |
| ZN654 | 25 | 47 | 23 | FACVICGRKFRNRGL |
| | | | | MQKHLKNH |
| | | | | (SEQ ID NO: 28) |
| ZN692 | 417 | 439 | 23 | LQCEICGFTCRQKAS |
| | | | | LNWHQRKH |
| | | | | (SEQ ID NO: 29) |
| ZN787 | 178 | 200 | 23 | FVCPRCGRGFSQPKS |
| | | | | LARHLRLH |
| | | | | (SEQ ID NO: 30) |
| ZN827 | 374 | 396 | 23 | FQCPICGLVIKRKSY |
| | | | | WKRHMVIH |
| | | | | (SEQ ID NO: 31) |

ZFP91/IKZF3 hybrid degron polypeptide, ZFP91 amino
acids 400-410 + IKZF3 amino acids 157-168:
(SEQ ID NO: 32)
LQCEICGFTCRQKGNLLRHIKLH
IKZF3/ZFP91 hybrid degron polypeptide, IKZF3 amino
acids 146-156 + ZFP91 amino acids 411-422:
(SEQ ID NO: 33)
FQCNQCGASFTQKASLNWHMKKH
ZFP91/IKZF3 hybrid degron in IKZF3 context
polypeptide, IKZF3 amino acids 130-145 +
ZFP91 amino acids 400-410 + IKZF3 amino
acids 157-189:
(SEQ ID NO: 34)
FNVLMVHKRSHTGERPLQCEICGFTCRQKGNLLRHIKLHTGEKPFKCHLC
NYACQRRDAL
IKZF3 degron polypeptide, amino acids 130-189
A153I:
(SEQ ID NO: 35)
FNVLMVHKRSHTGERPFQCNQCGISFTQKGNLLRHIKLHTGEKPFKCHLC
NYACQRRDAL
IKZF3 degron polypeptide, amino acids 130-189
A153M:
(SEQ ID NO: 36)
FNVLMVHKRSHTGERPFQCNQCGMSFTQKGNLLRHIKLHTGEKPFKCHLC
NYACQRRDAL
IKZF3 degron polypeptide, amino acids 130-189
A153T:
(SEQ ID NO: 37)
FNVLMVHKRSHTGERPFQCNQCGTSFTQKGNLLRHIKLHTGEKPFKCHLC
NYACQRRDAL
IKZF3 degron polypeptide, amino acids 130-189
A153N:
(SEQ ID NO: 38)
FNVLMVHKRSHTGERPFQCNQCGNSFTQKGNLLRHIKLHTGEKPFKCHLC
NYACQRRDAL
IKZF3 degron polypeptide, amino acids 130-189
A153Q:
(SEQ ID NO: 39)
FNVLMVHKRSHTGERPFQCNQCGQSFTQKGNLLRHIKLHTGEKPFKCHLC
NYACQRRDAL
IKZF3 degron polypeptide, amino acids 130-189
A153R:
(SEQ ID NO: 40)
FNVLMVHKRSHTGERPFQCNQCGRSFTQKGNLLRHIKLHTGEKPFKCHLC
NYACQRRDAL
IKZF3 degron polypeptide, amino acids 130-189
A153H:
(SEQ ID NO: 41)
FNVLMVHKRSHTGERPFQCNQCGHSFTQKGNLLRHIKLHTGEKPFKCHLC
NYACQRRDAL
IKZF3 degron polypeptide, amino acids 130-189
A153K:
(SEQ ID NO: 42)
FNVLMVHKRSHTGERPFQCNQCGKSFTQKGNLLRHIKLHTGEKPFKCHLC
NYACQRRDAL
IKZF3 degron polypeptide, amino acids 130-189
A153D:
(SEQ ID NO: 43)
FNVLMVHKRSHTGERPFQCNQCGDSFTQKGNLLRHIKLHTGEKPFKCHLC
NYACQRRDAL
IKZF3 degron polypeptide, amino acids 130-189
A153E:
(SEQ ID NO: 44)
FNVLMVHKRSHTGERPFQCNQCGESFTQKGNLLRHIKLHTGEKPFKCHLC
NYACQRRDAL
IKZF3 degron polypeptide, amino acids 130-189
A153C:
(SEQ ID NO: 45)
FNVLMVHKRSHTGERPFQCNQCGCSFTQKGNLLRHIKLHTGEKPFKCHLC
NYACQRRDAL

IKZF3-derived polypeptide, amino acids 130-189, with all lysine residues substituted to arginines (aka “K0”), thus generating a CRBN substrate polypeptide that can be bound but not ubiquitinated:

(SEQ ID NO: 46)
FNVLMVHRRSHTGERPFQCNQCGASFTQRGNLLRHIRLHTGERPFRCHLC
NYACQRRDAL

IKZF3/ZFP91/IKZF3 “d913” hybrid degron polypeptide, IKZF3 amino acids 130-145+ZFP91 amino acids 400-410+IKZF3 amino acids 157-189: FNVLMVHKRSHTGERPLQCEICGFTCRQKGNLLRHIKLHTGEKPFKCHLCNYACQRRDAL (SEQ ID NO: 95) (use of this sequence yielded successful generation of an OFF-switch CAR) IKZF3/ZFP91/IKZF3 “d913” hybrid degron polypeptide, IKZF3 amino acids 130-145+ZFP91 amino acids 400-410+IKZF3 amino acids 157-189, with all lysine residues substituted to arginines (aka “K0”), thus generating a CRBN substrate polypeptide that can be bound but not ubiquitinated: FNVLMVHRRSHTGERPLQCEICGFTCRQRGNLLRHIRLHTGERPFRCHLCNYACQRRDAL (SEQ ID NO: 96) (this derivative “K0” d913 improved the performance of the chemically-induced proximity system described elsewhere herein)

Corresponding “K0” forms of all above-referenced CRBN substrates and degron sequences can also be generated, to produce CRBN substrate polypeptides that can be bound but not ubiquitinated, as listed below.

IKZF3 “K0” degron polypeptide, amino acids 146-168:
(SEQ ID NO: 47)
FQCNQCGASFTQRGNLLRHIRLH
ZFP91 “K0” degron polypeptide, amino acids 400-422:
(SEQ ID NO: 48)
LQCEICGFTCRQRASLNWHMRRH
IKZF1 “K0” degron polypeptide, amino acids 145-167
(which are identical to IKZF3 amino acids 146-168):
(SEQ ID NO: 49)
FQCNQCGASFTQRGNLLRHIRLH
IKZF1 “K0” degron polypeptide, amino acids 129-188:
(SEQ ID NO: 50)
PNVLMVHRRSHTGERPFQCNQCGASFTQRGNLLRHIRLHSGERPFRCHLCN
YACRRRDAL

“K0” zinc finger sequences of Table 2 are found in Table 3 below.

TABLE 3

| | AA. | AA. | AA. | AA. |
|---|---|---|---|---|
| Gene | Start | Stop | Length | Sequence |
| E4F1 | 220 | 242 | 23 | HECRLCGASFRTRGS |
| | | | | LIRHHRRH |
| | | | | (SEQ ID NO: 51) |
| ZN276 | 524 | 546 | 23 | LQCEVCGFQCRQRAS |
| | | | | LRYHMTRH |
| | | | | (SEQ ID NO: 52) |
| ZN517 | 452 | 474 | 23 | YRCRACGRACSRLST |
| | | | | LIQHQRVH |
| | | | | (SEQ ID NO: 53) |
| ZN582 | 395 | 417 | 23 | YQCRVCGRAFRRVSH |
| | | | | LTVHYRIH |
| | | | | (SEQ ID NO: 54) |
| ZN653 | 556 | 578 | 23 | LQCEICGYQCRQRAS |
| | | | | LNWHMRRH |
| | | | | (SEQ ID NO: 55) |
| ZN654 | 25 | 47 | 23 | FACVICGRRFRNRGL |
| | | | | MQRHLRNH |
| | | | | (SEQ ID NO: 56) |
| ZN692 | 417 | 439 | 23 | LQCEICGFTCRQRAS |
| | | | | LNWHQRRH |
| | | | | (SEQ ID NO: 57) |
| ZN787 | 178 | 200 | 23 | FVCPRCGRGFSQPRS |
| | | | | LARHLRLH |
| | | | | (SEQ ID NO: 58) |
| ZN827 | 374 | 396 | 23 | FQCPICGLVIRRRSY |
| | | | | WRRHMVIH |
| | | | | (SEQ ID NO: 59) |

ZFP91/IKZF3 hybrid “K0” degron polypeptide, ZFP91
amino acids 400-410 + IKZF3 amino acids 157-168:
(SEQ ID NO: 60)
LQCEICGFTCRQRGNLLRHIRLH
IKZF3/ZFP91 hybrid “K0” degron polypeptide, IKZF3
amino acids 146-156 + ZFP91 amino acids 411-422:
(SEQ ID NO: 61)
FQCNQCGASFTQRASLNWHMRRH
ZFP91/IKZF3 hybrid “K0” degron in IKZF3 context
polypeptide, IKZF3 amino acids 130-145 + ZFP91
amino acids 400-410 + IKZF3 amino acids 157-189:
(SEQ ID NO: 62)
FNVLMVHRRSHTGERPLQCEICGFTCRQRGNLLRHIRLHTGERPFRCHLC
NYACQRRDAL
“K0” form of native Homo sapiens IKZF1
polypeptide:
(SEQ ID NO: 63)
MDADEGQDMSQVSGRESPPVSDTPDEGDEPMPIPEDLSTTSGGQQSSRSD
RVVASNVRVETQSDEENGRACEMNGEECAEDLRMLDASGERMNGSHRDQG
SSALSGVGGIRLPNGRLRCDICGIICIGPNVLMVHRRSHTGERPFQCNQC
GASFTQRGNLLRHIRLHSGERPFRCHLCNYACRRRDALTGHLRTHSVIRE
ETNHSEMAEDLCRIGSERSLVLDRLASNVARRRSSMPQRFLGDRGLSDTP
YDSSASYERENEMMRSHVMDQAINNAINYLGAESLRPLVQTPPGGSEVVP
VISPMYQLHRPLAEGTPRSNHSAQDSAVENLLLLSRARLVPSEREASPSN
SCQDSTDTESNNEEQRSGLIYLTNHIAPHARNGLSLREEHRAYDLLRAAS
ENSQDALRVVSTSGEQMRVYRCEHCRVLFLDHVMYTIHMGCHGFRDPFEC
NMCGYHSQDRYEFSSHITRGEHRFHMS
“K0” form of native Homo sapiens IKZF3
polypeptide:
(SEQ ID NO: 64)
MGSERALVLDRLASNVARRRSSMPQRFIGERRHCFDVNYNSSYMYERESE
LIQTRMMDQAINNAISYLGAEALRPLVQTPPAPTSEMVPVISSMYPIALT
RAEMSNGAPQELERRSIHLPERSVPSERGLSPNNSGHDSTDTDSNHEERQ
NHIYQQNHMVLSRARNGMPLLREVPRSYELLRPPPICPRDSVRVINREGE
VMDVYRCDHCRVLFLDYVMFTIHMGCHGFRDPFECNMCGYRSHDRYEFSS
HIARGEHRALLR
“K0” form of native Homo sapiens CK1α
polypeptide
(SEQ ID NO: 65)
MASSSGSRAEFIVGGRYRLVRRIGSGSFGDIYLAINITNGEEVAVRLESQ
RARHPQLLYESRLYRILQGGVGIPHIRWYGQERDYNVLVMDLLGPSLEDL
FNFCSRRFTMRTVLMLADQMISRIEYVHTRNFIHRDIRPDNFLMGIGRHC
NRCLESPVGRRRRSMTVSTSQDPSFSGLNQLFLIDFGLARRYRDNRTRQH
IPYREDRNLTGTARYASINAHLGIEQSRRDDMESLGYVLMYFNRTSLPWQ
GLRAATRRQRYERISERRMSTPVEVLCRGFPAEFAMYLNYCRGLRFEEAP
DYMYLRQLFRILFRTLNHQYDYTFDWTMLRQRAAQQAASSSGQGQQAQTP
TGRQTDRTRSNMRGF
“K0” form of native Homo sapiens ZFP91
polypeptide:
(SEQ ID NO: 66)
MPGETEEPRPPEQQDQEGGEAARAAPEEPQQRPPEAVAAAPAGTTSSRVL
RGGRDRGRAAAAAAAAAVSRRRRAEYPRRRRSSPSARPPDVPGQQPQAAR
SPSPVQGRRSPRLLCIERVTTDRDPREEREEEDDSALPQEVSIAASRPSR
GWRSSRTSVSRHRDTENTRSSRSRTGSLQLICRSEPNTDQLDYDVGEEHQ
SPGGISSEEEEEEEEEMLISEEEIPFRDDPRDETYRPHLERETPRPRRRS
GRVREERERREIRVEVEVEVREEENEIREDEEPPRRRGRRRRDDRSPRLP
RRRRRPPIQYVRCEMEGCGTVLAHPRYLQHHIRYQHLLRRRYVCPHPSCG
RLFRLQRQLLRHARHHTDQRDYICEYCARAFRSSHNLAVHRMIHTGERPL
QCEICGFTCRQRASLNWHMRRHDADSFYQFSCNICGRRFERRDSVVAHRA
RSHPEVLIAEALAANAGALITSTDILGTNPESLTQPSDGQGLPLLPEPLG
NSTSGECLLLEAEGMSRSYCSGTERVSLMADGRIFVGSGSSGGTEGLVMN
SDILGATTEVLIEDSDSAGP
“K0” form of native Homo sapiens GSPT1
polypeptide:
(SEQ ID NO: 67)
MDPGSGGGGGGGGSSSGSSSSDSAPDCWDQADMEAPGPGPCGGGGSLAAA
AEAQRENLSAAFSRQLNVNARPFVPNVHAAEFVPSFLRCPAAPPPPAGGA
ANNHGAGSGAGGRAAPVESSQEEQSLCEGSNSAVSMELSEPIENGETEMS
PEESWEHREEISEAEPGGGSLGDGRPPEESAHEMMEEEEEIPRPRSVVAP
PGAPRREHVNVVFIGHVDAGRSTIGGQIMYLTGMVDRRTLERYEREARER
NRETWYLSWALDTNQEERDRGRTVEVGRAYFETERRHFTILDAPGHRSFV
PNMIGGASQADLAVLVISARRGEFETGFERGGQTREHAMLARTAGVRHLI
VLINRMDDPTVNWSNERYEECRERLVPFLRRVGFNPRRDIHFMPCSGLTG
ANLREQSDFCPWYIGLPFIPYLDNLPNFNRSVDGPIRLPIVDRYRDMGTV
VLGRLESGSICRGQQLVMMPNRHNVEVLGILSDDVETDTVAPGENLRIRL
RGIEEEEILPGFILCDPNNLCHSGRTFDAQIVIIEHRSIICPGYNAVLHI
HTCIEEVEITALICLVDRRSGERSRTRPRFVRQDQVCIARLRTAGTICLE
TFRDFPQMGRFTLRDEGRTIAIGRVLRLVPERD
“K0” form of native Homo sapiens Glutamine
Synthetase polypeptide:
(SEQ ID NO: 68)
MATNWGSLLQDRQQLEELARQAVDRALAEGVLLRTSQEPTSSEVVSYAPF
TLFPSLVPSALLEQAYAVQMDFNLLVDAVSQNAAFLEQTLSSTIRQDDFT
ARLFDIHRQVLREGIAQTVFLGLNRSDYMFQRSADGSPALRQIEINTISA
SFGGLASRTPAVHRHVLSVLSRTREAGRILSNNPSRGLALGIARAWELYG
SPNALVLLIAQERERNIFDQRAIENELLARNIHVIRRTFEDISERGSLDQ
DRRLFVDGQEIAVVYFRDGYMPRQYSLQNWEARLLLERSHAARCPDIATQ
LAGTRRVQQELSRPGMLEMLLPGQPEAVARLRATFAGLYSLDVGEEGDQA
IAEALAAPSRFVLRPQREGGGNNLYGEEMVQALRQLRDSEERASYILMER
IEPEPFENCLLRPGSPARVVQCISELGIFGVYVRQERTLVMNRHVGHLLR
TRAIEHADGGVAAGVAVLDNPYPV
“K0” form of native Homo sapiens MEIS2
polypeptide:
(SEQ ID NO: 69)
MAQRYDELPHYGGMDGVGVPASMYGDPHAPRPIPPVHHLNHGPPLHATQH
YGAHAPHPNVMPASMGSAVNDALRRDRDAIYGHPLFPLLALVFERCELAT
CTPREPGVAGGDVCSSDSFNEDIAVFARQVRAERPLFSSNPELDNLMIQA
IQVLRFHLLELERVHELCDNFCHRYISCLRGRMPIDLVIDERDGSSRSDH
EELSGSSTNLADHNPSSWRDHDDATSTHSAGTPGPSSGGHASQSGDNSSE
QGDGLDNSVASPGTGDDDDPDRDRRRQRRRGIFPRVATNIMRAWLFQHLT
HPYPSEEQRRQLAQDTGLTILQVNNWFINARRRIVQPMIDQSNRAGFLLD
PSVSQGAAYSPEGQPMGSFVLDGQQHMGIRPAGPMSGMGMNMGMDGQWH
YM

### C2H2 Zinc Finger Polypeptides

The Cys2His2-like fold group (C2H2) is by far the best-characterized class of zinc fingers and are extremely common in mammalian transcription factors. These domains adopt a simple ββα fold and have the amino acid Sequence motif (Pabo et al. Annual Review of Biochemistry. 70: 313-40):
X2-Cys-X2,4-Cys-X12-His-X3,4,5-His
   This class of zinc fingers can have a variety of functions such as binding RNA and mediating protein-protein interactions, but is best known for its role in sequence-specific DNA-binding proteins such as Zif268 (Egr1). In such proteins, individual zinc finger domains typically occur as tandem repeats with two, three, or more fingers comprising the DNA-binding domain of the protein. These tandem arrays can bind in the major groove of DNA and are typically spaced at 3-bp intervals. The α-helix of each domain (often called the “recognition helix”) can make sequence-specific contacts to DNA bases; residues from a single recognition helix can contact 4 or more bases to yield an overlapping pattern of contacts with adjacent zinc fingers.

Exemplary known C2H2 zinc finger polypeptides include: BNC1, BNC2, CTCF, CTCFL, EGR2, EGR3, EGR4, E4F1, GFI1B, GLIS1, GLIS2, GLI1, GLI2, GLI3, GTF3A, IKZF1, IKZF2, IKZF3, IKZF4, IKZF5, INSM2, KLF1, KLF2, KLF3, KLF4, KLF5, KLF6, KLF7, KLF9, KLF10, KLF11, KLF12, KLF13, KLF14, KLF15, KLF16, MECOM, OSR1, OSR2, OVOL1, PLAGL1, PRDM1, PRDM4, PRDM5, PRDM6, PRDM10, PRDM12, PRDM14, PRDM16, PRKRIP1, RLF, RREB1, SNAI1, SNAI2, SP1, SP2, SP3, SP4, SP5, SP6, SP7, SP8, SUZ12, TRPS1, TSHZ1, WT1, YY1, ZBTB3, ZBTB5, ZBTB10, ZBTB12, ZEB1, ZEB2, ZFP28, ZHX1, ZHX2, ZHX3, ZIC2, ZIC4, ZIC5, ZNF2, ZNF3, ZNF7, ZNF8, ZNF10, ZNF12, ZNF14, ZNF16, ZNF17, ZNF18, ZNF19, ZNF20, ZNF22, ZNF23, ZNF24, ZNF25, ZNF26, ZNF28, ZNF30, ZNF32, ZNF33A, ZNF33B, ZNF34, ZNF35, ZKSCAN1, ZNF37A, ZSCAN21, HIVEP1, HIVEP2, HIVEP3, ZNF41, MZF1, ZNF43, ZNF44, ZNF45, ZBTB25, ZNF48, ZSCAN22, BCL6, ZNF56, ZNF57, ZBTB17, BCL6B, ZKSCAN7, ZNF69, ZNF70, ZNF71, ZNF73, ZNF74, ZNF75A, ZNF75D, ZNF76, ZNF77, ZNF79, ZNF80, ZNF81, ZNF83, ZNF84, ZNF85, ZFPM1, ZFPM2, ZNF90, ZNF91, ZNF92, ZNF93, ZSCAN12, ZNF98, ZNF99, ZNF100, ZNF101, ZNF106, ZNF107, ZNF112, ZNF114, ZNF117, ZNF121, ZNF124, ZNF131, ZNF132, ZNF133, ZNF134, ZNF135, ZNF136, ZNF138, ZNF140, ZNF141, ZNF142, ZNF143, ZBTB16, ZNF146, ZNF148, ZNF154, ZNF155, ZNF157, ZNF160, VEZF1, GFI1, ZNF165, ZNF169, ZNF174, ZNF175, ZNF177, ZNF180, ZNF181, ZNF182, ZNF184, ZSCAN26, ZNF189, ZKSCAN8, ZSCAN9, ZNF195, ZNF197, ZNF200, ZIC1, ZNF202, ZIC3, ZNF205, ZSCAN10, ZNF207, ZNF208, ZNF211, ZNF212, ZNF213, ZNF214, ZNF215, ZNF217, TSHZ2, ZNF219, ZNF221, ZNF222, ZNF223, ZNF224, EGR1, ZNF225, ZNF226, ZNF227, ZNF229, ZNF230, ZNF232, ZNF233, ZNF234, ZNF235, ZNF236, ZBTB18, ZNF239, ZNF248, ZNF250, ZNF251, ZNF253, ZNF254, ZNF256, ZNF257, ZNF260, ZNF263, ZNF264, ZNF266, ZNF267, ZNF268, ZNF271P, ZNF273, ZNF274, ZNF275, ZNF276, PATZ1, ZNF281, ZNF282, ZNF283, ZNF284, ZNF285, ZNF286A, ZNF286B, ZNF287, ZBTB20, SCAPER, ZNF292, SNAI3, ZBTB21, ZNF296, ZBTB22, ZBTB43, PRDM15, ZNF300, ZNF302, ZNF304, ZKSCAN3, ZKSCAN4, ZNF311, FEZF2, FEZF1, ZNF316, ZNF317, ZNF318, ZNF319, ZNF320, ZNF322, ZSCAN31, ZNF324, ZNF324B, ZNF326, ZNF329, ZNF330, ZNF331, ZNF333, ZNF334, ZNF335, GZF1, ZNF337, ZFP64, OVOL2, ZBTB46, ZNF341, ZNF343, ZNF345, ZNF347, ZBTB33, ZNF350, ZNF354A, ZNF354B, ZNF354C, ZNF358, ZSCAN20, ZNF362, ZNF365, ZNF366, ZNF367, ZNF382, ZNF383, ZNF384, ZSCAN23, ZNF391, ZSCAN16, KLF17, ZNF394, ZNF395, ZNF396, ZNF397, ZNF398, ZNF404, ZFAT, ZNF407, ZNF408, ZFHX2, ZNF410, ZNF414, ZNF415, ZNF416, ZNF417, ZNF418, ZNF419, ZNF420, ZNF423, ZNF425, ZNF426, ZNF428, ZNF429, ZNF430, ZNF431, ZNF432, ZNF433, ZSCAN32, ZNF436, ZBTB2, ZNF438, ZNF439, ZNF440, ZNF441, ZNF442, ZNF443, ZNF444, ZNF445, ZNF446, ZSCAN18, ZNF449, ZBTB24, ZNF451, ZNF454, ZNF460, ZNF461, ZNF462, REPINI, ZNF467, ZNF468, ZNF469, ZNF470, ZNF471, ZNF473, ZNF474, ZFP1, ZBTB14, ZNF479, ZNF480, ZBTB26, ZBTB6, ZNF483, ZNF484, ZNF485, ZNF486, ZNF487, ZNF488, ZNF490, ZNF491, ZNF492, ZNF493, ZSCAN4, ZSCAN5A, ZSCAN5B, ZSCAN5C, ZSCAN5DP, ZNF496, ZNF497, ZSCAN25, ZBTB45, ZNF500, ZNF501, ZNF502, ZNF503, ZNF506, ZNF507, ADNP2, ZBTB49, ZNF510, ZNF511, ZNF512, ZNF513, ZNF514, GLIS3, ZNF516, ZNF517, ZNF518A, ZNF518B, ZNF519, ZNF521, ZNF524, ZNF525, ZNF526, ZNF527, ZNF528, ZNF529, ZNF530, ZFP14, ZNF532, ZNF534, ZNF536, TSHZ3, ZBTB32, ZNF540, ZNF541, ZNF542P, ZNF543, ZNF544, ZFP82, ZNF546, ZNF547, ZNF548, ZNF549, ZNF550, ZNF551, ZNF552, ZNF554, ZNF555, ZNF556, ZNF557, ZNF558, ZNF559, ZNF560, ZNF561, ZNF562, ZNF563, ZNF564, ZNF565, ZNF566, ZNF567, ZNF568, ZNF569, ZNF570, ZNF571, ZNF572, ZNF573, ZNF574, ZNF575, ZNF576, ZNF577, ZNF578, ZNF579, ZNF580, ZNF581, ZNF582, ZNF583, ZNF584, ZNF585A, ZNF585B, ZNF586, ZNF587, ZNF587B, ZNF589, ZNF592, ZNF594, ZNF595, ZNF596, ZNF597, ZNF598, ZNF599, ZNF600, ZNF605, ZNF606, ZNF607, ZNF608, ZNF609, ZNF610, ZNF611, ZNF613, ZNF614, ZNF615, ZNF616, ZNF618, ZNF619, ZNF620, ZNF621, ZNF623, ZNF624, ZNF625, ZNF626, ZNF627, ZNF628, ZNF629, ZNF630, YY2, ZNF639, ZNF641, ZFP69, ZFP69B, ZNF646, ZNF648, ZNF649, ZBTB47, ZNF652, ZNF653, ZNF655, ZIM2, ZIM3, ZNF658, ZNF660, ZNF662, ZNF663P, ZNF664, ZNF665, ZNF667, ZNF668, ZNF669, ZNF670, ZNF671, ZNF672, ZNF674, ZNF675, ZNF676, ZNF677, ZNF678, ZNF679, ZNF680, ZNF681, ZNF682, ZNF683, ZNF684, ZNF688, ZNF689, ZSCAN29, ZNF691, ZNF692, CASZ1, ZKSCAN2, ZNF695, ZNF696, ZNF697, ZFP57, ZNF699, ZNF700, ZNF701, ZNF705A, ZNF705B, ZNF705D, ZNF705F, ZNF705G, ZNF707, ZNF708, ZNF709, ZNF710, ZNF711, ZNF713, ZNF714, ZNF716, ZNF717, ZNF718, ZNF720, ZNF721, ZNF726, ZNF727, ZNF728, ZNF729, ZNF730, ZNF732, ZNF735, ZNF736, ZNF737, ZNF740, KLF8, HINFP, ANKZF1, ZFP30, ZNF746, ZNF747, ZNF749, ZFP2, ZFP3, ZFP41, ZFP42, ZFP62, ZFP90, ZFP91, ZNF761, ZIK1, ZNF763, ZNF764, ZNF765, ZNF766, ZNF767P, ZNF768, RBAK, ZNF770, ZNF771, ZNF772, ZNF773, ZNF774, ZNF775, ZNF776, ZNF777, ZNF778, ZNF780A, ZNF780B, ZNF781, ZNF782, ZNF783, ZNF784, ZNF785, ZNF786, ZNF787, ZNF788P, ZNF789, ZNF790, ZNF791, ZNF792, ZNF793, SALL1, SALL2, SALL3, SALL4, FIZ1, ZNF799, ZNF800, MAZ, JAZF1, WIZ, ZNF805, ZNF806, ZNF807, ZNF808, ZNF812P, ZNF813, ZNF814, ZNF816, ZNF821, ATMIN, ZNF823, ZNF827, CHAMPI, ZNF829, ZNF835, ZNF836, ZNF837, ZNF841, ZNF843, ZNF844, ZNF845, ZNF846, ZNF850, ZBTB44, ZNF852, ZNF853, ZSCAN2, ZBTB48, BCL11A, BCL11B, ZBTB7A, ZBTB7B, ZBTB7C, ZNF860, ZNF862, ZNF865, HKR1, ZNF878, ZNF879, ZNF880, ZNF883, ZNF888, ZNF891, ZXDA, ZFP92, SCRT1, SCRT2, PRDM9, PLAGL2, HIC1, MYNN, ZBTB4, PEG3, ZXDB, ZFP37, HIC2, ZBTB37, ZBTB1, PRDM7, ZFY, PLAG1, ZBTB11, ZKSCAN5, ZSCAN1, ZBTB8A, ZBTB8B, ZSCAN30, ZBTB34, ZBTB9, ZBTB38, ZBTB39, ZBTB40, ZBTB41, ZBTB42, ZFX, ZFHX3, GLI4, SP9, ZUP1 and ZXDC.

### Thalidomide Analog Immunomodulatory Drugs (IMiDs)

Immunomodulatory drugs (IMiDs) include thalidomide and a recently developed class of anti-cancer drug derived from thalidomide, which have been developed and exert potent anti-cancer effects. Exemplary IMiDs include:

### Thalidomide:

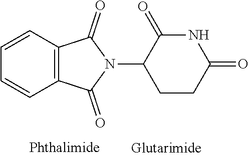 [SMILES: O=C1CCC(N2C(=O)c3ccccc3C2=O)C(=O)N1]

Thalidomide can be administered orally and is available in 50 mg, 100 mg, 150 mg, and 200 mg dose capsules. An exemplary on-label dose of thalidomide is 200 mg per day to a human subject.

### Lenalidomide:

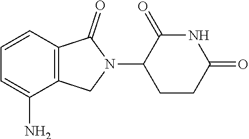 [SMILES: Nc1cccc2c1CN(C1CCC(=O)NC1=O)C2=O]

Common adult human oral lenalidomide dosages include: 2.5 mg once a day, 5 ng once a day, 10 mg once a day. 15 mg once a day, 15 mg every other day, 25 mg once a day and 25 ng every other day.

### Pomalidomide:

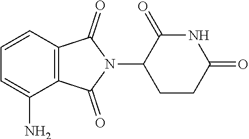 [SMILES: Nc1cccc2c1C(=O)N(C1CCC(=O)NC1=O)C2=O]

Common adult human oral pomalidomide dosages include: 1-5 mg once a day, including 3 mg once a day and 4 mg once a day.

### CC-122:

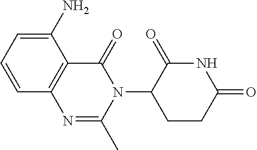 [SMILES: Cc1nc2cccc(N)c2c(=O)n1C1CCC(=O)NC1=O]

Exemplary adult human oral CC-122 dosages include: 0.5-5 mg once a day, including 0.5, 1, 1.5, 2, 2.5, 3 and 3.5 mg once a day.

CC-885 targets GSPT1 to CRBN (Nature, 2016 vol. 535 (7611) pp. 252-257).

As shown above, thalidomide is composed of a glutarimide ring and a pthalimide ring. Lenalidomide and pomalidomide are very similar to thalidomide. Lenalidomide is a 4-amino analog of thalidomide lacking a carbonyl group of pthalidmide. Pomalidomide is a 4-amino analog of thalidomide. Although CC-122 possesses a glutarimide ring, the structure is quite different from other IMiDs.

The thalidomide analog immunomodulatory drugs (IMiDs) lenalidomide and pomalidomide are well-tolerated, FDA-approved drugs for hematologic malignancies that induce the degradation of proteins required for myeloma and MDS cell survival via binding to CRBN, the substrate adapter for the CRL^{CRBN} E3 ubiquitin ligase (Krönke et al. Science 343.6168: 301-305; Krönke et al. Nature 523.7559: 183-188).

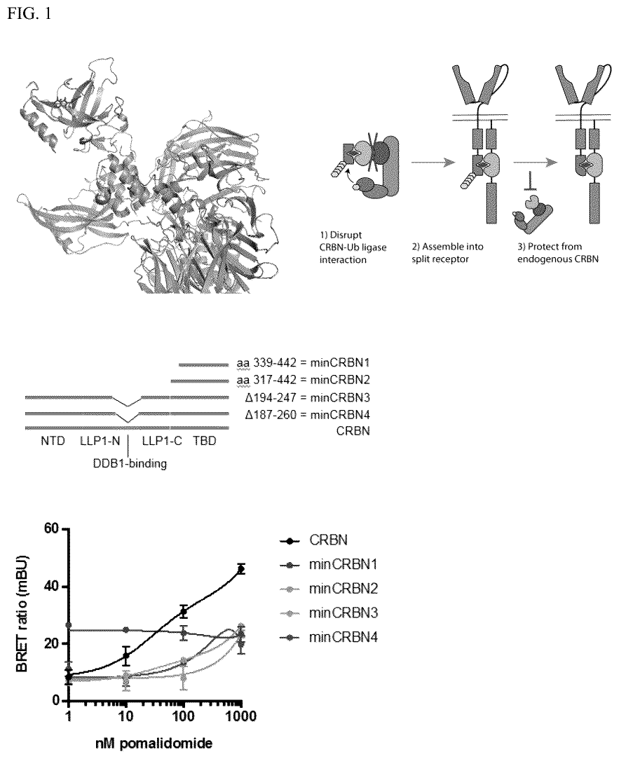

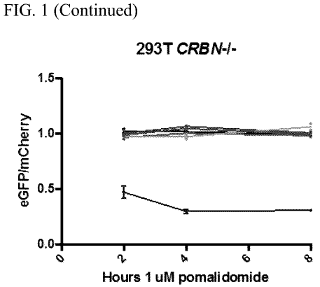

Controlled, reversible CAR-T activation with pomalidomide (Pom)-gated control switches can help prevent or suppress CAR-T cell hyperactivation syndromes. Disclosed herein is an engineered, chemically induced dimerization domain from the IKZF3-Pom-CRBN interaction with application to medical uses, including IMiD-gated split CARs as schematically demonstrated in, e.g., FIGS. 1-4, and as exemplified herein. It is expressly contemplated that a range of IMiD compounds can be used in such systems, in addition to the specifically exemplified pomalidomide, with similar effect.

### OFF-Switch—CRBN Substrate Degron-Mediated

Certain aspects of the instant disclosure employ the polypeptide sequence of a CRBN substrate or fragment thereof as a IMiD-inducible element that promotes ubiquitination and degradation of the polypeptide to which the CRBN substrate sequence (thereby termed a “degron”) is attached. Exemplary CRBN substrate polypeptide sequences used and/or contemplated for use in such OFF-switches of the instant disclosure include those recited above (e.g., IKZF1, IKZF3, and Ck1α polypeptide sequences, including fragments and variants thereof).

In certain OFF-switch configurations, multiple degron elements (e.g., CRBN polypeptide substrate domains capable of binding CRBN in response to drug, thereby promoting ubiquitin pathway-mediated degradation of a drug-responsive CAR) can be employed, and optionally can be joined to each other in series or array configuration, e.g., joined to one another via polypeptide linkers. A range of polypeptide linkers are known in the art and can be employed within fusion polypeptides of the instant disclosure, including synthetic and natural polypeptide linkers, e.g., synthetic flexible polypeptide sequences such as (G4S)ₙ, synthetic rigid polypeptide sequences such as A(EAAAK)₃A, and/or natural linkers such as those that join zinc fingers in IKZF3, such as TGEKP or TGERP.

Examples of specific linkers include those set forth in Klein et al. (Protein Engineering, Design & Selection. 27: 325-30), e.g., linkers L1-L24 below (SEQ ID NOs: 114-137):

| Linker | Name | Complete sequence |
|---|---|---|
| L1 | GPcPcPc | AGSGGSGGSGGSPVPSTPPTNSSSTPPTPSPSPVPSTPPTNSSSTPPTPSPSPVPSTPPTNSS |
| | | STPPTPSPSAS |
| L2 | GPPcP | AGSGGSGGSGGSPVPSTPPTPSPSTPPTPSPSPVPSTPPTNSSSTPPTPSPSPVPSTPPTPSP |
| | | STPPTPSPSAS |
| L3 | GPGcP | AGSGGSGGSGGSPVPSTPPTPSPSTPPTPSPSGGSGNSSGSGGSPVPSTPPTPSPSTPPTPSP |
| | | SAS |
| L4 | GPPP | AGSGGSGGSGGSPVPSTPPTPSPSTPPTPSPSPVPSTPPTPSPSTPPTPSPSPVPSTPPTPSP |
| | | STPPTPSPSAS |
| L5 | GPbP | AGSGGSGGSGGSPVPSTPPTPSPSTPPTPSPSIQRTPKIQVYSRHPAENGKSNFLNCYVSGFH |
| | | PSDIEVDLLKNGERIEKVEHSDLSFSKDWSFYLLYYTEFTPTEKDEYACRVNHVTLSQPKIVK |
| | | WDRDPVPSTPPTPSPSTPPTPSPSAS |
| L6 | GPbG | AGSGGSGGSGGSPVPSTPPTPSPSTPPTPSPSIQRTPKIQVYSRHPAENGKSNFLNCYVSGFH |
| | | PSDIEVDLLKNGERIEKVEHSDLSFSKDWSFYLLYYTEFTPTEKDEYACRVNHVTLSQPKIVK |
| | | WDRDGGSGGSGGSGGSAS |
| L7 | PbGbG | AGPVPSTPPTPSPSTPPTPSPSIQRTPKIQVYSRHPAENGKSNFLNCYVSGFHPSDIEVDLLK |
| | | NGERIEKVEHSDLSFSKDWSFYLLYYTEFTPTEKDEYACRVNHVTLSQPKIVKWDRDGGSGGS |
| | | GGSGGSIQRTPKIQVYSRHPAENGKSNFLNCYVSGFHPSDIEVDLLKNGERIEKVEHSDLSFS |
| | | KDWSFYLLYYTEFTPTEKDEYACRVNHVTLSQPKIVKWDRDGGSGGSGGSGAS |
| L8 | GPbGbP | AGSGGSGGSGGSPVPSTPPTPSPSTPPTPSPSIQRTPKIQVYSRHPAENGKSNFLNCYVSGFH |
| | | PSDIEVDLLKNGERIEKVEHSDLSFSKDWSFYLLYYTEFTPTEKDEYACRVNHVTLSQPKIVK |
| | | WDRDGGSGGSGGSGGSIQRTPKIQVYSRHPAENGKSNFLNCYVSGFHPSDIEVDLLKNGERIE |
| | | KVEHSDLSFSKDWSFYLLYYTEFTPTEKDEYACRVNHVTLSQPKIVKWDRDPVPSTPPTPSPS |
| | | TPPTPSPSAS |
| L9 | GPUG | AGSGGSGGSGGSPVPSTPPTPSPSTPPTPSPSQIFVKTLTGKTITLEVEPSDTIENVKAKIQD |
| | | KEGIPPDQQRLIFAGKQLEDGRTLSDYNIQKESTLHLVLRLRGGGGSGGSGGSGGSAS |
| L10 | GPZP | AGSGGSGGSGGSPVPSTPPTPSPSTPPTPSPSDGRYSLTYIYTGLSKHVEDVPAFQALGSLND |
| | | LQFFRYNSKDRKSQPMGLWRQVEGMEDWKQDSQLQKAREDIFMETLKDIVEYYNDSNGSHVLQ |
| | | GRFGCEIENNRSSGAFWKYYYDGKDYIEFNKEIPAWVPFDPAAQITKQKWEAEPVYVQRAKAY |
| | | LEEECPATLRKYLKYSKNILDRQDPPSVVVTSHQAPGEKKKLKCLAYDFYPGKIDVHWTRAGE |
| | | VQEPELRGDVLHNGNGTYQSWVVVAVPPQDTAPYSCHVQHSSLAQPLVVPWEASPVPSTPPTP |
| | | SPSTPPTPSAS |
| L11 | GGZGZP | AGSGGSGGSGGSGGSGGSGGSGGSDGRYSLTYIYTGLSKHVEDVPAFQALGSLNDLQFFRYNS |
| | | KDRKSQPMGLWRQVEGMEDWKQDSQLQKAREDIFMETLKDIVEYYNDSNGSHVLQGRFGCEIE |
| | | NNRSSGAFWKYYYDGKDYIEFNKEIPAWVPFDPAAQITKQKWEAEPVYVQRAKAYLEEECPAT |
| | | LRKYLKYSKNILDRQDPPSVVVTSHQAPGEKKKLKCLAYDFYPGKIDVHWTRAGEVQEPELRG |
| | | DVLHNGNGTYQSWVVVAVPPQDTAPYSCHVQHSSLAQPLVVPWEASGGSGGSGGSGGSDGRYS |
| | | LTYIYTGLSKHVEDVPAFQALGSLNDLQFFRYNSKDRKSQPMGLWRQVEGMEDWKQDSQLQKA |
| | | REDIFMETLKDIVEYYNDSNGSHVLQGRFGCEIENNRSSGAFWKYYYDGKDYIEFNKEIPAWV |
| | | PFDPAAQITKQKWEAEPVYVQRAKAYLEEECPATLRKYLKYSKNILDRQDPPSVVVTSHQAPG |
| | | EKKKLKCLAYDFYPGKIDVHWTRAGEVQEPELRGDVLHNGNGTYQSWVVVAVPPQDTAPYSCH |
| | | VQHSSLAQPLVVPWEASPVPSTPPTPSPSTPPTPSPSAS |
| L12 | GcGcP | AGSGNSSGSGGSGGSGNSSGSGGSPVPSTPPTPSPSTPPTPSPSAS |
| L13 | cTPR3 | KLSGGGGSGGGGSGGGGSAEAWYNLGNAYYKQGDYQKAIEYYQKALELDPNNAEAWYNLGNAY |
| | | YKQGDYQKAIEYYQKALELDPNNAEAWYNLGNAYYKQGDYQKAIEDYQKALELDPNNLQRSAG |
| | | GGGSGGGGSGGGGAS |
| L14 | cTPR6 | KLSGGGGSGGGGSGGGGSAEAWYNLGNAYYKQGDYQKAIEYYQKALELDPNNAEAWYNLGNAY |
| | | YKQGDYQKAIEYYQKALELDPNNAEAWYNLGNAYYKQGDYQKAIEDYQKALELDPNNLQAEAW |
| | | KNLGNAYYKQGDYQKAIEYYQKALELDPNNASAWYNLGNAYYKQGDYQKAIEYYQKALELDPN |
| | | NAKAWYRRGNAYYKQGDYQKAIEDYQKALELDPNNRSRSAGGGGSGGGGSGGGGAS |
| L15 | cTPR9 | KLSGGGGSGGGGSGGGGSAEAWYNLGNAYYKQGDYQKAIEYYQKALELDPNNAEAWYNLGNAY |
| | | YKQGDYQKAIEYYQKALELDPNNAEAWYNLGNAYYKQGDYQKAIEDYQKALELDPNNLQAEAW |
| | | KNLGNAYYKQGDYQKAIEYYQKALELDPNNASAWYNLGNAYYKQGDYQKAIEYYQKALELDPN |
| | | NAKAWYRRGNAYYKQGDYQKAIEDYQKALELDPNNRSAEAWYNLGNAYYKQGDYQKAIEYYQK |
| | | ALELDPNNAEAWYNLGNAYYKQGDYQKAIEYYQKALELDPNNAEAWYNLGNAYYKQGDYQKAI |
| | | EDYQKALELDPNNLQRSAGGGGSGGGGSGGGGAS |
| L16 | cTPR12 | KLSGGGGSGGGGSGGGGSAEAWYNLGNAYYKQGDYQKAIEYYQKALELDPNNAEAWYNLGNAY |
| | | YKQGDYQKAIEYYQKALELDPNNAEAWYNLGNAYYKQGDYQKAIEDYQKALELDPNNLQAEAW |
| | | KNLGNAYYKQGDYQKAIEYYQKALELDPNNASAWYNLGNAYYKQGDYQKAIEYYQKALELDPN |
| | | NAKAWYRRGNAYYKQGDYQKAIEDYQKALELDPNNRSAEAWYNLGNAYYKQGDYQKAIEYYQK |
| | | ALELDPNNAEAWYNLGNAYYKQGDYQKAIEYYQKALELDPNNAEAWYNLGNAYYKQGDYQKAI |
| | | EDYQKALELDPNNLQAEAWKNLGNAYYKQGDYQKAIEYYQKALELDPNNASAWYNLGNAYYKQ |
| | | GDYQKAIEYYQKALELDPNNAKAWYRRGNAYYKQGDYQKAIEDYQKALELDPNNRSAGGGGSG |
| | | GGGSGGGGAS |
| L17 | GS1 | GGGGSAS |
| L18 | GS2 | GGGGSGGGGSAS |
| L19 | GS3 | GGGGSGGGGSGGGGSAS |
| L20 | GS5 | GGGGSGGGGSGGGGSGGGGSGGGGSAS |
| L21 | GS6 | GGGGSGGGGSGGGGSGGGGSGGGGSGGGGSAS |
| L22 | GS7 | AGGGSGGGGSGGGGSGGGGSGGGGSGGGGSGGGGSAS |
| L23 | GS8 | AGGGSGGGGSGGGGSGGGGSGGGGSGGGGSGGGGSGGGGSAS |
| L24 | GS9 | AGGGSGGGGSGGGGSGGGGSGGGGSGGGGSGGGGSGGGGSGGGGSAS |

Other exemplary linker polypeptides include β2-microglobulin, Zn-α2-glycoprotein and tetratricopeptide repeats, among many other forms of polypeptide linkers known in the art. For degron-mediated switch configurations (whether a degron-mediated OFF-switch or a degron-mediated ON-switch, as described below) it is expressly contemplated that the depth and/or speed of CRBN-mediated ubiquitylation and degradation of a degron-tagged CAR or CAR inhibitory polypeptide can be enhanced by overexpressing CRBN in a mammalian cell harboring such degron-tagged fusion polypeptide, thereby increasing the concentration of the ubiquitin ligase CRL4^{CRBN}.

It is further contemplated for degron-mediated switch configurations that the depth and/or speed of CRBN-mediated ubiquitylation and degradation of a degron-tagged CAR or CAR inhibitory polypeptide can be enhanced by overexpressing a CRBN-containing polypeptide that is targeted to the plasma membrane via a targeting sequence of or derived from LAT, PAG, LCK, FYN, LAX, CD2, CD3, CD4, CD5, CD7, CD8a, PD1, SRC and/or LYN, thereby increasing the local concentration of the ubiquitin ligase CRL4^{CRBN} at the plasma membrane.

### Degron-Mediated ON-Switch Configuration

In certain aspects of the instant disclosure, one or more degrons (e.g., a CRBN polypeptide substrate domain capable of binding CRBN in response to drug, thereby promoting ubiquitin pathway-mediated degradation of a drug-responsive CAR) is fused to a CAR inhibitor, such that a degron-mediated, drug-responsive ON-switch is formed. As for other degron-presenting fusion polypeptides, degron polypeptide domains can be included as single degron polypeptide domains or as multiple degron polypeptide domains, optionally where multiple degron polypeptide domains are joined in a series or an array, optionally using polypeptide linkers, such as those known in the art and/or described above.

The fusion polypeptide that includes a CAR inhibitory domain and a degron can also be referred to as a chimeric degradable inhibitor (CDI). In certain embodiments that CDI is a constitutive inhibitor of CAR signaling absent administration of a degron-targeting drug. Upon administration of the drug, degradation of the inhibitor licenses the CAR for antigen-dependent signal transduction.

In such degron-mediated ON-switch configurations, it is contemplated that the inhibitor may be a proximal, pan-CAR/TCR signal transduction inhibitor, such as CSK. The inhibitor may also selectively abrogate specific signal transduction pathways and/or effector functions, such as Ras signaling, PKC, calcium-dependent signaling, NF-kappaB, NFAT, actin and cytoskeletal responses, cytokine secretion, cell proliferation, degranulation, tumor cell killing, differentiation, or exhaustion.

The inhibitor may be a ubiquitin ligase involved in TCR/CAR signal transduction, such as c-CBL, CBL-B, ITCH, RNF125, RNF128, or WWP2.

The inhibitor may be a TCR/CAR negative regulatory enzyme such as SHP1, SHP2, SHIP1, SHIP2, CD45, CSK, CD148, PTPN22, DGKalpha, DGKzeta, DRAK2, HPK1, HPK1, STS1, STS2, or SLAT.

The inhibitor may be a TCR/CAR negative regulatory scaffold/adapter protein such as PAG, LIME, NTAL, LAX31, SIT, GAB2, GRAP, ALX, SLAP, SLAP2, DOK1, or DOK2.

The inhibitor may be a dominant negative version of an activating TCR signaling component, such ZAP70, LCK, FYN, NCK, VAV1, SLP76, ITK, ADAP, GADS, PLCgamma1, LAT, p85, SOS, GRB2, NFAT, p50, p65, AP1, RAP1, CRKII, C3G, WAVE2, ARP2/3, ABL, ADAP, RIAM, or SKAP55.

The inhibitor may contain the cytoplasmic tail of TCR/CAR negative co-regulatory receptors such as CD5, PD1, CTLA4, BTLA, LAG3, B7-H1, B7-1, CD160, TIM3, 2B4, or TIGIT.

The inhibitor may be targeted to the plasma membrane with a targeting sequence derived from LAT, PAG, LCK, FYN, LAX, CD2, CD3, CD4, CD5, CD7, CD8a, PD1, SRC, or LYN.

Both OFF-switch and ON-switch compositions of the instant disclosure can be readily applied in pre-clinical models of immune and stem cell therapy, as a prelude to clinical application.

More generally, FDA-approved drug-controllable activation and/or degradation of engineered proteins in cellular therapies can have broad applications, including the immunotherapeutic and stem cell therapies expressly presented herein.

### Chimeric Antigen Receptors (CARs)

CAR-T cells can act to kill targeted tumor cells, but can also elicit negative effects capable of propagating in an unchecked manner. Conventional CAR-T design uses a single polypeptide that includes tumor antigen-binding domains (e.g., an scFv), a transmembrane domain, a costimulatory domain and a CD3ζ domain (TCR ITAMs), with this single polypeptide therefore capable of specifically binding a tumor antigen and propagating an activated T cell response. (see, e.g., Wu et al. Science 350: aab4077.)

As contemplated herein, the CARs (including split CAR systems) of the present disclosure include an extracellular ligand binding domain capable of binding a targeted protein, typically an antigen, for example a tumor antigen. In one embodiment, the extracellular ligand binding domain is an antigen binding domain, for example, an antibody or an antigen binding fragment thereof. In particular embodiments, the antigen-binding fragment is a Fab or scFv. In one embodiment, the extracellular ligand binding domain is a ligand for a tumor marker, for example, a ligand that binds a marker expressed on the cell surface of a tumor, for example IL13 which binds to the IL13 receptor (IL13R) on glioma cells or heregulin which binds to erb B2, B3, and B4 on breast cancer cells. In one embodiment, the extracellular ligand binding domain targets a labeled or tagged protein or molecule, for example biotin or fluorescein isothiocyanate, which is bound to an antibody targeting a tumor expressed protein. For example, the extracellular ligand binding domain can target a label on a tumor-specific antibody, for example biotin, so that when the antibody-label binds to the tumor cell, the extracellular binding ligand of the CAR T-cell binds the label, activating the T-cell, and killing the tumor cell. In this regard, a “universal CAR” can be generated capable of binding any tagged or labeled antibody. See, e.g., Abate Daga et al., “CAR models: next generation CAR modifications for enhanced T-cell function,” Molecular Therapy-Oncolytics (2016)3:1-7.

In certain embodiments, the antigen binding domain in the CAR binds to a tumor antigen, for example, a tumor antigen associated with a hematological malignancy or a solid tumor. Tumor antigens capable of being targeted by CAR T-cells are known, and include, for example, but are not limited to, CD19, CD20, CD22, CD30, CD40, CD70, CD123, ErbB2 (HER2/neu), epithelial cell adhesion molecule (EpCAM), Epidermal growth factor receptor (EGFR), epidermal growth factor receptor variant III (EGFRvIII). Disialoganglioside GD2, disialoganglioside GD3, mesothelian, ROR1, mesothelin, CD33/IL3Ra, C-Met, PSMA, Glycolipid, F77, EGFRvIII, GD-2, NY-ESO-1 TCR, melanoma-associated antigen (MAGE) A3 TCR, melanoma-associated antigen (MAGE) A1 TCR, alphafetapotein (AFP), carcinoembryonic antigen (CEA), CA-125, MUC-1, epithelial tumor antigen (ETA), tyrosinase, CA15-3, CA27-29, CA19-9, calcitonin, calretinin CD34, CD99MIC2, CD7, chromogranin, cytokeratin, desmin, CD31 FL1, glial fibrillary acidic protein, gross cystid disease fluid protein, HMB-45, human chorionic gonadotropin inhibin, MART-1, Myo D1, neuron-specific enolast, placental alkaline phosphatase, prostate specific antigens, PSCA. PTPRC, S100 protein, synaptophysin, thyroglobulin, thyroid transcription factor 1, tumor M2-PK, vimentin, human telomerase reverse transcriptase (hTERT), surviving, mouse double minute 2 homolog (MDM2), kappa-light chain, LeY, L1 cell adhesion molecule, oncofetal antigen (h5T4), TAG-72, VEGF-R2, and combinations thereof, as well as others described herein. Other antigens to which the antigen binding domain of the CAR can be directed include, but are not limited to, tissue or cell lineage specific antigens including, but not limited to, CD3, CD4, CD8, CD24, CD25, CD33, CD34, CD133, CD138, or a combination thereof.

In certain embodiments, a CAR (or split CAR system) of the disclosure includes an extracellular domain having an antigen recognition domain, a transmembrane domain, and a cytoplasmic domain. In one embodiment, the transmembrane domain that naturally is associated with one of the domains in the CAR is used. In another embodiment, the transmembrane domain can be selected or modified by amino acid substitution to avoid binding of such domains to the transmembrane domains of the same or different surface membrane proteins to minimize interactions with other members of the receptor complex. In one embodiment, the transmembrane domain is the CD8a hinge domain.

With respect to the cytoplasmic domain, the CAR (or split CAR system) of the disclosure is designed to include at least one signaling domain, at least one co-stimulatory domain and either a FDA-approved drug-responsive heterodimer or a FDA-approved drug-inducible degron moiety. The heterodimer components and/or degron of the CAR or CAR system are amino acid sequences to which the FDA-approved drug can be bound, leading to activation in the presence of both drug and an antigen in the ON-switch split CAR system of the disclosure or to the degradation of the CAR when in contact with the drug in the OFF-switch aspects of the disclosure.

In OFF-switch aspects, the degron should not interfere with the function of the CAR. In certain embodiments, the OFF-switch degron and/or ON-switch heterodimer components are amino acid sequences derived from an endogenous protein which has been modified so that the FDA-approved compound selectively binds to modified degron and/or modified heterodimer components, relative to endogenously expressed proteins.

As contemplated herein, the CARs (including split CAR systems) of the present disclosure include a transmembrane domain spanning the extracellular ligand binding domain and the at least one intracellular co-stimulatory and/or signaling domain. Transmembrane domains useful in the construction of CARs are known in the art, and can be derived from natural or synthetic sources. For example, transmembrane regions contemplated herein include, but are not limited to, those derived from (i.e. comprise at least the transmembrane region(s) of) the alpha, beta or zeta chain of the T-cell receptor, CD28, CD3 epsilon, CD8, CD45, CD4, CD5, CDS, CD9, CD 16, CD22, CD33, CD37, CD64, CD80, CD86, CD 134, CD137, CD 154, or KIR2DS2. Alternatively the transmembrane domain in some embodiments is synthetic, for example, comprising predominantly hydrophobic residues such as leucine and valine. In some aspects, a triplet of phenylalanine, tryptophan and valine will be found at each end of a synthetic transmembrane domain.

As further contemplated herein, the CARs (or split CAR systems) of the present disclosure include at least one intracellular (or cytoplasmic) signaling domain. The intracellular signaling domain of the CAR activates at least one of the normal effector functions or responses of the immune cell. For example, upon binding of the extracellular ligand domain to a target antigen, the signaling domain may act to activate the CAR T-cell, for example, by inducing a function of a T-cell such as cytolytic activity or T-helper activity, including the secretion of cytokines or other factors. In some embodiments, the CAR includes an intracellular component of the TCR complex, such as a TCR CD3+ chain that mediates T-cell activation and cytotoxicity, e.g., the immunoreceptor tyrosine-based activation motif (ITAM) domain CD3 zeta chain (CD3ζ). Thus, in some aspects as contemplated herein, the antigen binding molecule is linked to one or more cell signaling domains. In some embodiments, cell signaling domains include CD3 transmembrane domain, CD3 intracellular signaling domains, and/or other CD transmembrane domains. In some embodiments, the CAR further includes a portion of one or more additional molecules such as Fc receptor 7, for example FcεRIγ, CD8, CD4, CD25, or CD16. For example, in some aspects, the CAR includes a chimeric molecule between CD3-zeta (CD3-ζ) or Fc receptor 7 and CD8, CD4, CD25 or CD16. In one embodiment, the intracellular signaling domain is a Dap-12 derived signaling domain.

The intracellular signaling domain, or cytoplasmic signaling domain, used interchangeably herein, of the CAR (or split CAR system) of the disclosure is responsible for activation of at least one of the normal effector functions of the immune cell in which the CAR has been placed. The term “effector function” refers to a specialized function of a cell. Effector function of a T-cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Examples of intracellular signaling domains for use in the CAR (and/or split CAR system) of the disclosure include the cytoplasmic sequences of the T-cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone may not be sufficient for full activation of the T-cell and that a secondary or co-stimulatory signal may also be required. Thus, T-cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences that are of particular use in the disclosure include those derived from TCR zeta, FcR gamma, FcR beta, CD3 gamma, CD3 delta, CD3 epsilon, CD5, CD22, CD79a, CD79b, and CD66d. In one embodiment, the cytoplasmic signaling molecule in the CAR of the disclosure comprises a cytoplasmic signaling sequence derived from CD3 zeta.

The cytoplasmic domain of the CAR (and/or split CAR system) can be designed to comprise the CD3-zeta signaling domain by itself or combined with any other desired cytoplasmic domain(s) useful in the context of the CAR of the disclosure. For example, the cytoplasmic domain of the CAR can comprise a CD3 zeta chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. Thus, any of the costimulatory elements known in the art as useful in the construction of CARs are within the scope of the disclosure.

The cytoplasmic signaling sequences within the cytoplasmic signaling portion of the CAR of the disclosure may be linked to each other in a random or specified order. Optionally, a short oligo- or polypeptide linker, preferably between 2 and 10 amino acids in length may form the linkage. A glycine-serine doublet provides a particularly suitable linker.

In one embodiment, the cytoplasmic domain (optionally including cytoplasmic domains in a split CAR system) is designed to comprise the signaling domain of CD3-zeta and the signaling domain of CD28. In another embodiment, the cytoplasmic domain (optionally including cytoplasmic domains in a split CAR system) is designed to comprise the signaling domain of CD3-zeta and the signaling domain of 4-1BB. In yet another embodiment, the cytoplasmic domain (optionally including cytoplasmic domains in a split CAR system) is designed to comprise the signaling domain of CD3-zeta and the signaling domain of CD28 and 4-1BB. In some embodiments, the intracellular signaling domain comprises a chimeric CD28 and OX40 co-stimulatory domain. In some embodiments, the intracellular signaling domain comprises a chimeric CD27 co-stimulatory domain. In some embodiments, the intracellular signaling domain comprises a chimeric CD27 and DAP10 co-stimulatory domain.

In some embodiments, the intracellular signaling domain or domains include the cytoplasmic sequences of the T-cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability. In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

In some aspects, the cell comprises a first CAR which contains signaling domains to induce the primary signal and a second CAR which binds to a second antigen and contains the component for generating a costimulatory signal. For example, a first CAR can be an activating CAR and the second CAR can be a costimulatory CAR. In some aspects, both CARs must be ligated in order to induce a particular effector function in the cell, which can provide specificity and selectivity for the cell type being targeted. In one embodiment, the cell comprises a first CAR which contains signaling domains to induce the primary signal and a costimulatory ligand molecule to stimulate other immune cells. See, e.g., Abate Daga et al., “CAR models: next generation CAR modifications for enhanced T-cell function,” Molecular Therapy-Oncolytics (2016)3:1-7. In certain split CAR systems, such as exemplified herein, one or more co-stimulatory domains might be presented upon a first polypeptide that is distinct from a second polypeptide comprising antigen binding domains and/or signaling domains, pending drug-induced heterodimeric association between the first and second polypeptides.

In some embodiments, the CAR (or split CAR system) includes a signaling domain and/or transmembrane portion of a costimulatory receptor, such as CD28, 4-1BB, OX40, DAP10, and ICOS. In some aspects, the same CAR includes both the activating and costimulatory components; in other aspects, the activating domain is provided by one polypeptide (e.g., a CAR) whereas the costimulatory component is provided by another polypeptide (e.g., a CAR or ligand recognizing another antigen).

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD 137 (4-1BB, TNFRSF9) co-stimulatory domain, linked to a CD3 zeta intracellular domain. In some embodiments, the intracellular signaling domain comprises a chimeric CD28 or CD 137 (4-1BB, TNFRSF9) co-stimulatory domain. In some embodiments, the intracellular signaling domain comprises a chimeric CD28 and OX40 co-stimulatory domain. In some embodiments, the intracellular signaling domain comprises a chimeric CD27 co-stimulatory domain. In some embodiments, the intracellular signaling domain comprises a chimeric CD27 and DAP10 co-stimulatory domain.

In some embodiments, the CAR (or split CAR system) encompasses two or more costimulatory domain combined with an activation domain, e.g., primary activation domain, in the cytoplasmic portion. One example is a receptor including intracellular components of CD3-zeta, CD28, and 4-1BB. Other examples include a receptor including intracellular components of CD3-zeta, CD28, and OX40.

While the instant disclosure exemplifies ON-switch and OFF-switch drug-inducible modalities as integral components of cellular polypeptides, molecular formats in which the drug-responsive ON-switch or OFF-switch polypeptides of the instant disclosure are joined to receptor- and/or signaling domain-presenting polypeptides using any of an array of art-recognized linkers (e.g., linker sequences, linker compounds, etc., e.g., as described in WO 2017/024318) while retaining switch functionality are also expressly envisioned herein.

### Immune Effector Cells

As contemplated herein, the CARs (including split CAR systems) of the present disclosure are expressed by an immune effector cell, for example a T-cell, and administered to a subject in order to treat a disease or disorder, for example, a cancer. Among the cell types that may be used to express the CARs of the present disclosure include, but are not limited to, T-cells, NK cells, CD4+T-cells, CD8⁺ cells, and stem cells, such as an induced pluripotent stem cell (iPS cell). In one embodiment, the cell is an autologous T-cell. In one embodiment, the cell shows anti-tumor activity when cross-reacted with a tumor cell containing an antigen capable of being bound by the extracellular ligand binding domain.

Further contemplated herein is the use of an FDA-approved compound capable of binding to a degron presented by a CAR of the present disclosure to induce degradation through ubiquitination. By administering to a subject an FDA-approved compound directed to the degron polypeptide, the immune effector cell response can be modulated in a subject who has previously received an immune effector cell expressing the CARs of the present disclosure. The FDA-approved compounds for use in the present disclosure tend to be small molecules, in certain aspects capable of disabling the biological function of the CAR through binding to the degron polypeptide. The FDA-approved compounds for use in the present disclosure in certain aspects therefore provide prompt ligand-dependent target protein degradation via chemical conjugation with a degron moiety that recruits the function of the Cereblon E3 ubiquitin ligase complex. Using this “OFF”-switch approach, certain CARs of the present disclosure can be degraded rapidly with a high specificity and efficiency.

Moreover, in such OFF-switch aspects, by combining the chemical strategy of protein degradation via the FDA-approved molecules of the present application with the effectiveness of CAR T-cell therapy, the activity of the CAR T-cell, and thus the side effects, can be regulated in a precise, temporal manner by rapidly turning on and off ubiquitination, and proteasomal degradation of the CAR.

Similarly, in “ON”-switch aspects of the disclosure, signaling is only activated upon administration of an FDA-approved agent that promotes formation of a heterodimer, thereby activating, e.g., a split CAR system of the disclosure and effecting CAR T-cell therapy. In such a system, any side effects of CAR T-cell therapy can be regulated in a precise, temporal manner by rapidly removing and/or (re-)administering the FDA-approved agent that induces CAR T-cell activation.

The generation of CAR T-cells is known in the art. For example, see Wang et al, “Clinical manufacturing of CAR T cells: foundation of a promising therapy,” Oncolytics (2016)3:1-7 (and incorporated herein). In general, the CAR T-cells of the disclosure can be generated by introducing a lentiviral vector including a desired CAR and/or CAR system, for example a CAR comprising anti-CD19, CD8a hinge and transmembrane domain, human CD28 and CD3zeta signaling domains, and degron OFF-switch into the cells. The CAR T-cells of the disclosure are able to replicate in vivo resulting in long-term persistence that can lead to sustained tumor control, and are subject to modulation of activation via administration of a FDA-approved drug to which the degron (OFF-switch) or heterodimer (ON-switch) is responsive.

In certain embodiments, genetically modified T-cells expressing a CAR or split CAR system of the disclosure for the treatment of a patient having cancer or at risk of having cancer are administered using lymphocyte infusion. Autologous lymphocyte infusion is used in the treatment. Autologous PBMCs are collected from a patient in need of treatment and T-cells are activated and expanded using the methods described herein and known in the art and then infused back into the patient.

In yet another embodiment, the treatment of a patient at risk of developing CLL is provided. The disclosure also includes treating a malignancy or an autoimmune disease in which chemotherapy and/or immunotherapy in a patient results in significant immunosuppression in the patient, thereby increasing the risk of the patient of developing CLL.

The disclosure includes using CAR T-cells that express a CAR or split CAR system of the disclosure. The CAR T-cells of the disclosure can undergo robust in vivo CAR T-cell expansion and can establish targeted antigen-specific memory cells that persist at high levels for an extended amount of time in blood and bone marrow. In some instances, the CAR T-cells of the disclosure infused into a patient can be modulated by administering to the subject an FDA-approved compound that is capable of activating the CAR (ON-switch split CAR system aspects), thereby activating the CAR T-cell, or provoking degradation of the CAR (OFF-switch aspects) and down regulation of the CAR T-cell activation without destroying the CAR T-cell.

In one aspect, a nucleic acid is provided that encodes a CAR having an extracellular ligand binding domain, a transmembrane domain, and a cytoplasmic domain having at least one intracellular signaling domain and a degron capable of being bound by an FDA-approved agent and a component of the E3 ubiquitin/proteasome degradation system, e.g., cereblon (CRBN).

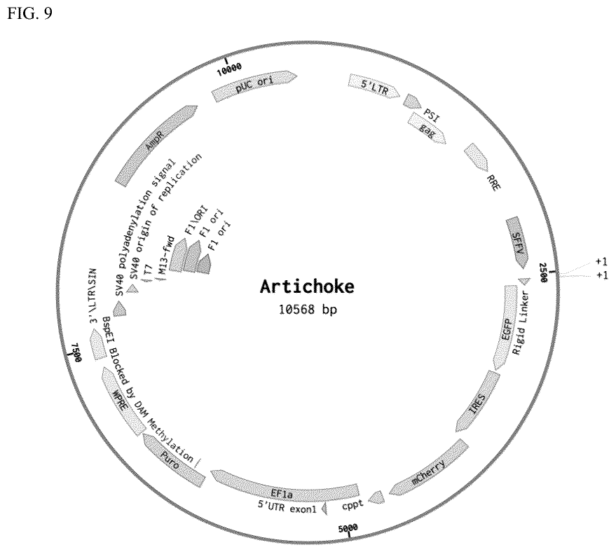

Specific sequences of certain aspects of the instant disclosure include the following: “Artichoke” (SFFV.BsmBICloneSite-17AARigidLinker-EGFP.IRES.mCherry.cppt.EF1a.PuroR; see FIG. 9):

(SEQ ID NO: 70)
CGAACGACCGAGCGCAGCGAGTCAGTGAGCGAGGAAGCGGAAGAGCGCCCAATACG
CAAACCGCCTCTCCCCGCGCGTTGGCCGATTCATTAATGCAGCTGGCACGACAGGTTT
CCCGACTGGAAAGCGGGCAGTGAGCGCAACGCAATTAATGTGAGTTAGCTCACTCAT
TAGGCACCCCAGGCTTTACACTTTATGCTTCCGGCTCGTATGTTGTGTGGAATTGTGAG
CGGATAACAATTTCACACAGGAAACAGCTATGACCATGATTACGCCAAGCGCGCAAT
TAACCCTCACTAAAGGGAACAAAAGCTGGAGCTGCAAGCTTAATGTAGTCTTATGCA
ATACTCTTGTAGTCTTGCAACATGGTAACGATGAGTTAGCAACATGCCTTACAAGGAG
AGAAAAAGCACCGTGCATGCCGATTGGTGGAAGTAAGGTGGTACGATCGTGCCTTAT
TAGGAAGGCAACAGACGGGTCTGACATGGATTGGACGAACCACTGAATTGCCGCATT
GCAGAGATATTGTATTTAAGTGCCTAGCTCGATACATAAACGGGTCTCTCTGGTTAGA
CCAGATCTGAGCCTGGGAGCTCTCTGGCTAACTAGGGAACCCACTGCTTAAGCCTCAA
TAAAGCTTGCCTTGAGTGCTTCAAGTAGTGTGTGCCCGTCTGTTGTGTGACTCTGGTAA
CTAGAGATCCCTCAGACCCTTTTAGTCAGTGTGGAAAATCTCTAGCAGTGGCGCCCGA
ACAGGGACTTGAAAGCGAAAGGGAAACCAGAGGAGCTCTCTCGACGCAGGACTCGG
CTTGCTGAAGCGCGCACGGCAAGAGGCGAGGGGCGGCGACTGGTGAGTACGCCAAA
AATTTTGACTAGCGGAGGCTAGAAGGAGAGAGATGGGTGCGAGAGCGTCAGTATTAA
GCGGGGGAGAATTAGATCGCGATGGGAAAAAATTCGGTTAAGGCCAGGGGGAAAGA
AAAAATATAAATTAAAACATATAGTATGGGCAAGCAGGGAGCTAGAACGATTCGCAG
TTAATCCTGGCCTGTTAGAAACATCAGAAGGCTGTAGACAAATACTGGGACAGCTAC
AACCATCCCTTCAGACAGGATCAGAAGAACTTAGATCATTATATAATACAGTAGCAA
CCCTCTATTGTGTGCATCAAAGGATAGAGATAAAAGACACCAAGGAAGCTTTAGACA
AGATAGAGGAAGAGCAAAACAAAAGTAAGACCACCGCACAGCAAGCGGCCGCTGAT
CTTCAGACCTGGAGGAGGAGATATGAGGGACAATTGGAGAAGTGAATTATATAAATA
TAAAGTAGTAAAAATTGAACCATTAGGAGTAGCACCCACCAAGGCAAAGAGAAGAGT
GGTGCAGAGAGAAAAAAGAGCAGTGGGAATAGGAGCTTTGTTCCTTGGGTTCTTGGG
AGCAGCAGGAAGCACTATGGGCGCAGCGTCAATGACGCTGACGGTACAGGCCAGACA
ATTATTGTCTGGTATAGTGCAGCAGCAGAACAATTTGCTGAGGGCTATTGAGGCGCAA
CAGCATCTGTTGCAACTCACAGTCTGGGGCATCAAGCAGCTCCAGGCAAGAATCCTG
GCTGTGGAAAGATACCTAAAGGATCAACAGCTCCTGGGGATTTGGGGTTGCTCTGGA
AAACTCATTTGCACCACTGCTGTGCCTTGGAATGCTAGTTGGAGTAATAAATCTCTGG
AACAGATTTGGAATCACACGACCTGGATGGAGTGGGACAGAGAAATTAACAATTACA
CAAGCTTAATACACTCCTTAATTGAAGAATCGCAAAACCAGCAAGAAAAGAATGAAC
AAGAATTATTGGAATTAGATAAATGGGCAAGTTTGTGGAATTGGTTTAACATAACAAA
TTGGCTGTGGTATATAAAATTATTCATAATGATAGTAGGAGGCTTGGTAGGTTTAAGA
ATAGTTTTTGCTGTACTTTCTATAGTGAATAGAGTTAGGCAGGGATATTCACCATTATC
GTTTCAGACCCACCTCCCAACCCCGAGGGGACCCTATTCCAGCACATATGAGCTAGCT
GCAGTAACGCCATTTTGCAAGGCATGGAAAAATACCAAACCAAGAATAGAGAAGTTC
AGATCAAGGGCGGGTACATGAAAATAGCTAACGTTGGGCCAAACAGGATATCTGCGG
TGAGCAGTTTCGGCCCCGGCCCGGGGCCAAGAACAGATGGTCACCGCAGTTTCGGCC
CCGGCCCGAGGCCAAGAACAGATGGTCCCCAGATATGGCCCAACCCTCAGCAGTTTC
TTAAGACCCATCAGATGTTTCCAGGCTCCCCCAAGGACCTGAAATGACCCTGCGCCTT
ATTTGAATTAACCAATCAGCCTGCTTCTCGCTTCTGTTCGCGCGCTTCTGCTTCCCGAG
CTCTATAAAAGAGCTCACAACCCCTCACTCGGCGCGCCAGTCCTCCGACAGACTGAGT
CGGCCGGTCGAATCAAGCTTATCGATACCGTCGACTCCGGAATAGCCACCATGGAGA
CGGACGTCTCAgctgaagctgctgcaAAGgaagctgcagctAAGgaggctgcagctA
AGgctGTGAGCAAGGGCGAGGAGCTGTTCACCGGGGTGGTGCCCATCCTGGTCGAGCTG
GACGGCGACGTAAACGGCCACAAGTTCAGCGTGTCCGGCGAGGGCGAGGGCGATGCCAC
CTACGGCAAGCTGACCCTGAAGTTCATCTGCACCACCGGCAAGCTGCCCGTGCCCTGGC
CCACCCTCGTGACCACCCTGACCTACGGCGTGCAGTGCTTCAGCCGCTACCCCGACCAC
ATGAAGCAGCACGACTTCTTCAAGTCCGCCATGCCCGAAGGCTACGTCCAGGAGCGCAC
CATCTTCTTCAAGGACGACGGCAACTACAAGACCCGCGCCGAGGTGAAGTTCGAGGGCG
ACACCCTGGTGAACCGCATCGAGCTGAAGGGCATCGACTTCAAGGAGGACGGCAACATC
CTGGGGCACAAGCTGGAGTACAACTACAACAGCCACAACGTCTATATCATGGCCGACAA
GCAGAAGAACGGCATCAAGGTGAACTTCAAGATCCGCCACAACATCGAGGACGGCAGC
GTGCAGCTCGCCGACCACTACCAGCAGAACACCCCCATCGGCGACGGCCCCGTGCTG
CTGCCCGACAACCACTACCTGAGCACCCAGTCCGCCCTGAGCAAAGACCCCAACGAG
AAGCGCGATCACATGGTCCTGCTGGAGTTCGTGACCGCCGCCGGGATCACTCTCGGCA
TGGACGAGCTGTACAAGTAAATGCATGAGTAACTGAGGATCCGCCCCTCTCCCTCCCC
CCCCCCTAACGTTACTGGCCGAAGCCGCTTGGAATAAGGCCGGTGTGCGTTTGTCTAT
ATGTTATTTTCCACCATATTGCCGTCTTTTGGCAATGTGAGGGCCCGGAAACCTGGCC
CTGTCTTCTTGACGAGCATTCCTAGGGGTCTTTCCCCTCTCGCCAAAGGAATGCAAGG
TCTGTTGAATGTCGTGAAGGAAGCAGTTCCTCTGGAAGCTTCTTGAAGACAAACAACG
TCTGTAGCGACCCTTTGCAGGCAGCGGAACCCCCCACCTGGCGACAGGTGCCTCTGCG
GCCAAAAGCCACGTGTATAAGATACACCTGCAAAGGCGGCACAACCCCAGTGCCACG
TTGTGAGTTGGATAGTTGTGGAAAGAGTCAAATGGCTCTCCTCAAGCGTATTCAACAA
GGGGCTGAAGGATGCCCAGAAGGTACCCCATTGTATGGGATCTGATCTGGGGCCTCG
GTACACATGCTTTACATGTGTTTAGTCGAGGTTAAAAAAACGTCTAGGCCCCCCGAAC
CACGGGGACGTGGTTTTCCTTTGAAAAACACGATGATAATATGGCCACAACCCTGGA
ATTCGCCACCATGGTGAGCAAGGGCGAGGAGGATAACATGGCCATCATCAAGGAGTT
CATGCGCTTCAAGGTGCACATGGAGGGCTCCGTGAACGGCCACGAGTTCGAGATCGA
GGGCGAGGGCGAGGGCCGCCCCTACGAGGGCACCCAGACCGCCAAGCTGAAGGTGA
CCAAGGGTGGCCCCCTGCCCTTCGCCTGGGACATCCTGTCCCCTCAGTTCATGTACGG
CTCCAAGGCCTACGTGAAGCACCCCGCCGACATCCCCGACTACTTGAAGCTGTCCTTC
CCCGAGGGCTTCAAGTGGGAGCGCGTGATGAACTTCGAGGACGGCGGCGTGGTGACC
GTGACCCAGGACTCCTCCCTGCAGGACGGCGAGTTCATCTACAAGGTGAAGCTGCGC
GGCACCAACTTCCCCTCCGACGGCCCCGTAATGCAGAAGAAGACCATGGGCTGGGAG
GCCTCCTCCGAGCGGATGTACCCCGAGGACGGCGCCCTGAAGGGCGAGATCAAGCAG
AGGCTGAAGCTGAAGGACGGCGGCCACTACGACGCTGAGGTCAAGACCACCTACAAG
GCCAAGAAGCCCGTGCAGCTGCCCGGCGCCTACAACGTCAACATCAAGTTGGACATC
ACCTCCCACAACGAGGACTACACCATCGTGGAACAGTACGAACGCGCCGAGGGCCGC
CACTCCACCGGCGGCATGGACGAGCTGTACAAGTAAACTAGTAAGCTTGGCGTAACT
AGATCTTGAGACAAATGGCAGTATTCATCCACAATTTTAAAAGAAAAGGGGGGATTG
GGGGGTACAGTGCAGGGGAAAGAATAGTAGACATAATAGCAACAGACATACAAACT
AAAGAATTACAAAAACAAATTACAAAAATTCAAAATTTTCGGGTTTATTACAGGGAC
AGCAGAGATCCACTTTGGGCTCGAGGGGGCCCGGGTGCAAAGATGGATAAAGTTTTA
AACAGAGAGGAATCTTTGCAGCTAATGGACCTTCTAGGTCTTGAAAGGAGTGGGAAT
TGGCTCCGGTGCCCGTCAGTGGGCAGAGCGCACATCGCCCACAGTCCCCGAGAAGTT
GGGGGGAGGGGTCGGCAATTGATCCGGTGCCTAGAGAAGGTGGCGCGGGGTAAACTG
GGAAAGTGATGTCGTGTACTGGCTCCGCCTTTTTCCCGAGGGTGGGGGAGAACCGTAT
ATAAGTGCAGTAGTCGCCGTGAACGTTCTTTTTCGCAACGGGTTTGCCGCCAGAACAC
AGGTAAGTGCCGTGTGTGGTTCCCGCGGGCCTGGCCTCTTTACGGGTTATGGCCCTTG
CGTGCCTTGAATTACTTCCACCTGGCTGCAGTACGTGATTCTTGATCCCGAGCTTCGGG
TTGGAAGTGGGTGGGAGAGTTCGAGGCCTTGCGCTTAAGGAGCCCCTTCGCCTCGTGC
TTGAGTTGAGGCCTGGCCTGGGCGCTGGGGCCGCCGCGTGCGAATCTGGTGGCACCTT
CGCGCCTGTCTCGCTGCTTTCGATAAGTCTCTAGCCATTTAAAATTTTTGATGACCTGC
TGCGACGCTTTTTTTCTGGCAAGATAGTCTTGTAAATGCGGGCCAAGATCTGCACACT
GGTATTTCGGTTTTTGGGGCCGCGGGCGGCGACGGGGCCCGTGCGTCCCAGCGCACAT
GTTCGGCGAGGCGGGGCCTGCGAGCGCGGCCACCGAGAATCGGACGGGGGTAGTCTC
AAGCTGGCCGGCCTGCTCTGGTGCCTGGCCTCGCGCCGCCGTGTATCGCCCCGCCCTG
GGCGGCAAGGCTGGCCCGGTCGGCACCAGTTGCGTGAGCGGAAAGATGGCCGCTTCC
CGGCCCTGCTGCAGGGAGCTCAAAATGGAGGACGCGGCGCTCGGGAGAGCGGGCGG
GTGAGTCACCCACACAAAGGAAAAGGGCCTTTCCGTCCTCAGCCGTCGCTTCATGTGA
CTCCACGGAGTACCGGGCGCCGTCCAGGCACCTCGATTAGTTCTCGAGCTTTTGGAGT
ACGTCGTCTTTAGGTTGGGGGGAGGGGTTTTATGCGATGGAGTTTCCCCACACTGAGT
GGGTGGAGACTGAAGTTAGGCCAGCTTGGCACTTGATGTAATTCTCCTTGGAATTTGC
CCTTTTTGAGTTTGGATCTTGGTTCATTCTCAAGCCTCAGACAGTGGTTCAAAGTTTTT
TTCTTCCATTTCAGGTGTCGTGACGTACGGCCACCATGACCGAGTACAAGCCCACGGT
GCGCCTCGCCACCCGCGACGACGTCCCCAGGGCCGTACGCACCCTCGCCGCCGCGTTC
GCCGACTACCCCGCCACGCGCCACACCGTCGATCCGGACCGCCACATCGAGCGGGTC
ACCGAGCTGCAAGAACTCTTCCTCACGCGCGTCGGGCTCGACATCGGCAAGGTGTGG
GTCGCGGACGACGGCGCCGCCGTGGCGGTCTGGACCACGCCGGAGAGCGTCGAAGCG
GGGGCGGTGTTCGCCGAGATCGGCCCGCGCATGGCCGAGTTGAGCGGTTCCCGGCTG
GCCGCGCAGCAACAGATGGAAGGCCTCCTGGCGCCGCACCGGCCCAAGGAGCCCGCG
TGGTTCCTGGCCACCGTCGGAGTCTCGCCCGACCACCAGGGCAAGGGTCTGGGCAGC
GCCGTCGTGCTCCCCGGAGTGGAGGCGGCCGAGCGCGCCGGGGTGCCCGCCTTCCTG
GAGACCTCCGCGCCCCGCAACCTCCCCTTCTACGAGCGGCTCGGCTTCACCGTCACCG
CCGACGTCGAGGTGCCCGAAGGACCGCGCACCTGGTGCATGACCCGCAAGCCCGGTG
CCTGAACGCGTTAAGTCGACAATCAACCTCTGGATTACAAAATTTGTGAAAGATTGAC
TGGTATTCTTAACTATGTTGCTCCTTTTACGCTATGTGGATACGCTGCTTTAATGCCTTT
GTATCATGCTATTGCTTCCCGTATGGCTTTCATTTTCTCCTCCTTGTATAAATCCTGGTT
GCTGTCTCTTTATGAGGAGTTGTGGCCCGTTGTCAGGCAACGTGGCGTGGTGTGCACT
GTGTTTGCTGACGCAACCCCCACTGGTTGGGGCATTGCCACCACCTGTCAGCTCCTTTC
CGGGACTTTCGCTTTCCCCCTCCCTATTGCCACGGCGGAACTCATCGCCGCCTGCCTTG
CCCGCTGCTGGACAGGGGCTCGGCTGTTGGGCACTGACAATTCCGTGGTGTTGTCGGG
GAAATCATCGTCCTTTCCTTGGCTGCTCGCCTGTGTTGCCACCTGGATTCTGCGCGGGA
CGTCCTTCTGCTACGTCCCTTCGGCCCTCAATCCAGCGGACCTTCCTTCCCGCGGCCTG
CTGCCGGCTCTGCGGCCTCTTCCGCGTCTTCGCCTTCGCCCTCAGACGAGTCGGATCTC
CCTTTGGGCCGCCTCCCCGCGTCGACTTTAAGACCAATGACTTACAAGGCAGCTGTAG
ATCTTAGCCACTTTTTAAAAGAAAAGGGGGGACTGGAAGGGCTAATTCACTCCCAAC
GAAGACAAGATCTGCTTTTTGCTTGTACTGGGTCTCTCTGGTTAGACCAGATCTGAGC
CTGGGAGCTCTCTGGCTAACTAGGGAACCCACTGCTTAAGCCTCAATAAAGCTTGCCT
TGAGTGCTTCAAGTAGTGTGTGCCCGTCTGTTGTGTGACTCTGGTAACTAGAGATCCC
TCAGACCCTTTTAGTCAGTGTGGAAAATCTCTAGCAGTACGTATAGTAGTTCATGTCA
TCTTATTATTCAGTATTTATAACTTGCAAAGAAATGAATATCAGAGAGTGAGAGGAAC
TTGTTTATTGCAGCTTATAATGGTTACAAATAAAGCAATAGCATCACAAATTTCACAA
ATAAAGCATTTTTTTCACTGCATTCTAGTTGTGGTTTGTCCAAACTCATCAATGTATCT
TATCATGTCTGGCTCTAGCTATCCCGCCCCTAACTCCGCCCATCCCGCCCCTAACTCCG
CCCAGTTCCGCCCATTCTCCGCCCCATGGCTGACTAATTTTTTTTATTTATGCAGAGGC
CGAGGCCGCCTCGGCCTCTGAGCTATTCCAGAAGTAGTGAGGAGGCTTTTTTGGAGGC
CTAGGGACGTACCCAATTCGCCCTATAGTGAGTCGTATTACGCGCGCTCACTGGCCGT
CGTTTTACAACGTCGTGACTGGGAAAACCCTGGCGTTACCCAACTTAATCGCCTTGCA
GCACATCCCCCTTTCGCCAGCTGGCGTAATAGCGAAGAGGCCCGCACCGATCGCCCTT
CCCAACAGTTGCGCAGCCTGAATGGCGAATGGGACGCGCCCTGTAGCGGCGCATTAA
GCGCGGCGGGTGTGGTGGTTACGCGCAGCGTGACCGCTACACTTGCCAGCGCCCTAG
CGCCCGCTCCTTTCGCTTTCTTCCCTTCCTTTCTCGCCACGTTCGCCGGCTTTCCCCGTC
AAGCTCTAAATCGGGGGCTCCCTTTAGGGTTCCGATTTAGTGCTTTACGGCACCTCGA
CCCCAAAAAACTTGATTAGGGTGATGGTTCACGTAGTGGGCCATCGCCCTGATAGACG
GTTTTTCGCCCTTTGACGTTGGAGTCCACGTTCTTTAATAGTGGACTCTTGTTCCAAAC
TGGAACAACACTCAACCCTATCTCGGTCTATTCTTTTGATTTATAAGGGATTTTGCCGA
TTTCGGCCTATTGGTTAAAAAATGAGCTGATTTAACAAAAATTTAACGCGAATTTTAA
CAAAATATTAACGCTTACAATTTAGGTGGCACTTTTCGGGGAAATGTGCGCGGAACCC
CTATTTGTTTATTTTTCTAAATACATTCAAATATGTATCCGCTCATGAGACAATAACCC
TGATAAATGCTTCAATAATATTGAAAAAGGAAGAGTATGAGTATTCAACATTTCCGTG
TCGCCCTTATTCCCTTTTTTGCGGCATTTTGCCTTCCTGTTTTTGCTCACCCAGAAACGC
TGGTGAAAGTAAAAGATGCTGAAGATCAGTTGGGTGCACGAGTGGGTTACATCGAAC
TGGATCTCAACAGCGGTAAGATCCTTGAGAGTTTTCGCCCCGAAGAACGTTTTCCAAT
GATGAGCACTTTTAAAGTTCTGCTATGTGGCGCGGTATTATCCCGTATTGACGCCGGG
CAAGAGCAACTCGGTCGCCGCATACACTATTCTCAGAATGACTTGGTTGAGTACTCAC
CAGTCACAGAAAAGCATCTTACGGATGGCATGACAGTAAGAGAATTATGCAGTGCTG
CCATAACCATGAGTGATAACACTGCGGCCAACTTACTTCTGACAACGATCGGAGGAC
CGAAGGAGCTAACCGCTTTTTTGCACAACATGGGGGATCATGTAACTCGCCTTGATCG
TTGGGAACCGGAGCTGAATGAAGCCATACCAAACGACGAGCGTGACACCACGATGCC
TGTAGCAATGGCAACAACGTTGCGCAAACTATTAACTGGCGAACTACTTACTCTAGCT
TCCCGGCAACAATTAATAGACTGGATGGAGGCGGATAAAGTTGCAGGACCACTTCTG
CGCTCGGCCCTTCCGGCTGGCTGGTTTATTGCTGATAAATCTGGAGCCGGTGAGCGTG
GGTCTCGCGGTATCATTGCAGCACTGGGGCCAGATGGTAAGCCCTCCCGTATCGTAGT
TATCTACACGACGGGGAGTCAGGCAACTATGGATGAACGAAATAGACAGATCGCTGA
GATAGGTGCCTCACTGATTAAGCATTGGTAACTGTCAGACCAAGTTTACTCATATATA
CTTTAGATTGATTTAAAACTTCATTTTTAATTTAAAAGGATCTAGGTGAAGATCCTTTT
TGATAATCTCATGACCAAAATCCCTTAACGTGAGTTTTCGTTCCACTGAGCGTCAGAC
CCCGTAGAAAAGATCAAAGGATCTTCTTGAGATCCTTTTTTTCTGCGCGTAATCTGCTG
CTTGCAAACAAAAAAACCACCGCTACCAGCGGTGGTTTGTTTGCCGGATCAAGAGCT
ACCAACTCTTTTTCCGAAGGTAACTGGCTTCAGCAGAGCGCAGATACCAAATACTGTT
CTTCTAGTGTAGCCGTAGTTAGGCCACCACTTCAAGAACTCTGTAGCACCGCCTACAT
ACCTCGCTCTGCTAATCCTGTTACCAGTGGCTGCTGCCAGTGGCGATAAGTCGTGTCTT
ACCGGGTTGGACTCAAGACGATAGTTACCGGATAAGGCGCAGCGGTCGGGCTGAACG
GGGGGTTCGTGCACACAGCCCAGCTTGGAGCGAACGACCTACACCGAACTGAGATAC
CTACAGCGTGAGCTATGAGAAAGCGCCACGCTTCCCGAAGGGAGAAAGGCGGACAG
GTATCCGGTAAGCGGCAGGGTCGGAACAGGAGAGCGCACGAGGGAGCTTCCAGGGG
GAAACGCCTGGTATCTTTATAGTCCTGTCGGGTTTCGCCACCTCTGACTTGAGCGTCG
ATTTTTGTGATGCTCGTCAGGGGGGCGGAGCCTATGGAAAAACGCCAGCAACGCGGC
CTTTTTACGGTTCCTGGCCTTTTGCTGGCCTTTTGCTCACATGTTCTTTCCTGCGTTATC
CCCTGATTCTGTGGATAACCGTATTACCGCCTTTGAGTGAGCTGATACCGCTCGCCGC
AGC
pFC14K (Promega)
(SEQ ID NO: 71)
TCAATATTGGCCATTAGCCATATTATTCATTGGTTATATAGCATAAATCAATATTGGCT
ATTGGCCATTGCATACGTTGTATCTATATCATAATATGTACATTTATATTGGCTCATGT
CCAATATGACCGCCATGTTGGCATTGATTATTGACTAGTTATTAATAGTAATCAATTA
CGGGGTCATTAGTTCATAGCCCATATATGGAGTTCCGCGTTACATAACTTACGGTAAA
TGGCCCGCCTGGCTGACCGCCCAACGACCCCCGCCCATTGACGTCAATAATGACGTAT
GTTCCCATAGTAACGCCAATAGGGACTTTCCATTGACGTCAATGGGTGGAGTATTTAC
GGTAAACTGCCCACTTGGCAGTACATCAAGTGTATCATATGCCAAGTCCGCCCCCTAT
TGACGTCAATGACGGTAAATGGCCCGCCTGGCATTATGCCCAGTACATGACCTTACGG
GACTTTCCTACTTGGCAGTACATCTACGTATTAGTCATCGCTATTACCATGGTGATGCG
GTTTTGGCAGTACACCAATGGGCGTGGATAGCGGTTTGACTCACGGGGATTTCCAAGT
CTCCACCCCATTGACGTCAATGGGAGTTTGTTTTGGCACCAAAATCAACGGGACTTTC
CAAAATGTCGTAATAACCCCGCCCCGTTGACGCAAATGGGCGGTAGGCGTGTACGGT
GGGAGGTCTATATAAGCAGAGCTGGTTTAGTGAACCGTCAGATCACTAGAAGCTTTAT
TGCGGTAGTTTATCACAGTTAAATTGCTAACGCAGTCAGTGCTTCTGACACAACAGTC
TCGAACTTAAGCTGCAGAAGTTGGTCGTGAGGCACTGGGCAGGTAAGTATCAAGGTT
ACAAGACAGGTTTAAGGAGACCAATAGAAACTGGGCTTGTCGAGACAGAGAAGACTC
TTGCGTTTCTGATAGGCACCTATTGGTCTTACTGACATCCACTTTGCCTTTCTCTCCAC
AGGTGTCCACTCCCAGTTCAATTACAGCTCTTAAGGCTAGAGTATTAATACGACTCAC
TATAGGGCTAGCGATCGCCATGGAATAAGTAAGGAATCCACATGGCACAGGTTATCA
ACACGTTTGACGGGGTTGCGGATTATCTTCAGACATATCATAAGCTACCTGATAATTA
CATTACAAAATCAGAAGCACAAGCCCTCGGCTGGGTGGCATCAAAAGGGAACCTTGC
AGACGTCGCTCCGGGGAAAAGCATCGGCGGAGACATCTTCTCAAACAGGGAAGGCAA
ACTCCCGGGCAAAAGCGGACGAACATGGCGTGAAGCGGATATTAACTATACATCAGG
CTTCAGAAATTCAGACCGGATTCTTTACTCAAGCGACTGGCTGATTTACAAAACAACG
GACCATTATCAGACCTTTACAAAAATCAGATAATGTTTAATGACCCCGTGTCGAGCTC
TCGAGCCAACCACTGAGGATCTGTACTTTCAGAGCGATAACGATGGATCCGAAATCG
GTACTGGCTTTCCATTCGACCCCCATTATGTGGAAGTCCTGGGCGAGCGCATGCACTA
CGTCGATGTTGGTCCGCGCGATGGCACCCCTGTGCTGTTCCTGCACGGTAACCCGACC
TCCTCCTACGTGTGGCGCAACATCATCCCGCATGTTGCACCGACCCATCGCTGCATTG
CTCCAGACCTGATCGGTATGGGCAAATCCGACAAACCAGACCTGGGTTATTTCTTCGA
CGACCACGTCCGCTTCATGGATGCCTTCATCGAAGCCCTGGGTCTGGAAGAGGTCGTC
CTGGTCATTCACGACTGGGGCTCCGCTCTGGGTTTCCACTGGGCCAAGCGCAATCCAG
AGCGCGTCAAAGGTATTGCATTTATGGAGTTCATCCGCCCTATCCCGACCTGGGACGA
ATGGCCAGAATTTGCCCGCGAGACCTTCCAGGCCTTCCGCACCACCGACGTCGGCCGC
AAGCTGATCATCGATCAGAACGTTTTTATCGAGGGTACGCTGCCGATGGGTGTCGTCC
GCCCGCTGACTGAAGTCGAGATGGACCATTACCGCGAGCCGTTCCTGAATCCTGTTGA
CCGCGAGCCACTGTGGCGCTTCCCAAACGAGCTGCCAATCGCCGGTGAGCCAGCGAA
CATCGTCGCGCTGGTCGAAGAATACATGGACTGGCTGCACCAGTCCCCTGTCCCGAAG
CTGCTGTTCTGGGGCACCCCAGGCGTTCTGATCCCACCGGCCGAAGCCGCTCGCCTGG
CCAAAAGCCTGCCTAACTGCAAGGCTGTGGACATCGGCCCGGGTCTGAATCTGCTGCA
AGAAGACAACCCGGACCTGATCGGCAGCGAGATCGCGCGCTGGCTGTCTACTCTGGA
GATTTCCGGTTAATAGAATTCTAGAGTCGACCTGCAGGCATGCAAGCTGATCCGGCTG
CTAACAAAGCCCGAAAGGAAGCTGAGTTGGCTGCTGCCACCGCTGAGCAATAACTAG
CATAACCCCTTGGGGCGGCCGCTTCGAGCAGACATGATAAGATACATTGATGAGTTTG
GACAAACCACAACTAGAATGCAGTGAAAAAAATGCTTTATTTGTGAAATTTGTGATGC
TATTGCTTTATTTGTAACCATTATAAGCTGCAATAAACAAGTTAACAACAACAATTGC
ATTCATTTTATGTTTCAGGTTCAGGGGGAGATGTGGGAGGTTTTTTTAAGCAAGTAAA
ACCTCTACAAATGTGGTAAAATCGAATTCTAATGGATCCTCTTTGCGCTTGCGTTTTCC
CTTGTCCAGATAGCCCAGTAGCTGACATTCATCCGGGGTCAGCACCGTTTCTGCGGAC
TGGCTTTCTACGTGTTCCGCTTCCTTTAGCAGCCCTTGCGCCCTGAGTGCTTGCGGCAG
CGTGAGCTTCAAAAGAATTGCCAGCTGGGGCGCCCTCTGGTAAGGTTGGGAAGCCCT
GCAAAGTAAACTGGATGGCTTTCTTGCCGCCAAGGATCTGATGGCGCAGGGGATCAA
GATCTGATCAAGAGACAGGATGACGGTCGTTTCGCATGCTTGAACAAGATGGATTGC
ACGCAGGTTCTCCGGCCGCTTGGGTGGAGAGGCTATTCGGCTATGACTGGGCACAAC
AGACAATCGGCTGCTCTGATGCCGCCGTGTTCCGGCTGTCAGCGCAGGGGCGCCCGGT
TCTTTTTGTCAAGACCGACCTGTCCGGTGCCCTGAATGAACTGCAGGACGAGGCAGCG
CGGCTATCGTGGCTGGCCACGACGGGCGTTCCTTGCGCAGCTGTGCTCGACGTTGTCA
CTGAAGCGGGAAGGGACTGGCTGCTATTGGGCGAAGTGCCGGGGCAGGATCTCCTGT
CATCTCACCTTGCTCCTGCCGAGAAAGTATCCATCATGGCTGATGCAATGCGGCGGCT
GCATACGCTTGATCCGGCTACCTGCCCATTCGACCACCAAGCGAAACATCGCATCGAG
CGAGCACGCACTCGGATGGAAGCCGGTCTTGTCGATCAGGATGATCTGGACGAAGAG
CATCAGGGGCTCGCGCCAGCCGAACTGTTCGCCAGGCTCAAGGCGCGTATGCCGGAT
GGTGAGGATCTCGTCGTGACTCATGGCGATGCCTGCTTGCCGAATATCATGGTGGAAA
ATGGCCGCTTTTCTGGATTCATCGACTGTGGCCGGCTGGGTGTGGCGGACCGCTATCA
GGACATAGCGTTGGCTACCCGTGATATTGCTGAAGAGCTTGGCGGCGAATGGGCTGA
CCGCTTCCTCGTGCTTTACGGTATCGCCGCTCCCGATTCGCAGCGCATCGCCTTCTATC
GCCTTCTTGACGAGTTCTTCTGAGCGGGACTCTGGGGTTCGAAATGACCGACCAAGCG
ACGCCCAACCGGTATCAGCTCACTCAAAGGCGGTAATACGGTTATCCACAGAATCAG
GGGATAACGCAGGAAAGAACATGTGAGCAAAAGGCCAGCAAAAGGCCAGGAACCGT
AAAAAGGCCGCGTTGCTGGCGTTTTTCCATAGGCTCCGCCCCCCTGACGAGCATCACA
AAAATCGACGCTCAAGTCAGAGGTGGCGAAACCCGACAGGACTATAAAGATACCAGG
CGTTTCCCCCTGGAAGCTCCCTCGTGCGCTCTCCTGTTCCGACCCTGCCGCTTACCGGA
TACCTGTCCGCCTTTCTCCCTTCGGGAAGCGTGGCGCTTTCTCATAGCTCACGCTGTAG
GTATCTCAGTTCGGTGTAGGTCGTTCGCTCCAAGCTGGGCTGTGTGCACGAACCCCCC
GTTCAGCCCGACCGCTGCGCCTTATCCGGTAACTATCGTCTTGAGTCCAACCCGGTAA
GACACGACTTATCGCCACTGGCAGCAGCCACTGGTAACAGGATTAGCAGAGCGAGGT
ATGTAGGCGGTGCTACAGAGTTCTTGAAGTGGTGGCCTAACTACGGCTACACTAGAA
GGACAGTATTTGGTATCTGCGCTCTGCTGAAGCCAGTTACCTTCGGAAAAAGAGTTGG
TAGCTCTTGATCCGGCAAACAAACCACCGCTGGTAGCGGTGGTTTTTTTGTTTGCAAG
CAGCAGATTACGCGCAGAAAAAAAGGATTTCAAGAAGATCCTTTGATCTTTTCTACGG
GGTCTGACGCTCAGTGGAACGAAAACTCACGTTAAGGGATTTTGGTCATGAGATTATC
AAAAAGGATCTTCACCTAGATCCTTTTATAGTCCGGAAATACAGGAACGCACGCTGG
ATGGCCCTTCGCTGGGATGGTGAAACCATGAAAAATGGCAGCTTCAGTGGATTAAGT
GGGGGTAATGTGGCCTGTACCCTCTGGTTGCATAGGTATTCATACGGTTAAAATTTAT
CAGGCGCGATTGCGGCAGTTTTTCGGGTGGTTTGTTGCCATTTTTACCTGTCTGCTGCC
GTGATCGCGCTGAACGCGTTTTAGCGGTGCGTACAATTAAGGGATTATGGTAAATCCA
CTTACTGTCTGCCCTCGTAGCCATCGAGATAAACCGCAGTACTCCGGCCACGATGCGT
CCGGCGTAGAGGATCGAGATCT
pFC32K (Promega)
(SEQ ID NO: 72)
TCAATATTGGCCATTAGCCATATTATTCATTGGTTATATAGCATAAATCAATATTGGCT
ATTGGCCATTGCATACGTTGTATCTATATCATAATATGTACATTTATATTGGCTCATGT
CCAATATGACCGCCATGTTGGCATTGATTATTGACTAGTTATTAATAGTAATCAATTA
CGGGGTCATTAGTTCATAGCCCATATATGGAGTTCCGCGTTACATAACTTACGGTAAA
TGGCCCGCCTGGCTGACCGCCCAACGACCCCCGCCCATTGACGTCAATAATGACGTAT
GTTCCCATAGTAACGCCAATAGGGACTTTCCATTGACGTCAATGGGTGGAGTATTTAC
GGTAAACTGCCCACTTGGCAGTACATCAAGTGTATCATATGCCAAGTCCGCCCCCTAT
TGACGTCAATGACGGTAAATGGCCCGCCTGGCATTATGCCCAGTACATGACCTTACGG
GACTTTCCTACTTGGCAGTACATCTACGTATTAGTCATCGCTATTACCATGGTGATGCG
GTTTTGGCAGTACACCAATGGGCGTGGATAGCGGTTTGACTCACGGGGATTTCCAAGT
CTCCACCCCATTGACGTCAATGGGAGTTTGTTTTGGCACCAAAATCAACGGGACTTTC
CAAAATGTCGTAATAACCCCGCCCCGTTGACGCAAATGGGCGGTAGGCGTGTACGGT
GGGAGGTCTATATAAGCAGAGCTGGTTTAGTGAACCGTCAGATCACTAGAAGCTTTAT
TGCGGTAGTTTATCACAGTTAAATTGCTAACGCAGTCAGTGCTTCTGACACAACAGTC
TCGAACTTAAGCTGCAGAAGTTGGTCGTGAGGCACTGGGCAGGTAAGTATCAAGGTT
ACAAGACAGGTTTAAGGAGACCAATAGAAACTGGGCTTGTCGAGACAGAGAAGACTC
TTGCGTTTCTGATAGGCACCTATTGGTCTTACTGACATCCACTTTGCCTTTCTCTCCAC
AGGTGTCCACTCCCAGTTCAATTACAGCTCTTAAGGCTAGAGTATTAATACGACTCAC
TATAGGGCTAGCGATCGCCATGGAATAAGTAAGGAATCCACATGGCACAGGTTATCA
ACACGTTTGACGGGGTTGCGGATTATCTTCAGACATATCATAAGCTACCTGATAATTA
CATTACAAAATCAGAAGCACAAGCCCTCGGCTGGGTGGCATCAAAAGGGAACCTTGC
AGACGTCGCTCCGGGGAAAAGCATCGGCGGAGACATCTTCTCAAACAGGGAAGGCAA
ACTCCCGGGCAAAAGCGGACGAACATGGCGTGAAGCGGATATTAACTATACATCAGG
CTTCAGAAATTCAGACCGGATTCTTTACTCAAGCGACTGGCTGATTTACAAAACAACG
GACCATTATCAGACCTTTACAAAAATCAGATAATGTTTAATGACCCCGTGTCGAGCTC
TCGGCTCGAGCGGCGTCTTCACACTCGAAGATTTCGTTGGGGACTGGCGACAGACAGC
CGGCTACAACCTGGACCAAGTCCTTGAACAGGGAGGTGTGTCCAGTTTGTTTCAGAAT
CTCGGGGTGTCCGTAACTCCGATCCAAAGGATTGTCCTGAGCGGTGAAAATGGGCTG
AAGATCGACATCCATGTCATCATCCCGTATGAAGGTCTGAGCGGCGACCAAATGGGC
CAGATCGAAAAAATTTTTAAGGTGGTGTACCCTGTGGATGATCATCACTTTAAGGTGA
TCCTGCACTATGGCACACTGGTAATCGACGGGGTTACGCCGAACATGATCGACTATTT
CGGACGGCCGTATGAAGGCATCGCCGTGTTCGACGGCAAAAAGATCACTGTAACAGG
GACCCTGTGGAACGGCAACAAAATTATCGACGAGCGCCTGATCAACCCCGACGGCTC
CCTGCTGTTCCGAGTAACCATCAACGGAGTGACCGGCTGGCGGCTGTGCGAACGCATT
CTGGCGTAAGGCCGCGACTCTAGAGTCGACCTGCAGGCATGCAAGCTGATCCGGCTG
CTAACAAAGCCCGAAAGGAAGCTGAGTTGGCTGCTGCCACCGCTGAGCAATAACTAG
CATAACCCCTTGGGGCGGCCGCTTCGAGCAGACATGATAAGATACATTGATGAGTTTG
GACAAACCACAACTAGAATGCAGTGAAAAAAATGCTTTATTTGTGAAATTTGTGATGC
TATTGCTTTATTTGTAACCATTATAAGCTGCAATAAACAAGTTAACAACAACAATTGC
ATTCATTTTATGTTTCAGGTTCAGGGGGAGATGTGGGAGGTTTTTTTAAGCAAGTAAA
ACCTCTACAAATGTGGTAAAATCGAATTTTAACAAAATATTAACGCTTACAATTTCCT
GATGCGGTATTTTCTCCTTACGCATCTGTGCGGTATTTCACACCGCATACGCGGATCTG
CGCAGCACCATGGCCTGAAATAACCTCTGAAAGAGGAACTTGGTTAGGTACCTTCTGA
GGCGGAAAGAACCAGCTGTGGAATGTGTGTCAGTTAGGGTGTGGAAAGTCCCCAGGC
TCCCCAGCAGGCAGAAGTATGCAAAGCATGCATCTCAATTAGTCAGCAACCAGGTGT
GGAAAGTCCCCAGGCTCCCCAGCAGGCAGAAGTATGCAAAGCATGCATCTCAATTAG
TCAGCAACCATAGTCCCGCCCCTAACTCCGCCCATCCCGCCCCTAACTCCGCCCAGTT
CCGCCCATTCTCCGCCCCATGGCTGACTAATTTTTTTTATTTATGCAGAGGCCGAGGCC
GCCTCGGCCTCTGAGCTATTCCAGAAGTAGTGAGGAGGCTTTTTTGGAGGCCTAGGCT
TTTGCAAAAAGCTTAATTAACTGTTGACAATTAATCATCGGCATAGTATATCGGCATA
GTATAATACGACAAGGTGAGGAACTAAACCCAGGAGGCAGATCATGATTGAACAAGA
TGGATTGCACGCAGGTTCTCCGGCCGCTTGGGTGGAGAGGCTATTCGGCTATGACTGG
GCACAACAGACAATCGGCTGCTCTGATGCCGCCGTGTTCCGGCTGTCAGCGCAGGGG
CGCCCGGTTCTTTTTGTCAAGACCGACCTGTCCGGTGCCCTGAATGAACTGCAGGACG
AGGCAGCGCGGCTATCGTGGCTGGCCACGACGGGCGTTCCTTGCGCAGCTGTGCTCGA
CGTTGTCACTGAAGCGGGAAGGGACTGGCTGCTATTGGGCGAAGTGCCGGGGCAGGA
TCTCCTGTCATCTCACCTTGCTCCTGCCGAGAAAGTATCCATCATGGCTGATGCAATGC
GGCGGCTGCATACGCTTGATCCGGCTACCTGCCCATTCGACCACCAAGCGAAACATCG
CATCGAGCGAGCACGTACTCGGATGGAAGCCGGTCTTGTCGATCAGGATGATCTGGA
CGAAGAGCATCAGGGGCTCGCGCCAGCCGAACTGTTCGCCAGGCTCAAGGCGCGCAT
GCCCGACGGCGAGGATCTCGTCGTGACCCATGGCGATGCCTGCTTGCCGAATATCATG
GTGGAAAATGGCCGCTTTTCTGGATTCATCGACTGTGGCCGGCTGGGTGTGGCGGACC
GCTATCAGGACATAGCGTTGGCTACCCGTGATATTGCTGAAGAGCTTGGCGGCGAATG
GGCTGACCGCTTCCTCGTGCTTTACGGTATCGCCGCTCCCGATTCGCAGCGCATCGCCT
TCTATCGCCTTCTTGACGAGTTCTTCTGAGCGGGACTCTGGGGTTCGAAATGACCGAC
CAAGCGACGCCCAACCTGCCATCACGATGGCCGCAATAAAATATCTTTATTTTCATTA
CATCTGTGTGTTGGTTTTTTGTGTGAATCGATAGCGATAAGGATCCTCTTTGCGCTTGC
GTTTTCCCTTGTCCAGATAGCCCAGTAGCTGACATTCATCCGGGGTCAGCACCGTTTCT
GCGGACTGGCTTTCTACCCGGTATCAGCTCACTCAAAGGCGGTAATACGGTTATCCAC
AGAATCAGGGGATAACGCAGGAAAGAACATGTGAGCAAAAGGCCAGCAAAAGGCCA
GGAACCGTAAAAAGGCCGCGTTGCTGGCGTTTTTCCATAGGCTCCGCCCCCCTGACGA
GCATCACAAAAATCGACGCTCAAGTCAGAGGTGGCGAAACCCGACAGGACTATAAAG
ATACCAGGCGTTTCCCCCTGGAAGCTCCCTCGTGCGCTCTCCTGTTCCGACCCTGCCGC
TTACCGGATACCTGTCCGCCTTTCTCCCTTCGGGAAGCGTGGCGCTTTCTCATAGCTCA
CGCTGTAGGTATCTCAGTTCGGTGTAGGTCGTTCGCTCCAAGCTGGGCTGTGTGCACG
AACCCCCCGTTCAGCCCGACCGCTGCGCCTTATCCGGTAACTATCGTCTTGAGTCCAA
CCCGGTAAGACACGACTTATCGCCACTGGCAGCAGCCACTGGTAACAGGATTAGCAG
AGCGAGGTATGTAGGCGGTGCTACAGAGTTCTTGAAGTGGTGGCCTAACTACGGCTA
CACTAGAAGGACAGTATTTGGTATCTGCGCTCTGCTGAAGCCAGTTACCTTCGGAAAA
AGAGTTGGTAGCTCTTGATCCGGCAAACAAACCACCGCTGGTAGCGGTGGTTTTTTTG
TTTGCAAGCAGCAGATTACGCGCAGAAAAAAAGGATTTCAAGAAGATCCTTTGATCTT
TTCTACGGGGTCTGACGCTCAGTGGAACGAAAACTCACGTTAAGGGATTTTGGTCATG
AGATTATCAAAAAGGATCTTCACCTAGATCCTTTTATAGTCCGGAAATACAGGAACGC
ACGCTGGATGGCCCTTCGCTGGGATGGTGAAACCATGAAAAATGGCAGCTTCAGTGG
ATTAAGTGGGGGTAATGTGGCCTGTACCCTCTGGTTGCATAGGTATTCATACGGTTAA
AATTTATCAGGCGCGATTGCGGCAGTTTTTCGGGTGGTTTGTTGCCATTTTTACCTGTC
TGCTGCCGTGATCGCGCTGAACGCGTTTTAGCGGTGCGTACAATTAAGGGATTATGGT
AAATCCACTTACTGTCTGCCCTCGTAGCCATCGAGATAAACCGCAGTACTCCGGCCAC
GATGCGTCCGGCGTAGAGGATCGAGATCT

For BRET dimerization experiments, genes were cloned into pFC14K or pFC32K vectors at Sgf1/EcoICRI restriction sites per manufacturer directions (Promega). For degradation eGFP/mCherry reporter experiments, genes were cloned into the Artichoke vector at BsmBI restriction sites (see FIG. 9).

FMC63-CD28-IKZF3: This sequence corresponds to the anti-CD19 scFv FMC63 (www.ebi.ac.uk/ena/data/view/ADM64594), the CD28 hinge, transmembrane, and cytoplasmic domains (amino acids 114-220), and IKZF3 amino acids 130-189.

(SEQ ID NO: 73)
MLLLVTSLLLCELPHPAFLLIPDIQMTQTTSSLSASLGDRVTISCRASQD
ISKYLNWYQQKPDGTVKLLIYHTSRLHSGVPSRFSGSGSGTDYSLTISNL
EQEDIATYFCQQGNTLPYTEGGGTKLEITGSTSGSGKPGSGEGSTKGEVK
LQESGPGLVAPSQSLSVTCTVSGVSLPDYGVSWIRQPPRKGLEWLGVIWG
SETTYYNSALKSRLTIIKDNSKSQVFLKMNSLQTDDTAIYYCAKHYYYGG
SYAMDYWGQGTSVTVSSAAAIEVMYPPPYLDNEKSNGTIIHVKGKHLCPS
PLFPGPSKPFWVLVVVGGVLACYSLLVTVAFIIFWVRSKRSRLLHSDYMN
MTPRRPGPTRKHYQPYAPPRDFAAYRSSGFNVLMVHKRSHTGERPFQCNQ
CGASFTQKGNLLRHIKLHTGEKPFKCHLCNYACQRRDAL

FMC63-CD28-IKZF3 intracellular K0 (iK0): This sequence corresponds to the anti-CD19 scFv FMC63 (www.ebi.ac.uk/ena/data/view/ADM64594), the CD28 hinge, transmembrane, and cytoplasmic domains (amino acids 114-220), and IKZF3 amino acids 130-189, wherein all intracellular lysines are substituted to arginine.

(SEQ ID NO: 74)
MLLLVTSLLLCELPHPAFLLIPDIQMTQTTSSLSASLGDRVTISCRASQD
ISKYLNWYQQKPDGTVKLLIYHTSRLHSGVPSRFSGSGSGTDYSLTISNL
EQEDIATYFCQQGNTLPYTFGGGTKLEITGSTSGSGKPGSGEGSTKGEVK
LQESGPGLVAPSQSLSVTCTVSGVSLPDYGVSWIRQPPRKGLEWLGVIWG
SETTYYNSALKSRLTIIKDNSKSQVFLKMNSLQTDDTAIYYCAKHYYYGG
SYAMDYWGQGTSVTVSSAAAIEVMYPPPYLDNEKSNGTIIHVKGKHLCPS
PLFPGPSKPFWVLVVVGGVLACYSLLVTVAFIIFWVRSRRSRLLHSDYMN
MTPRRPGPTRRHYQPYAPPRDFAAYRSSGFNVLMVHRRSHTGERPFQCNQ
CGASFTQRGNLLRHIRLHTGERPFRCHLCNYACQRRDAL

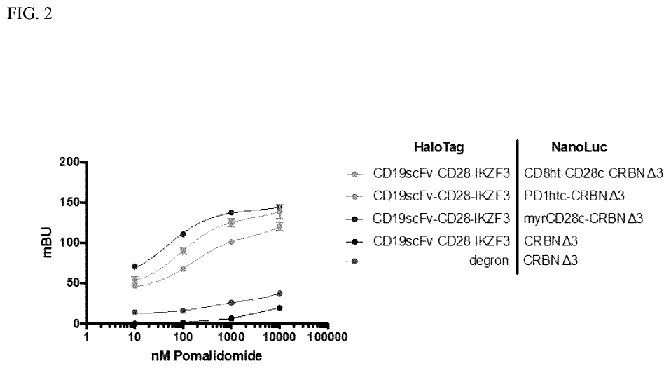

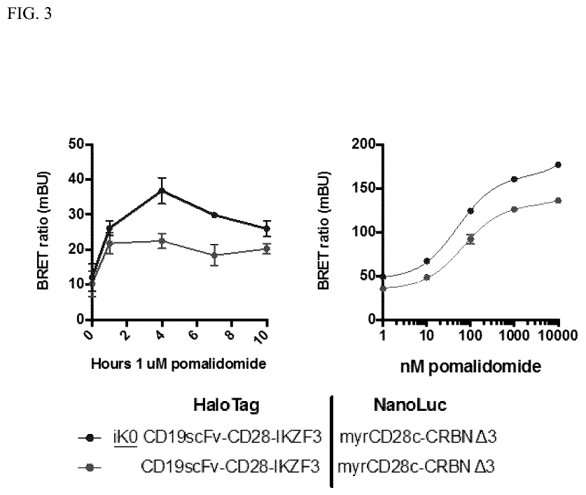

The following 3 constructs were the polypeptide sequences used in FIG. 2 and FIG. 3. They differed from other split CAR designs in that they lacked a CD3ζ chain (and have been confirmed to dimerize).

Split CAR component B1 (BRET): The sequence corresponds to the fusion of the CD8 alpha signal sequence (amino acids 1-21), hinge and transmembrane domains (amino acids 138-206), CD28 co-stimulatory domain (amino acids 180-220), a G4S linker, and minCRBN3 (see SEQ ID NO: 3).

(SEQ ID NO: 75)
MALPVTALLLPLALLLHAARPTTTPAPRPPTPAPTIASQPLSLRPEACRP
AAGGAVHTRGLDFACDIYIWAPLAGTCGVLLLSLVITLYCRSKRSRLLHS
DYMNMTPRRPGPTRKHYQPYAPPRDFAAYRSGGGGSAGEGDQQDAAHNMG
NHLPLLPESEEEDEMEVEDQDSKEAKKPNIINFDTSLPTSHTYLGADMEE
FHGRTLHDDDSCQVIPVLPQVMMILIPGQTLPLQLFHPQEVSMVRNLIQK
DRTFAVLAYSNVQEREAQFGTTAEIYAYREEQDFGIEIVKVKAIGRQRFK
VLELRTQSDGIQQAKVQILPECVLPSTYDAETLMDRIKKQLREWDENLKD
DSLPSNPIDFSYRVAACLPIDDVLRIQLLKIGSAIQRLRCELDIMNKCTS
LCCKQCQETEITTKNEIFSLSLCGPMAAYVNPHGYVHETLTVYKACNLNL
IGRPSTEHSWFPGYAWTVAQCKICASHIGWKFTATKKDMSPQKFWGLTRS
ALLPTIPDTEDEISPDKVILCL

Split CAR component B2 (BRET): The sequence corresponds to the fusion of the LYN palmitoylation/myristoylation domain, CD28 co-stimulatory domain (amino acids 180-220), a G4S linker, and minCRBN3 (see SEQ ID NO: 3).

(SEQ ID NO: 76)
MGCIKSKRKDNLNDDGVDMKTRSKRSRLLHSDYMNMTPRRPGPTRKHYQP
YAPPRDFAAYRSGGGGSAGEGDQQDAAHNMGNHLPLLPESEEEDEMEVED
QDSKEAKKPNIINFDTSLPTSHTYLGADMEEFHGRTLHDDDSCQVIPVLP
QVMMILIPGQTLPLQLFHPQEVSMVRNLIQKDRTFAVLAYSNVQEREAQF
GTTAEIYAYREEQDFGIEIVKVKAIGRQRFKVLELRTQSDGIQQAKVQIL
PECVLPSTYDAETLMDRIKKQLREWDENLKDDSLPSNPIDFSYRVAACLP
IDDVLRIQLLKIGSAIQRLRCELDIMNKCTSLCCKQCQETEITTKNEIFS
LSLCGPMAAYVNPHGYVHETLTVYKACNLNLIGRPSTEHSWFPGYAWTVA
QCKICASHIGWKFTATKKDMSPQKFWGLTRSALLPTIPDTEDEISPDKVI
LCL

Split CAR component 3 (BRET): The sequence corresponds to the fusion of the CD8 alpha signal sequence (amino acids 1-21), PD1 hinge, transmembrane, and intracellular domains, with the substitutions Y223F and Y248F (amino acids 146-288), and minCRBN3 (see SEQ ID NO: 3).

(SEQ ID NO: 77)
MALPVTALLLPLALLLHAARPERRAEVPTAHPSPSPRPAGQFQTLVVGVV
GGLLGSLVLLVWVLAVICSRAARGTIGARRTGQPLKEDPSAVPVFSVDFG
ELDFQWREKTPEPPVPCVPEQTEFATIVFPSGMGTSSPARRGSADGPRSA
QPLRPEDGHCSWPLGGGGSAGEGDQQDAAHNMGNHLPLLPESEEEDEMEV
EDQDSKEAKKPNIINFDTSLPTSHTYLGADMEEFHGRTLHDDDSCQVIPV
LPQVMMILIPGQTLPLQLFHPQEVSMVRNLIQKDRTFAVLAYSNVQEREA
QFGTTAEIYAYREEQDFGIEIVKVKAIGRQRFKVLELRTQSDGIQQAKVQ
ILPECVLPSTYDAETLMDRIKKQLREWDENLKDDSLPSNPIDFSYRVAAC
LPIDDVLRIQLLKIGSAIQRLRCELDIMNKCTSLCCKQCQETEITTKNEI
FSLSLCGPMAAYVNPHGYVHETLTVYKACNLNLIGRPSTEHSWFPGYAWT
VAQCKICASHIGWKFTATKKDMSPQKFWGLTRSALLPTIPDTEDEISPDK
VILCL

Split CAR component B1 (complete): The sequence corresponds to the fusion of the CD8 alpha signal sequence (amino acids 1-21), hinge and transmembrane domains (amino acids 138-206), CD28 co-stimulatory domain (amino acids 180-220), a G4S linker, minCRBN3 (see SEQ ID NO: 3), and the intracellular portion of CD3z (amino acids 55-164).

(SEQ ID NO: 78)
MALPVTALLLPLALLLHAARPTTTPAPRPPTPAPTIASQPLSLRPEACRP
AAGGAVHTRGLDFACDIYIWAPLAGTCGVLLLSLVITLYCRSKRSRLLHS
DYMNMTPRRPGPTRKHYQPYAPPRDFAAYRSGGGGSAGEGDQQDAAHNMG
NHLPLLPESEEEDEMEVEDQDSKEAKKPNIINFDTSLPTSHTYLGADMEE
FHGRTLHDDDSCQVIPVLPQVMMILIPGQTLPLQLFHPQEVSMVRNLIQK
DRTFAVLAYSNVQEREAQFGTTAEIYAYREEQDFGIEIVKVKAIGRQRFK
VLELRTQSDGIQQAKVQILPECVLPSTYDAETLMDRIKKQLREWDENLKD
DSLPSNPIDFSYRVAACLPIDDVLRIQLLKIGSAIQRLRCELDIMNKCTS
LCCKQCQETEITTKNEIFSLSLCGPMAAYVNPHGYVHETLTVYKACNLNL
IGRPSTEHSWFPGYAWTVAQCKICASHIGWKFTATKKDMSPQKFWGLTRS
ALLPTIPDTEDEISPDKVILCLSLGFSRSADAPAYQQGQNQLYNELNLGR
REEYDVLDKRRGRDPEMGGKPQRRKNPQEGLYNELQKDKMAEAYSEIGMK
GERRRGKGHDGLYQGLSTATKDTYDALHMQALPPR

Split CAR component B2 (complete): The sequence corresponds to the fusion of the LYN palmitoylation/myristoylation domain, CD28 co-stimulatory domain (amino acids 180-220), a G4S linker, minCRBN3 (see SEQ ID NO: 3), and the intracellular portion of CD3z (amino acids 55-164)

(SEQ ID NO: 79)
MGCIKSKRKDNLNDDGVDMKTRSKRSRLLHSDYMNMTPRRPGPTRKHYQP
YAPPRDFAAYRSGGGGSAGEGDQQDAAHNMGNHLPLLPESEEEDEMEVED
QDSKEAKKPNIINFDTSLPTSHTYLGADMEEFHGRTLHDDDSCQVIPVLP
QVMMILIPGQTLPLQLFHPQEVSMVRNLIQKDRTFAVLAYSNVQEREAQF
GTTAEIYAYREEQDFGIEIVKVKAIGRQRFKVLELRTQSDGIQQAKVQIL
PECVLPSTYDAETLMDRIKKQLREWDENLKDDSLPSNPIDFSYRVAACLP
IDDVLRIQLLKIGSAIQRLRCELDIMNKCTSLCCKQCQETEITTKNEIFS
LSLCGPMAAYVNPHGYVHETLTVYKACNLNLIGRPSTEHSWFPGYAWTVA
QCKICASHIGWKFTATKKDMSPQKFWGLTRSALLPTIPDTEDEISPDKVI
LCLSLGFSRSADAPAYQQGQNQLYNELNLGRREEYDVLDKRRGRDPEMGG
KPQRRKNPQEGLYNELQKDKMAEAYSEIGMKGERRRGKGHDGLYQGLSTA
TKDTYDALHMQALPPR

Split CAR component 3 (complete): The sequence corresponds to the fusion of the CD8 alpha signal sequence (amino acids 1-21), PD1 hinge, transmembrane, and intracellular domains, with the substitutions Y223F and Y248F (amino acids 146-288), minCRBN3 (see SEQ ID NO: 3), and the intracellular portion of CD3z (amino acids 55-164).

(SEQ ID NO: 80)
MALPVTALLLPLALLLHAARPERRAEVPTARPSPSPRPAGQFQTLVVGVV
GGLLGSLVLLVWVLAVICSRAARGTIGARRTGQPLKEDPSAVPVFSVDFG
ELDFQWREKTPEPPVPCVPEQTEFATIVFPSGMGTSSPARRGSADGPRSA
QPLRPEDGHCSWPLGGGGSAGEGDQQDAAHNMGNHLPLLPESEEEDEMEV
EDQDSKEAKKPNIINFDTSLPTSHTYLGADMEEFHGRTLHDDDSCQVIPV
LPQVMMILIPGQTLPLQLFHPQEVSMVRNLIQKDRTFAVLAYSNVQEREA
QFGTTAEIYAYREEQDFGIEIVKVKAIGRQRFKVLELRTQSDGIQQAKVQ
ILPECVLPSTYDAETLMDRIKKQLREWDENLKDDSLPSNPIDFSYRVAAC
LPIDDVLRIQLLKIGSAIQRLRCELDIMNKCTSLCCKQCQETEITTKNEI
FSLSLCGPMAAYVNPHGYVHETLTVYKACNLNLIGRPSTEHSWFPGYAWT
VAQCKICASHIGWKFTATKKDMSPQKFWGLTRSALLPTIPDTEDEISPDK
VILCLSLGFSRSADAPAYQQGQNQLYNELNLGRREEYDVLDKRRGRDPEM
GGKPQRRKNPQEGLYNELQKDKMAEAYSEIGMKGERRRGKGHDGLYQGLS
TATKDTYDALHMQALPPR

FMC63-CD28-CD3z-IKZF3: This sequence corresponds to the in frame fusion of the CSF2RA signal sequence (amino acids 1-22), anti-CD19 scFv FMC63 (www.ebi.ac.uk/ena/data/view/ADM64594), the CD28 hinge, transmembrane, and cytoplasmic domains (amino acids 114-220), the CD3z intracellular ITAM domains (amino acids 55-164), a SG linker, and IKZF3 amino acids 130-189.

(SEQ ID NO: 81)
MLLLVTSLLLCELPHPAFLLIPEQKLISEEDLDIQMTQTTSSLSASLGDR
VTISCRASQDISKYLNWYQQKPDGTVKLLIYHTSRLHSGVPSRFSGSGSG
TDYSLTISNLEQEDIATYFCQQGNTLPYTFGGGTKLEITGSTSGSGKPGS
GEGSTKGEVKLQESGPGLVAPSQSLSVTCTVSGVSLPDYGVSWIRQPPRK
GLEWLGVIWGSETTYYNSALKSRLTIIKDNSKSQVFLKMNSLQTDDTAIY
YCAKHYYYGGSYAMDYWGQGTSVTVSSAAAIEVMYPPPYLDNEKSNGTII
HVKGKHLCPSPLFPGPSKPFWVLVVVGGVLACYSLLVTVAFIIFWVRSKR
SRLLHSDYMNMTPRRPGPTRKHYQPYAPPRDFAAYRSRVKFSRSADAPAY
QQGQNQLYNELNLGRREEYDVLDKRRGRDPEMGGKPRRKNPQEGLYNELQ
KDKMAEAYSEIGMKGERRRGKGHDGLYQGLSTATKDTYDALHMQALPPRS
GFNVLMVHKRSHTGERPFQCNQCGASFTQKGNLLRHIKLHTGEKPFKCHL
CNYACQRRDAL

Proof of concept fusion polypeptides of the IKZF3 degron (amino acids 130-189) that retained IMiD-dependent degradation when fused to the C termini of CD28 and CD3z are included below:

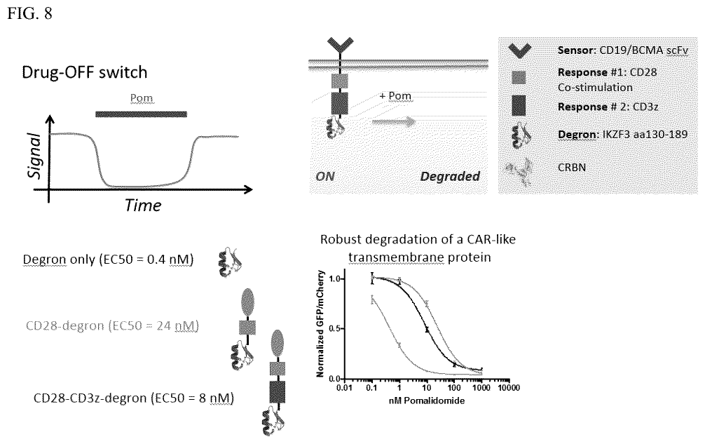

CD28-IKZF3: This sequence corresponds to the in frame fusion of full length CD28, a SG linker, and IKZF3 (amino acids 130-189), relating to FIG. 8:

(SEQ ID NO: 82)
MLRLLLALNLFPSIQVTGNKILVKQSPMLVAYDNAVNLSCKYSYNLFSRE
FRASLHKGLDSAVEVCVVYGNYSQQLQVYSKTGFNCDGKLGNESVTFYLQ
NLYVNQTDIYFCKIEVMYPPPYLDNEKSNGTIIHVKGKHLCPSPLFPGPS
KPFWVLVVVGGVLACYSLLVTVAFIIFWVRSKRSRLLHSDYMNMTPRRPG
PTRKHYQPYAPPRDFAAYRSSGFNVLMVHKRSHTGERPFQCNQCGASFTQ
KGNLLRHIKLHTGEKPFKCHLCNYACQRRDAL

CD28-CD3z-IKZF3: This sequence corresponds to the in frame fusion of full length CD28, the intracellular domain of CD3z (amino acids 55-164), and IKZF3 (amino acids 130-189), relating to FIG. 8:

(SEQ ID NO: 83)
MLRLLLALNLFPSIQVTGNKILVKQSPMLVAYDNAVNLSCKYSYNLFSRE
FRASLHKGLDSAVEVCVVYGNYSQQLQVYSKTGFNCDGKLGNESVTFYLQ
NLYVNQTDIYFCKIEVMYPPPYLDNEKSNGTIIHVKGKHLCPSPLFPGPS
KPFWVLVVVGGVLACYSLLVTVAFIIFWVRSKRSRLLHSDYMNMTPRRPG
PTRKHYQPYAPPRDFAAYRSFSRSADAPAYQQGQNQLYNELNLGRREEYD
VLDKRRGRDPEMGGKPQRRKNPQEGLYNELQKDKMAEAYSEIGMKGERRR
GKGHDGLYQGLSTATKDTYDALHMQALPPRSGFNVLMVHKRSHTGERPFQ
CNQCGASFTQKGNLLRHIKLHTGEKPFKCHLCNYACQRRDAL

Other exemplary sequences include: Chimeric Degradable Inhibitor LAT-CSK-IKZF3: The sequence corresponds to the fusion of the LAT Signal-Anchor (amino acids 1-33), CSK (full length, amino acids 1-450), and IKZF3 degron (amino acids 130-189).

(SEQ ID NO: 84)
ATGGAGGAGGCCATCCTGGTCCCCTGCGTGCTGGGGCTCCTGCTGCTGC
CCATCCTGGCCATGTTGATGGCACTGTGTGTGCACTGCCACAGACTGCC
AATGTCAGCAATACAGGCCGCCTGGCCATCCGGTACAGAATGTATTGCC
AAGTACAACTTCCACGGCACTGCCGAGCAGGACCTGCCCTTCTGCAAAG
GgGACGTGCTCACCATTGTGGCCGTCACCAAGGACCCCAACTGGTACAA
AGCCAAAAACAAGGTGGGCCGTGAGGGCATCATCCCAGCCAACTACGTC
CAGAAGCGGGAGGGCGTGAAGGCGGGTACCAAACTCAGCCTCATGCCTT
GGTTCCACGGCAAGATCACACGGGAGCAGGCTGAGCGGCTTCTGTACCC
GCCGGAGACAGGCCTGTTCCTGGTGCGGGAGAGCACCAACTACCCCGGA
GACTACACGCTGTGCGTGAGCTGCGACGGCAAGGTGGAGCACTACCGCA
TCATGTACCATGCCAGCAAGCTCAGCATCGACGAGGAGGTGTACTTTGA
GAACCTCATGCAGCTGGTGGAGCACTACACCTCAGACGCAGATGGACTC
TGTACGCGCCTCATTAAACCAAAGGTCATGGAGGGCACAGTGGCGGCCC
AGGATGAGTTCTACCGCAGCGGCTGGGCCCTGAACATGAAGGAGCTGAA
GCTGCTGCAGACCATCGGGAAGGGGGAGTTCGGgGACGTGATGCTGGGC
GATTACCGAGGGAACAAAGTCGCCGTCAAGTGCATTAAGAACGACGCCA
CTGCCCAGGCCTTCCTGGCTGAAGCCTCAGTCATGACGCAACTGCGGCA
TAGCAACCTGGTGCAGCTCCTGGGCGTGATCGTGGAGGAGAAGGGCGGG
CTCTACATCGTCACTGAGTACATGGCCAAGGGGAGCCTTGTGGACTACC
TGCGGTCTAGGGGTCGGTCAGTGCTGGGCGGAGACTGTCTCCTCAAGTT
CTCGCTAGATGTCTGCGAGGCCATGGAATACCTGGAGGGCAACAATTTC
GTGCATCGAGACCTGGCTGCCCGCAATGTGCTGGTGTCTGAGGACAACG
TGGCCAAGGTCAGCGACTTTGGTCTCACCAAGGAGGCGTCCAGCACCCA
GGACACGGGCAAGCTGCCAGTCAAGTGGACAGCCCCTGAGGCCCTGAGA
GAGAAGAAATTCTCCACTAAGTCTGACGTGTGGAGTTTCGGAATCCTTC
TCTGGGAAATCTACTCCTTTGGGCGAGTGCCTTATCCAAGAATTCCCCT
GAAGGACGTCGTCCCTCGGGTGGAGAAGGGCTACAAGATGGATGCCCCC
GACGGCTGCCCGCCCGCAGTCTATGAAGTCATGAAGAACTGCTGGCACC
TGGACGCCGCCATGCGGCCCTCCTTCCTACAGCTCCGAGAGCAGCTTGA
GCACATCAAAACCCACGAGCTGCACCTGTCCGGATTCAATGTCTTAATG
GTTCATAAGCGAAGCCATACTGGTGAACGCCCATTCCAGTGTAATCAGT
GTGGGGCATCTTTTACTCAGAAAGGTAACCTCCTCCGCCACATTAAACT
GCACACAGGGGAAAAACCTTTTAAGTGTCACCTCTGCAACTATGCATGC
CAAAGAAGAGATGCGCTC

Chimeric Degradable Inhibitor LAT-CSK(E154A)-IKZF3: The sequence corresponds to the fusion of the LAT Signal-Anchor (amino acids 1-33), CSK (full length, amino acids 1-450, substitution E154A), and IKZF3 degron (amino acids 130-189).

(SEQ ID NO: 85)
ATGGAGGAGGCCATCCTGGTCCCCTGCGTGCTGGGGCTCCTGCTGCTGC
CCATCCTGGCCATGTTGATGGCACTGTGTGTGCACTGCCACAGACTGCC
AATGTCAGCAATACAGGCCGCCTGGCCATCCGGTACAGAATGTATTGCC
AAGTACAACTTCCACGGCACTGCCGAGCAGGACCTGCCCTTCTGCAAAG
GgGACGTGCTCACCATTGTGGCCGTCACCAAGGACCCCAACTGGTACAA
AGCCAAAAACAAGGTGGGCCGTGAGGGCATCATCCCAGCCAACTACGTC
CAGAAGCGGGAGGGCGTGAAGGCGGGTACCAAACTCAGCCTCATGCCTT
GGTTCCACGGCAAGATCACACGGGAGCAGGCTGAGCGGCTTCTGTACCC
GCCGGAGACAGGCCTGTTCCTGGTGCGGGAGAGCACCAACTACCCCGGA
GACTACACGCTGTGCGTGAGCTGCGACGGCAAGGTGGAGCACTACCGCA
TCATGTACCATGCCAGCAAGCTCAGCATCGACGAGGAGGTGTACTTTGA
GAACCTCATGCAGCTGGTGGcGCACTACACCTCAGACGCAGATGGACTC
TGTACGCGCCTCATTAAACCAAAGGTCATGGAGGGCACAGTGGCGGCCC
AGGATGAGTTCTACCGCAGCGGCTGGGCCCTGAACATGAAGGAGCTGAA
GCTGCTGCAGACCATCGGGAAGGGGGAGTTCGGgGACGTGATGCTGGGC
GATTACCGAGGGAACAAAGTCGCCGTCAAGTGCATTAAGAACGACGCCA
CTGCCCAGGCCTTCCTGGCTGAAGCCTCAGTCATGACGCAACTGCGGCA
TAGCAACCTGGTGCAGCTCCTGGGCGTGATCGTGGAGGAGAAGGGCGGG
CTCTACATCGTCACTGAGTACATGGCCAAGGGGAGCCTTGTGGACTACC
TGCGGTCTAGGGGTCGGTCAGTGCTGGGCGGAGACTGTCTCCTCAAGTT
CTCGCTAGATGTCTGCGAGGCCATGGAATACCTGGAGGGCAACAATTTC
GTGCATCGAGACCTGGCTGCCCGCAATGTGCTGGTGTCTGAGGACAACG
TGGCCAAGGTCAGCGACTTTGGTCTCACCAAGGAGGCGTCCAGCACCCA
GGACACGGGCAAGCTGCCAGTCAAGTGGACAGCCCCTGAGGCCCTGAGA
GAGAAGAAATTCTCCACTAAGTCTGACGTGTGGAGTTTCGGAATCCTTC
TCTGGGAAATCTACTCCTTTGGGCGAGTGCCTTATCCAAGAATTCCCCT
GAAGGACGTCGTCCCTCGGGTGGAGAAGGGCTACAAGATGGATGCCCCC
GACGGCTGCCCGCCCGCAGTCTATGAAGTCATGAAGAACTGCTGGCACC
TGGACGCCGCCATGCGGCCCTCCTTCCTACAGCTCCGAGAGCAGCTTGA
GCACATCAAAACCCACGAGCTGCACCTGTCCGGATTCAATGTCTTAATG
GTTCATAAGCGAAGCCATACTGGTGAACGCCCATTCCAGTGTAATCAGT
GTGGGGCATCTTTTACTCAGAAAGGTAACCTCCTCCGCCACATTAAACT
GCACACAGGGGAAAAACCTTTTAAGTGTCACCTCTGCAACTATGCATGC
CAAAGAAGAGATGCGCTC

Chimeric Degradable Inhibitor LAT-CSK(W47A/R107K/E154A)-IKZF3: The sequence corresponds to the fusion of the LAT Signal-Anchor (amino acids 1-33), CSK (full length, amino acids 1-450, substitutions W47A, R107K, and E154A), and IKZF3 degron (amino acids 130-189).

(SEQ ID NO: 86)
ATGGAGGAGGCCATCCTGGTCCCCTGCGTGCTGGGGCTCCTGCTGCTGC
CCATCCTGGCCATGTTGATGGCACTGTGTGTGCACTGCCACAGACTGCC
AATGTCAGCAATACAGGCCGCCTGGCCATCCGGTACAGAATGTATTGCC
AAGTACAACTTCCACGGCACTGCCGAGCAGGACCTGCCCTTCTGCAAAG
GgGACGTGCTCACCATTGTGGCCGTCACCAAGGACCCCAACgcGTACAA
AGCCAAAAACAAGGTGGGCCGTGAGGGCATCATCCCAGCCAACTACGTC
CAGAAGCGGGAGGGCGTGAAGGCGGGTACCAAACTCAGCCTCATGCCTT
GGTTCCACGGCAAGATCACACGGGAGCAGGCTGAGCGGCTTCTGTACCC
GCCGGAGACAGGCCTGTTCCTGGTGaaGGAGAGCACCAACTACCCCGGA
GACTACACGCTGTGCGTGAGCTGCGACGGCAAGGTGGAGCACTACCGCA
TCATGTACCATGCCAGCAAGCTCAGCATCGACGAGGAGGTGTACTTTGA
GAACCTCATGCAGCTGGTGGcGCACTACACCTCAGACGCAGATGGACTC
TGTACGCGCCTCATTAAACCAAAGGTCATGGAGGGCACAGTGGCGGCCC
AGGATGAGTTCTACCGCAGCGGCTGGGCCCTGAACATGAAGGAGCTGAA
GCTGCTGCAGACCATCGGGAAGGGGGAGTTCGGgGACGTGATGCTGGGC
GATTACCGAGGGAACAAAGTCGCCGTCAAGTGCATTAAGAACGACGCCA
CTGCCCAGGCCTTCCTGGCTGAAGCCTCAGTCATGACGCAACTGCGGCA
TAGCAACCTGGTGCAGCTCCTGGGCGTGATCGTGGAGGAGAAGGGCGGG
CTCTACATCGTCACTGAGTACATGGCCAAGGGGAGCCTTGTGGACTACC
TGCGGTCTAGGGGTCGGTCAGTGCTGGGCGGAGACTGTCTCCTCAAGTT
CTCGCTAGATGTCTGCGAGGCCATGGAATACCTGGAGGGCAACAATTTC
GTGCATCGAGACCTGGCTGCCCGCAATGTGCTGGTGTCTGAGGACAACG
TGGCCAAGGTCAGCGACTTTGGTCTCACCAAGGAGGCGTCCAGCACCCA
GGACACGGGCAAGCTGCCAGTCAAGTGGACAGCCCCTGAGGCCCTGAGA
GAGAAGAAATTCTCCACTAAGTCTGACGTGTGGAGTTTCGGAATCCTTC
TCTGGGAAATCTACTCCTTTGGGCGAGTGCCTTATCCAAGAATTCCCCT
GAAGGACGTCGTCCCTCGGGTGGAGAAGGGCTACAAGATGGATGCCCCC
GACGGCTGCCCGCCCGCAGTCTATGAAGTCATGAAGAACTGCTGGCACC
TGGACGCCGCCATGCGGCCCTCCTTCCTACAGCTCCGAGAGCAGCTTGA
GCACATCAAAACCCACGAGCTGCACCTGTCCGGATTCAATGTCTTAATG
GTTCATAAGCGAAGCCATACTGGTGAACGCCCATTCCAGTGTAATCAGT
GTGGGGCATCTTTTACTCAGAAAGGTAACCTCCTCCGCCACATTAAACT
GCACACAGGGGAAAAACCTTTTAAGTGTCACCTCTGCAACTATGCATGC
CAAAGAAGAGATGCGCTC

Chimeric Degradable Inhibitor LAT-SHP1-IKZF3: The sequence corresponds to the fusion of the LAT Signal-Anchor (amino acids 1-33), SHP1 (amino acids 203-595), and IKZF3 degron (amino acids 130-189).

(SEQ ID NO: 87)
ATGGAGGAGGCCATCCTGGTCCCCTGCGTGCTGGGGCTCCTGCTGCTGC
CCATCCTGGCCATGTTGATGGCACTGTGTGTGCACTGCCACAGACTGCC
ATCAGGCGCCTTTGTCTACCTGCGGCAGCCGTACTATGCCACGAGGGTG
AATGCGGCTGACATTGAGAACCGAGTGTTGGAACTGAACAAGAAGCAGG
AGTCCGAGGATACAGCCAAGGCTGGCTTCTGGGAGGAGTTTGAGAGTTT
GCAGAAGCAGGAGGTGAAGAACTTGCACCAGCGTCTGGAAGGGCAGCGG
CCAGAGAACAAGGGCAAGAACCGCTACAAGAACATTCTCCCCTTTGACC
ACAGCCGAGTGATCCTGCAGGGACGGGACAGTAACATCCCCGGGTCCGA
CTACATCAATGCCAACTACATCAAGAACCAGCTGCTAGGCCCTGATGAG
AACGCTAAGACCTACATCGCCAGCCAGGGCTGTCTGGAGGCCACGGTCA
ATGACTTCTGGCAGATGGCGTGGCAGGAGAACAGCCGTGTCATCGTCAT
GACCACCCGAGAGGTGGAGAAAGGCCGGAACAAATGCGTCCCATACTGG
CCCGAGGTGGGCATGCAGCGTGCTTATGGGCCCTACTCTGTGACCAACT
GCGGGGAGCATGACACAACCGAATACAAACTCCGTACCTTACAGGTCTC
CCCGCTGGACAATGGAGACCTGATTCGGGAGATCTGGCATTACCAGTAC
CTGAGCTGGCCCGACCATGGGGTCCCCAGTGAGCCTGGGGGTGTCCTCA
GCTTCCTGGACCAGATCAACCAGCGGCAGGAAAGTCTGCCTCACGCAGG
GCCCATCATCGTGCACTGCAGCGCCGGCATCGGCCGCACAGGCACCATC
ATTGTCATCGACATGCTCATGGAGAACATCTCCACCAAGGGCCTGGACT
GTGACATTGACATCCAGAAGACCATCCAGATGGTGCGGGCGCAGCGCTC
GGGCATGGTGCAGACGGAGGCGCAGTACAAGTTCATCTACGTGGCCATC
GCCCAGTTCATTGAAACCACTAAGAAGAAGCTGGAGGTCCTGCAGTCGC
AGAAGGGCCAGGAGTCGGAGTACGGGAACATCACCTATCCCCCAGCCAT
GAAGAATGCCCATGCCAAGGCCTCCCGCACCTCGTCCAAACACAAGGAG
GATGTGTATGAGAACCTGCACACTAAGAACAAGAGGGAGGAGAAAGTGA
AGAAGCAGCGGTCAGCAGACAAGGAGAAGAGCAAGGGTTCCCTCAAGAG
GAAGTCCGGATTCAATGTCTTAATGGTTCATAAGCGAAGCCATACTGGT
GAACGCCCATTCCAGTGTAATCAGTGTGGGGCATCTTTTACTCAGAAAG
GTAACCTCCTCCGCCACATTAAACTGCACACAGGGGAAAAACCTTTTAA
GTGTCACCTCTGCAACTATGCATGCCAAAGAAGAGATGCGCTC

Split CAR component B1a (complete): The sequence corresponds to the fusion of the CD8 alpha signal sequence (amino acids 1-21), hinge and transmembrane domains (amino acids 138-206), CD28 co-stimulatory domain (amino acids 180-220), a G4S linker, minCRBN2 (see SEQ ID NO: 2), and the intracellular portion of CD3z (amino acids 55-164).

(SEQ ID NO: 88)
ATGGCGCTCCCAGTCACTGCCCTGCTTTTGCCCCTGGCACTTCTTCTTCACGCTGCCAG
ACCCACAACGACCCCAGCTCCACGCCCGCCGACTCCCGCGCCAACTATAGCCAGTCAGC
CCCTGTCACTGCGGCCGGAGGCGTGTCGCCCTGCAGCGGGGGGAGCCGTCCACACACGA
GGTCTTGACTTCGCCTGTGACATCTATATCTGGGCGCCTCTGGCCGGTACATGCGGCGT
GTTGTTGCTTAGCCTCGTGATAACACTCTATTGCAGGAGTAAGAGGAGCAGGCTCCTGC
ACAGTGACTACATGAACATGACTCCTAGAAGGCCTGGACCCACCCGCAAGCATTACCAG
CCCTATGCCCCACCACGCGACTTCGCAGCCTATCGCTCCGGAGGGGGTGGTTCTTGTAC
TTCCCTTTGCTGTAAACAATGTCAAGAAACAGAAATAACAACCAAAAATGAAATATTCA
GTTTATCCTTATGTGGGCCGATGGCAGCTTATGTGAATCCTCATGGATATGTGCATGAG
ACACTTACTGTGTATAAGGCTTGCAACTTGAATCTGATAGGCCGGCCTTCTACAGAACA
CAGCTGGTTTCCTGGGTATGCCTGGACTGTTGCCCAGTGTAAGATCTGTGCAAGCCATA
TTGGATGGAAGTTTACGGCCACCAAAAAAGACATGTCACCTCAAAAATTTTGGGGCTTA
ACGCGATCTGCTCTGTTGCCCACGATCCCAGACACTGAAGATGAAATAAGTCCAGACAA
AGTAATACTTTGCTTGTCTCTCGGCTTCAGTCGATCAGCAGATGCTCCAGCGTACCAGC
AAGGCCAGAACCAACTTTATAATGAACTGAATTTGGGCCGACGCGAGGAATACGACGTT
CTTGACAAGCGGAGGGGCCGCGATCCAGAAATGGGGGGGAAACCGCAACGAAGAAAGAA
TCCCCAGGAGGGGCTCTACAATGAACTTCAAAAGGATAAAATGGCAGAGGCCTATAGCG
AAATCGGTATGAAGGGGGAGCGAAGGCGAGGTAAGGGGCATGATGGGTTGTATCAAGGC
CTGTCCACGGCGACCAAGGATACCTATGACGCTCTTCACATGCAAGCATTGCCTCCTAG
A
“Eureka”: pEF1α-BsmBI cloning site-17aaRigidLinker-eGFP
(SEQ ID NO: 89)
TTTTGCACAACATGGGGGATCATGTAACTCGCCTTGATCGTTGGGAACCGGAGCTGAAT
GAAGCCATACCAAACGACGAGCGTGACACCACGATGCCTGTAGCAATGGCAACAACGTT
GCGCAAACTATTAACTGGCGAACTACTTACTCTAGCTTCCCGGCAACAATTAATAGACT
GGATGGAGGCGGATAAAGTTGCAGGACCACTTCTGCGCTCGGCCCTTCCGGCTGGCTGG
TTTATTGCTGATAAATCTGGAGCCGGTGAGCGTGGGTCTCGCGGTATCATTGCAGCACT
GGGGCCAGATGGTAAGCCCTCCCGTATCGTAGTTATCTACACGACGGGGAGTCAGGCAA
CTATGGATGAACGAAATAGACAGATCGCTGAGATAGGTGCCTCACTGATTAAGCATTGG
TAACTGTCAGACCAAGTTTACTCATATATACTTTAGATTGATTTAAAACTTCATTTTTA
ATTTAAAAGGATCTAGGTGAAGATCCTTTTTGATAATCTCATGACCAAAATCCCTTAAC
GTGAGTTTTCGTTCCACTGAGCGTCAGACCCCGTAGAAAAGATCAAAGGATCTTCTTGA
GATCCTTTTTTTCTGCGCGTAATCTGCTGCTTGCAAACAAAAAAACCACCGCTACCAGC
GGTGGTTTGTTTGCCGGATCAAGAGCTACCAACTCTTTTTCCGAAGGTAACTGGCTTCA
GCAGAGCGCAGATACCAAATACTGTTCTTCTAGTGTAGCCGTAGTTAGGCCACCACTTC
AAGAACTCTGTAGCACCGCCTACATACCTCGCTCTGCTAATCCTGTTACCAGTGGCTGC
TGCCAGTGGCGATAAGTCGTGTCTTACCGGGTTGGACTCAAGACGATAGTTACCGGATA
AGGCGCAGCGGTCGGGCTGAACGGGGGGTTCGTGCACACAGCCCAGCTTGGAGCGAACG
ACCTACACCGAACTGAGATACCTACAGCGTGAGCTATGAGAAAGCGCCACGCTTCCCGA
AGGGAGAAAGGCGGACAGGTATCCGGTAAGCGGCAGGGTCGGAACAGGAGAGCGCACGA
GGGAGCTTCCAGGGGGAAACGCCTGGTATCTTTATAGTCCTGTCGGGTTTCGCCACCTC
TGACTTGAGCGTCGATTTTTGTGATGCTCGTCAGGGGGGCGGAGCCTATGGAAAAACGC
CAGCAACGCGGCCTTTTTACGGTTCCTGGCCTTTTGCTGGCCTTTTGCTCACATGTTCT
TTCCTGCGTTATCCCCTGATTCTGTGGATAACCGTATTACCGCCTTTGAGTGAGCTGAT
ACCGCTCGCCGCAGCCGAACGACCGAGCGCAGCGAGTCAGTGAGCGAGGAAGCGGAAGA
GCGCCCAATACGCAAACCGCCTCTCCCCGCGCGTTGGCCGATTCATTAATGCAGCTGGC
ACGACAGGTTTCCCGACTGGAAAGCGGGCAGTGAGCGCAACGCAATTAATGTGAGTTAG
CTCACTCATTAGGCACCCCAGGCTTTACACTTTATGCTTCCGGCTCGTATGTTGTGTGG
AATTGTGAGCGGATAACAATTTCACACAGGAAACAGCTATGACCATGATTACGCCAAGC
GCGCAATTAACCCTCACTAAAGGGAACAAAAGCTGGAGCTGCAAGCTTAATGTAGTCTT
ATGCAATACTCTTGTAGTCTTGCAACATGGTAACGATGAGTTAGCAACATGCCTTACAA
GGAGAGAAAAAGCACCGTGCATGCCGATTGGTGGAAGTAAGGTGGTACGATCGTGCCTT
ATTAGGAAGGCAACAGACGGGTCTGACATGGATTGGACGAACCACTGAATTGCCGCATT
GCAGAGATATTGTATTTAAGTGCCTAGCTCGATACATAAACGGGTCTCTCTGGTTAGAC
CAGATCTGAGCCTGGGAGCTCTCTGGCTAACTAGGGAACCCACTGCTTAAGCCTCAATA
AAGCTTGCCTTGAGTGCTTCAAGTAGTGTGTGCCCGTCTGTTGTGTGACTCTGGTAACT
AGAGATCCCTCAGACCCTTTTAGTCAGTGTGGAAAATCTCTAGCAGTGGCGCCCGAACA
GGGACTTGAAAGCGAAAGGGAAACCAGAGGAGCTCTCTCGACGCAGGACTCGGCTTGCT
GAAGCGCGCACGGCAAGAGGCGAGGGGCGGCGACTGGTGAGTACGCCAAAAATTTTGAC
TAGCGGAGGCTAGAAGGAGAGAGATGGGTGCGAGAGCGTCAGTATTAAGCGGGGGAGAA
TTAGATCGCGATGGGAAAAAATTCGGTTAAGGCCAGGGGGAAAGAAAAAATATAAATTA
AAACATATAGTATGGGCAAGCAGGGAGCTAGAACGATTCGCAGTTAATCCTGGCCTGTT
AGAAACATCAGAAGGCTGTAGACAAATACTGGGACAGCTACAACCATCCCTTCAGACAG
GATCAGAAGAACTTAGATCATTATATAATACAGTAGCAACCCTCTATTGTGTGCATCAA
AGGATAGAGATAAAAGACACCAAGGAAGCTTTAGACAAGATAGAGGAAGAGCAAAACAA
AAGTAAGACCACCGCACAGCAAGCGGCCGCTGATCTTCAGACCTGGAGGAGGAGATATG
AGGGACAATTGGAGAAGTGAATTATATAAATATAAAGTAGTAAAAATTGAACCATTAGG
AGTAGCACCCACCAAGGCAAAGAGAAGAGTGGTGCAGAGAGAAAAAAGAGCAGTGGGAA
TAGGAGCTTTGTTCCTTGGGTTCTTGGGAGCAGCAGGAAGCACTATGGGCGCAGCGTCA
ATGACGCTGACGGTACAGGCCAGACAATTATTGTCTGGTATAGTGCAGCAGCAGAACAA
TTTGCTGAGGGCTATTGAGGCGCAACAGCATCTGTTGCAACTCACAGTCTGGGGCATCA
AGCAGCTCCAGGCAAGAATCCTGGCTGTGGAAAGATACCTAAAGGATCAACAGCTCCTG
GGGATTTGGGGTTGCTCTGGAAAACTCATTTGCACCACTGCTGTGCCTTGGAATGCTAG
TTGGAGTAATAAATCTCTGGAACAGATTTGGAATCACACGACCTGGATGGAGTGGGACA
GAGAAATTAACAATTACACAAGCTTAATACACTCCTTAATTGAAGAATCGCAAAACCAG
CAAGAAAAGAATGAACAAGAATTATTGGAATTAGATAAATGGGCAAGTTTGTGGAATTG
GTTTAACATAACAAATTGGCTGTGGTATATAAAATTATTCATAATGATAGTAGGAGGCT
TGGTAGGTTTAAGAATAGTTTTTGCTGTACTTTCTATAGTGAATAGAGTTAGGCAGGGA
TATTCACCATTATCGTTTCAGACCCACCTCCCAACCCCGAGGGGACCCTATTCCAGCAC
ATATGAGGCTTGGCGTAACTAGATCTTGAGACAAATGGCAGTATTCATCCACAATTTTA
AAAGAAAAGGGGGGATTGGGGGGTACAGTGCAGGGGAAAGAATAGTAGACATAATAGCA
ACAGACATACAAACTAAAGAATTACAAAAACAAATTACAAAAATTCAAAATTTTCGGGT
TTATTACAGGGACAGCAGAGATCCACTTTGGGCTCGAGGGGGCCCGGGTGCAAAGATGG
ATAAAGTTTTAAACAGAGAGGAATCTTTGCAGCTAATGGACCTTCTAGGTCTTGAAAGG
AGTGGGAATTGGCTCCGGTGCCCGTCAGTGGGCAGAGCGCACATCGCCCACAGTCCCCG
AGAAGTTGGGGGGAGGGGTCGGCAATTGATCCGGTGCCTAGAGAAGGTGGCGCGGGGTA
AACTGGGAAAGTGATGTCGTGTACTGGCTCCGCCTTTTTCCCGAGGGTGGGGGAGAACC
GTATATAAGTGCAGTAGTCGCCGTGAACGTTCTTTTTCGCAACGGGTTTGCCGCCAGAA
CACAGGTAAGTGCCGTGTGTGGTTCCCGCGGGCCTGGCCTCTTTACGGGTTATGGCCCT
TGCGTGCCTTGAATTACTTCCACCTGGCTGCAGTACGTGATTCTTGATCCCGAGCTTCG
GGTTGGAAGTGGGTGGGAGAGTTCGAGGCCTTGCGCTTAAGGAGCCCCTTCGCCTCGTG
CTTGAGTTGAGGCCTGGCCTGGGCGCTGGGGCCGCCGCGTGCGAATCTGGTGGCACCTT
CGCGCCTGTCTCGCTGCTTTCGATAAGTCTCTAGCCATTTAAAATTTTTGATGACCTGC
TGCGACGCTTTTTTTCTGGCAAGATAGTCTTGTAAATGCGGGCCAAGATCTGCACACTG
GTATTTCGGTTTTTGGGGCCGCGGGCGGCGACGGGGCCCGTGCGTCCCAGCGCACATGT
TCGGCGAGGCGGGGCCTGCGAGCGCGGCCACCGAGAATCGGACGGGGGTAGTCTCAAGC
TGGCCGGCCTGCTCTGGTGCCTGGCCTCGCGCCGCCGTGTATCGCCCCGCCCTGGGCGG
CAAGGCTGGCCCGGTCGGCACCAGTTGCGTGAGCGGAAAGATGGCCGCTTCCCGGCCCT
GCTGCAGGGAGCTCAAAATGGAGGACGCGGCGCTCGGGAGAGCGGGCGGGTGAGTCACC
CACACAAAGGAAAAGGGCCTTTCCGTCCTCAGCCGTCGCTTCATGTGACTCCACGGAGT
ACCGGGCGCCGTCCAGGCACCTCGATTAGTTCTCGAGCTTTTGGAGTACGTCGTCTTTA
GGTTGGGGGGAGGGGTTTTATGCGATGGAGTTTCCCCACACTGAGTGGGTGGAGACTGA
AGTTAGGCCAGCTTGGCACTTGATGTAATTCTCCTTGGAATTTGCCCTTTTTGAGTTTG
GATCTTGGTTCATTCTCAAGCCTCAGACAGTGGTTCAAAGTTTTTTTCTTCCATTTCAG
GTGTCGTGAATACCGTCGACTCCGGAATAGCCACCATGGAGACGGACGTCTCAGCTGAA
GCTGCTGCAAAGGAAGCTGCAGCTAAGGAGGCTGCAGCTAAGGCTGTGAGCAAGGGCGA
GGAGCTGTTCACCGGGGTGGTGCCCATCCTGGTCGAGCTGGACGGCGACGTAAACGGCC
ACAAGTTCAGCGTGTCCGGCGAGGGCGAGGGCGATGCCACCTACGGCAAGCTGACCCTG
AAGTTCATCTGCACCACCGGCAAGCTGCCCGTGCCCTGGCCCACCCTCGTGACCACCCT
GACCTACGGCGTGCAGTGCTTCAGCCGCTACCCCGACCACATGAAGCAGCACGACTTCT
TCAAGTCCGCCATGCCCGAAGGCTACGTCCAGGAGCGCACCATCTTCTTCAAGGACGAC
GGCAACTACAAGACCCGCGCCGAGGTGAAGTTCGAGGGCGACACCCTGGTGAACCGCAT
CGAGCTGAAGGGCATCGACTTCAAGGAGGACGGCAACATCCTGGGGCACAAGCTGGAGT
ACAACTACAACAGCCACAACGTCTATATCATGGCCGACAAGCAGAAGAACGGCATCAAG
GTGAACTTCAAGATCCGCCACAACATCGAGGACGGCAGCGTGCAGCTCGCCGACCACTA
CCAGCAGAACACCCCCATCGGCGACGGCCCCGTGCTGCTGCCCGACAACCACTACCTGA
GCACCCAGTCCGCCCTGAGCAAAGACCCCAACGAGAAGCGCGATCACATGGTCCTGCTG
GAGTTCGTGACCGCCGCCGGGATCACTCTCGGCATGGACGAGCTGTACAAGTAAATGCA
TGAGTAACTGAGGATCCAGGGACAGCAGAGATACGCGTTAAGTCGACAATCAACCTCTG
GATTACAAAATTTGTGAAAGATTGACTGGTATTCTTAACTATGTTGCTCCTTTTACGCT
ATGTGGATACGCTGCTTTAATGCCTTTGTATCATGCTATTGCTTCCCGTATGGCTTTCA
TTTTCTCCTCCTTGTATAAATCCTGGTTGCTGTCTCTTTATGAGGAGTTGTGGCCCGTT
GTCAGGCAACGTGGCGTGGTGTGCACTGTGTTTGCTGACGCAACCCCCACTGGTTGGGG
CATTGCCACCACCTGTCAGCTCCTTTCCGGGACTTTCGCTTTCCCCCTCCCTATTGCCA
CGGCGGAACTCATCGCCGCCTGCCTTGCCCGCTGCTGGACAGGGGCTCGGCTGTTGGGC
ACTGACAATTCCGTGGTGTTGTCGGGGAAATCATCGTCCTTTCCTTGGCTGCTCGCCTG
TGTTGCCACCTGGATTCTGCGCGGGACGTCCTTCTGCTACGTCCCTTCGGCCCTCAATC
CAGCGGACCTTCCTTCCCGCGGCCTGCTGCCGGCTCTGCGGCCTCTTCCGCGTCTTCGC
CTTCGCCCTCAGACGAGTCGGATCTCCCTTTGGGCCGCCTCCCCGCGTCGACTTTAAGA
CCAATGACTTACAAGGCAGCTGTAGATCTTAGCCACTTTTTAAAAGAAAAGGGGGGACT
GGAAGGGCTAATTCACTCCCAACGAAGACAAGATCTGCTTTTTGCTTGTACTGGGTCTC
TCTGGTTAGACCAGATCTGAGCCTGGGAGCTCTCTGGCTAACTAGGGAACCCACTGCTT
AAGCCTCAATAAAGCTTGCCTTGAGTGCTTCAAGTAGTGTGTGCCCGTCTGTTGTGTGA
CTCTGGTAACTAGAGATCCCTCAGACCCTTTTAGTCAGTGTGGAAAATCTCTAGCAGTA
CGTATAGTAGTTCATGTCATCTTATTATTCAGTATTTATAACTTGCAAAGAAATGAATA
TCAGAGAGTGAGAGGAACTTGTTTATTGCAGCTTATAATGGTTACAAATAAAGCAATAG
CATCACAAATTTCACAAATAAAGCATTTTTTTCACTGCATTCTAGTTGTGGTTTGTCCA
AACTCATCAATGTATCTTATCATGTCTGGCTCTAGCTATCCCGCCCCTAACTCCGCCCA
TCCCGCCCCTAACTCCGCCCAGTTCCGCCCATTCTCCGCCCCATGGCTGACTAATTTTT
TTTATTTATGCAGAGGCCGAGGCCGCCTCGGCCTCTGAGCTATTCCAGAAGTAGTGAGG
AGGCTTTTTTGGAGGCCTAGGGACGTACCCAATTCGCCCTATAGTGAGTCGTATTACGC
GCGCTCACTGGCCGTCGTTTTACAACGTCGTGACTGGGAAAACCCTGGCGTTACCCAAC
TTAATCGCCTTGCAGCACATCCCCCTTTCGCCAGCTGGCGTAATAGCGAAGAGGCCCGC
ACCGATCGCCCTTCCCAACAGTTGCGCAGCCTGAATGGCGAATGGGACGCGCCCTGTAG
CGGCGCATTAAGCGCGGCGGGTGTGGTGGTTACGCGCAGCGTGACCGCTACACTTGCCA
GCGCCCTAGCGCCCGCTCCTTTCGCTTTCTTCCCTTCCTTTCTCGCCACGTTCGCCGGC
TTTCCCCGTCAAGCTCTAAATCGGGGGCTCCCTTTAGGGTTCCGATTTAGTGCTTTACG
GCACCTCGACCCCAAAAAACTTGATTAGGGTGATGGTTCACGTAGTGGGCCATCGCCCT
GATAGACGGTTTTTCGCCCTTTGACGTTGGAGTCCACGTTCTTTAATAGTGGACTCTTG
TTCCAAACTGGAACAACACTCAACCCTATCTCGGTCTATTCTTTTGATTTATAAGGGAT
TTTGCCGATTTCGGCCTATTGGTTAAAAAATGAGCTGATTTAACAAAAATTTAACGCGA
ATTTTAACAAAATATTAACGCTTACAATTTAGGTGGCACTTTTCGGGGAAATGTGCGCG
GAACCCCTATTTGTTTATTTTTCTAAATACATTCAAATATGTATCCGCTCATGAGACAA
TAACCCTGATAAATGCTTCAATAATATTGAAAAAGGAAGAGTATGAGTATTCAACATTT
CCGTGTCGCCCTTATTCCCTTTTTTGCGGCATTTTGCCTTCCTGTTTTTGCTCACCCAG
AAACGCTGGTGAAAGTAAAAGATGCTGAAGATCAGTTGGGTGCACGAGTGGGTTACATC
GAACTGGATCTCAACAGCGGTAAGATCCTTGAGAGTTTTCGCCCCGAAGAACGTTTTCC
AATGATGAGCACTTTTAAAGTTCTGCTATGTGGCGCGGTATTATCCCGTATTGACGCCG
GGCAAGAGCAACTCGGTCGCCGCATACACTATTCTCAGAATGACTTGGTTGAGTACTCA
CCAGTCACAGAAAAGCATCTTACGGATGGCATGACAGTAAGAGAATTATGCAGTGCTGC
CATAACCATGAGTGATAACACTGCGGCCAACTTACTTCTGACAACGATCGGAGGACCGA
AGGAGCTAACCGCTT
“Elk”: pEF1α-BsmBI cloning site-17aaRigidLinker-mTagBFP2
(SEQ ID NO: 90)
CTGTCAGACCAAGTTTACTCATATATACTTTAGATTGATTTAAAACTTCATTTTTAATT
TAAAAGGATCTAGGTGAAGATCCTTTTTGATAATCTCATGACCAAAATCCCTTAACGTG
AGTTTTCGTTCCACTGAGCGTCAGACCCCGTAGAAAAGATCAAAGGATCTTCTTGAGAT
CCTTTTTTTCTGCGCGTAATCTGCTGCTTGCAAACAAAAAAACCACCGCTACCAGCGGT
GGTTTGTTTGCCGGATCAAGAGCTACCAACTCTTTTTCCGAAGGTAACTGGCTTCAGCA
GAGCGCAGATACCAAATACTGTTCTTCTAGTGTAGCCGTAGTTAGGCCACCACTTCAAG
AACTCTGTAGCACCGCCTACATACCTCGCTCTGCTAATCCTGTTACCAGTGGCTGCTGC
CAGTGGCGATAAGTCGTGTCTTACCGGGTTGGACTCAAGACGATAGTTACCGGATAAGG
CGCAGCGGTCGGGCTGAACGGGGGGTTCGTGCACACAGCCCAGCTTGGAGCGAACGACC
TACACCGAACTGAGATACCTACAGCGTGAGCTATGAGAAAGCGCCACGCTTCCCGAAGG
GAGAAAGGCGGACAGGTATCCGGTAAGCGGCAGGGTCGGAACAGGAGAGCGCACGAGGG
AGCTTCCAGGGGGAAACGCCTGGTATCTTTATAGTCCTGTCGGGTTTCGCCACCTCTGA
CTTGAGCGTCGATTTTTGTGATGCTCGTCAGGGGGGCGGAGCCTATGGAAAAACGCCAG
CAACGCGGCCTTTTTACGGTTCCTGGCCTTTTGCTGGCCTTTTGCTCACATGTTCTTTC
CTGCGTTATCCCCTGATTCTGTGGATAACCGTATTACCGCCTTTGAGTGAGCTGATACC
GCTCGCCGCAGCCGAACGACCGAGCGCAGCGAGTCAGTGAGCGAGGAAGCGGAAGAGCG
CCCAATACGCAAACCGCCTCTCCCCGCGCGTTGGCCGATTCATTAATGCAGCTGGCACG
ACAGGTTTCCCGACTGGAAAGCGGGCAGTGAGCGCAACGCAATTAATGTGAGTTAGCTC
ACTCATTAGGCACCCCAGGCTTTACACTTTATGCTTCCGGCTCGTATGTTGTGTGGAAT
TGTGAGCGGATAACAATTTCACACAGGAAACAGCTATGACCATGATTACGCCAAGCGCG
CAATTAACCCTCACTAAAGGGAACAAAAGCTGGAGCTGCAAGCTTAATGTAGTCTTATG
CAATACTCTTGTAGTCTTGCAACATGGTAACGATGAGTTAGCAACATGCCTTACAAGGA
GAGAAAAAGCACCGTGCATGCCGATTGGTGGAAGTAAGGTGGTACGATCGTGCCTTATT
AGGAAGGCAACAGACGGGTCTGACATGGATTGGACGAACCACTGAATTGCCGCATTGCA
GAGATATTGTATTTAAGTGCCTAGCTCGATACATAAACGGGTCTCTCTGGTTAGACCAG
ATCTGAGCCTGGGAGCTCTCTGGCTAACTAGGGAACCCACTGCTTAAGCCTCAATAAAG
CTTGCCTTGAGTGCTTCAAGTAGTGTGTGCCCGTCTGTTGTGTGACTCTGGTAACTAGA
GATCCCTCAGACCCTTTTAGTCAGTGTGGAAAATCTCTAGCAGTGGCGCCCGAACAGGG
ACTTGAAAGCGAAAGGGAAACCAGAGGAGCTCTCTCGACGCAGGACTCGGCTTGCTGAA
GCGCGCACGGCAAGAGGCGAGGGGCGGCGACTGGTGAGTACGCCAAAAATTTTGACTAG
CGGAGGCTAGAAGGAGAGAGATGGGTGCGAGAGCGTCAGTATTAAGCGGGGGAGAATTA
GATCGCGATGGGAAAAAATTCGGTTAAGGCCAGGGGGAAAGAAAAAATATAAATTAAAA
CATATAGTATGGGCAAGCAGGGAGCTAGAACGATTCGCAGTTAATCCTGGCCTGTTAGA
AACATCAGAAGGCTGTAGACAAATACTGGGACAGCTACAACCATCCCTTCAGACAGGAT
CAGAAGAACTTAGATCATTATATAATACAGTAGCAACCCTCTATTGTGTGCATCAAAGG
ATAGAGATAAAAGACACCAAGGAAGCTTTAGACAAGATAGAGGAAGAGCAAAACAAAAG
TAAGACCACCGCACAGCAAGCGGCCGCTGATCTTCAGACCTGGAGGAGGAGATATGAGG
GACAATTGGAGAAGTGAATTATATAAATATAAAGTAGTAAAAATTGAACCATTAGGAGT
AGCACCCACCAAGGCAAAGAGAAGAGTGGTGCAGAGAGAAAAAAGAGCAGTGGGAATAG
GAGCTTTGTTCCTTGGGTTCTTGGGAGCAGCAGGAAGCACTATGGGCGCAGCGTCAATG
ACGCTGACGGTACAGGCCAGACAATTATTGTCTGGTATAGTGCAGCAGCAGAACAATTT
GCTGAGGGCTATTGAGGCGCAACAGCATCTGTTGCAACTCACAGTCTGGGGCATCAAGC
AGCTCCAGGCAAGAATCCTGGCTGTGGAAAGATACCTAAAGGATCAACAGCTCCTGGGG
ATTTGGGGTTGCTCTGGAAAACTCATTTGCACCACTGCTGTGCCTTGGAATGCTAGTTG
GAGTAATAAATCTCTGGAACAGATTTGGAATCACACGACCTGGATGGAGTGGGACAGAG
AAATTAACAATTACACAAGCTTAATACACTCCTTAATTGAAGAATCGCAAAACCAGCAA
GAAAAGAATGAACAAGAATTATTGGAATTAGATAAATGGGCAAGTTTGTGGAATTGGTT
TAACATAACAAATTGGCTGTGGTATATAAAATTATTCATAATGATAGTAGGAGGCTTGG
TAGGTTTAAGAATAGTTTTTGCTGTACTTTCTATAGTGAATAGAGTTAGGCAGGGATAT
TCACCATTATCGTTTCAGACCCACCTCCCAACCCCGAGGGGACCCTATTCCAGCACATA
TGAGGCTTGGCGTAACTAGATCTTGAGACAAATGGCAGTATTCATCCACAATTTTAAAA
GAAAAGGGGGGATTGGGGGGTACAGTGCAGGGGAAAGAATAGTAGACATAATAGCAACA
GACATACAAACTAAAGAATTACAAAAACAAATTACAAAAATTCAAAATTTTCGGGTTTA
TTACAGGGACAGCAGAGATCCACTTTGGGCTCGAGGGGGCCCGGGTGCAAAGATGGATA
AAGTTTTAAACAGAGAGGAATCTTTGCAGCTAATGGACCTTCTAGGTCTTGAAAGGAGT
GGGAATTGGCTCCGGTGCCCGTCAGTGGGCAGAGCGCACATCGCCCACAGTCCCCGAGA
AGTTGGGGGGAGGGGTCGGCAATTGATCCGGTGCCTAGAGAAGGTGGCGCGGGGTAAAC
TGGGAAAGTGATGTCGTGTACTGGCTCCGCCTTTTTCCCGAGGGTGGGGGAGAACCGTA
TATAAGTGCAGTAGTCGCCGTGAACGTTCTTTTTCGCAACGGGTTTGCCGCCAGAACAC
AGGTAAGTGCCGTGTGTGGTTCCCGCGGGCCTGGCCTCTTTACGGGTTATGGCCCTTGC
GTGCCTTGAATTACTTCCACCTGGCTGCAGTACGTGATTCTTGATCCCGAGCTTCGGGT
TGGAAGTGGGTGGGAGAGTTCGAGGCCTTGCGCTTAAGGAGCCCCTTCGCCTCGTGCTT
GAGTTGAGGCCTGGCCTGGGCGCTGGGGCCGCCGCGTGCGAATCTGGTGGCACCTTCGC
GCCTGTCTCGCTGCTTTCGATAAGTCTCTAGCCATTTAAAATTTTTGATGACCTGCTGC
GACGCTTTTTTTCTGGCAAGATAGTCTTGTAAATGCGGGCCAAGATCTGCACACTGGTA
TTTCGGTTTTTGGGGCCGCGGGCGGCGACGGGGCCCGTGCGTCCCAGCGCACATGTTCG
GCGAGGCGGGGCCTGCGAGCGCGGCCACCGAGAATCGGACGGGGGTAGTCTCAAGCTGG
CCGGCCTGCTCTGGTGCCTGGCCTCGCGCCGCCGTGTATCGCCCCGCCCTGGGCGGCAA
GGCTGGCCCGGTCGGCACCAGTTGCGTGAGCGGAAAGATGGCCGCTTCCCGGCCCTGCT
GCAGGGAGCTCAAAATGGAGGACGCGGCGCTCGGGAGAGCGGGCGGGTGAGTCACCCAC
ACAAAGGAAAAGGGCCTTTCCGTCCTCAGCCGTCGCTTCATGTGACTCCACGGAGTACC
GGGCGCCGTCCAGGCACCTCGATTAGTTCTCGAGCTTTTGGAGTACGTCGTCTTTAGGT
TGGGGGGAGGGGTTTTATGCGATGGAGTTTCCCCACACTGAGTGGGTGGAGACTGAAGT
TAGGCCAGCTTGGCACTTGATGTAATTCTCCTTGGAATTTGCCCTTTTTGAGTTTGGAT
CTTGGTTCATTCTCAAGCCTCAGACAGTGGTTCAAAGTTTTTTTCTTCCATTTCAGGTG
TCGTGAGGATCTACCGGTCGCCACCATGGAGACGGACGTCTCAgctgaagctgctgcaA
AGgaagctgcagctAAGgaggctgcagctAAGgctATGGTGTCTAAGGGCGAAGAGCTG
ATTAAGGAGAACATGCACATGAAGCTGTACATGGAGGGCACCGTGGACAACCATCACTT
CAAGTGCACATCCGAGGGCGAAGGCAAGCCCTACGAGGGCACCCAGACCATGAGAATCA
AGGTGGTCGAGGGCGGCCCTCTCCCCTTCGCCTTCGACATCCTGGCTACTAGCTTCCTC
TACGGCAGCAAGACCTTCATCAACCACACCCAGGGCATCCCCGACTTCTTCAAGCAGTC
CTTCCCTGAGGGCTTCACATGGGAGAGAGTCACCACATACGAAGACGGGGGCGTGCTGA
CCGCTACCCAGGACACCAGCCTCCAGGACGGCTGCCTCATCTACAACGTCAAGATCAGA
GGGGTGAACTTCACATCCAACGGCCCTGTGATGCAGAAGAAAACACTCGGCTGGGAGGC
CTTCACCGAGACcCTGTACCCCGCTGACGGCGGCCTGGAAGGCAGAAACGACATGGCCC
TGAAGCTCGTGGGCGGGAGCCATCTGATCGCAAACGCCAAGACCACATATAGATCCAAG
AAACCCGCTAAGAACCTCAAGATGCCTGGCGTCTACTATGTGGACTACAGACTGGAAAG
AATCAAGGAGGCCAACAACGAGACCTACGTCGAGCAGCACGAGGTGGCAGTGGCCAGAT
ACTGCGACCTCCCTAGCAAACTGGGGCACAAGCTTAATtaaCAGGGACAGCAGAGATAC
GCGTTAAGTCGACAATCAACCTCTGGATTACAAAATTTGTGAAAGATTGACTGGTATTC
TTAACTATGTTGCTCCTTTTACGCTATGTGGATACGCTGCTTTAATGCCTTTGTATCAT
GCTATTGCTTCCCGTATGGCTTTCATTTTCTCCTCCTTGTATAAATCCTGGTTGCTGTC
TCTTTATGAGGAGTTGTGGCCCGTTGTCAGGCAACGTGGCGTGGTGTGCACTGTGTTTG
CTGACGCAACCCCCACTGGTTGGGGCATTGCCACCACCTGTCAGCTCCTTTCCGGGACT
TTCGCTTTCCCCCTCCCTATTGCCACGGCGGAACTCATCGCCGCCTGCCTTGCCCGCTG
CTGGACAGGGGCTCGGCTGTTGGGCACTGACAATTCCGTGGTGTTGTCGGGGAAATCAT
CGTCCTTTCCTTGGCTGCTCGCCTGTGTTGCCACCTGGATTCTGCGCGGGACGTCCTTC
TGCTACGTCCCTTCGGCCCTCAATCCAGCGGACCTTCCTTCCCGCGGCCTGCTGCCGGC
TCTGCGGCCTCTTCCGCGTCTTCGCCTTCGCCCTCAGACGAGTCGGATCTCCCTTTGGG
CCGCCTCCCCGCGTCGACTTTAAGACCAATGACTTACAAGGCAGCTGTAGATCTTAGCC
ACTTTTTAAAAGAAAAGGGGGGACTGGAAGGGCTAATTCACTCCCAACGAAGACAAGAT
CTGCTTTTTGCTTGTACTGGGTCTCTCTGGTTAGACCAGATCTGAGCCTGGGAGCTCTC
TGGCTAACTAGGGAACCCACTGCTTAAGCCTCAATAAAGCTTGCCTTGAGTGCTTCAAG
TAGTGTGTGCCCGTCTGTTGTGTGACTCTGGTAACTAGAGATCCCTCAGACCCTTTTAG
TCAGTGTGGAAAATCTCTAGCAGTACGTATAGTAGTTCATGTCATCTTATTATTCAGTA
TTTATAACTTGCAAAGAAATGAATATCAGAGAGTGAGAGGAACTTGTTTATTGCAGCTT
ATAATGGTTACAAATAAAGCAATAGCATCACAAATTTCACAAATAAAGCATTTTTTTCA
CTGCATTCTAGTTGTGGTTTGTCCAAACTCATCAATGTATCTTATCATGTCTGGCTCTA
GCTATCCCGCCCCTAACTCCGCCCATCCCGCCCCTAACTCCGCCCAGTTCCGCCCATTC
TCCGCCCCATGGCTGACTAATTTTTTTTATTTATGCAGAGGCCGAGGCCGCCTCGGCCT
CTGAGCTATTCCAGAAGTAGTGAGGAGGCTTTTTTGGAGGCCTAGGGACGTACCCAATT
CGCCCTATAGTGAGTCGTATTACGCGCGCTCACTGGCCGTCGTTTTACAACGTCGTGAC
TGGGAAAACCCTGGCGTTACCCAACTTAATCGCCTTGCAGCACATCCCCCTTTCGCCAG
CTGGCGTAATAGCGAAGAGGCCCGCACCGATCGCCCTTCCCAACAGTTGCGCAGCCTGA
ATGGCGAATGGGACGCGCCCTGTAGCGGCGCATTAAGCGCGGCGGGTGTGGTGGTTACG
CGCAGCGTGACCGCTACACTTGCCAGCGCCCTAGCGCCCGCTCCTTTCGCTTTCTTCCC
TTCCTTTCTCGCCACGTTCGCCGGCTTTCCCCGTCAAGCTCTAAATCGGGGGCTCCCTT
TAGGGTTCCGATTTAGTGCTTTACGGCACCTCGACCCCAAAAAACTTGATTAGGGTGAT
GGTTCACGTAGTGGGCCATCGCCCTGATAGACGGTTTTTCGCCCTTTGACGTTGGAGTC
CACGTTCTTTAATAGTGGACTCTTGTTCCAAACTGGAACAACACTCAACCCTATCTCGG
TCTATTCTTTTGATTTATAAGGGATTTTGCCGATTTCGGCCTATTGGTTAAAAAATGAG
CTGATTTAACAAAAATTTAACGCGAATTTTAACAAAATATTAACGCTTACAATTTAGGT
GGCACTTTTCGGGGAAATGTGCGCGGAACCCCTATTTGTTTATTTTTCTAAATACATTC
AAATATGTATCCGCTCATGAGACAATAACCCTGATAAATGCTTCAATAATATTGAAAAA
GGAAGAGTATGAGTATTCAACATTTCCGTGTCGCCCTTATTCCCTTTTTTGCGGCATTT
TGCCTTCCTGTTTTTGCTCACCCAGAAACGCTGGTGAAAGTAAAAGATGCTGAAGATCA
GTTGGGTGCACGAGTGGGTTACATCGAACTGGATCTCAACAGCGGTAAGATCCTTGAGA
GTTTTCGCCCCGAAGAACGTTTTCCAATGATGAGCACTTTTAAAGTTCTGCTATGTGGC
GCGGTATTATCCCGTATTGACGCCGGGCAAGAGCAACTCGGTCGCCGCATACACTATTC
TCAGAATGACTTGGTTGAGTACTCACCAGTCACAGAAAAGCATCTTACGGATGGCATGA
CAGTAAGAGAATTATGCAGTGCTGCCATAACCATGAGTGATAACACTGCGGCCAACTTA
CTTCTGACAACGATCGGAGGACCGAAGGAGCTAACCGCTTTTTTGCACAACATGGGGGA
TCATGTAACTCGCCTTGATCGTTGGGAACCGGAGCTGAATGAAGCCATACCAAACGACG
AGCGTGACACCACGATGCCTGTAGCAATGGCAACAACGTTGCGCAAACTATTAACTGGC
GAACTACTTACTCTAGCTTCCCGGCAACAATTAATAGACTGGATGGAGGCGGATAAAGT
TGCAGGACCACTTCTGCGCTCGGCCCTTCCGGCTGGCTGGTTTATTGCTGATAAATCTG
GAGCCGGTGAGCGTGGGTCTCGCGGTATCATTGCAGCACTGGGGCCAGATGGTAAGCCC
TCCCGTATCGTAGTTATCTACACGACGGGGAGTCAGGCAACTATGGATGAACGAAATAG
ACAGATCGCTGAGATAGGTGCCTCACTGATTAAGCATTGGTAA
“Bolinas”: pEF1α-BsmBI cloning
site-17aaRigidLinker-mTagBFP2-P2A-Blasticidin
(SEQ ID NO: 91)
TTTTGCACAACATGGGGGATCATGTAACTCGCCTTGATCGTTGGGAACCGGAGCTGAAT
GAAGCCATACCAAACGACGAGCGTGACACCACGATGCCTGTAGCAATGGCAACAACGTT
GCGCAAACTATTAACTGGCGAACTACTTACTCTAGCTTCCCGGCAACAATTAATAGACT
GGATGGAGGCGGATAAAGTTGCAGGACCACTTCTGCGCTCGGCCCTTCCGGCTGGCTGG
TTTATTGCTGATAAATCTGGAGCCGGTGAGCGTGGGTCTCGCGGTATCATTGCAGCACT
GGGGCCAGATGGTAAGCCCTCCCGTATCGTAGTTATCTACACGACGGGGAGTCAGGCAA
CTATGGATGAACGAAATAGACAGATCGCTGAGATAGGTGCCTCACTGATTAAGCATTGG
TAACTGTCAGACCAAGTTTACTCATATATACTTTAGATTGATTTAAAACTTCATTTTTA
ATTTAAAAGGATCTAGGTGAAGATCCTTTTTGATAATCTCATGACCAAAATCCCTTAAC
GTGAGTTTTCGTTCCACTGAGCGTCAGACCCCGTAGAAAAGATCAAAGGATCTTCTTGA
GATCCTTTTTTTCTGCGCGTAATCTGCTGCTTGCAAACAAAAAAACCACCGCTACCAGC
GGTGGTTTGTTTGCCGGATCAAGAGCTACCAACTCTTTTTCCGAAGGTAACTGGCTTCA
GCAGAGCGCAGATACCAAATACTGTTCTTCTAGTGTAGCCGTAGTTAGGCCACCACTTC
AAGAACTCTGTAGCACCGCCTACATACCTCGCTCTGCTAATCCTGTTACCAGTGGCTGC
TGCCAGTGGCGATAAGTCGTGTCTTACCGGGTTGGACTCAAGACGATAGTTACCGGATA
AGGCGCAGCGGTCGGGCTGAACGGGGGGTTCGTGCACACAGCCCAGCTTGGAGCGAACG
ACCTACACCGAACTGAGATACCTACAGCGTGAGCTATGAGAAAGCGCCACGCTTCCCGA
AGGGAGAAAGGCGGACAGGTATCCGGTAAGCGGCAGGGTCGGAACAGGAGAGCGCACGA
GGGAGCTTCCAGGGGGAAACGCCTGGTATCTTTATAGTCCTGTCGGGTTTCGCCACCTC
TGACTTGAGCGTCGATTTTTGTGATGCTCGTCAGGGGGGCGGAGCCTATGGAAAAACGC
CAGCAACGCGGCCTTTTTACGGTTCCTGGCCTTTTGCTGGCCTTTTGCTCACATGTTCT
TTCCTGCGTTATCCCCTGATTCTGTGGATAACCGTATTACCGCCTTTGAGTGAGCTGAT
ACCGCTCGCCGCAGCCGAACGACCGAGCGCAGCGAGTCAGTGAGCGAGGAAGCGGAAGA
GCGCCCAATACGCAAACCGCCTCTCCCCGCGCGTTGGCCGATTCATTAATGCAGCTGGC
ACGACAGGTTTCCCGACTGGAAAGCGGGCAGTGAGCGCAACGCAATTAATGTGAGTTAG
CTCACTCATTAGGCACCCCAGGCTTTACACTTTATGCTTCCGGCTCGTATGTTGTGTGG
AATTGTGAGCGGATAACAATTTCACACAGGAAACAGCTATGACCATGATTACGCCAAGC
GCGCAATTAACCCTCACTAAAGGGAACAAAAGCTGGAGCTGCAAGCTTAATGTAGTCTT
ATGCAATACTCTTGTAGTCTTGCAACATGGTAACGATGAGTTAGCAACATGCCTTACAA
GGAGAGAAAAAGCACCGTGCATGCCGATTGGTGGAAGTAAGGTGGTACGATCGTGCCTT
ATTAGGAAGGCAACAGACGGGTCTGACATGGATTGGACGAACCACTGAATTGCCGCATT
GCAGAGATATTGTATTTAAGTGCCTAGCTCGATACATAAACGGGTCTCTCTGGTTAGAC
CAGATCTGAGCCTGGGAGCTCTCTGGCTAACTAGGGAACCCACTGCTTAAGCCTCAATA
AAGCTTGCCTTGAGTGCTTCAAGTAGTGTGTGCCCGTCTGTTGTGTGACTCTGGTAACT
AGAGATCCCTCAGACCCTTTTAGTCAGTGTGGAAAATCTCTAGCAGTGGCGCCCGAACA
GGGACTTGAAAGCGAAAGGGAAACCAGAGGAGCTCTCTCGACGCAGGACTCGGCTTGCT
GAAGCGCGCACGGCAAGAGGCGAGGGGCGGCGACTGGTGAGTACGCCAAAAATTTTGAC
TAGCGGAGGCTAGAAGGAGAGAGATGGGTGCGAGAGCGTCAGTATTAAGCGGGGGAGAA
TTAGATCGCGATGGGAAAAAATTCGGTTAAGGCCAGGGGGAAAGAAAAAATATAAATTA
AAACATATAGTATGGGCAAGCAGGGAGCTAGAACGATTCGCAGTTAATCCTGGCCTGTT
AGAAACATCAGAAGGCTGTAGACAAATACTGGGACAGCTACAACCATCCCTTCAGACAG
GATCAGAAGAACTTAGATCATTATATAATACAGTAGCAACCCTCTATTGTGTGCATCAA
AGGATAGAGATAAAAGACACCAAGGAAGCTTTAGACAAGATAGAGGAAGAGCAAAACAA
AAGTAAGACCACCGCACAGCAAGCGGCCGCTGATCTTCAGACCTGGAGGAGGAGATATG
AGGGACAATTGGAGAAGTGAATTATATAAATATAAAGTAGTAAAAATTGAACCATTAGG
AGTAGCACCCACCAAGGCAAAGAGAAGAGTGGTGCAGAGAGAAAAAAGAGCAGTGGGAA
TAGGAGCTTTGTTCCTTGGGTTCTTGGGAGCAGCAGGAAGCACTATGGGCGCAGCGTCA
ATGACGCTGACGGTACAGGCCAGACAATTATTGTCTGGTATAGTGCAGCAGCAGAACAA
TTTGCTGAGGGCTATTGAGGCGCAACAGCATCTGTTGCAACTCACAGTCTGGGGCATCA
AGCAGCTCCAGGCAAGAATCCTGGCTGTGGAAAGATACCTAAAGGATCAACAGCTCCTG
GGGATTTGGGGTTGCTCTGGAAAACTCATTTGCACCACTGCTGTGCCTTGGAATGCTAG
TTGGAGTAATAAATCTCTGGAACAGATTTGGAATCACACGACCTGGATGGAGTGGGACA
GAGAAATTAACAATTACACAAGCTTAATACACTCCTTAATTGAAGAATCGCAAAACCAG
CAAGAAAAGAATGAACAAGAATTATTGGAATTAGATAAATGGGCAAGTTTGTGGAATTG
GTTTAACATAACAAATTGGCTGTGGTATATAAAATTATTCATAATGATAGTAGGAGGCT
TGGTAGGTTTAAGAATAGTTTTTGCTGTACTTTCTATAGTGAATAGAGTTAGGCAGGGA
TATTCACCATTATCGTTTCAGACCCACCTCCCAACCCCGAGGGGACCCTATTCCAGCAC
ATATGAGGCTTGGCGTAACTAGATCTTGAGACAAATGGCAGTATTCATCCACAATTTTA
AAAGAAAAGGGGGGATTGGGGGGTACAGTGCAGGGGAAAGAATAGTAGACATAATAGCA
ACAGACATACAAACTAAAGAATTACAAAAACAAATTACAAAAATTCAAAATTTTCGGGT
TTATTACAGGGACAGCAGAGATCCACTTTGGGCTCGAGGGGGCCCGGGTGCAAAGATGG
ATAAAGTTTTAAACAGAGAGGAATCTTTGCAGCTAATGGACCTTCTAGGTCTTGAAAGG
AGTGGGAATTGGCTCCGGTGCCCGTCAGTGGGCAGAGCGCACATCGCCCACAGTCCCCG
AGAAGTTGGGGGGAGGGGTCGGCAATTGATCCGGTGCCTAGAGAAGGTGGCGCGGGGTA
AACTGGGAAAGTGATGTCGTGTACTGGCTCCGCCTTTTTCCCGAGGGTGGGGGAGAACC
GTATATAAGTGCAGTAGTCGCCGTGAACGTTCTTTTTCGCAACGGGTTTGCCGCCAGAA
CACAGGTAAGTGCCGTGTGTGGTTCCCGCGGGCCTGGCCTCTTTACGGGTTATGGCCCT
TGCGTGCCTTGAATTACTTCCACCTGGCTGCAGTACGTGATTCTTGATCCCGAGCTTCG
GGTTGGAAGTGGGTGGGAGAGTTCGAGGCCTTGCGCTTAAGGAGCCCCTTCGCCTCGTG
CTTGAGTTGAGGCCTGGCCTGGGCGCTGGGGCCGCCGCGTGCGAATCTGGTGGCACCTT
CGCGCCTGTCTCGCTGCTTTCGATAAGTCTCTAGCCATTTAAAATTTTTGATGACCTGC
TGCGACGCTTTTTTTCTGGCAAGATAGTCTTGTAAATGCGGGCCAAGATCTGCACACTG
GTATTTCGGTTTTTGGGGCCGCGGGCGGCGACGGGGCCCGTGCGTCCCAGCGCACATGT
TCGGCGAGGCGGGGCCTGCGAGCGCGGCCACCGAGAATCGGACGGGGGTAGTCTCAAGC
TGGCCGGCCTGCTCTGGTGCCTGGCCTCGCGCCGCCGTGTATCGCCCCGCCCTGGGCGG
CAAGGCTGGCCCGGTCGGCACCAGTTGCGTGAGCGGAAAGATGGCCGCTTCCCGGCCCT
GCTGCAGGGAGCTCAAAATGGAGGACGCGGCGCTCGGGAGAGCGGGCGGGTGAGTCACC
CACACAAAGGAAAAGGGCCTTTCCGTCCTCAGCCGTCGCTTCATGTGACTCCACGGAGT
ACCGGGCGCCGTCCAGGCACCTCGATTAGTTCTCGAGCTTTTGGAGTACGTCGTCTTTA
GGTTGGGGGGAGGGGTTTTATGCGATGGAGTTTCCCCACACTGAGTGGGTGGAGACTGA
AGTTAGGCCAGCTTGGCACTTGATGTAATTCTCCTTGGAATTTGCCCTTTTTGAGTTTG
GATCTTGGTTCATTCTCAAGCCTCAGACAGTGGTTCAAAGTTTTTTTCTTCCATTTCAG
GTGTCGTGAATACCGTCGACTCCGGAATAGCCACCATGGAGACGGACGTCTCAGCTGAA
GCTGCTGCAAAGGAAGCTGCAGCTAAGGAGGCTGCAGCTAAGGCTATGGTGTCTAAGGG
CGAAGAGCTGATTAAGGAGAACATGCACATGAAGCTGTACATGGAGGGCACCGTGGACA
ACCATCACTTCAAGTGCACATCCGAGGGCGAAGGCAAGCCCTACGAGGGCACCCAGACC
ATGAGAATCAAGGTGGTCGAGGGCGGCCCTCTCCCCTTCGCCTTCGACATCCTGGCTAC
TAGCTTCCTCTACGGCAGCAAGACCTTCATCAACCACACCCAGGGCATCCCCGACTTCT
TCAAGCAGTCCTTCCCTGAGGGCTTCACATGGGAGAGAGTCACCACATACGAAGACGGG
GGCGTGCTGACCGCTACCCAGGACACCAGCCTCCAGGACGGCTGCCTCATCTACAACGT
CAAGATCAGAGGGGTGAACTTCACATCCAACGGCCCTGTGATGCAGAAGAAAACACTCG
GCTGGGAGGCCTTCACCGAGACcCTGTACCCCGCTGACGGCGGCCTGGAAGGCAGAAAC
GACATGGCCCTGAAGCTCGTGGGCGGGAGCCATCTGATCGCAAACGCCAAGACCACATA
TAGATCCAAGAAACCCGCTAAGAACCTCAAGATGCCTGGCGTCTACTATGTGGACTACA
GACTGGAAAGAATCAAGGAGGCCAACAACGAGACCTACGTCGAGCAGCACGAGGTGGCA
GTGGCCAGATACTGCGACCTCCCTAGCAAACTGGGGCACAAGCTTAATGGACTCAGAGT
TTGGGTAGGAAGCGGAGCTACTAACTTCAGCCTGCTGAAGCAGGCTGGAGAtGTGGAGG
AGAACCCTGGACCTATGGCCAAGCCTTTGTCTCAAGAAGAATCCACCCTCATTGAAAGA
GCAACGGCTACAATCAACAGCATCCCCATCTCTGAAGACTACAGCGTCGCCAGCGCAGC
TCTCTCTAGCGACGGCCGCATCTTCACTGGTGTCAATGTATATCATTTTACTGGGGGAC
CTTGTGCAGAACTCGTGGTGCTGGGCACTGCTGCTGCTGCGGCAGCTGGCAACCTGACT
TGTATCGTCGCGATCGGAAATGAGAACAGGGGCATCTTGAGCCCCTGCGGACGGTGCCG
ACAGGTGCTTCTCGATCTGCATCCTGGGATCAAAGCCATAGTGAAGGACAGTGATGGAC
AGCCGACGGCAGTTGGGATTCGTGAATTGCTGCCCTCTGGTTATGTGTGGGAGGGCTAA
ATGCATGAGTAACTGAGGATCCAGGGACAGCAGAGATACGCGTTAAGTCGACAATCAAC
CTCTGGATTACAAAATTTGTGAAAGATTGACTGGTATTCTTAACTATGTTGCTCCTTTT
ACGCTATGTGGATACGCTGCTTTAATGCCTTTGTATCATGCTATTGCTTCCCGTATGGC
TTTCATTTTCTCCTCCTTGTATAAATCCTGGTTGCTGTCTCTTTATGAGGAGTTGTGGC
CCGTTGTCAGGCAACGTGGCGTGGTGTGCACTGTGTTTGCTGACGCAACCCCCACTGGT
TGGGGCATTGCCACCACCTGTCAGCTCCTTTCCGGGACTTTCGCTTTCCCCCTCCCTAT
TGCCACGGCGGAACTCATCGCCGCCTGCCTTGCCCGCTGCTGGACAGGGGCTCGGCTGT
TGGGCACTGACAATTCCGTGGTGTTGTCGGGGAAATCATCGTCCTTTCCTTGGCTGCTC
GCCTGTGTTGCCACCTGGATTCTGCGCGGGACGTCCTTCTGCTACGTCCCTTCGGCCCT
CAATCCAGCGGACCTTCCTTCCCGCGGCCTGCTGCCGGCTCTGCGGCCTCTTCCGCGTC
TTCGCCTTCGCCCTCAGACGAGTCGGATCTCCCTTTGGGCCGCCTCCCCGCGTCGACTT
TAAGACCAATGACTTACAAGGCAGCTGTAGATCTTAGCCACTTTTTAAAAGAAAAGGGG
GGACTGGAAGGGCTAATTCACTCCCAACGAAGACAAGATCTGCTTTTTGCTTGTACTGG
GTCTCTCTGGTTAGACCAGATCTGAGCCTGGGAGCTCTCTGGCTAACTAGGGAACCCAC
TGCTTAAGCCTCAATAAAGCTTGCCTTGAGTGCTTCAAGTAGTGTGTGCCCGTCTGTTG
TGTGACTCTGGTAACTAGAGATCCCTCAGACCCTTTTAGTCAGTGTGGAAAATCTCTAG
CAGTACGTATAGTAGTTCATGTCATCTTATTATTCAGTATTTATAACTTGCAAAGAAAT
GAATATCAGAGAGTGAGAGGAACTTGTTTATTGCAGCTTATAATGGTTACAAATAAAGC
AATAGCATCACAAATTTCACAAATAAAGCATTTTTTTCACTGCATTCTAGTTGTGGTTT
GTCCAAACTCATCAATGTATCTTATCATGTCTGGCTCTAGCTATCCCGCCCCTAACTCC
GCCCATCCCGCCCCTAACTCCGCCCAGTTCCGCCCATTCTCCGCCCCATGGCTGACTAA
TTTTTTTTATTTATGCAGAGGCCGAGGCCGCCTCGGCCTCTGAGCTATTCCAGAAGTAG
TGAGGAGGCTTTTTTGGAGGCCTAGGGACGTACCCAATTCGCCCTATAGTGAGTCGTAT
TACGCGCGCTCACTGGCCGTCGTTTTACAACGTCGTGACTGGGAAAACCCTGGCGTTAC
CCAACTTAATCGCCTTGCAGCACATCCCCCTTTCGCCAGCTGGCGTAATAGCGAAGAGG
CCCGCACCGATCGCCCTTCCCAACAGTTGCGCAGCCTGAATGGCGAATGGGACGCGCCC
TGTAGCGGCGCATTAAGCGCGGCGGGTGTGGTGGTTACGCGCAGCGTGACCGCTACACT
TGCCAGCGCCCTAGCGCCCGCTCCTTTCGCTTTCTTCCCTTCCTTTCTCGCCACGTTCG
CCGGCTTTCCCCGTCAAGCTCTAAATCGGGGGCTCCCTTTAGGGTTCCGATTTAGTGCT
TTACGGCACCTCGACCCCAAAAAACTTGATTAGGGTGATGGTTCACGTAGTGGGCCATC
GCCCTGATAGACGGTTTTTCGCCCTTTGACGTTGGAGTCCACGTTCTTTAATAGTGGAC
TCTTGTTCCAAACTGGAACAACACTCAACCCTATCTCGGTCTATTCTTTTGATTTATAA
GGGATTTTGCCGATTTCGGCCTATTGGTTAAAAAATGAGCTGATTTAACAAAAATTTAA
CGCGAATTTTAACAAAATATTAACGCTTACAATTTAGGTGGCACTTTTCGGGGAAATGT
GCGCGGAACCCCTATTTGTTTATTTTTCTAAATACATTCAAATATGTATCCGCTCATGA
GACAATAACCCTGATAAATGCTTCAATAATATTGAAAAAGGAAGAGTATGAGTATTCAA
CATTTCCGTGTCGCCCTTATTCCCTTTTTTGCGGCATTTTGCCTTCCTGTTTTTGCTCA
CCCAGAAACGCTGGTGAAAGTAAAAGATGCTGAAGATCAGTTGGGTGCACGAGTGGGTT
ACATCGAACTGGATCTCAACAGCGGTAAGATCCTTGAGAGTTTTCGCCCCGAAGAACGT
TTTCCAATGATGAGCACTTTTAAAGTTCTGCTATGTGGCGCGGTATTATCCCGTATTGA
CGCCGGGCAAGAGCAACTCGGTCGCCGCATACACTATTCTCAGAATGACTTGGTTGAGT
ACTCACCAGTCACAGAAAAGCATCTTACGGATGGCATGACAGTAAGAGAATTATGCAGT
GCTGCCATAACCATGAGTGATAACACTGCGGCCAACTTACTTCTGACAACGATCGGAGG
ACCGAAGGAGCTAACCGCTT
“Jenner”: pEF1α-BsmBI cloning
site-17aaRigidLinker-mCherry-P2A-neo
(SEQ ID NO: 92)
TTTTGCACAACATGGGGGATCATGTAACTCGCCTTGATCGTTGGGAACCGGAGCTGAAT
GAAGCCATACCAAACGACGAGCGTGACACCACGATGCCTGTAGCAATGGCAACAACGTT
GCGCAAACTATTAACTGGCGAACTACTTACTCTAGCTTCCCGGCAACAATTAATAGACT
GGATGGAGGCGGATAAAGTTGCAGGACCACTTCTGCGCTCGGCCCTTCCGGCTGGCTGG
TTTATTGCTGATAAATCTGGAGCCGGTGAGCGTGGGTCTCGCGGTATCATTGCAGCACT
GGGGCCAGATGGTAAGCCCTCCCGTATCGTAGTTATCTACACGACGGGGAGTCAGGCAA
CTATGGATGAACGAAATAGACAGATCGCTGAGATAGGTGCCTCACTGATTAAGCATTGG
TAACTGTCAGACCAAGTTTACTCATATATACTTTAGATTGATTTAAAACTTCATTTTTA
ATTTAAAAGGATCTAGGTGAAGATCCTTTTTGATAATCTCATGACCAAAATCCCTTAAC
GTGAGTTTTCGTTCCACTGAGCGTCAGACCCCGTAGAAAAGATCAAAGGATCTTCTTGA
GATCCTTTTTTTCTGCGCGTAATCTGCTGCTTGCAAACAAAAAAACCACCGCTACCAGC
GGTGGTTTGTTTGCCGGATCAAGAGCTACCAACTCTTTTTCCGAAGGTAACTGGCTTCA
GCAGAGCGCAGATACCAAATACTGTTCTTCTAGTGTAGCCGTAGTTAGGCCACCACTTC
AAGAACTCTGTAGCACCGCCTACATACCTCGCTCTGCTAATCCTGTTACCAGTGGCTGC
TGCCAGTGGCGATAAGTCGTGTCTTACCGGGTTGGACTCAAGACGATAGTTACCGGATA
AGGCGCAGCGGTCGGGCTGAACGGGGGGTTCGTGCACACAGCCCAGCTTGGAGCGAACG
ACCTACACCGAACTGAGATACCTACAGCGTGAGCTATGAGAAAGCGCCACGCTTCCCGA
AGGGAGAAAGGCGGACAGGTATCCGGTAAGCGGCAGGGTCGGAACAGGAGAGCGCACGA
GGGAGCTTCCAGGGGGAAACGCCTGGTATCTTTATAGTCCTGTCGGGTTTCGCCACCTC
TGACTTGAGCGTCGATTTTTGTGATGCTCGTCAGGGGGGCGGAGCCTATGGAAAAACGC
CAGCAACGCGGCCTTTTTACGGTTCCTGGCCTTTTGCTGGCCTTTTGCTCACATGTTCT
TTCCTGCGTTATCCCCTGATTCTGTGGATAACCGTATTACCGCCTTTGAGTGAGCTGAT
ACCGCTCGCCGCAGCCGAACGACCGAGCGCAGCGAGTCAGTGAGCGAGGAAGCGGAAGA
GCGCCCAATACGCAAACCGCCTCTCCCCGCGCGTTGGCCGATTCATTAATGCAGCTGGC
ACGACAGGTTTCCCGACTGGAAAGCGGGCAGTGAGCGCAACGCAATTAATGTGAGTTAG
CTCACTCATTAGGCACCCCAGGCTTTACACTTTATGCTTCCGGCTCGTATGTTGTGTGG
AATTGTGAGCGGATAACAATTTCACACAGGAAACAGCTATGACCATGATTACGCCAAGC
GCGCAATTAACCCTCACTAAAGGGAACAAAAGCTGGAGCTGCAAGCTTAATGTAGTCTT
ATGCAATACTCTTGTAGTCTTGCAACATGGTAACGATGAGTTAGCAACATGCCTTACAA
GGAGAGAAAAAGCACCGTGCATGCCGATTGGTGGAAGTAAGGTGGTACGATCGTGCCTT
ATTAGGAAGGCAACAGACGGGTCTGACATGGATTGGACGAACCACTGAATTGCCGCATT
GCAGAGATATTGTATTTAAGTGCCTAGCTCGATACATAAACGGGTCTCTCTGGTTAGAC
CAGATCTGAGCCTGGGAGCTCTCTGGCTAACTAGGGAACCCACTGCTTAAGCCTCAATA
AAGCTTGCCTTGAGTGCTTCAAGTAGTGTGTGCCCGTCTGTTGTGTGACTCTGGTAACT
AGAGATCCCTCAGACCCTTTTAGTCAGTGTGGAAAATCTCTAGCAGTGGCGCCCGAACA
GGGACTTGAAAGCGAAAGGGAAACCAGAGGAGCTCTCTCGACGCAGGACTCGGCTTGCT
GAAGCGCGCACGGCAAGAGGCGAGGGGCGGCGACTGGTGAGTACGCCAAAAATTTTGAC
TAGCGGAGGCTAGAAGGAGAGAGATGGGTGCGAGAGCGTCAGTATTAAGCGGGGGAGAA
TTAGATCGCGATGGGAAAAAATTCGGTTAAGGCCAGGGGGAAAGAAAAAATATAAATTA
AAACATATAGTATGGGCAAGCAGGGAGCTAGAACGATTCGCAGTTAATCCTGGCCTGTT
AGAAACATCAGAAGGCTGTAGACAAATACTGGGACAGCTACAACCATCCCTTCAGACAG
GATCAGAAGAACTTAGATCATTATATAATACAGTAGCAACCCTCTATTGTGTGCATCAA
AGGATAGAGATAAAAGACACCAAGGAAGCTTTAGACAAGATAGAGGAAGAGCAAAACAA
AAGTAAGACCACCGCACAGCAAGCGGCCGCTGATCTTCAGACCTGGAGGAGGAGATATG
AGGGACAATTGGAGAAGTGAATTATATAAATATAAAGTAGTAAAAATTGAACCATTAGG
AGTAGCACCCACCAAGGCAAAGAGAAGAGTGGTGCAGAGAGAAAAAAGAGCAGTGGGAA
TAGGAGCTTTGTTCCTTGGGTTCTTGGGAGCAGCAGGAAGCACTATGGGCGCAGCGTCA
ATGACGCTGACGGTACAGGCCAGACAATTATTGTCTGGTATAGTGCAGCAGCAGAACAA
TTTGCTGAGGGCTATTGAGGCGCAACAGCATCTGTTGCAACTCACAGTCTGGGGCATCA
AGCAGCTCCAGGCAAGAATCCTGGCTGTGGAAAGATACCTAAAGGATCAACAGCTCCTG
GGGATTTGGGGTTGCTCTGGAAAACTCATTTGCACCACTGCTGTGCCTTGGAATGCTAG
TTGGAGTAATAAATCTCTGGAACAGATTTGGAATCACACGACCTGGATGGAGTGGGACA
GAGAAATTAACAATTACACAAGCTTAATACACTCCTTAATTGAAGAATCGCAAAACCAG
CAAGAAAAGAATGAACAAGAATTATTGGAATTAGATAAATGGGCAAGTTTGTGGAATTG
GTTTAACATAACAAATTGGCTGTGGTATATAAAATTATTCATAATGATAGTAGGAGGCT
TGGTAGGTTTAAGAATAGTTTTTGCTGTACTTTCTATAGTGAATAGAGTTAGGCAGGGA
TATTCACCATTATCGTTTCAGACCCACCTCCCAACCCCGAGGGGACCCTATTCCAGCAC
ATATGAGGCTTGGCGTAACTAGATCTTGAGACAAATGGCAGTATTCATCCACAATTTTA
AAAGAAAAGGGGGGATTGGGGGGTACAGTGCAGGGGAAAGAATAGTAGACATAATAGCA
ACAGACATACAAACTAAAGAATTACAAAAACAAATTACAAAAATTCAAAATTTTCGGGT
TTATTACAGGGACAGCAGAGATCCACTTTGGGCTCGAGGGGGCCCGGGTGCAAAGATGG
ATAAAGTTTTAAACAGAGAGGAATCTTTGCAGCTAATGGACCTTCTAGGTCTTGAAAGG
AGTGGGAATTGGCTCCGGTGCCCGTCAGTGGGCAGAGCGCACATCGCCCACAGTCCCCG
AGAAGTTGGGGGGAGGGGTCGGCAATTGATCCGGTGCCTAGAGAAGGTGGCGCGGGGTA
AACTGGGAAAGTGATGTCGTGTACTGGCTCCGCCTTTTTCCCGAGGGTGGGGGAGAACC
GTATATAAGTGCAGTAGTCGCCGTGAACGTTCTTTTTCGCAACGGGTTTGCCGCCAGAA
CACAGGTAAGTGCCGTGTGTGGTTCCCGCGGGCCTGGCCTCTTTACGGGTTATGGCCCT
TGCGTGCCTTGAATTACTTCCACCTGGCTGCAGTACGTGATTCTTGATCCCGAGCTTCG
GGTTGGAAGTGGGTGGGAGAGTTCGAGGCCTTGCGCTTAAGGAGCCCCTTCGCCTCGTG
CTTGAGTTGAGGCCTGGCCTGGGCGCTGGGGCCGCCGCGTGCGAATCTGGTGGCACCTT
CGCGCCTGTCTCGCTGCTTTCGATAAGTCTCTAGCCATTTAAAATTTTTGATGACCTGC
TGCGACGCTTTTTTTCTGGCAAGATAGTCTTGTAAATGCGGGCCAAGATCTGCACACTG
GTATTTCGGTTTTTGGGGCCGCGGGCGGCGACGGGGCCCGTGCGTCCCAGCGCACATGT
TCGGCGAGGCGGGGCCTGCGAGCGCGGCCACCGAGAATCGGACGGGGGTAGTCTCAAGC
TGGCCGGCCTGCTCTGGTGCCTGGCCTCGCGCCGCCGTGTATCGCCCCGCCCTGGGCGG
CAAGGCTGGCCCGGTCGGCACCAGTTGCGTGAGCGGAAAGATGGCCGCTTCCCGGCCCT
GCTGCAGGGAGCTCAAAATGGAGGACGCGGCGCTCGGGAGAGCGGGCGGGTGAGTCACC
CACACAAAGGAAAAGGGCCTTTCCGTCCTCAGCCGTCGCTTCATGTGACTCCACGGAGT
ACCGGGCGCCGTCCAGGCACCTCGATTAGTTCTCGAGCTTTTGGAGTACGTCGTCTTTA
GGTTGGGGGGAGGGGTTTTATGCGATGGAGTTTCCCCACACTGAGTGGGTGGAGACTGA
AGTTAGGCCAGCTTGGCACTTGATGTAATTCTCCTTGGAATTTGCCCTTTTTGAGTTTG
GATCTTGGTTCATTCTCAAGCCTCAGACAGTGGTTCAAAGTTTTTTTCTTCCATTTCAG
GTGTCGTGAATACCGTCGACTCCGGAATAGCCACCATGGAGACGGACGTCTCAGCTGAA
GCTGCTGCAAAGGAAGCTGCAGCTAAGGAGGCTGCAGCTAAGGCTATGGTGAGCAAGGG
CGAGGAGGATAACATGGCCATCATCAAGGAGTTCATGCGCTTCAAGGTGCACATGGAGG
GCTCCGTGAACGGCCACGAGTTCGAGATCGAGGGCGAGGGCGAGGGCCGCCCCTACGAG
GGCACCCAGACCGCCAAGCTGAAGGTGACCAAGGGTGGCCCCCTGCCCTTCGCCTGGGA
CATCCTGTCCCCTCAGTTCATGTACGGCTCCAAGGCCTACGTGAAGCACCCCGCCGACA
TCCCCGACTACTTGAAGCTGTCCTTCCCCGAGGGCTTCAAGTGGGAGCGCGTGATGAAC
TTCGAGGACGGCGGCGTGGTGACCGTGACCCAGGACTCCTCCCTGCAGGACGGCGAGTT
CATCTACAAGGTGAAGCTGCGCGGCACCAACTTCCCCTCCGACGGCCCCGTAATGCAGA
AGAAGACCATGGGCTGGGAGGCCTCCTCCGAGCGGATGTACCCCGAGGACGGCGCCCTG
AAGGGCGAGATCAAGCAGAGGCTGAAGCTGAAGGACGGCGGCCACTACGACGCTGAGGT
CAAGACCACCTACAAGGCCAAGAAGCCCGTGCAGCTGCCCGGCGCCTACAACGTCAACA
TCAAGTTGGACATCACCTCCCACAACGAGGACTACACCATCGTGGAACAGTACGAACGC
GCCGAGGGCCGCCACTCCACCGGCGGCATGGACGAGCTGTACAAGGGACTCAGAGTTTG
GGTAGGAAGCGGAGCTACTAACTTCAGCCTGCTGAAGCAGGCTGGAGAtGTGGAGGAGA
ACCCTGGACCTATGATTGAACAAGATGGATTGCACGCAGGTTCTCCGGCCGCTTGGGTG
GAGAGGCTATTCGGCTATGACTGGGCACAACAGACAATCGGCTGCTCTGATGCCGCCGT
GTTCCGGCTGTCAGCGCAGGGGCGCCCGGTTCTTTTTGTCAAGACCGACCTGTCCGGTG
CCCTGAATGAACTGCAGGACGAGGCAGCGCGGCTATCGTGGCTGGCCACGACGGGCGTT
CCTTGCGCAGCTGTGCTCGACGTTGTCACTGAAGCGGGAAGGGACTGGCTGCTATTGGG
CGAAGTGCCGGGGCAGGATCTCCTGTCATCTCACCTTGCTCCTGCCGAGAAAGTATCCA
TCATGGCTGATGCAATGCGGCGGCTGCATACGCTTGATCCGGCTACCTGCCCATTCGAC
CACCAAGCGAAACATCGCATCGAGCGAGCACGTACTCGGATGGAAGCCGGTCTTGTCGA
TCAGGATGATCTGGACGAAGAGCATCAGGGGCTCGCGCCAGCCGAACTGTTCGCCAGGC
TCAAGGCGCGCATGCCCGACGGCGAGGATCTCGTCGTGACCCATGGCGATGCCTGCTTG
CCGAATATCATGGTGGAAAATGGCCGCTTTTCTGGATTCATCGACTGTGGCCGGCTGGG
TGTGGCCGACCGCTATCAGGACATAGCGTTGGCTACCCGTGATATTGCTGAAGAGCTTG
GCGGCGAATGGGCTGACCGCTTCCTCGTGCTTTACGGTATCGCCGCTCCCGATTCGCAG
CGCATCGCCTTCTATCGCCTTCTTGACGAGTTCTTCTGAATGCATGAGTAACTGAGGAT
CCAGGGACAGCAGAGATACGCGTTAAGTCGACAATCAACCTCTGGATTACAAAATTTGT
GAAAGATTGACTGGTATTCTTAACTATGTTGCTCCTTTTACGCTATGTGGATACGCTGC
TTTAATGCCTTTGTATCATGCTATTGCTTCCCGTATGGCTTTCATTTTCTCCTCCTTGT
ATAAATCCTGGTTGCTGTCTCTTTATGAGGAGTTGTGGCCCGTTGTCAGGCAACGTGGC
GTGGTGTGCACTGTGTTTGCTGACGCAACCCCCACTGGTTGGGGCATTGCCACCACCTG
TCAGCTCCTTTCCGGGACTTTCGCTTTCCCCCTCCCTATTGCCACGGCGGAACTCATCG
CCGCCTGCCTTGCCCGCTGCTGGACAGGGGCTCGGCTGTTGGGCACTGACAATTCCGTG
GTGTTGTCGGGGAAATCATCGTCCTTTCCTTGGCTGCTCGCCTGTGTTGCCACCTGGAT
TCTGCGCGGGACGTCCTTCTGCTACGTCCCTTCGGCCCTCAATCCAGCGGACCTTCCTT
CCCGCGGCCTGCTGCCGGCTCTGCGGCCTCTTCCGCGTCTTCGCCTTCGCCCTCAGACG
AGTCGGATCTCCCTTTGGGCCGCCTCCCCGCGTCGACTTTAAGACCAATGACTTACAAG
GCAGCTGTAGATCTTAGCCACTTTTTAAAAGAAAAGGGGGGACTGGAAGGGCTAATTCA
CTCCCAACGAAGACAAGATCTGCTTTTTGCTTGTACTGGGTCTCTCTGGTTAGACCAGA
TCTGAGCCTGGGAGCTCTCTGGCTAACTAGGGAACCCACTGCTTAAGCCTCAATAAAGC
TTGCCTTGAGTGCTTCAAGTAGTGTGTGCCCGTCTGTTGTGTGACTCTGGTAACTAGAG
ATCCCTCAGACCCTTTTAGTCAGTGTGGAAAATCTCTAGCAGTACGTATAGTAGTTCAT
GTCATCTTATTATTCAGTATTTATAACTTGCAAAGAAATGAATATCAGAGAGTGAGAGG
AACTTGTTTATTGCAGCTTATAATGGTTACAAATAAAGCAATAGCATCACAAATTTCAC
AAATAAAGCATTTTTTTCACTGCATTCTAGTTGTGGTTTGTCCAAACTCATCAATGTAT
CTTATCATGTCTGGCTCTAGCTATCCCGCCCCTAACTCCGCCCATCCCGCCCCTAACTC
CGCCCAGTTCCGCCCATTCTCCGCCCCATGGCTGACTAATTTTTTTTATTTATGCAGAG
GCCGAGGCCGCCTCGGCCTCTGAGCTATTCCAGAAGTAGTGAGGAGGCTTTTTTGGAGG
CCTAGGGACGTACCCAATTCGCCCTATAGTGAGTCGTATTACGCGCGCTCACTGGCCGT
CGTTTTACAACGTCGTGACTGGGAAAACCCTGGCGTTACCCAACTTAATCGCCTTGCAG
CACATCCCCCTTTCGCCAGCTGGCGTAATAGCGAAGAGGCCCGCACCGATCGCCCTTCC
CAACAGTTGCGCAGCCTGAATGGCGAATGGGACGCGCCCTGTAGCGGCGCATTAAGCGC
GGCGGGTGTGGTGGTTACGCGCAGCGTGACCGCTACACTTGCCAGCGCCCTAGCGCCCG
CTCCTTTCGCTTTCTTCCCTTCCTTTCTCGCCACGTTCGCCGGCTTTCCCCGTCAAGCT
CTAAATCGGGGGCTCCCTTTAGGGTTCCGATTTAGTGCTTTACGGCACCTCGACCCCAA
AAAACTTGATTAGGGTGATGGTTCACGTAGTGGGCCATCGCCCTGATAGACGGTTTTTC
GCCCTTTGACGTTGGAGTCCACGTTCTTTAATAGTGGACTCTTGTTCCAAACTGGAACA
ACACTCAACCCTATCTCGGTCTATTCTTTTGATTTATAAGGGATTTTGCCGATTTCGGC
CTATTGGTTAAAAAATGAGCTGATTTAACAAAAATTTAACGCGAATTTTAACAAAATAT
TAACGCTTACAATTTAGGTGGCACTTTTCGGGGAAATGTGCGCGGAACCCCTATTTGTT
TATTTTTCTAAATACATTCAAATATGTATCCGCTCATGAGACAATAACCCTGATAAATG
CTTCAATAATATTGAAAAAGGAAGAGTATGAGTATTCAACATTTCCGTGTCGCCCTTAT
TCCCTTTTTTGCGGCATTTTGCCTTCCTGTTTTTGCTCACCCAGAAACGCTGGTGAAAG
TAAAAGATGCTGAAGATCAGTTGGGTGCACGAGTGGGTTACATCGAACTGGATCTCAAC
AGCGGTAAGATCCTTGAGAGTTTTCGCCCCGAAGAACGTTTTCCAATGATGAGCACTTT
TAAAGTTCTGCTATGTGGCGCGGTATTATCCCGTATTGACGCCGGGCAAGAGCAACTCG
GTCGCCGCATACACTATTCTCAGAATGACTTGGTTGAGTACTCACCAGTCACAGAAAAG
CATCTTACGGATGGCATGACAGTAAGAGAATTATGCAGTGCTGCCATAACCATGAGTGA
TAACACTGCGGCCAACTTACTTCTGACAACGATCGGAGGACCGAAGGAGCTAACCGCTT

D913 degron proteins and vectors comprised the following sequences:

1928z-d913, aka FMC63-CD28-CD3z-d913: This sequence corresponds to the in frame fusion of the CSF2RA signal sequence (amino acids 1-22), anti-CD19 scFv FMC63 (www.ebi.ac.uk/ena/data/view/ADM64594), the CD28 hinge, transmembrane, and cytoplasmic domains (amino acids 114-220), the CD3z intracellular ITAM domains (amino acids 55-164), a SG linker, and the d913 degron sequence.

(SEQ ID NO: 138)
MLLLVTSLLLCELPHPAFLLIPEQKLISEEDLDIQMTQTTSSLSASLGDR
VTISCRASQDISKYLNWYQQKPDGTVKLLIYHTSRLHSGVPSRFSGSGSG
TDYSLTISNLEQEDIATYFCQQGNTLPYTFGGGTKLEITGSTSGSGKPGS
GEGSTKGEVKLQESGPGLVAPSQSLSVTCTVSGVSLPDYGVSWIRQPPRK
GLEWLGVIWGSETTYYNSALKSRLTIIKDNSKSQVFLKMNSLQTDDTAIY
YCAKHYYYGGSYAMDYWGQGTSVTVSSAAAIEVMYPPPYLDNEKSNGTII
HVKGKHLCPSPLFPGPSKPFWVLVVVGGVLACYSLLVTVAFIIFWVRSKR
SRLLHSDYMNMTPRRPGPTRKHYQPYAPPRDFAAYRSRVKFSRSADAPAY
QQGQNQLYNELNLGRREEYDVLDKRRGRDPEMGGKPRRKNPQEGLYNELQ
KDKMAEAYSEIGMKGERRRGKGHDGLYQGLSTATKDTYDALHMQALPPRS
GFNVLMVHKRSHTGERPLQCEICGFTCRQKGNLLRHIKLHTGEKPFKCHL
CNYACQRRDAL

sCARA913 aka FMC63-CD28-d913 intracellular K0 (iK0): This sequence corresponds to the anti-CD19 scFv FMC63 (www.ebi.ac.uk/ena/data/view/ADM64594), the CD28 hinge, transmembrane, and cytoplasmic domains (amino acids 114-220), and d913 degron sequence, wherein all intracellular lysines are substituted to arginine.

(SEQ ID NO: 139)
MLLLVTSLLLCELPHPAFLLIPEQKLISEEDLDIQMTQTTSSLSASLGDRVTISCRASQ
DISKYLNWYQQKPDGTVKLLIYHTSRLHSGVPSRFSGSGSGTDYSLTISNLEQEDIATY
FCQQGNTLPYTFGGGTKLEITGSTSGSGKPGSGEGSTKGEVKLQESGPGLVAPSQSLSV
TCTVSGVSLPDYGVSWIRQPPRKGLEWLGVIWGSETTYYNSALKSRLTIIKDNSKSQVF
LKMNSLQTDDTAIYYCAKHYYYGGSYAMDYWGQGTSVTVSSAAAIEVIVIYPPPYLDNE
KSNGTIIHVKGKHLCPSPLFPGPSKPFWVLVVVGGVLACYSLLVTVAFIIFWVRSRRSR
LLHSDYMNMTPRRPGPTRRHYQPYAPPRDFAAYRSSGFNVLMVHRRSHTGERPLQCEIC
GFTCRQRGNLLRHIRLHTGERPFRCHLCNYACQRRDAL
BigSur-1928z-d913
(SEQ ID NO: 140)
TTTTGCACAACATGGGGGATCATGTAACTCGCCTTGATCGTTGGGAACCGGAGCTGAAT
GAAGCCATACCAAACGACGAGCGTGACACCACGATGCCTGTAGCAATGGCAACAACGTT
GCGCAAACTATTAACTGGCGAACTACTTACTCTAGCTTCCCGGCAACAATTAATAGACT
GGATGGAGGCGGATAAAGTTGCAGGACCACTTCTGCGCTCGGCCCTTCCGGCTGGCTGG
TTTATTGCTGATAAATCTGGAGCCGGTGAGCGTGGGTCTCGCGGTATCATTGCAGCACT
GGGGCCAGATGGTAAGCCCTCCCGTATCGTAGTTATCTACACGACGGGGAGTCAGGCAA
CTATGGATGAACGAAATAGACAGATCGCTGAGATAGGTGCCTCACTGATTAAGCATTGG
TAACTGTCAGACCAAGTTTACTCATATATACTTTAGATTGATTTAAAACTTCATTTTTA
ATTTAAAAGGATCTAGGTGAAGATCCTTTTTGATAATCTCATGACCAAAATCCCTTAAC
GTGAGTTTTCGTTCCACTGAGCGTCAGACCCCGTAGAAAAGATCAAAGGATCTTCTTGA
GATCCTTTTTTTCTGCGCGTAATCTGCTGCTTGCAAACAAAAAAACCACCGCTACCAGC
GGTGGTTTGTTTGCCGGATCAAGAGCTACCAACTCTTTTTCCGAAGGTAACTGGCTTCA
GCAGAGCGCAGATACCAAATACTGTTCTTCTAGTGTAGCCGTAGTTAGGCCACCACTTC
AAGAACTCTGTAGCACCGCCTACATACCTCGCTCTGCTAATCCTGTTACCAGTGGCTGC
TGCCAGTGGCGATAAGTCGTGTCTTACCGGGTTGGACTCAAGACGATAGTTACCGGATA
AGGCGCAGCGGTCGGGCTGAACGGGGGGTTCGTGCACACAGCCCAGCTTGGAGCGAACG
ACCTACACCGAACTGAGATACCTACAGCGTGAGCTATGAGAAAGCGCCACGCTTCCCGA
AGGGAGAAAGGCGGACAGGTATCCGGTAAGCGGCAGGGTCGGAACAGGAGAGCGCACGA
GGGAGCTTCCAGGGGGAAACGCCTGGTATCTTTATAGTCCTGTCGGGTTTCGCCACCTC
TGACTTGAGCGTCGATTTTTGTGATGCTCGTCAGGGGGGCGGAGCCTATGGAAAAACGC
CAGCAACGCGGCCTTTTTACGGTTCCTGGCCTTTTGCTGGCCTTTTGCTCACATGTTCT
TTCCTGCGTTATCCCCTGATTCTGTGGATAACCGTATTACCGCCTTTGAGTGAGCTGAT
ACCGCTCGCCGCAGCCGAACGACCGAGCGCAGCGAGTCAGTGAGCGAGGAAGCGGAAGA
GCGCCCAATACGCAAACCGCCTCTCCCCGCGCGTTGGCCGATTCATTAATGCAGCTGGC
ACGACAGGTTTCCCGACTGGAAAGCGGGCAGTGAGCGCAACGCAATTAATGTGAGTTAG
CTCACTCATTAGGCACCCCAGGCTTTACACTTTATGCTTCCGGCTCGTATGTTGTGTGG
AATTGTGAGCGGATAACAATTTCACACAGGAAACAGCTATGACCATGATTACGCCAAGC
GCGCAATTAACCCTCACTAAAGGGAACAAAAGCTGGAGCTGCAAGCTTAATGTAGTCTT
ATGCAATACTCTTGTAGTCTTGCAACATGGTAACGATGAGTTAGCAACATGCCTTACAA
GGAGAGAAAAAGCACCGTGCATGCCGATTGGTGGAAGTAAGGTGGTACGATCGTGCCTT
ATTAGGAAGGCAACAGACGGGTCTGACATGGATTGGACGAACCACTGAATTGCCGCATT
GCAGAGATATTGTATTTAAGTGCCTAGCTCGATACATAAACGGGTCTCTCTGGTTAGAC
CAGATCTGAGCCTGGGAGCTCTCTGGCTAACTAGGGAACCCACTGCTTAAGCCTCAATA
AAGCTTGCCTTGAGTGCTTCAAGTAGTGTGTGCCCGTCTGTTGTGTGACTCTGGTAACT
AGAGATCCCTCAGACCCTTTTAGTCAGTGTGGAAAATCTCTAGCAGTGGCGCCCGAACA
GGGACTTGAAAGCGAAAGGGAAACCAGAGGAGCTCTCTCGACGCAGGACTCGGCTTGCT
GAAGCGCGCACGGCAAGAGGCGAGGGGCGGCGACTGGTGAGTACGCCAAAAATTTTGAC
TAGCGGAGGCTAGAAGGAGAGAGATGGGTGCGAGAGCGTCAGTATTAAGCGGGGGAGAA
TTAGATCGCGATGGGAAAAAATTCGGTTAAGGCCAGGGGGAAAGAAAAAATATAAATTA
AAACATATAGTATGGGCAAGCAGGGAGCTAGAACGATTCGCAGTTAATCCTGGCCTGTT
AGAAACATCAGAAGGCTGTAGACAAATACTGGGACAGCTACAACCATCCCTTCAGACAG
GATCAGAAGAACTTAGATCATTATATAATACAGTAGCAACCCTCTATTGTGTGCATCAA
AGGATAGAGATAAAAGACACCAAGGAAGCTTTAGACAAGATAGAGGAAGAGCAAAACAA
AAGTAAGACCACCGCACAGCAAGCGGCCGCTGATCTTCAGACCTGGAGGAGGAGATATG
AGGGACAATTGGAGAAGTGAATTATATAAATATAAAGTAGTAAAAATTGAACCATTAGG
AGTAGCACCCACCAAGGCAAAGAGAAGAGTGGTGCAGAGAGAAAAAAGAGCAGTGGGAA
TAGGAGCTTTGTTCCTTGGGTTCTTGGGAGCAGCAGGAAGCACTATGGGCGCAGCGTCA
ATGACGCTGACGGTACAGGCCAGACAATTATTGTCTGGTATAGTGCAGCAGCAGAACAA
TTTGCTGAGGGCTATTGAGGCGCAACAGCATCTGTTGCAACTCACAGTCTGGGGCATCA
AGCAGCTCCAGGCAAGAATCCTGGCTGTGGAAAGATACCTAAAGGATCAACAGCTCCTG
GGGATTTGGGGTTGCTCTGGAAAACTCATTTGCACCACTGCTGTGCCTTGGAATGCTAG
TTGGAGTAATAAATCTCTGGAACAGATTTGGAATCACACGACCTGGATGGAGTGGGACA
GAGAAATTAACAATTACACAAGCTTAATACACTCCTTAATTGAAGAATCGCAAAACCAG
CAAGAAAAGAATGAACAAGAATTATTGGAATTAGATAAATGGGCAAGTTTGTGGAATTG
GTTTAACATAACAAATTGGCTGTGGTATATAAAATTATTCATAATGATAGTAGGAGGCT
TGGTAGGTTTAAGAATAGTTTTTGCTGTACTTTCTATAGTGAATAGAGTTAGGCAGGGA
TATTCACCATTATCGTTTCAGACCCACCTCCCAACCCCGAGGGGACCCTATTCCAGCAC
ATATGAGGCTTGGCGTAACTAGATCTTGAGACAAATGGCAGTATTCATCCACAATTTTA
AAAGAAAAGGGGGGATTGGGGGGTACAGTGCAGGGGAAAGAATAGTAGACATAATAGCA
ACAGACATACAAACTAAAGAATTACAAAAACAAATTACAAAAATTCAAAATTTTCGGGT
TTATTACAGGGACAGCAGAGATCCACTTTGGGCTCGAGGGGGCCCGGGTGCAAAGATGG
ATAAAGTTTTAAACAGAGAGGAATCTTTGCAGCTAATGGACCTTCTAGGTCTTGAAAGG
AGTGGGAATTGGCTCCGGTGCCCGTCAGTGGGCAGAGCGCACATCGCCCACAGTCCCCG
AGAAGTTGGGGGGAGGGGTCGGCAATTGATCCGGTGCCTAGAGAAGGTGGCGCGGGGTA
AACTGGGAAAGTGATGTCGTGTACTGGCTCCGCCTTTTTCCCGAGGGTGGGGGAGAACC
GTATATAAGTGCAGTAGTCGCCGTGAACGTTCTTTTTCGCAACGGGTTTGCCGCCAGAA
CACAGGTAAGTGCCGTGTGTGGTTCCCGCGGGCCTGGCCTCTTTACGGGTTATGGCCCT
TGCGTGCCTTGAATTACTTCCACCTGGCTGCAGTACGTGATTCTTGATCCCGAGCTTCG
GGTTGGAAGTGGGTGGGAGAGTTCGAGGCCTTGCGCTTAAGGAGCCCCTTCGCCTCGTG
CTTGAGTTGAGGCCTGGCCTGGGCGCTGGGGCCGCCGCGTGCGAATCTGGTGGCACCTT
CGCGCCTGTCTCGCTGCTTTCGATAAGTCTCTAGCCATTTAAAATTTTTGATGACCTGC
TGCGACGCTTTTTTTCTGGCAAGATAGTCTTGTAAATGCGGGCCAAGATCTGCACACTG
GTATTTCGGTTTTTGGGGCCGCGGGCGGCGACGGGGCCCGTGCGTCCCAGCGCACATGT
TCGGCGAGGCGGGGCCTGCGAGCGCGGCCACCGAGAATCGGACGGGGGTAGTCTCAAGC
TGGCCGGCCTGCTCTGGTGCCTGGCCTCGCGCCGCCGTGTATCGCCCCGCCCTGGGCGG
CAAGGCTGGCCCGGTCGGCACCAGTTGCGTGAGCGGAAAGATGGCCGCTTCCCGGCCCT
GCTGCAGGGAGCTCAAAATGGAGGACGCGGCGCTCGGGAGAGCGGGCGGGTGAGTCACC
CACACAAAGGAAAAGGGCCTTTCCGTCCTCAGCCGTCGCTTCATGTGACTCCACGGAGT
ACCGGGCGCCGTCCAGGCACCTCGATTAGTTCTCGAGCTTTTGGAGTACGTCGTCTTTA
GGTTGGGGGGAGGGGTTTTATGCGATGGAGTTTCCCCACACTGAGTGGGTGGAGACTGA
AGTTAGGCCAGCTTGGCACTTGATGTAATTCTCCTTGGAATTTGCCCTTTTTGAGTTTG
GATCTTGGTTCATTCTCAAGCCTCAGACAGTGGTTCAAAGTTTTTTTCTTCCATTTCAG
GTGTCGTGAATACCGTCGACTCCGGAATAGCCACCATGCTTCTCCTGGTGACAAGCCTT
CTGCTCTGTGAGTTACCACACCCAGCATTCCTCCTGATCCCAGAGCAGAAACTCATCTC
AGAAGAGGATCTGGACATCCAGATGACACAGACTACATCCTCCCTGTCTGCCTCTCTGG
GAGACAGAGTCACCATCAGTTGCAGGGCAAGTCAGGACATTAGTAAATATTTAAATTGG
TATCAGCAGAAACCAGATGGAACTGTTAAACTCCTGATCTACCATACATCAAGATTACA
CTCAGGAGTCCCATCAAGGTTCAGTGGCAGTGGGTCTGGAACAGATTATTCTCTCACCA
TTAGCAACCTGGAGCAAGAAGATATTGCCACTTACTTTTGCCAACAGGGTAATACGCTT
CCGTACACGTTCGGAGGGGGGACTAAGTTGGAAATAACAGGCTCCACCTCTGGATCCGG
CAAGCCCGGATCTGGCGAGGGATCCACCAAGGGCGAGGTGAAACTGCAGGAGTCAGGAC
CTGGCCTGGTGGCGCCCTCACAGAGCCTGTCCGTCACATGCACTGTCTCAGGGGTCTCA
TTACCCGACTATGGTGTAAGCTGGATTCGCCAGCCTCCACGAAAGGGTCTGGAGTGGCT
GGGAGTAATATGGGGTAGTGAAACCACATACTATAATTCAGCTCTCAAATCCAGACTGA
CCATCATCAAGGACAACTCCAAGAGCCAAGTTTTCTTAAAAATGAACAGTCTGCAAACT
GATGACACAGCCATTTACTACTGTGCCAAACATTATTACTACGGTGGTAGCTATGCTAT
GGACTACTGGGGTCAAGGAACCTCAGTCACaGTCTCCTCAGCGGCCGCAATTGAAGTTA
TGTATCCTCCTCCTTACCTAGACAATGAGAAGAGCAATGGAACCATTATCCATGTGAAA
GGGAAACACCTTTGTCCAAGTCCCCTATTTCCCGGACCTTCTAAGCCCTTTTGGGTGCT
GGTGGTGGTTGGGGGAGTCCTGGCTTGCTATAGCTTGCTAGTAACAGTGGCCTTTATTA
TTTTCTGGGTGAGGAGTAAGAGGAGCAGGCTCCTGCACAGTGACTACATGAACATGACT
CCTAGAAGGCCTGGACCCACCCGCAAGCATTACCAGCCCTATGCCCCACCACGCGACTT
CGCAGCCTATCGCTCCAGAGTGAAGTTCAGCAGGAGCGCAGACGCCCCCGCGTACCAGC
AGGGCCAGAACCAGCTCTATAACGAGCTCAATCTAGGACGAAGAGAGGAGTACGATGTT
TTGGACAAGAGgCGTGGCCGGGACCCTGAGATGGGGGGAAAGCCGAGAAGGAAGAACCC
TCAGGAAGGCCTGTACAATGAACTGCAGAAAGATAAGATGGCGGAGGCCTACAGTGAGA
TTGGGATGAAAGGCGAGCGCCGGAGGGGCAAGGGGCACGATGGCCTTTACCAGGGTCTC
AGTACAGCCACCAAGGACACCTACGACGCCCTTCACATGCAGGCCCTGCCCCCTCGCTC
CGGATTCAATGTCTTAATGGTTCATAAGCGAAGCCATACTGGTGAACGCCCATTGCAGT
GCGAAATATGCGGCTTTACCTGCCGCCAGAAAGGTAACCTCCTCCGCCACATTAAACTG
CACACAGGGGAAAAACCTTTTAAGTGTCACCTCTGCAACTATGCATGCCAAAGAAGAGA
TGCGCTCCGCAAAAGACGCGGATCCGGCGAGGGTAGAGGCAGTCTCCTCACATGTGGCG
ATGTGGAAGAAAACCCAGGCCCCATGGTGAGCAAGGGCGAGGAGGATAACATGGCCATC
ATCAAGGAGTTCATGCGCTTCAAGGTGCACATGGAGGGCTCCGTGAACGGCCACGAGTT
CGAGATCGAGGGCGAGGGCGAGGGCCGCCCCTACGAGGGCACCCAGACCGCCAAGCTGA
AGGTGACCAAGGGTGGCCCCCTGCCCTTCGCCTGGGACATCCTGTCCCCTCAGTTCATG
TACGGCTCCAAGGCCTACGTGAAGCACCCCGCCGACATCCCCGACTACTTGAAGCTGTC
CTTCCCCGAGGGCTTCAAGTGGGAGCGCGTGATGAACTTCGAGGACGGCGGCGTGGTGA
CCGTGACCCAGGACTCCTCCCTGCAGGACGGCGAGTTCATCTACAAGGTGAAGCTGCGC
GGCACCAACTTCCCCTCCGACGGCCCCGTAATGCAGAAGAAGACCATGGGCTGGGAGGC
CTCCTCCGAGCGGATGTACCCCGAGGACGGCGCCCTGAAGGGCGAGATCAAGCAGAGGC
TGAAGCTGAAGGACGGCGGCCACTACGACGCTGAGGTCAAGACCACCTACAAGGCCAAG
AAGCCCGTGCAGCTGCCCGGCGCCTACAACGTCAACATCAAGTTGGACATCACCTCCCA
CAACGAGGACTACACCATCGTGGAACAGTACGAACGCGCCGAGGGCCGCCACTCCACCG
GCGGCATGGACGAGCTGTACAAGtaaATGCATGAGTAACTGAGGATCCAGGGACAGCAG
AGATACGCGTTAAGTCGACAATCAACCTCTGGATTACAAAATTTGTGAAAGATTGACTG
GTATTCTTAACTATGTTGCTCCTTTTACGCTATGTGGATACGCTGCTTTAATGCCTTTG
TATCATGCTATTGCTTCCCGTATGGCTTTCATTTTCTCCTCCTTGTATAAATCCTGGTT
GCTGTCTCTTTATGAGGAGTTGTGGCCCGTTGTCAGGCAACGTGGCGTGGTGTGCACTG
TGTTTGCTGACGCAACCCCCACTGGTTGGGGCATTGCCACCACCTGTCAGCTCCTTTCC
GGGACTTTCGCTTTCCCCCTCCCTATTGCCACGGCGGAACTCATCGCCGCCTGCCTTGC
CCGCTGCTGGACAGGGGCTCGGCTGTTGGGCACTGACAATTCCGTGGTGTTGTCGGGGA
AATCATCGTCCTTTCCTTGGCTGCTCGCCTGTGTTGCCACCTGGATTCTGCGCGGGACG
TCCTTCTGCTACGTCCCTTCGGCCCTCAATCCAGCGGACCTTCCTTCCCGCGGCCTGCT
GCCGGCTCTGCGGCCTCTTCCGCGTCTTCGCCTTCGCCCTCAGACGAGTCGGATCTCCC
TTTGGGCCGCCTCCCCGCGTCGACTTTAAGACCAATGACTTACAAGGCAGCTGTAGATC
TTAGCCACTTTTTAAAAGAAAAGGGGGGACTGGAAGGGCTAATTCACTCCCAACGAAGA
CAAGATCTGCTTTTTGCTTGTACTGGGTCTCTCTGGTTAGACCAGATCTGAGCCTGGGA
GCTCTCTGGCTAACTAGGGAACCCACTGCTTAAGCCTCAATAAAGCTTGCCTTGAGTGC
TTCAAGTAGTGTGTGCCCGTCTGTTGTGTGACTCTGGTAACTAGAGATCCCTCAGACCC
TTTTAGTCAGTGTGGAAAATCTCTAGCAGTACGTATAGTAGTTCATGTCATCTTATTAT
TCAGTATTTATAACTTGCAAAGAAATGAATATCAGAGAGTGAGAGGAACTTGTTTATTG
CAGCTTATAATGGTTACAAATAAAGCAATAGCATCACAAATTTCACAAATAAAGCATTT
TTTTCACTGCATTCTAGTTGTGGTTTGTCCAAACTCATCAATGTATCTTATCATGTCTG
GCTCTAGCTATCCCGCCCCTAACTCCGCCCATCCCGCCCCTAACTCCGCCCAGTTCCGC
CCATTCTCCGCCCCATGGCTGACTAATTTTTTTTATTTATGCAGAGGCCGAGGCCGCCT
CGGCCTCTGAGCTATTCCAGAAGTAGTGAGGAGGCTTTTTTGGAGGCCTAGGGACGTAC
CCAATTCGCCCTATAGTGAGTCGTATTACGCGCGCTCACTGGCCGTCGTTTTACAACGT
CGTGACTGGGAAAACCCTGGCGTTACCCAACTTAATCGCCTTGCAGCACATCCCCCTTT
CGCCAGCTGGCGTAATAGCGAAGAGGCCCGCACCGATCGCCCTTCCCAACAGTTGCGCA
GCCTGAATGGCGAATGGGACGCGCCCTGTAGCGGCGCATTAAGCGCGGCGGGTGTGGTG
GTTACGCGCAGCGTGACCGCTACACTTGCCAGCGCCCTAGCGCCCGCTCCTTTCGCTTT
CTTCCCTTCCTTTCTCGCCACGTTCGCCGGCTTTCCCCGTCAAGCTCTAAATCGGGGGC
TCCCTTTAGGGTTCCGATTTAGTGCTTTACGGCACCTCGACCCCAAAAAACTTGATTAG
GGTGATGGTTCACGTAGTGGGCCATCGCCCTGATAGACGGTTTTTCGCCCTTTGACGTT
GGAGTCCACGTTCTTTAATAGTGGACTCTTGTTCCAAACTGGAACAACACTCAACCCTA
TCTCGGTCTATTCTTTTGATTTATAAGGGATTTTGCCGATTTCGGCCTATTGGTTAAAA
AATGAGCTGATTTAACAAAAATTTAACGCGAATTTTAACAAAATATTAACGCTTACAAT
TTAGGTGGCACTTTTCGGGGAAATGTGCGCGGAACCCCTATTTGTTTATTTTTCTAAAT
ACATTCAAATATGTATCCGCTCATGAGACAATAACCCTGATAAATGCTTCAATAATATT
GAAAAAGGAAGAGTATGAGTATTCAACATTTCCGTGTCGCCCTTATTCCCTTTTTTGCG
GCATTTTGCCTTCCTGTTTTTGCTCACCCAGAAACGCTGGTGAAAGTAAAAGATGCTGA
AGATCAGTTGGGTGCACGAGTGGGTTACATCGAACTGGATCTCAACAGCGGTAAGATCC
TTGAGAGTTTTCGCCCCGAAGAACGTTTTCCAATGATGAGCACTTTTAAAGTTCTGCTA
TGTGGCGCGGTATTATCCCGTATTGACGCCGGGCAAGAGCAACTCGGTCGCCGCATACA
CTATTCTCAGAATGACTTGGTTGAGTACTCACCAGTCACAGAAAAGCATCTTACGGATG
GCATGACAGTAAGAGAATTATGCAGTGCTGCCATAACCATGAGTGATAACACTGCGGCC
AACTTACTTCTGACAACGATCGGAGGACCGAAGGAGCTAACCGCTT
BigSur-sCARA913 (FMC63-CD28-d913)
(SEQ ID NO: 141)
TTTTGCACAACATGGGGGATCATGTAACTCGCCTTGATCGTTGGGAACCGGAGCTGAAT
GAAGCCATACCAAACGACGAGCGTGACACCACGATGCCTGTAGCAATGGCAACAACGTT
GCGCAAACTATTAACTGGCGAACTACTTACTCTAGCTTCCCGGCAACAATTAATAGACT
GGATGGAGGCGGATAAAGTTGCAGGACCACTTCTGCGCTCGGCCCTTCCGGCTGGCTGG
TTTATTGCTGATAAATCTGGAGCCGGTGAGCGTGGGTCTCGCGGTATCATTGCAGCACT
GGGGCCAGATGGTAAGCCCTCCCGTATCGTAGTTATCTACACGACGGGGAGTCAGGCAA
CTATGGATGAACGAAATAGACAGATCGCTGAGATAGGTGCCTCACTGATTAAGCATTGG
TAACTGTCAGACCAAGTTTACTCATATATACTTTAGATTGATTTAAAACTTCATTTTTA
ATTTAAAAGGATCTAGGTGAAGATCCTTTTTGATAATCTCATGACCAAAATCCCTTAAC
GTGAGTTTTCGTTCCACTGAGCGTCAGACCCCGTAGAAAAGATCAAAGGATCTTCTTGA
GATCCTTTTTTTCTGCGCGTAATCTGCTGCTTGCAAACAAAAAAACCACCGCTACCAGC
GGTGGTTTGTTTGCCGGATCAAGAGCTACCAACTCTTTTTCCGAAGGTAACTGGCTTCA
GCAGAGCGCAGATACCAAATACTGTTCTTCTAGTGTAGCCGTAGTTAGGCCACCACTTC
AAGAACTCTGTAGCACCGCCTACATACCTCGCTCTGCTAATCCTGTTACCAGTGGCTGC
TGCCAGTGGCGATAAGTCGTGTCTTACCGGGTTGGACTCAAGACGATAGTTACCGGATA
AGGCGCAGCGGTCGGGCTGAACGGGGGGTTCGTGCACACAGCCCAGCTTGGAGCGAACG
ACCTACACCGAACTGAGATACCTACAGCGTGAGCTATGAGAAAGCGCCACGCTTCCCGA
AGGGAGAAAGGCGGACAGGTATCCGGTAAGCGGCAGGGTCGGAACAGGAGAGCGCACGA
GGGAGCTTCCAGGGGGAAACGCCTGGTATCTTTATAGTCCTGTCGGGTTTCGCCACCTC
TGACTTGAGCGTCGATTTTTGTGATGCTCGTCAGGGGGGCGGAGCCTATGGAAAAACGC
CAGCAACGCGGCCTTTTTACGGTTCCTGGCCTTTTGCTGGCCTTTTGCTCACATGTTCT
TTCCTGCGTTATCCCCTGATTCTGTGGATAACCGTATTACCGCCTTTGAGTGAGCTGAT
ACCGCTCGCCGCAGCCGAACGACCGAGCGCAGCGAGTCAGTGAGCGAGGAAGCGGAAGA
GCGCCCAATACGCAAACCGCCTCTCCCCGCGCGTTGGCCGATTCATTAATGCAGCTGGC
ACGACAGGTTTCCCGACTGGAAAGCGGGCAGTGAGCGCAACGCAATTAATGTGAGTTAG
CTCACTCATTAGGCACCCCAGGCTTTACACTTTATGCTTCCGGCTCGTATGTTGTGTGG
AATTGTGAGCGGATAACAATTTCACACAGGAAACAGCTATGACCATGATTACGCCAAGC
GCGCAATTAACCCTCACTAAAGGGAACAAAAGCTGGAGCTGCAAGCTTAATGTAGTCTT
ATGCAATACTCTTGTAGTCTTGCAACATGGTAACGATGAGTTAGCAACATGCCTTACAA
GGAGAGAAAAAGCACCGTGCATGCCGATTGGTGGAAGTAAGGTGGTACGATCGTGCCTT
ATTAGGAAGGCAACAGACGGGTCTGACATGGATTGGACGAACCACTGAATTGCCGCATT
GCAGAGATATTGTATTTAAGTGCCTAGCTCGATACATAAACGGGTCTCTCTGGTTAGAC
CAGATCTGAGCCTGGGAGCTCTCTGGCTAACTAGGGAACCCACTGCTTAAGCCTCAATA
AAGCTTGCCTTGAGTGCTTCAAGTAGTGTGTGCCCGTCTGTTGTGTGACTCTGGTAACT
AGAGATCCCTCAGACCCTTTTAGTCAGTGTGGAAAATCTCTAGCAGTGGCGCCCGAACA
GGGACTTGAAAGCGAAAGGGAAACCAGAGGAGCTCTCTCGACGCAGGACTCGGCTTGCT
GAAGCGCGCACGGCAAGAGGCGAGGGGCGGCGACTGGTGAGTACGCCAAAAATTTTGAC
TAGCGGAGGCTAGAAGGAGAGAGATGGGTGCGAGAGCGTCAGTATTAAGCGGGGGAGAA
TTAGATCGCGATGGGAAAAAATTCGGTTAAGGCCAGGGGGAAAGAAAAAATATAAATTA
AAACATATAGTATGGGCAAGCAGGGAGCTAGAACGATTCGCAGTTAATCCTGGCCTGTT
AGAAACATCAGAAGGCTGTAGACAAATACTGGGACAGCTACAACCATCCCTTCAGACAG
GATCAGAAGAACTTAGATCATTATATAATACAGTAGCAACCCTCTATTGTGTGCATCAA
AGGATAGAGATAAAAGACACCAAGGAAGCTTTAGACAAGATAGAGGAAGAGCAAAACAA
AAGTAAGACCACCGCACAGCAAGCGGCCGCTGATCTTCAGACCTGGAGGAGGAGATATG
AGGGACAATTGGAGAAGTGAATTATATAAATATAAAGTAGTAAAAATTGAACCATTAGG
AGTAGCACCCACCAAGGCAAAGAGAAGAGTGGTGCAGAGAGAAAAAAGAGCAGTGGGAA
TAGGAGCTTTGTTCCTTGGGTTCTTGGGAGCAGCAGGAAGCACTATGGGCGCAGCGTCA
ATGACGCTGACGGTACAGGCCAGACAATTATTGTCTGGTATAGTGCAGCAGCAGAACAA
TTTGCTGAGGGCTATTGAGGCGCAACAGCATCTGTTGCAACTCACAGTCTGGGGCATCA
AGCAGCTCCAGGCAAGAATCCTGGCTGTGGAAAGATACCTAAAGGATCAACAGCTCCTG
GGGATTTGGGGTTGCTCTGGAAAACTCATTTGCACCACTGCTGTGCCTTGGAATGCTAG
TTGGAGTAATAAATCTCTGGAACAGATTTGGAATCACACGACCTGGATGGAGTGGGACA
GAGAAATTAACAATTACACAAGCTTAATACACTCCTTAATTGAAGAATCGCAAAACCAG
CAAGAAAAGAATGAACAAGAATTATTGGAATTAGATAAATGGGCAAGTTTGTGGAATTG
GTTTAACATAACAAATTGGCTGTGGTATATAAAATTATTCATAATGATAGTAGGAGGCT
TGGTAGGTTTAAGAATAGTTTTTGCTGTACTTTCTATAGTGAATAGAGTTAGGCAGGGA
TATTCACCATTATCGTTTCAGACCCACCTCCCAACCCCGAGGGGACCCTATTCCAGCAC
ATATGAGGCTTGGCGTAACTAGATCTTGAGACAAATGGCAGTATTCATCCACAATTTTA
AAAGAAAAGGGGGGATTGGGGGGTACAGTGCAGGGGAAAGAATAGTAGACATAATAGCA
ACAGACATACAAACTAAAGAATTACAAAAACAAATTACAAAAATTCAAAATTTTCGGGT
TTATTACAGGGACAGCAGAGATCCACTTTGGGCTCGAGGGGGCCCGGGTGCAAAGATGG
ATAAAGTTTTAAACAGAGAGGAATCTTTGCAGCTAATGGACCTTCTAGGTCTTGAAAGG
AGTGGGAATTGGCTCCGGTGCCCGTCAGTGGGCAGAGCGCACATCGCCCACAGTCCCCG
AGAAGTTGGGGGGAGGGGTCGGCAATTGATCCGGTGCCTAGAGAAGGTGGCGCGGGGTA
AACTGGGAAAGTGATGTCGTGTACTGGCTCCGCCTTTTTCCCGAGGGTGGGGGAGAACC
GTATATAAGTGCAGTAGTCGCCGTGAACGTTCTTTTTCGCAACGGGTTTGCCGCCAGAA
CACAGGTAAGTGCCGTGTGTGGTTCCCGCGGGCCTGGCCTCTTTACGGGTTATGGCCCT
TGCGTGCCTTGAATTACTTCCACCTGGCTGCAGTACGTGATTCTTGATCCCGAGCTTCG
GGTTGGAAGTGGGTGGGAGAGTTCGAGGCCTTGCGCTTAAGGAGCCCCTTCGCCTCGTG
CTTGAGTTGAGGCCTGGCCTGGGCGCTGGGGCCGCCGCGTGCGAATCTGGTGGCACCTT
CGCGCCTGTCTCGCTGCTTTCGATAAGTCTCTAGCCATTTAAAATTTTTGATGACCTGC
TGCGACGCTTTTTTTCTGGCAAGATAGTCTTGTAAATGCGGGCCAAGATCTGCACACTG
GTATTTCGGTTTTTGGGGCCGCGGGCGGCGACGGGGCCCGTGCGTCCCAGCGCACATGT
TCGGCGAGGCGGGGCCTGCGAGCGCGGCCACCGAGAATCGGACGGGGGTAGTCTCAAGC
TGGCCGGCCTGCTCTGGTGCCTGGCCTCGCGCCGCCGTGTATCGCCCCGCCCTGGGCGG
CAAGGCTGGCCCGGTCGGCACCAGTTGCGTGAGCGGAAAGATGGCCGCTTCCCGGCCCT
GCTGCAGGGAGCTCAAAATGGAGGACGCGGCGCTCGGGAGAGCGGGCGGGTGAGTCACC
CACACAAAGGAAAAGGGCCTTTCCGTCCTCAGCCGTCGCTTCATGTGACTCCACGGAGT
ACCGGGCGCCGTCCAGGCACCTCGATTAGTTCTCGAGCTTTTGGAGTACGTCGTCTTTA
GGTTGGGGGGAGGGGTTTTATGCGATGGAGTTTCCCCACACTGAGTGGGTGGAGACTGA
AGTTAGGCCAGCTTGGCACTTGATGTAATTCTCCTTGGAATTTGCCCTTTTTGAGTTTG
GATCTTGGTTCATTCTCAAGCCTCAGACAGTGGTTCAAAGTTTTTTTCTTCCATTTCAG
GTGTCGTGAATACCGTCGACTCCGGAATAGCCACCATGCTTCTCCTGGTGACAAGCCTT
CTGCTCTGTGAGTTACCACACCCAGCATTCCTCCTGATCCCAGAGCAGAAACTCATCTC
AGAAGAGGATCTGGACATCCAGATGACACAGACTACATCCTCCCTGTCTGCCTCTCTGG
GAGACAGAGTCACCATCAGTTGCAGGGCAAGTCAGGACATTAGTAAATATTTAAATTGG
TATCAGCAGAAACCAGATGGAACTGTTAAACTCCTGATCTACCATACATCAAGATTACA
CTCAGGAGTCCCATCAAGGTTCAGTGGCAGTGGGTCTGGAACAGATTATTCTCTCACCA
TTAGCAACCTGGAGCAAGAAGATATTGCCACTTACTTTTGCCAACAGGGTAATACGCTT
CCGTACACGTTCGGAGGGGGGACTAAGTTGGAAATAACAGGCTCCACCTCTGGATCCGG
CAAGCCCGGATCTGGCGAGGGATCCACCAAGGGCGAGGTGAAACTGCAGGAGTCAGGAC
CTGGCCTGGTGGCGCCCTCACAGAGCCTGTCCGTCACATGCACTGTCTCAGGGGTCTCA
TTACCCGACTATGGTGTAAGCTGGATTCGCCAGCCTCCACGAAAGGGTCTGGAGTGGCT
GGGAGTAATATGGGGTAGTGAAACCACATACTATAATTCAGCTCTCAAATCCAGACTGA
CCATCATCAAGGACAACTCCAAGAGCCAAGTTTTCTTAAAAATGAACAGTCTGCAAACT
GATGACACAGCCATTTACTACTGTGCCAAACATTATTACTACGGTGGTAGCTATGCTAT
GGACTACTGGGGTCAAGGAACCTCAGTCACaGTCTCCTCAGCGGCCGCAATTGAAGTTA
TGTATCCTCCTCCTTACCTAGACAATGAGAAGAGCAATGGAACCATTATCCATGTGAAA
GGGAAACACCTTTGTCCAAGTCCCCTATTTCCCGGACCTTCTAAGCCCTTTTGGGTGCT
GGTGGTGGTTGGGGGAGTCCTGGCTTGCTATAGCTTGCTAGTAACAGTGGCCTTTATTA
TTTTCTGGGTGAGGAGTcggAGGAGCAGGCTCCTGCACAGTGACTACATGAACATGACT
CCTAGAAGGCCTGGACCCACCCGCcggCATTACCAGCCCTATGCCCCACCACGCGACTT
CGCAGCCTATCGCTCCTCCGGATTCAATGTCTTAATGGTTCATcggCGAAGCCATACTG
GTGAACGCCCATTGCAGTGCGAAATATGCGGCTTTACCTGCCGCCAGcgcGGTAACCTC
CTCCGCCACATTcgtCTGCACACAGGGGAAcggCCTTTTcggTGTCACCTCTGCAACTA
TGCATGCCAAAGAAGAGATGCGCTCAGAAGGAGACGCGGATCCGGCGAGGGTAGAGGCA
GTCTCCTCACATGTGGCGATGTGGAAGAAAACCCAGGCCCCATGGTGAGCAAGGGCGAG
GAGGATAACATGGCCATCATCAAGGAGTTCATGCGCTTCAAGGTGCACATGGAGGGCTC
CGTGAACGGCCACGAGTTCGAGATCGAGGGCGAGGGCGAGGGCCGCCCCTACGAGGGCA
CCCAGACCGCCAAGCTGAAGGTGACCAAGGGTGGCCCCCTGCCCTTCGCCTGGGACATC
CTGTCCCCTCAGTTCATGTACGGCTCCAAGGCCTACGTGAAGCACCCCGCCGACATCCC
CGACTACTTGAAGCTGTCCTTCCCCGAGGGCTTCAAGTGGGAGCGCGTGATGAACTTCG
AGGACGGCGGCGTGGTGACCGTGACCCAGGACTCCTCCCTGCAGGACGGCGAGTTCATC
TACAAGGTGAAGCTGCGCGGCACCAACTTCCCCTCCGACGGCCCCGTAATGCAGAAGAA
GACCATGGGCTGGGAGGCCTCCTCCGAGCGGATGTACCCCGAGGACGGCGCCCTGAAGG
GCGAGATCAAGCAGAGGCTGAAGCTGAAGGACGGCGGCCACTACGACGCTGAGGTCAAG
ACCACCTACAAGGCCAAGAAGCCCGTGCAGCTGCCCGGCGCCTACAACGTCAACATCAA
GTTGGACATCACCTCCCACAACGAGGACTACACCATCGTGGAACAGTACGAACGCGCCG
AGGGCCGCCACTCCACCGGCGGCATGGACGAGCTGTACAAGtaaATGCATGAGTAACTG
AGGATCCAGGGACAGCAGAGATACGCGTTAAGTCGACAATCAACCTCTGGATTACAAAA
TTTGTGAAAGATTGACTGGTATTCTTAACTATGTTGCTCCTTTTACGCTATGTGGATAC
GCTGCTTTAATGCCTTTGTATCATGCTATTGCTTCCCGTATGGCTTTCATTTTCTCCTC
CTTGTATAAATCCTGGTTGCTGTCTCTTTATGAGGAGTTGTGGCCCGTTGTCAGGCAAC
GTGGCGTGGTGTGCACTGTGTTTGCTGACGCAACCCCCACTGGTTGGGGCATTGCCACC
ACCTGTCAGCTCCTTTCCGGGACTTTCGCTTTCCCCCTCCCTATTGCCACGGCGGAACT
CATCGCCGCCTGCCTTGCCCGCTGCTGGACAGGGGCTCGGCTGTTGGGCACTGACAATT
CCGTGGTGTTGTCGGGGAAATCATCGTCCTTTCCTTGGCTGCTCGCCTGTGTTGCCACC
TGGATTCTGCGCGGGACGTCCTTCTGCTACGTCCCTTCGGCCCTCAATCCAGCGGACCT
TCCTTCCCGCGGCCTGCTGCCGGCTCTGCGGCCTCTTCCGCGTCTTCGCCTTCGCCCTC
AGACGAGTCGGATCTCCCTTTGGGCCGCCTCCCCGCGTCGACTTTAAGACCAATGACTT
ACAAGGCAGCTGTAGATCTTAGCCACTTTTTAAAAGAAAAGGGGGGACTGGAAGGGCTA
ATTCACTCCCAACGAAGACAAGATCTGCTTTTTGCTTGTACTGGGTCTCTCTGGTTAGA
CCAGATCTGAGCCTGGGAGCTCTCTGGCTAACTAGGGAACCCACTGCTTAAGCCTCAAT
AAAGCTTGCCTTGAGTGCTTCAAGTAGTGTGTGCCCGTCTGTTGTGTGACTCTGGTAAC
TAGAGATCCCTCAGACCCTTTTAGTCAGTGTGGAAAATCTCTAGCAGTACGTATAGTAG
TTCATGTCATCTTATTATTCAGTATTTATAACTTGCAAAGAAATGAATATCAGAGAGTG
AGAGGAACTTGTTTATTGCAGCTTATAATGGTTACAAATAAAGCAATAGCATCACAAAT
TTCACAAATAAAGCATTTTTTTCACTGCATTCTAGTTGTGGTTTGTCCAAACTCATCAA
TGTATCTTATCATGTCTGGCTCTAGCTATCCCGCCCCTAACTCCGCCCATCCCGCCCCT
AACTCCGCCCAGTTCCGCCCATTCTCCGCCCCATGGCTGACTAATTTTTTTTATTTATG
CAGAGGCCGAGGCCGCCTCGGCCTCTGAGCTATTCCAGAAGTAGTGAGGAGGCTTTTTT
GGAGGCCTAGGGACGTACCCAATTCGCCCTATAGTGAGTCGTATTACGCGCGCTCACTG
GCCGTCGTTTTACAACGTCGTGACTGGGAAAACCCTGGCGTTACCCAACTTAATCGCCT
TGCAGCACATCCCCCTTTCGCCAGCTGGCGTAATAGCGAAGAGGCCCGCACCGATCGCC
CTTCCCAACAGTTGCGCAGCCTGAATGGCGAATGGGACGCGCCCTGTAGCGGCGCATTA
AGCGCGGCGGGTGTGGTGGTTACGCGCAGCGTGACCGCTACACTTGCCAGCGCCCTAGC
GCCCGCTCCTTTCGCTTTCTTCCCTTCCTTTCTCGCCACGTTCGCCGGCTTTCCCCGTC
AAGCTCTAAATCGGGGGCTCCCTTTAGGGTTCCGATTTAGTGCTTTACGGCACCTCGAC
CCCAAAAAACTTGATTAGGGTGATGGTTCACGTAGTGGGCCATCGCCCTGATAGACGGT
TTTTCGCCCTTTGACGTTGGAGTCCACGTTCTTTAATAGTGGACTCTTGTTCCAAACTG
GAACAACACTCAACCCTATCTCGGTCTATTCTTTTGATTTATAAGGGATTTTGCCGATT
TCGGCCTATTGGTTAAAAAATGAGCTGATTTAACAAAAATTTAACGCGAATTTTAACAA
AATATTAACGCTTACAATTTAGGTGGCACTTTTCGGGGAAATGTGCGCGGAACCCCTAT
TTGTTTATTTTTCTAAATACATTCAAATATGTATCCGCTCATGAGACAATAACCCTGAT
AAATGCTTCAATAATATTGAAAAAGGAAGAGTATGAGTATTCAACATTTCCGTGTCGCC
CTTATTCCCTTTTTTGCGGCATTTTGCCTTCCTGTTTTTGCTCACCCAGAAACGCTGGT
GAAAGTAAAAGATGCTGAAGATCAGTTGGGTGCACGAGTGGGTTACATCGAACTGGATC
TCAACAGCGGTAAGATCCTTGAGAGTTTTCGCCCCGAAGAACGTTTTCCAATGATGAGC
ACTTTTAAAGTTCTGCTATGTGGCGCGGTATTATCCCGTATTGACGCCGGGCAAGAGCA
ACTCGGTCGCCGCATACACTATTCTCAGAATGACTTGGTTGAGTACTCACCAGTCACAG
AAAAGCATCTTACGGATGGCATGACAGTAAGAGAATTATGCAGTGCTGCCATAACCATG
AGTGATAACACTGCGGCCAACTTACTTCTGACAACGATCGGAGGACCGAAGGAGCTAAC
CGCTT

In another aspect, a nucleic acid is provided (or multiple nucleic acids are provided) that encodes a split CAR system in which a first polypeptide has an extracellular ligand binding domain, a transmembrane domain, and a cytoplasmic domain having one of two heterodimer components, and a second polypeptide has at least one intracellular signaling domain and the second of two heterodimer components, where both heterodimer components are capable of being bound by an FDA-approved agent and where either or both the first and the second polypeptide include one or more co-stimulatory domains, and optionally the second polypeptide is membrane-attached/tethered.

In order to assess the expression of a CAR polypeptide or portion thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the hosT-cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

The cell expressing the CAR or split CAR system of the present disclosure is a cell in which the nucleic acid encoding a CAR or split CAR system described above is introduced and expressed by the cell. The cell of the present disclosure binds to a specific antigen via the CAR, and then a signal is transmitted into the cell, and as a result, the cell is activated. The activation of the cell expressing the CAR is varied depending on the kind of a host cell and an intracellular domain of the CAR, and can be confirmed based on, for example, release of a cytokine, improvement of a cell proliferation rate, change in a cell surface molecule, or the like as an index. For example, release of a cytotoxic cytokine (a tumor necrosis factor, lymphotoxin, etc.) from the activated cell causes destruction of a target cell expressing an antigen. In addition, release of a cytokine or change in a cell surface molecule stimulates other immune cells, for example, a B cell, a dendritic cell, a NK cell, and a macrophage. In order to confirm the presence of the recombinant DNA sequence in the cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

In certain aspects, a method of modulating the activity of a cell expressing the CARs (or split CAR system) of the present disclosure is provided that includes administering (or withdrawal of administration) to a subject administered the CAR (or split CAR system) expressing cell an FDA-approved agent.

Other aspects of the disclosure include polynucleotide sequences, plasmids, and vectors encoding the CARs (and/or split CAR system) of the present disclosure, and T-cells expressing the CARs (and/or split CAR system) of the present disclosure.

Additional aspects include methods of modulating T lymphocyte or natural killer (NK) cell activity in a patient and treating the patient suffering from cancer by introducing into the individual a T lymphocyte or NK cell that includes a CAR (and/or split CAR system) of the present disclosure, and subsequently administering to (or withdrawing administration from) the subject an FDA-approved agent of the disclosure, thereby activating, inactivating and/or degrading the CAR and/or split CAR system (depending upon whether ON-switch or OFF-switch modalities are employed). These aspects particularly include the treatment of renal cell carcinoma, cervical carcinoma, osteosarcoma, glioblastoma, lung cancer, melanoma, breast cancer, prostate cancer, bladder cancer, salivary gland cancer, endometrial cancer, colon cancer, renal cell carcinoma, ovarian cancer, neuroblastoma, rhabdomyosarcoma, leukemia, and lymphoma. Examples of cancer targets for use with the present disclosure are cancers of B cell origin, particularly including acute lymphoblastic leukemia, B-cell chronic lymphocytic leukemia and B-cell non-Hodgkin's lymphoma.

In one embodiment, a method is provided that includes at least the steps of:
(i) removing immune effector cells, for example T-cells, from a patient with a disorder of diseased cells that can be treated by increasing the ability of the patient's T-cells to recognize and bind to the diseased cells;
(ii) transforming the T-cells ex vivo by inserting a gene or genes encoding a CAR or split CAR system having at least a sequence targeting a diseased cell surface antigen and either a drug-responsive degron amino acid sequence or a drug-responsive heterodimer sequence capable of activating the split CAR system presenting such heterodimer;
(iii) administering to the patient the autologous CAR T-cells; and then
(iv) administering to the patient, as needed, an FDA-approved drug which either (in OFF-switch aspects) promotes CAR degradation (thereby minimizing any side effects observed) or (in ON-switch aspects) activates the CAR-T cell therapy, subject to withdrawal of the FDA-approved drug halting CAR-T cell activity in the subject, to minimize or prevent any deleterious side effects that might occur in the presence of activated CAR T-cells in the subject.

An immune effector cell such as lymphocytes including but not limited to cytotoxic lymphocytes, T-cells, cytotoxic T-cells, T helper cells, Thl7 T-cells, natural killer (NK) cells, natural killer T (NKT) cells, mast cells, dendritic cells, killer dendritic cells, or B cells derived from a mammal, for example, a human cell, or a cell derived from a non-human mammal such as a monkey, a mouse, a rat, a pig, a horse, or a dog can be used. For example, a cell collected, isolated, purified or induced from a body fluid, a tissue or an organ such as blood (peripheral blood, umbilical cord blood etc.) or bone marrow can be used. A peripheral blood mononuclear cell (PBMC), an immune cell (a dendritic cell, a B cell, a hematopoietic stem cell, a macrophage, a monocyte, a NK cell or a hematopoietic cell (a neutrophil, a basophil)), an umbilical cord blood mononuclear cell, a fibroblast, a precursor adipocyte, a hepatocyte, a skin keratinocyte, a mesenchymal stem cell, an adipose stem cell, various cancer cell strains, or a neural stem cell can be used. In the present disclosure, particularly, use of a T-cell, a precursor cell of a T-cell (a hematopoietic stem cell, a lymphocyte precursor cell etc.) or a cell population containing them is contemplated. Examples of the T-cell include a CD8-positive T-cell, a CD4-positive T-cell, a regulatory T-cell, a cytotoxic T-cell, and a tumor infiltrating lymphocyte. The cell population containing a T-cell and a precursor cell of a T-cell includes a PBMC. The aforementioned cells may be collected from a living body, obtained by expansion culture of a cell collected from a living body, or established as a cell strain. When transplantation of the produced CAR-expressing cell or a cell differentiated from the produced CAR-expressing cell into a living body is desired, it is preferable to introduce the nucleic acid into a cell collected from the living body itself or a conspecific living body thereof.

In one embodiment, the CAR expressing cell is a T-cell isolated from a subject for autologous therapy. Typically, prior to expansion and genetic modification of the T-cells of the disclosure, a source of T-cells is obtained from a subject. T-cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present disclosure, any number of T-cell lines available in the art, may be used. In certain embodiments of the present disclosure, T-cells can be obtained from a unit of blood collected from a subject using any number of techniques known to the skilled artisan, such as Ficoll™ separation. In one embodiment, cells from the circulating blood of an individual are obtained by apheresis. The apheresis product typically contains lymphocytes, including T-cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the disclosure, the cells are washed with phosphate buffered saline (PBS). In an alternative embodiment, the wash solution lacks calcium and may lack magnesium or may lack many if not all divalent cations. Initial activation steps in the absence of calcium may lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca2+-free, Mg2+-free PBS, PlasmaLyte A, or other saline solution with or without buffer. Alternatively, the undesirable components of the apheresis sample may be removed and the cells directly resuspended in culture media.

In another embodiment, T-cells are isolated from peripheral blood lymphocytes by lysing the red blood cells and depleting the monocytes, for example, by centrifugation through a PERCOLL™ gradient or by counterflow centrifugal elutriation. A specific subpopulation of T-cells, such as CD3+, CD28+, CD4⁺, CD8⁺, CD45RA+, and CD45RO+ T-cells, can be further isolated by positive or negative selection techniques. For example, in one embodiment, T-cells are isolated by incubation with anti-CD3/anti-CD28 (i.e., 3×28)-conjugated beads, such as DYNABEADS® M-450 CD3/CD28 T, for a time period sufficient for positive selection of the desired T-cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another embodiment, the time period is 10 to 24 hours. In one embodiment, the incubation time period is 24 hours. For isolation of T-cells from patients with leukemia, use of longer incubation times, such as 24 hours, can increase cell yield. Longer incubation times may be used to isolate T-cells in any situation where there are few T-cells as compared to other cell types, such in isolating tumor infiltrating lymphocytes (TIL) from tumor tissue or from immune-compromised individuals. Further, use of longer incubation times can increase the efficiency of capture of CD8⁺ T-cells. Thus, by simply shortening or lengthening the time T-cells are allowed to bind to the CD3/CD28 beads and/or by increasing or decreasing the ratio of beads to T-cells, subpopulations of T-cells can be preferentially selected for or against at culture initiation or at other time points during the process. Additionally, by increasing or decreasing the ratio of anti-CD3 and/or anti-CD28 antibodies on the beads or other surface, subpopulations of T-cells can be preferentially selected for or against at culture initiation or at other desired time points. The skilled artisan would recognize that multiple rounds of selection can also be used in the context of this disclosure. In certain embodiments, it may be desirable to perform the selection procedure and use the “unselected” cells in the activation and expansion process. “Unselected” cells can also be subjected to further rounds of selection.

Enrichment of a T-cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8. In certain embodiments, it may be desirable to enrich for or positively select for regulatory T-cells which typically express CD4⁺, CD25+, CD62Lhi, GITR+, and FoxP3+. Alternatively, in certain embodiments, T regulatory cells are depleted by anti-C25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T-cells, or from samples where there are many tumor cells present (i.e., leukemic blood, tumor tissue, etc.). Such populations of cells may have therapeutic value and would be desirable to obtain. For example, using high concentration of cells allows more efficient selection of CD8⁺ T-cells that normally have weaker CD28 expression.

In a related embodiment, it may be desirable to use lower concentrations of cells. By significantly diluting the mixture of T-cells and surface (e.g., particles such as beads), interactions between the particles and cells is minimized. This selects for cells that express high amounts of desired antigens to be bound to the particles. For example, CD4⁺ T-cells express higher levels of CD28 and are more efficiently captured than CD8⁺ T-cells in dilute concentrations. In one embodiment, the concentration of cells used is 5×10⁶/ml. In other embodiments, the concentration used can be from about 1×10⁵/ml to 1×10⁶/ml, and any integer value in between.

In other embodiments, the cells may be incubated on a rotator for varying lengths of time at varying speeds at either 2-10° C. or at room temperature.

T-cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen. In certain embodiments, cryopreserved cells are thawed and washed as described herein and allowed to rest for one hour at room temperature prior to activation using the methods of the present disclosure.

Also contemplated in the context of the disclosure is the collection of blood samples or apheresis product from a subject at a time period prior to when the expanded cells as described herein might be needed. As such, the source of the cells to be expanded can be collected at any time point necessary, and desired cells, such as T-cells, isolated and frozen for later use in T-cell therapy for any number of diseases or conditions that would benefit from T-cell therapy, such as those described herein. In one embodiment a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T-cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66 (1991):807-815; Henderson et al., Immun 73 (1991):316-321; Bierer et al., Curr. Opin. Immun 5 (1993):763-773). In a further embodiment, the cells are isolated for a patient and frozen for later use in conjunction with (e.g., before, simultaneously or following) bone marrow or stem cell transplantation, T-cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy such as agents that react with CD20, e.g., Rituxan.

In a further embodiment of the present disclosure, T-cells are obtained from a patient directly following treatment. In this regard, it has been observed that following certain cancer treatments, in particular treatments with drugs that damage the immune system, shortly after treatment during the period when patients would normally be recovering from the treatment, the quality of T-cells obtained may be optimal or improved for their ability to expand ex vivo. Likewise, following ex vivo manipulation using the methods described herein, these cells may be in a state for enhanced engraftment and in vivo expansion. Thus, it is contemplated within the context of the present disclosure to collect blood cells, including T-cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T-cells, B cells, dendritic cells, and other cells of the immune system.

Whether prior to or after genetic modification of the T-cells to express a desirable CAR (e.g., CAR with degron, split CAR system with FDA-responsive heterodimer, etc.), the T-cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T-cells of the disclosure are expanded by contact with a surface having attached thereto an agent that stimulates a CD3/TCR complex associated signal and a ligand that stimulates a co-stimulatory molecule on the surface of the T-cells. In particular, T-cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T-cells, a ligand that binds the accessory molecule is used. For example, a population of T-cells can be contacted with an anti-CD3 antibody and an anti-CD28 antibody, under conditions appropriate for stimulating proliferation of the T-cells. To stimulate proliferation of either CD4⁺ T-cells or CD8⁺ T-cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berge et al., Transplant Proc. 30(8) (1998):3975-3977; Haanen et al., J. Exp. Med. 190(9) (1999):1319-1328, 1999; and Garland et al., J. Immunol Meth. 227(1-2) (1999):53-63).

The cell expressing the CAR (e.g., CAR with drug-responsive degron, split CAR system with drug-responsive heterodimer, etc.) can be used as a therapeutic agent for a disease. The therapeutic agent can be the cell expressing the CAR as an active ingredient, and may further include a suitable excipient. The disease against which the cell expressing the CAR is administered is not limited as long as the disease shows sensitivity to the cell. Examples of the disease include a cancer (blood cancer (leukemia), solid tumor etc.), an inflammatory disease/autoimmune disease (asthma, eczema), hepatitis, and an infectious disease, the cause of which is a virus such as influenza and HIV, a bacterium, or a fungus, for example, tuberculosis, MRSA, VRE, and deep mycosis. The cell expressing the CAR of the present disclosure that binds to an antigen possessed by a cell that is desired to be decreased or eliminated for treatment of the aforementioned diseases, that is, a tumor antigen, a viral antigen, a bacterial antigen or the like is administered for treatment of these diseases. The cell of the present disclosure can also be utilized for prevention of an infectious disease after bone marrow transplantation or exposure to radiation, donor lymphocyte transfusion for the purpose of remission of recurrent leukemia, and the like. The therapeutic agent comprising the cell expressing the CAR as an active ingredient can be administered intradermally, intramuscularly, subcutaneously, intraperitoneally, intranasally, intraarterially, intravenously, intratumorally, or into an afferent lymph vessel, by parenteral administration, for example, by injection or infusion, although the administration route is not limited.

In a particular embodiment, the CAR expressing cell is an autologous T-cell from a subject with cancer. Cancers that may be treated include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the CARs of the disclosure include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Other hematological cancers include T-cell or NK-cell lymphoma, for example, but not limited to: peripheral T-cell lymphoma; anaplastic large cell lymphoma, for example anaplastic lymphoma kinase (ALK) positive, ALK negative anaplastic large cell lymphoma, or primary cutaneous anaplastic large cell lymphoma; angioimmunoblastic lymphoma; cutaneous T-cell lymphoma, for example mycosis fungoides, Sézary syndrome, primary cutaneous anaplastic large cell lymphoma, primary cutaneous CD30+ T-cell lymphoproliferative disorder; primary cutaneous aggressive epidermotropic CD8⁺ cytotoxic T-cell lymphoma; primary cutaneous gamma-delta T-cell lymphoma; primary cutaneous small/medium CD4⁺ T-cell lymphoma, and lymphomatoid papulosis; Adult T-cell Leukemia/Lymphoma (ATLL); Blastic NK-cell Lymphoma; Enteropathy-type T-cell lymphoma; Hematosplenic gamma-delta T-cell Lymphoma; Lymphoblastic Lymphoma; Nasal NK/T-cell Lymphomas; Treatment-related T-cell lymphomas; for example lymphomas that appear after solid organ or bone marrow transplantation; T-cell prolymphocytic leukemia; T-cell large granular lymphocytic leukemia; Chronic lymphoproliferative disorder of NK− cells; Aggressive NK cell leukemia; Systemic EBV+ T-cell lymphoproliferative disease of childhood (associated with chronic active EBV infection); Hydroa vacciniforme-like lymphoma; Adult T-cell leukemia/lymphoma; Enteropathy-associated T-cell lymphoma; Hepatosplenic T-cell lymphoma; or Subcutaneous panniculitis-like T-cell lymphoma.

In one embodiment, the CAR expressing cells can be used in an effective amount to treat a host, for example a human, with a lymphoma or lymphocytic or myelocytic proliferation disorder or abnormality. For example, the CAR expressing cells as described herein can be administered to a host suffering from a Hodgkin Lymphoma or a Non-Hodgkin Lymphoma. For example, the host can be suffering from a Non-Hodgkin Lymphoma such as, but not limited to: an AIDS-Related Lymphoma; Anaplastic Large-Cell Lymphoma; Angioimmunoblastic Lymphoma; Blastic NK-Cell Lymphoma; Burkitt's Lymphoma; Burkitt-like Lymphoma (Small Non-Cleaved Cell Lymphoma); Chronic Lymphocytic Leukemia/Small Lymphocytic Lymphoma; Cutaneous T-Cell Lymphoma; Diffuse Large B-Cell Lymphoma; Enteropathy-Type T-Cell Lymphoma; Follicular Lymphoma; Hepatosplenic Gamma-Delta T-Cell Lymphoma; Lymphoblastic Lymphoma; Mantle Cell Lymphoma; Marginal Zone Lymphoma; Nasal T-Cell Lymphoma; Pediatric Lymphoma; Peripheral T-Cell Lymphomas; Primary Central Nervous System Lymphoma; T-Cell Leukemias; Transformed Lymphomas; Treatment-Related T-Cell Lymphomas; or Waldenstrom's Macroglobulinemia.

Alternatively, CAR expressing cells disclosed herein can be used in an effective amount to treat a host, for example a human, with a Hodgkin Lymphoma, such as, but not limited to: Nodular Sclerosis Classical Hodgkin's Lymphoma (CHL); Mixed Cellularity CHL; Lymphocyte-depletion CHL; Lymphocyte-rich CHL; Lymphocyte Predominant Hodgkin Lymphoma; or Nodular Lymphocyte Predominant HL.

Alternatively, CAR expressing cells disclosed herein can be used in an effective amount to treat a host, for example a human with a specific B-cell lymphoma or proliferative disorder such as, but not limited to: multiple myeloma; Diffuse large B cell lymphoma; Follicular lymphoma; Mucosa-Associated Lymphatic Tissue lymphoma (MALT); Small cell lymphocytic lymphoma; Mediastinal large B cell lymphoma; Nodal marginal zone B cell lymphoma (NMZL); Splenic marginal zone lymphoma (SMZL); Intravascular large B-cell lymphoma; Primary effusion lymphoma; or Lymphomatoid granulomatosis; B-cell prolymphocytic leukemia; Hairy cell leukemia; Splenic lymphoma/leukemia, unclassifiable; Splenic diffuse red pulp small B-cell lymphoma; Hairy cell leukemia-variant; Lymphoplasmacytic lymphoma; Heavy chain diseases, for example, Alpha heavy chain disease, Gamma heavy chain disease, Mu heavy chain disease; Plasma cell myeloma; Solitary plasmacytoma of bone; Extraosseous plasmacytoma; Primary cutaneous follicle center lymphoma; T-cell/histiocyte rich large B-cell lymphoma; DLBCL associated with chronic inflammation; Epstein-Barr virus (EBV)+ DLBCL of the elderly; Primary mediastinal (thymic) large B-cell lymphoma; Primary cutaneous DLBCL, leg type; ALK+ large B-cell lymphoma; Plasmablastic lymphoma; Large B-cell lymphoma arising in HHV8-associated multicentric; Castleman disease; B-cell lymphoma, unclassifiable, with features intermediate between diffuse large B-cell lymphoma; or B-cell lymphoma, unclassifiable, with features intermediate between diffuse large B-cell lymphoma and classical Hodgkin lymphoma.

In one embodiment, CAR expressing cells disclosed herein can be used in an effective amount to treat a host, for example a human with leukemia. For example, the host may be suffering from an acute or chronic leukemia of a lymphocytic or myelogenous origin, such as, but not limited to: Acute lymphoblastic leukemia (ALL); Acute myelogenous leukemia (AML); Chronic lymphocytic leukemia (CLL); Chronic myelogenous leukemia (CML); juvenile myelomonocytic leukemia (JMML); hairy cell leukemia (HCL); acute promyelocytic leukemia (a subtype of AML); large granular lymphocytic leukemia; or Adult T-cell chronic leukemia. In one embodiment, the patient suffers from an acute myelogenous leukemia, for example an undifferentiated AML (M0); myeloblastic leukemia (M1; with/without minimal cell maturation); myeloblastic leukemia (M2; with cell maturation); promyelocytic leukemia (M3 or M3 variant [M3V]); myelomonocytic leukemia (M4 or M4 variant with eosinophilia [M4E]); monocytic leukemia (M5); erythroleukemia (M6); or megakaryoblastic leukemia (M7).

In one embodiment, CAR expressing cells disclosed herein can be used in an effective amount to treat a host, for example a human with a solid tumor. Examples include, but are not limited to, but are not limited to: estrogen-receptor positive, HER2-negative advanced breast cancer, late-line metastatic breast cancer, liposarcoma, non-small cell lung cancer, liver cancer, ovarian cancer, glioblastoma, refractory solid tumors, retinoblastoma positive breast cancer as well as retinoblastoma positive endometrial, vaginal and ovarian cancers and lung and bronchial cancers, adenocarcinoma of the colon, adenocarcinoma of the rectum, central nervous system germ cell tumors, teratomas, estrogen receptor-negative breast cancer, estrogen receptor-positive breast cancer, familial testicular germ cell tumors, HER2-negative breast cancer, HER2-positive breast cancer, male breast cancer, ovarian immature teratomas, ovarian mature teratoma, ovarian monodermal and highly specialized teratomas, progesterone receptor-negative breast cancer, progesterone receptor-positive breast cancer, recurrent breast cancer, recurrent colon cancer, recurrent extragonadal germ cell tumors, recurrent extragonadal non-seminomatous germ cell tumor, recurrent extragonadal seminomas, recurrent malignant testicular germ cell tumors, recurrent melanomas, recurrent ovarian germ cell tumors, recurrent rectal cancer, stage III extragonadal non-seminomatous germ cell tumors, stage III extragonadal seminomas, stage III malignant testicular germ cell tumors, stage III ovarian germ cell tumors, stage IV breast cancers, stage IV colon cancers, stage IV extragonadal non-seminomatous germ cell tumors, stage IV extragonadal seminoma, stage IV melanomas, stage IV ovarian germ cell tumors, stage IV rectal cancers, testicular immature teratomas, testicular mature teratomas, estrogen-receptor positive, HER2-negative advanced breast cancer, late-line metastatic breast cancer, liposarcoma, non-small cell lung cancer, liver cancer, ovarian cancer, glioblastoma, refractory solid tumors, retinoblastoma positive breast cancer as well as retinoblastoma positive endometrial, vaginal and ovarian cancers and lung and bronchial cancers, metastatic colorectal cancer, metastatic melanoma, or cisplatin-refractory, unresectable germ cell tumors, carcinoma, sarcoma, including, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, fibrosarcoma, myxosarcoma, chondrosarcoma, osteosarcoma, chordoma, malignant fibrous histiocytoma, hemangiosarcoma, angiosarcoma, lymphangiosarcoma. Mesothelioma, leiomyosarcoma, rhabdomyosarcoma, squamous cell carcinoma; epidermoid carcinoma, malignant skin adnexal tumors, adenocarcinoma, hepatoma, hepatocellular carcinoma, renal cell carcinoma, hypernephroma, cholangiocarcinoma, transitional cell carcinoma, choriocarcinoma, seminoma, embryonal cell carcinoma, glioma anaplastic; glioblastoma multiforme, neuroblastoma, medulloblastoma, malignant meningioma, malignant schwannoma, neurofibrosarcoma, parathyroid carcinoma, medullary carcinoma of thyroid, bronchial carcinoid, pheochromocytoma, IsleT-cell carcinoma, malignant carcinoid, malignant paraganglioma, melanoma, Merkel cell neoplasm, cystosarcoma phylloide, salivary cancers, thymic carcinomas, bladder cancer, and Wilms tumor, a blood disorder or a hematologic malignancy, including, but not limited to, myeloid disorder, lymphoid disorder, leukemia, lymphoma, myelodysplastic syndrome (MDS), myeloproliferative disease (MPD), masT-cell disorder, and myeloma (e.g., multiple myeloma).

In another embodiment, a CAR expressing cell disclosed herein can be used in an effective amount to treat a host, for example a human with an autoimmune disorder. Examples include, but are not limited to: Acute disseminated encephalomyelitis (ADEM); Addison's disease; Agammaglobulinemia; Alopecia areata; Amyotrophic lateral sclerosis (Also Lou Gehrig's disease; Motor Neuron Disease); Ankylosing Spondylitis; Antiphospholipid syndrome; Antisynthetase syndrome; Atopic allergy; Atopic dermatitis; Autoimmune aplastic anemia; Autoimmune arthritis; Autoimmune cardiomyopathy; Autoimmune enteropathy; Autoimmune granulocytopenia; Autoimmune hemolytic anemia; Autoimmune hepatitis; Autoimmune hypoparathyroidism; Autoimmune inner ear disease; Autoimmune lymphoproliferative syndrome; Autoimmune myocarditis; Autoimmune pancreatitis; Autoimmune peripheral neuropathy; Autoimmune ovarian failure; Autoimmune polyendocrine syndrome; Autoimmune progesterone dermatitis; Autoimmune thrombocytopenic purpura; Autoimmune thyroid disorders; Autoimmune urticarial; Autoimmune uveitis; Autoimmune vasculitis; Balo disease/Balo concentric sclerosis; Behget's disease; Berger's disease; Bickerstaffs encephalitis; Blau syndrome; Bullous pemphigoid; Cancer; Castleman's disease; Celiac disease; Chagas disease; Chronic inflammatory demyelinating polyneuropathy; Chronic inflammatory demyelinating polyneuropathy; Chronic obstructive pulmonary disease; Chronic recurrent multifocal osteomyelitis; Churg-Strauss syndrome; Cicatricial pemphigoid; Cogan syndrome; Cold agglutinin disease; Complement component 2 deficiency; Contact dermatitis; Cranial arteritis; CREST syndrome; Crohn's disease; Cushing's Syndrome; Cutaneous leukocytoclastic angiitis; Dego's disease; Dercum's disease; Dermatitis herpetiformis; Dermatomyositis; Diabetes mellitus type 1; Diffuse cutaneous systemic sclerosis; Discoid lupus erythematosus; Dressler's syndrome; Drug-induced lupus; Eczema; Endometriosis; Enthesitis-related arthritis; Eosinophilic fasciitis; Eosinophilic gastroenteritis; Eosinophilic pneumonia; Epidermolysis bullosa acquisita; Erythema nodosum; Erythroblastosis fetalis; Essential mixed cryoglobulinemia; Evan's syndrome; Extrinsic and intrinsic reactive airways disease (asthma); Fibrodysplasia ossificans progressive; Fibrosing alveolitis (or Idiopathic pulmonary fibrosis); Gastritis; Gastrointestinal pemphigoid; Glomerulonephritis; Goodpasture's syndrome; Graves' disease; Guillain-Barre syndrome (GBS); Hashimoto's encephalopathy; Hashimoto's thyroiditis; Hemolytic anemia; Henoch-Schonlein purpura; Herpes gestationis (Gestational Pemphigoid); Hidradenitis suppurativa; Hughes-Stovin syndrome; Hypogammaglobulinemia; Idiopathic inflammatory demyelinating diseases; Idiopathic pulmonary fibrosis; Idiopathic thrombocytopenic purpura; IgA nephropathy; Immune glomerulonephritis; Immune nephritis; Immune pneumonitis; Inclusion body myositis; inflammatory bowel disease; Interstitial cystitis; Juvenile idiopathic arthritis aka Juvenile rheumatoid arthritis; Kawasaki's disease; Lambert-Eaton myasthenic syndrome; Leukocytoclastic vasculitis; Lichen planus; Lichen sclerosus; Linear IgA disease (LAD); Lupoid hepatitis aka Autoimmune hepatitis; Lupus erythematosus; Majeed syndrome; microscopic polyangiitis; Miller-Fisher syndrome; mixed connective tissue disease; Morphea; Mucha-Habermann disease aka Pityriasis lichenoides et varioliformis acuta; Multiple sclerosis; Myasthenia gravis; Myositis; Ménière's disease; Narcolepsy; Neuromyelitis optica (also Devic's disease); Neuromyotonia; Occular cicatricial pemphigoid; Opsoclonus myoclonus syndrome; Ord's thyroiditis; Palindromic rheumatism; PANDAS (pediatric autoimmune neuropsychiatric disorders associated with Streptococcus); Paraneoplastic cerebellar degeneration; Paroxysmal nocturnal hemoglobinuria (PNH); Parry Romberg syndrome; Pars planitis; Parsonage-Turner syndrome; Pemphigus vulgaris; Perivenous encephalomyelitis; Pernicious anaemia; POEMS syndrome; Polyarteritis nodosa; Polymyalgia rheumatic; Polymyositis; Primary biliary cirrhosis; Primary sclerosing cholangitis; Progressive inflammatory neuropathy; Psoriasis; Psoriatic arthritis; pure red cell aplasia; Pyoderma gangrenosum; Rasmussen's encephalitis; Raynaud phenomenon; Reiter's syndrome; relapsing polychondritis; restless leg syndrome; retroperitoneal fibrosis; rheumatic fever; rheumatoid arthritis; Sarcoidosis; Schizophrenia; Schmidt syndrome; Schnitzler syndrome; Scleritis; Scleroderma; Sclerosing cholangitis; serum sickness; Sjögren's syndrome; Spondyloarthropathy; Stiff person syndrome; Still's disease; Subacute bacterial endocarditis (SBE); Susac's syndrome; Sweet's syndrome; Sydenham chorea; sympathetic ophthalmia; systemic lupus erythematosus; Takayasu's arteritis; temporal arteritis (also known as “gianT-cell arteritis”); thrombocytopenia; Tolosa-Hunt syndrome; transverse myelitis; ulcerative colitis; undifferentiated connective tissue disease; undifferentiated spondyloarthropathy; urticarial vasculitis; vasculitis; vitiligo; viral diseases such as Epstein Barr Virus (EBV), Hepatitis B, Hepatitis C, HIV, HTLV 1, Varicella-Zoster Virus (VZV) and Human Papilloma Virus (HPV); or Wegener's granulomatosis. In some embodiments, the autoimmune disease is an allergic condition, including those from asthma, food allergies, atopic dermatitis, and rhinitis.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

In one embodiment, the antigen binding moiety portion of the CAR of the disclosure is designed to treat a particular cancer. For example, a CAR designed to target CD19 can be used to treat cancers and disorders including but are not limited to pre-B ALL (pediatric indication), adult ALL, mantle cell lymphoma, diffuse large B-cell lymphoma, salvage post allogenic bone marrow transplantation, and the like.

In another embodiment, the CAR can be designed to target CD22 to treat diffuse large B-cell lymphoma.

In one embodiment, cancers and disorders include but are not limited to pre-B ALL (pediatric indication), adult ALL, mantle cell lymphoma, diffuse large B-cell lymphoma, salvage post allogenic bone marrow transplantation, and the like can be treated using a combination of CARs that target CD19, CD20, CD22, and ROR1.

In one embodiment, the CAR can be designed to target mesothelin to treat mesothelioma, pancreatic cancer, ovarian cancer, and the like.

In one embodiment, the CAR can be designed to target CD33/IL3Ra to treat acute myelogenous leukemia and the like.

In one embodiment, the CAR can be designed to target CD30 to treat lymphoma, for example Hodgkin lymphoma, and the like.

In one embodiment, the CAR can be designed to target c-Met to treat triple negative breast cancer, non-small cell lung cancer, and the like.

In one embodiment, the CAR can be designed to target PSMA to treat prostate cancer and the like.

In one embodiment, the CAR can be designed to target Glycolipid F77 to treat prostate cancer and the like.

In one embodiment, the CAR can be designed to target EGFRvIII to treat gliobastoma and the like.

In one embodiment, the CAR can be designed to target GD-2 to treat neuroblastoma, melanoma, and the like.

In one embodiment, the CAR can be designed to target NY-ESO-1 TCR to treat myeloma, sarcoma, melanoma, and the like.

In one embodiment, the CAR can be designed to target MAGE A3 TCR to treat myeloma, sarcoma, melanoma, and the like.

In one embodiment, the CAR can be designed to target CEA to treat colorectal cancer and the like.

In one embodiment, the CAR can be designed to target erb-B2, erb-B3, and/or erb-B4 to treat breast cancer, and the like.

In one embodiment, the CAR can be designed to target IL-13R-a2 to treat glioma, glioblastoma, or medulloblastoma, and the like.

However, the disclosure should not be construed to be limited to solely to the antigen targets and diseases disclosed herein. Rather, the disclosure should be construed to include any antigenic or ligand target that is associated with a disease where a CAR or split CAR system having an element (e.g., degron, heterodimer, etc.) that is responsive to a FDA-approved drug can be used to treat the disease or disorder.

The CAR-expressing cells of the disclosure may also serve as a type of vaccine for ex vivo immunization and/or in vivo therapy in a mammal. Optionally, the mammal is a human.

With respect to ex vivo immunization, at least one of the following occurs in vitro prior to administering the cell into a mammal: i) expansion of the cells, ii) introducing a nucleic acid encoding a CAR to the cells, and/or iii) cryopreservation of the cells.

The CAR-expressing cells of the present disclosure can be administered either alone, or as a pharmaceutical composition in combination with diluents and/or with other components such as IL-2 or other cytokines or cell populations. Briefly, pharmaceutical compositions of the present disclosure may comprise a target T-cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are optionally formulated for intravenous administration.

### Pharmaceutical Compositions, Kits, and Administration

Pharmaceutical compositions of CAR expressing cells of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-tumor effective amount”, “a tumor-inhibiting effective amount”, or “therapeutic amount” is indicated, the precise amount of the compositions of the present disclosure to be administered can be determined by a physician with consideration of individual differences in age, weight, tumor size, extent of infection or metastasis, and condition of the patient (subject). It can generally be stated that a pharmaceutical composition comprising the T-cells described herein may be administered at a dosage of 10⁴ to 10′ cells/kg body weight, preferably 10⁵ to 10⁶ cells/kg body weight, including all integer values within those ranges. T-cell compositions may also be administered multiple times at these dosages. The cells can be administered by using infusion techniques that are commonly known in immunotherapy (see, e.g., Rosenberg et al., New Eng. J. of Med. 319 (1988):1676).

The optimal dosage and treatment regime for a particular patient can readily be determined by one skilled in the art of medicine by monitoring the patient for signs of disease and adjusting the treatment accordingly.

The administration of the CAR expressing cells may be carried out in any convenient manner, including by aerosol inhalation, injection, ingestion, transfusion, implantation or transplantation. The CAR expressing cells described herein may be administered to a patient subcutaneously, intradermally, intratumorally, intranodally, intramedullary, intramuscularly, by intravenous (i.v.) injection, or intraperitoneally. In one embodiment, the CAR expressing cells of the present disclosure are administered to a patient by intradermal or subcutaneous injection. In another embodiment, the CAR expressing cells of the present disclosure are optionally administered by i.v. injection. The CAR expressing cells may be injected directly into a tumor, lymph node, or site of infection.

The dosage of the above treatments to be administered to a patient will vary with the precise nature of the condition being treated and the recipient of the treatment. The scaling of dosages for human administration can be performed according to art-accepted practices.

Certain aspects of the present application provide pharmaceutical compositions which comprise, e.g., the FDA-approved drugs described herein (or a prodrug, pharmaceutically acceptable salt or other pharmaceutically acceptable derivative thereof), and optionally comprise a pharmaceutically acceptable carrier, optionally also including CAR and/or split CAR nucleic acid vectors of the disclosure and/or CAR- and/or split CAR system-containing cells of the disclosure. It will also be appreciated that certain of the FDA-approved compounds of the present application can exist in free form for treatment, or where appropriate, as a pharmaceutically acceptable derivative thereof. According to the present application, a pharmaceutically acceptable derivative includes, but is not limited to, pharmaceutically acceptable salts, esters, salts of such esters, or a pro-drug or other adduct or derivative of a compound of this application which upon administration to a patient in need is capable of providing, directly or indirectly, an FDA-approved compound possessing qualities (i.e., CAR modulatory properties) as otherwise described herein, or a metabolite or residue thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J Pharmaceutical Sciences 66 (1977):1-19, incorporated herein by reference. The salts can be prepared in situ during the final isolation and purification of the FDA-approved compounds of the application, or separately by reacting a free base or free acid function with a suitable reagent, as described generally below. For example, a free base function can be reacted with a suitable acid. Furthermore, where the FDA-approved compounds of the application carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Additionally, as used herein, the term “pharmaceutically acceptable ester” refers to esters that hydrolyze in vivo and include those that break down readily in the human body to leave the parent FDA-approved compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moeity advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of the FDA-approved compounds of the present application which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the application. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, (1987), both of which are incorporated herein by reference.

In general, methods of using the FDA-approved compounds for modulating the activity of a CAR expressing cell as described in the present application comprise administering to a subject in need thereof a therapeutically effective amount of a FDA-approved compound of the present application, wherein the FDA-approved compound is administered in an amount sufficient to activate the CAR system (ON-switch) or induce degradation of the CAR (OFF-switch).

In certain embodiments, FDA-approved drugs are useful to modulate or downregulate the activation of the CAR expressing cell, for example a CAR T-cell, for example by degrading the intracellular signaling pathway of the CAR and thus reducing, for example, the release of cytokines by the CAR T-cell due to its activated state. In certain embodiments, according to the methods of treatment of the present application, levels of the CAR in the CAR expressing cell are modulated by contacting CAR expressing cells with a FDA-approved drug, as described herein.

Thus, in another aspect of the application, methods for the modulating of the activity of a CAR expressing cell, for example a CAR T-cell, are provided comprising administering a therapeutically effective amount of a FDA-approved drug to a subject in need thereof. In certain embodiments, a method for the modulation of a CAR expressing cell, for example a CAR T-cell, is provided comprising administering a therapeutically effective amount of FDA-approved compound, or a pharmaceutical composition comprising FDA-approved drug to a subject in need thereof, in such amounts and for such time as is necessary to achieve the desired result. Preferably, the FDA-approved compound is administered orally or intravenously. In certain embodiments of the present application a “therapeutically effective amount” of the FDA-approved drug is that amount effective for reducing the activity of a CAR expressing cell so that an adverse inflammatory or immune response is modulated or reduced. The FDA-approved drug, according to the method of the present application, may be administered using any amount and any route of administration effective for modulating the activity of a CAR expressing cell. The exact amount required will vary from subject to subject, depending on the species, age, and general condition of the subject, the activity of the CAR expressing cell, the particular CAR expressing cell, and the like. In certain embodiments of the present application a “therapeutically effective amount” of the FDA-approved drug is that amount effective for reducing the levels of CARs in a CAR expressing cell. In other embodiments of the present application, a “therapeutically effective amount” of the FDA-approved drug is that amount effective for activating a CAR T-cell therapy in a subject.

An effective amount of an agent of the instant disclosure may vary from about 0.001 mg/kg to about 1000 mg/kg or more in one or more dose administrations for one or several days (depending on the mode of administration). In certain embodiments, the effective amount per dose varies from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, and from about 10.0 mg/kg to about 150 mg/kg.

In certain embodiments, the effective amount is an amount effective to selectively enhance T cell-mediated killing of target cells displaying a targeted (e.g., tumor) antigen by at least about 10%, at least about 20%, at least about 30%, at least about 40%, at least about 50%, at least about 60%, at least about 70%, at least about 80%, at least about 90%, at least about 100%, at least about 200%, at least about 300%, at least about 500%, or at least about 1000%. In certain embodiments, the effective amount is an amount effective for inhibiting T cell-mediated killing of target cells displaying a targeted (e.g., tumor) antigen (or inhibiting an observed side effect of CAR T-cell therapy) by at least about by at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99%.

Pharmaceutical compositions described herein can be prepared by any method known in the art of pharmacology. In general, such preparatory methods include the steps of bringing the agent or compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, Poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof.

Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, Eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of an agent (e.g., an IMiD) described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

FDA-approved drugs provided herein are typically formulated in dosage unit form for ease of administration and uniformity of dosage. It will be understood, however, that the total daily usage of the agents described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The agents and compositions provided herein can be administered by any route, including enteral (e.g., oral), parenteral, intravenous, intramuscular, intra-arterial, intramedullary, intrathecal, subcutaneous, intraventricular, transdermal, intradermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the agent or pharmaceutical composition described herein is suitable for topical administration to the eye of a subject.

The exact amount of an agent required to achieve an effective amount will vary from subject to subject, depending, for example, on species, age, and general condition of a subject, severity of the side effects or disorder, identity of the particular agent, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of an agent (e.g., an IMiD) described herein.

As noted elsewhere herein, a drug or CAR T-cell agent of the instant disclosure may be administered via a number of routes of administration, including but not limited to: subcutaneous, intravenous, intrathecal, intramuscular, intranasal, oral, transepidermal, parenteral, by inhalation, or intracerebroventricular.

The term “injection” or “injectable” as used herein refers to a bolus injection (administration of a discrete amount of an agent for raising its concentration in a bodily fluid), slow bolus injection over several minutes, or prolonged infusion, or several consecutive injections/infusions that are given at spaced apart intervals.

In some embodiments of the present disclosure, a formulation as herein defined is administered to the subject by bolus administration.

The modulatory cell therapy or FDA-approved drug is administered to the subject in an amount sufficient to achieve a desired effect at a desired site (e.g., enhanced T cell-mediated killing of target cells, inhibited side-effects observed upon administration/activation of CAR T-cell treatment, etc.) determined by a skilled clinician to be effective. In some embodiments of the disclosure, the agent is administered at least once a year. In other embodiments of the disclosure, the agent is administered at least once a day. In other embodiments of the disclosure, the agent is administered at least once a week. In some embodiments of the disclosure, the agent is administered at least once a month.

Exemplary doses for administration of an agent of the disclosure to a subject include, but are not limited to, the following: 1-20 mg/kg/day, 2-15 mg/kg/day, 5-12 mg/kg/day, 10 mg/kg/day, 1-500 mg/kg/day, 2-250 mg/kg/day, 5-150 mg/kg/day, 20-125 mg/kg/day, 50-120 mg/kg/day, 100 mg/kg/day, at least 10 μg/kg/day, at least 100 μg/kg/day, at least 250 μg/kg/day, at least 500 μg/kg/day, at least 1 mg/kg/day, at least 2 mg/kg/day, at least 5 mg/kg/day, at least 10 mg/kg/day, at least 20 mg/kg/day, at least 50 mg/kg/day, at least 75 mg/kg/day, at least 100 mg/kg/day, at least 200 mg/kg/day, at least 500 mg/kg/day, at least 1 g/kg/day, and an imaging and/or therapeutically effective dose that is less than 500 mg/kg/day, less than 200 mg/kg/day, less than 100 mg/kg/day, less than 50 mg/kg/day, less than 20 mg/kg/day, less than 10 mg/kg/day, less than 5 mg/kg/day, less than 2 mg/kg/day, less than 1 mg/kg/day, less than 500 μg/kg/day, and less than 500 μg/kg/day.

In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 g and 1 g, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of an agent (e.g., an IMiD) described herein. In certain embodiments, a dose described herein includes independently between 1 mg and 3 mg, inclusive, of an agent (e.g., an IMiD) described herein. In certain embodiments, a dose described herein includes independently between 3 mg and 10 mg, inclusive, of an agent (e.g., an IMiD) described herein. In certain embodiments, a dose described herein includes independently between 10 mg and 30 mg, inclusive, of an agent (e.g., an IMiD) described herein. In certain embodiments, a dose described herein includes independently between 30 mg and 100 mg, inclusive, of an agent (e.g., an IMiD) described herein.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

It will be also appreciated that an agent (e.g., an IMiD) or composition, as described herein, can be administered in combination with one or more additional pharmaceutical agents (e.g., therapeutically and/or prophylactically active agents), which are different from the agent or composition and may be useful as, e.g., combination therapies. The agents or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk of developing a disease in a subject in need thereof, in inhibiting the replication of a virus, in killing a virus, etc. a subject or cell. In certain embodiments, a pharmaceutical composition described herein including an agent (e.g., an IMiD) described herein and an additional pharmaceutical agent shows a synergistic effect that is absent in a pharmaceutical composition including one of the agent and the additional pharmaceutical agent, but not both.

In some embodiments of the disclosure, a therapeutic agent distinct from a drug-responsive system of the disclosure is administered prior to, in combination with, at the same time, or after administration of the drug and/or drug-responsive system of the disclosure. In some embodiments, the second therapeutic agent is selected from the group consisting of a chemotherapeutic, an antioxidant, an antiinflammatory agent, an antimicrobial, a steroid, etc.

The agent or composition can be administered concurrently with, prior to, or subsequent to one or more additional pharmaceutical agents, which may be useful as, e.g., combination therapies.

Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease described herein. Each additional pharmaceutical agent may be administered at a dose and/or on a time schedule determined for that pharmaceutical agent. The additional pharmaceutical agents may also be administered together with each other and/or with the agent or composition described herein in a single dose or administered separately in different doses. The particular combination to employ in a regimen will take into account compatibility of the agent described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, immunomodulatory agents, anti-cancer agents, anti-proliferative agents, cytotoxic agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, and pain-relieving agents. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine kinase inhibitors), modulators of protein stability (e.g., proteasome inhibitors), Hsp90 inhibitors, glucocorticoids, all-trans retinoic acids, and other agents that promote differentiation. In certain embodiments, the agents described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy.

Also encompassed by the disclosure are kits (e.g., pharmaceutical packs). The kits provided may comprise a pharmaceutical composition or agent described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or agent described herein. In some embodiments, the pharmaceutical composition or agent described herein provided in the first container and the second container are combined to form one unit dosage form.

Thus, in one aspect, provided are kits including a first container comprising an agent (e.g., an FDA-approved IMiD) described herein, or a pharmaceutically acceptable salt, solvate, hydrate, polymorph, co-crystal, tautomer, stereoisomer, isotopically labeled derivative, or prodrug thereof, or a pharmaceutical composition thereof. In certain embodiments, the kits are useful for treating and/or preventing a disease described herein in a subject in need thereof. In certain embodiments, the kits are useful for treating a disease described herein in a subject in need thereof. In certain embodiments, the kits are useful for preventing a disease described herein in a subject in need thereof. In certain embodiments, the kits are useful for reducing the risk of developing a disease described herein in a subject in need thereof. In certain embodiments, the kits are useful for male contraception. In certain embodiments, the kits are useful for inhibiting sperm formation. In certain embodiments, the kits are useful for in inhibiting the replication of a virus. In certain embodiments, the kits are useful for killing a virus. In certain embodiments, the kits are useful for enhancing the activity (e.g., activating CAR T cell-mediated target cell killing) in a subject or cell. In certain embodiments, the kits are useful for inhibiting the activity (e.g., halting CAR T cell-mediated target cell killing) of CAR T cells in a subject or cell, optionally to minimize or abolish a side-effect of CAR T-cell therapy.

In certain embodiments, the kits are useful for screening a library of agents to identify an agent that is useful in a method of the disclosure.

In certain embodiments, a kit described herein further includes instructions for using the kit, such as instructions for using the kit in a method of the disclosure (e.g., instructions for administering an agent (e.g., an IMiD) or pharmaceutical composition described herein to a subject). A kit described herein may also include information as required by a regulatory agency such as the U.S. Food and Drug Administration (FDA). In certain embodiments, the information included in the kits is prescribing information. In certain embodiments, the kits and instructions provide for treating and/or preventing a disease described herein in a subject in need thereof. In certain embodiments, the kits and instructions provide for treating a disease described herein in a subject in need thereof. In certain embodiments, the kits and instructions provide for preventing a disease described herein in a subject in need thereof. In certain embodiments, the kits and instructions provide for reducing the risk of developing a disease described herein in a subject in need thereof. In certain embodiments, the kits and instructions provide for male contraception. In certain embodiments, the kits and instructions provide for inhibiting the replication of a virus. In certain embodiments, the kits and instructions provide for killing a virus. In certain embodiments, the kits and instructions provide for inducing apoptosis of an in vitro cell. In certain embodiments, the kits and instructions provide for inducing apoptosis of a cell in a subject. In certain embodiments, the kits and instructions provide for inducing G1 arrest in a subject or cell. In certain embodiments, the kits and instructions provide for screening a library of agents to identify an agent (e.g., an IMiD) that is useful in a method of the disclosure. A kit described herein may include one or more additional pharmaceutical agents described herein as a separate composition.

The practice of the present disclosure employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims. Standard techniques well known in the art or the techniques specifically described below were utilized.

### EXAMPLES

### Example 1: Materials and Methods

### Constructs

For targeted protein degradation experiments, IKZF3 degron derivatives were synthesized as gene fragments (IDT) and cloned into the Artichoke lentiviral expression marker (pSFFV-insert-linker-eGFP-IRES-mCherry-cppt-EF1a-Puro-WPRE; FIG. 9). For BRET dimerization experiments, IKZF3 degron derivatives were cloned into pFC14K (pCMV-insert-HaloTag) and CRBN derivatives were cloned into pFC32K (pCMV-insert-Nanoluciferase) per manufacturer's instructions (Promega™)

### Cell Lines

293T and Jurkat human cell lines were used. 293T cells were maintained in DMEM (Invitrogen) supplemented with 10% FBS, 2 mM L-glutamine, 100 μg/mL penicillin and 100 U/mL streptomycin. Jurkat cells were maintained in RPMI (Invitrogen) supplemented with 10% FBS, 2 mM L-glutamine, 100 μg/mL penicillin and 100 U/mL streptomycin.

### Lentivirus Production and Transduction

Lentiviral packaging was performed by co-transfection of expression, psPAX2, and VSV-G plasmids into 293T cells with TransIT-LT1 (MirusBio™) per manufacturer's instructions. 24 hours after transfection, cell culture supernatant containing lentivirus were flash frozen to −80° C. Jurkat, 293T cells, and single cell 293T cell clones with biallelic CRISPR/CAS9-mediated frameshift disruption of CRBN exon 5 (293T-CRBNΔe5) and confirmed by Sanger sequencing and Western blotting for CRBN were performed to a target transduction efficiency of 20-50%.

### GFP/mCherry Reporter Assay for Protein Degradation and Internalization

Transduced cells were incubated with a range of Pomalidomide concentrations or DMSO for 20 hours. Cells were analyzed by flow cytometry for eGFP and mCherry fluorescence. 1000 mCherry+ cells were analyzed per sample, and the geometric mean of the per-cell eGFP/mCherry ratio was calculated in Flowjo. In Pomalidomide dose titration studies, the EC50 was calculated by non-linear regression in Prism. Experiments were performed in triplicate. In some cases cell surface protein expression was assayed in parallel with the above reporter assay; cells were stained with anti-CD28-BV421 antibody (BD Biosciences 562613) at a 1:50 dilution in FACS buffer (PBS+2% FCS+2 mM EDTA) for 10 minutes on ice. Cells were washed with FACS buffer and analyzed by flow cytometry.

### Bioluminescence Resonance Energy Transfer (BRET) Assay for Protein-Protein Interaction

293T cells were co-transfected with pFC14K and pFC32K plasmids expressing IKZF3-HaloTag and CRBN-Nanoluciferase fusion proteins and incubated overnight. Cells were then trypsinized and seeded into 96 well plates, at 40,000 cells per well. The BRET assay was performed per manufacturer's instructions (Promega™). In brief, cells were incubated for 2 hours with varying concentrations of Pomalidomide or DMSO, 10 μM MG132, and 100 nM HaloTag NanoBRET 618 Ligand. NanoBRET Nano-Glo substrate was added and donor (460 nm) and acceptor (618 nm) emission were measured within 10 minutes (EnVision, Perkin Elmer). Mean corrected milliBRET units (mBU) was calculated.

### Jurkat Target Cell Co-Culture Assay

Jurkat T cells lentivirally transduced with the genetic components described herein were co-cultured for 24 hours with K562 cells (human blast crisis Chronic Myeloid Leukemia cell line) lentivirally transduced with an expression vector encoding CD19 (referred to as K562-CD19) (G&P Biosciences LTV-CD19) or isogenic K562 cells lacking the target antigen. Both K562-CD19 and K562 cell lines were engineered to express homogenous high level mCherry expression via transduction with the Jenner lentivirus, allowing discrimination of K562 and Jurkat cells by flow cytometry. Unless otherwise specified, 50,000 Jurkat cells were co-cultured with 10,000 K562 cells in 110 ul of RPMI supplemented with Pen/Strep and 10% fetal calf serum for 24 hours. The supernatant was then harvested for cytokine secretion assays and IL2 concentration in the supernatant was performed according to manufacturer specifications (BD Biosciences 555190). The cells were analyzed by flow cytometry. Expression of the early activation marker CD69 (BioLegend 310910), myc tagged cell surface proteins (Cell Signaling Technologies 2233S), and fluorescent protein markers were assessed.

### Primary Human T Cell Experiments

Primary human T cells from normal donors were expanded with CD3/CD28 Dynabead stimulation for 7 days. On day 1, the T cells were transduced with lentivirus encoding chimeric antigen receptors. Flow cytometry for eGFP or mCherry was used to assess transduction efficiency. On day 10, the CAR-T cells were used in functional assays or viably frozen.

### Killing Assay

Target tumor cell lines engineered to express Click Beetle Green (CBG) Luciferase were co-cultured with CAR-T cells for 18 hours. Tumor cell killing was determined from endpoint CBG Luciferase signal in surviving cells (Promega BioGlo Luciferase Assay).

### Live Cell Imaging

Adherent target tumor cell lines were seeded in 6-well plates, after which CAR-T cells were added and co-cultured for 24 hours. Fluorescence (eGFP and mCherry) live-cell imaging was performed throughout this time course (Incucyte S3).

### Example 2: Basis for Molecular Switch Integration into CAR T Cell Design

CAR-T cells can act to kill targeted tumor cells, but can also elicit negative effects that are capable of propagating in an unchecked manner. As noted above, conventional CAR-T design uses a single polypeptide that includes tumor antigen-binding domains (e.g., an scFv), a transmembrane domain, a costimulatory domain and a CD3ζ domain (TCR ITAMs), with this single polypeptide therefore capable of both specifically binding a tumor antigen and propagating an activated T cell response. Chimeric antigen receptors (CARs) are recombinant receptor constructs composed of an extracellular single-chain variable fragment (scFv) derived from an antibody, joined to a hinge/spacer peptide and a transmembrane domain, which is further linked to the intracellular T cell signaling domains of the T cell receptor. CAR T cells combine the specificity of an antibody with the cytotoxic and memory functions of T cells. The specific domains of a CAR construct include: (1) the target element, which is the single-chain variable fragment (scFv) expressed on the surface of a CAR T cell, which confers antigen specificity (the scFv is derived from the portion of an antibody that specifically recognizes a target protein; (2) the spacer domain, which connects the extracellular targeting element to the transmembrane domain (TMD) and affects CAR function and scFv flexibility; (3) the TMD, which traverses the cell membrane and anchors the CAR to the cell surface, also thereby connecting the extracellular domain to the intracellular signaling domain, thereby impacting expression of the CAR on the cell surface; (4) the costimulatory domain, which is derived from the intracellular signaling domains of costimulatory proteins, such as CD28 and 4-1BB, which enhances cytokine production; and (5) the signaling domain, i.e., the CD3ζ domain, which is derived from the intracellular signaling portion of the T cell receptor, which mediates downstream signaling during T cell activation. To control the potentially negative effects of CAR T cell signaling observed in certain instances, it was contemplated that small molecule-responsive switches might be designed and introduced into CAR constructs, thereby enabling greater control over CAR T cell activities than heretofore available.

### Example 3: Clinically Useful Molecular ON-Switch Design and Implementation

It was herein contemplated that small molecule gating could enable custom tuning, increased safety and expanded applicability of CAR-T cells, as specifically shown in FIG. 1 for an “ON”-switch applied to CAR-T cells. The exemplified ON-switch CAR of the instant disclosure is a split CAR system that requires small molecule-inducible dimerization of domain A and domain B for activation, which can allow oncologists to tune the activity of CAR-T cells along individualized risk/benefit criteria. Prior to the instant disclosure, while a rapamycin-induced dimerization system had been described (see, e.g., Hubbard et al. Front. Physiol. 5: 478, which shows the structural basis for rapamycin-induced dimerization of FRB and FKBP as the canonical example of chemically-induced dimerization), a clinically valid drug-inducible heterodimerization system had not yet been disclosed.

In view of the FDA-approved status of thalidomide analog immunomodulatory drugs (IMiDs), and noting the recently described structural basis for lenalidomide-induced CRBN-IKZF3 degron interaction (see Petzold et al. Nature 532: 127-130, which shows the structural basis for lenalidomide-induced CRBN-IKZF3 degron interaction, further showing thalidomide analog immunomodulatory drug (IMiD)-dependent binding between CRBN and substrates), it was specifically contemplated that CRBN-IKZF3 degron interaction could be repurposed as a chemically-inducible dimerization (CID) system, through the engineering of multiple novel properties into CRBN- and IKZF3-derived polypeptides. Conserved design features of CID systems include: (1) specific drug-dependent interaction (confirmed for CRBN-IKZF3 degron interaction); (2) modular domain structures (confirmed for CRBN-IKZF3 degron interaction); (3) minimal dimerization domains (identified herein for CRBN-IKZF3 degron interaction and under continuing investigation); and (4) minimal interaction with endogenous proteins (CRBN-IKZF3 degron interaction has been adapted herein to avoid certain endogenous protein interactions; however, such attributes also remain under continuing investigation).

It was contemplated that additional requirements for clinical use of a small molecule-responsive heterodimeric system could also be met by an engineered CRBN-IKZF3 degron system of the instant disclosure, including (1) non-toxicity, e.g., non-immunosuppressive and non-immunogenic attributes (with the caveat that IMiDs, while FDA-approved molecules, remain teratogenic, which would likely limit medical applications of the presently-described heterodimerization system to highly morbid clinical indications such as cancer and severe autoimmunity); (2) availability, including both pharmacodynamics and pharmacokinetic availability; and (3) freedom from regulatory impediment, i.e., the IMiD modulatory agent(s) of the instant disclosure are FDA-approved.

To make an optimized ON-switch that employed the recently characterized IMiD-responsive (CID) CRBN-IKZF3 system, it was specifically identified that such a CRBN-IKZF3-derived CID system would need to have minimal drug-dependent heterodimerization domains, would also need to be disrupted for recruitment of the CRBN-dependent E3 ubiquitin ligase (E3UL)—to thereby effect adaptation of what would otherwise be an OFF-switch to an ON-switch configuration, might further need to disrupt endogenous CRBN-dependent degradation of the IKZF3-derived dimer pair (IKZF3^{orthogonal}), yet would also need to retain heterodimerization between the IKZF3^{orthogonal} and CRBN-derived dimer pair (thereby retaining the IMiD-responsiveness of the heterodimer).

Engineering of a CRBN-IKZF3-derived CID system was commenced in a CAR T-cell system. As shown in FIG. 2, minimal heterodimerization domains of CRBN and a CRBN substrate (here, an IKZF3 degron comprising amino acids 130-189 of IKZF3) were engineered to associate in IMiD-dependent fashion, thereby bringing together a scFv- and CD28 co-stimulatory domain-containing first polypeptide with a second polypeptide containing a CD28 co-stimulatory domain and a CD3ζ domain. In the FIG. 2 schematic, the IMiD used was pomalidomide (“Pom”) and the second polypeptide comprised a transmembrane domain (TMD). The function of this CRBN-IKZF3-derived CID system within a CAR T-cell context is further shown in FIG. 3, where the scFv of an IKZF3 degron-presenting CAR binds a tumor cell-presented antigen, effecting CAR-T cell activation in the form of cytokine and cytotoxic granule release and T cell proliferation, resulting in tumor cell killing. Withdrawal of the IMiD can then be used to turn off T cell activation, thereby reducing and/or preventing any toxicity that might have otherwise been induced during the (prolonged) activated CAR-T cell response.

Having identified an IKZF3 degron comprising amino acids 130-189 of IKZF3 as functional for pomalidomide-mediated interaction with CRBN, variant forms of CRBN polypeptide were now examined, with the goal of identifying a variant form of CRBN that retained pomalidomide-mediated heterodimerization with IKZF3 degron while also disrupting the mechanism by which native CRBN can interact with and promote ubiquitin-mediated degradation of CRBN substrates (e.g., IKZF3) in the presence of IMiD. As shown in FIG. 1, minimal CRBN polypeptide variants for an ON-switch of the instant disclosure were designed that retained binding to IMiD (pomalidomide as exemplified herein) and the CRBN substrate (here, IKZF3 degron polypeptide) but were also engineered to remove/disrupt the wild-type CRBN domain that binds DDB1/CUL4 (where DDB1/CUL4 binding of wild-type CRBN mediates ubiquitination and degradation of CRBN substrates, via a molecular mechanism also involving ROC and E2). Design/assembly of such a functional cell surface split receptor as exemplified involved introduction of compound that was modeled to bring together a heterodimer that included an antigen-binding scFv-TMD-costimulatory domain-compound-binding component #1 on a first polypeptide and compound-binding component #2 fused to a CD3ζ domain on a second polypeptide. Such a split configuration was modeled to achieve a T cell response only upon both antigen and compound (small molecule) binding. Connection of the exemplified ON-switch system to the wild-type CRBN/DDB1/CUL4A/ROC1/E2/Ubiquitin-mediated system of IMiD (e.g., lenalidomide) substrate degradation was broken, e.g., by use of minimal CRBN polypeptides, thereby removing such a system, when activated, from proteasomal degradation of ubiquitinated substrate that would otherwise have been mediated via CRBN in the presence of the IMiD. Specifically, the ON-switch of the current disclosure has importantly removed IMiD-mediated binding between CRBN and a CRBN substrate from the threat of attack by the native ubiquitin/proteasome pathway, via engineering and use of minimal substrate-binding forms of CRBN from which DDB1/CUL4A binding elements have been removed.

To assess the extent and dose-responsiveness of interactions between various minimal forms of CRBN and the IKZF3 degron, Bioluminescence Resonance Energy Transfer (BRET) was employed (FIG. 1). BRET was specifically used to detect energy transfer from a minimal CRBN polypeptide to the IKZF3 degron. Tested forms of minimal CRBN included minCRBN1, minCRBN2, minCRBN3 and minCRBN4, each of which were aligned against the wild-type CRBN (FIG. 1; all tested minCRBNs lacked the DDB1-binding domain—minCRBN1 comprised amino acids 339-442 of CRBN (including the TBD); minCRBN2 comprised amino acids 317-442 of CRBN (including TBD and an N-terminal extended sequence relative to minCRBN1); minCRBN3 included all CRBN sequence other than amino acids 194-207 (deletion of the DDB1-binding domain); and minCRBN4 included all sequences other than amino acids 187-260 (deletion of the DDB1-binding domain)). BRET assays that tested associations between minCRBN polypeptides and the IKZF3 degron (FIG. 1, where milliBRET units (mBU) were detected) revealed pomalidomide-dependent interactions between IKZF3 degron and any of minCRBN2, minCRBN3 and minCRBN4, as well as between the IKZF3 degron and wild-type CRBN (positive control). Among minCRBN polypeptides tested, pomalidomide dose-dependence was only not observed for the interaction between minCRBN1 and the IKZF3 degron (FIG. 1).

To employ the minCRBN-IKZF3 degron IMiD-responsive engineered system in cells without confronting confounding (i.e., degradative) effects of native CRBN, CRISPR/Cas9 was used to disrupt CRBN in mammalian cells (via introduction of a biallelic CRBNΔe5 disruption of native CRBN, which is described in greater detail below), into which the minCRBN-IKZF3 degron system was also introduced. As shown in FIG. 1, while 293T cells possessing a biallelic CRBN disruption (biallelic CRBNΔe5) to which a wild-type CRBN-IKZF3 degron system was added successfully promoted degradation of the IKZF3 degron in the presence of 1 μM pomalidomide (as tracked by assessing the eGFP/mCherry ratio in these cells), the eGFP/mCherry ratio remained unitary and stable over time for all minCRBNs-presenting systems tested (minCBRN1-4), indicating that none of minCRBN1-4 polypeptides were capable of rescuing IMiD-responsive degradation of the IKZF3 degron (consistent with each of minCRBN1-4 lacking the DDB1-binding domain believed to be critical for activation of the ubiquitination pathway). Thus, minimal heterodimerization domains associated in human cells and did not recruit CRL4^{CRBN}.

The importance of cell surface localization of non-scFv-presenting polypeptides of the minCRBN-IKZF3 degron CAR system was then examined. As shown in FIG. 2, the IMiD-responsive ON-switch was modified such that different forms of tethering the minCRBN-presenting polypeptide (minCRBN3 polypeptide as depicted and exemplified herein) to the plasma membrane were assessed for IKZF3 degron interaction capability, as compared to an untethered minCRBN3. Specific minCRBN-presenting polypeptide tethers to the plasma membrane that were tested included: (1) a CD8/CD28 system in which about 36 amino acids of TMD and cytosolic sequence connected to the co-stimulatory/minCRBN domains of the thereby membrane-tethered polypeptide; (2) a PD1 Y→F ITAM/ITSM construct, in which minCRBN was tethered to the plasma membrane via a TMD and cytosolic domain of approximately 100 amino acids in length; and (3) a LYN-CD28 construct, in which membrane tethering was achieved via myristoyl/palmitoylation. Cytosolic minCRBN (untethered) interacted in a pomalidomide-dependent manner with IKZF3 degron (as assessed via BRET assay, as above) to a significantly lesser extent than any of the various plasma membrane-tethered versions of minCRBN-presenting polypeptide. Strikingly, little difference was observed among the various membrane-tethered forms of minCRBN-presenting polypeptides (all three membrane-tethered minCRBN formats displayed similar levels of IKZF3 degron interaction in the presence of pomalidomide). Cell surface localization of IKZF3 degron and minCRBN3 therefore decreased the observed Kd of pomalidomide-dependent dimerization.

It was next examined whether the IZKF3 degron of the minCRBN-IKZF3 degron system could be further stabilized against ubiquitin pathway-mediated degradation via removal of potential sites of ubiquitination (i.e., lysine residues) within the IKZF3 degron polypeptide sequence. As shown in FIG. 3, five lysine-to-arginine (K to R) substitutions within the IKZF3 polypeptide sequence were performed upon the original (“wild-type”) scFv-TMD-CD28-IKZF3 degron polypeptide, to produce an “iK0” polypeptide lacking these sites of potential ubiquitination. When tested by BRET assay for minCRBN3-IKZF3 degron interaction (FIG. 3), the iK0 split receptor demonstrated decreased pomalidomide-dependent Kd and increased Bmax relative to the original (“wild-type”) split receptor system. The iK0 split receptor system also exhibited increased BRET signal retention over prolonged exposures to pomalidomide (FIG. 3). Thus additional stabilization of the IKZF3 degron was achieved via elimination of six lysine residues within the IKZF3 degron polypeptide sequence.

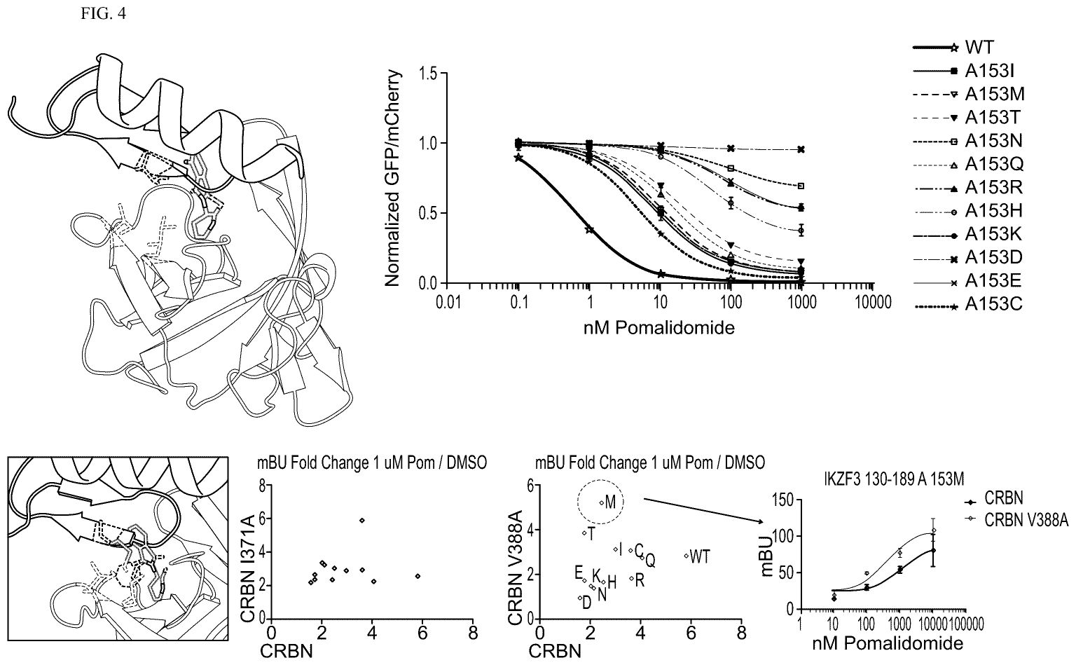

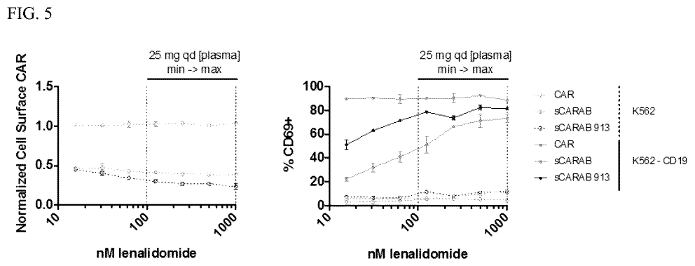

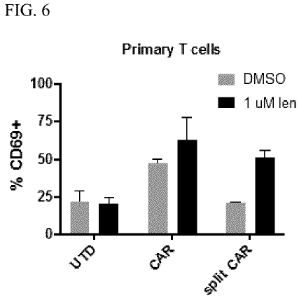

While disruption of native CRBN within a cell presenting the IMiD-responsive minCRBN-CRBN substrate system of the instant disclosure was shown to shield the instant CRBN substrate degron from native CRBN-mediated degradation (see above), the prospect was also contemplated of shielding a minCRBN-CRBN system of the instant disclosure from effects mediated by native CRBN by altering the respective IMiD binding pocket residues of both the CRBN substrate of the system (here, IKZF3 degron) and the minCRBN employed by the system, such that a heterodimer of residue-substituted minCRBN and residue-substituted CRBN substrate (e.g., residue substituted IKZF3 degron) associates with one another in IMiD-dependent fashion, yet binding of the residue-substituted CRBN substrate with native forms of CRBN is dramatically reduced or eliminated (thereby possibly even removing the need to perform a disruption of native CRBN within cells that utilize a minCRBN-CRBN substrate system of the instant disclosure). As shown in FIG. 4, strategic residue substitutions were made respectively within the IMiD (pomalidomide) binding pocket of the IKZF3 degron and a minCRBN polypeptide of the instant system. As noted above, such alterations could be used to make the IKZF3 degron less responsive and/or effectively non-responsive to pomalidomide-dependent degradation by native forms of CRBN polypeptide, while it was reasoned that “offsetting” substituted forms of CRBN polypeptide could also be identified that restored pomalidomide-dependent association to the substituted IKZF3 degron-substituted CRBN interaction (thereby effectively freeing this substituted IKZF3 degron-substituted CRBN dimerization from the potential degradative impacts of wild-type CRBN, where present, thus reducing or eliminating the need for genomic disruption of native CRBN in candidate therapeutic cells that employ such a substituted IKZF3 degron-substituted CRBN dimer system). Initially, various substitutions made within the IKZF3 degron at the A153 site (which was shown by crystal structure to interact with IMiD/pomalidomide) were identified that disrupted the pomalidomide-dependent, wild-type CRBN-mediated degradation of the IKZF3 degron in wtCRBN-expressing cells (refer to dose-response curves of the middle panel of FIG. 4). Among all forms of substituted IKZF3 degron tested, the A153D substitution was identified as the most effective at disrupting pomalidomide-dependent IKZF3 degron degradation.

As shown in the lower panels of FIG. 4, an A153M-substituted form of the IKZF3 degron was then examined for whether crystal structure-implicated CRBN substitutions at residues 371 and 388 of CRBN could restore pomalidomide-dependent binding between the newly-substituted CRBN polypeptide and the A153M-substituted form of the IKZF3 degron. It was strikingly identified that the CRBN V388A substitution elicited the greatest restoration of pomalidomide-dependent binding between the newly-substituted CRBN polypeptide and the A153M-substituted form of the IKZF3 degron, as initially assessed by BRET assay at 1 μM pomalidomide, and, as then shown in the dose-response curves at lower right of FIG. 4, pomalidomide dose-responsiveness was also enhanced/restored to interactions with the A153M-substituted form of the IKZF3 degron by the CRBN V388A substituted polypeptide, as compared to the significantly less robust interaction of the A153M-substituted form of the IKZF3 degron with wtCRBN, as assessed by BRET assay across a range of pomalidomide concentrations.

### Example 4: Clinically Useful Molecular OFF-Switch Design and Implementation

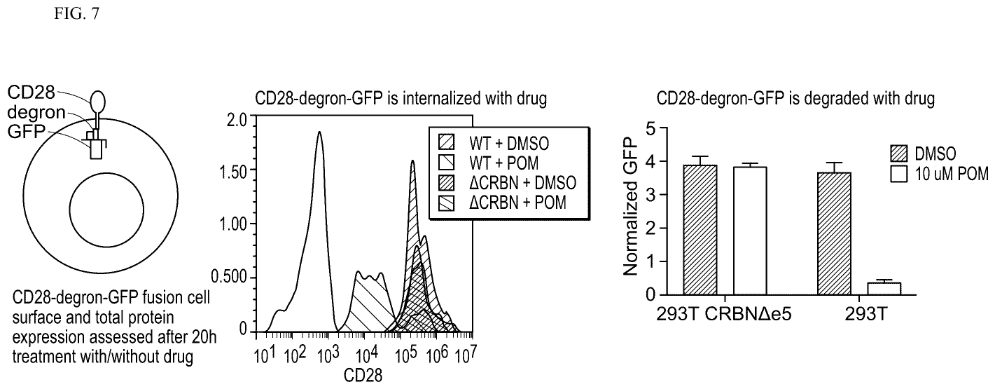

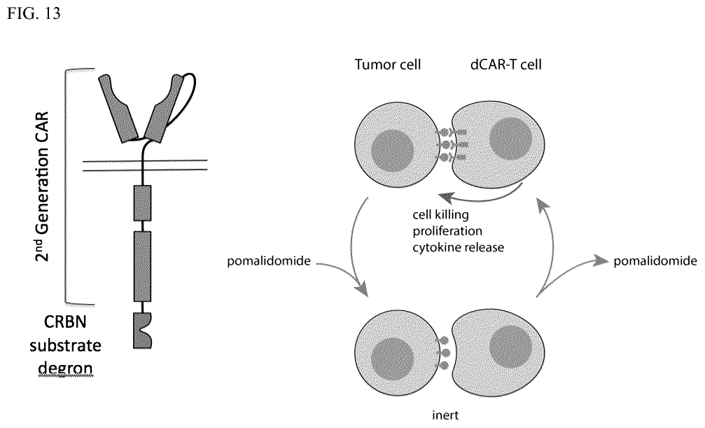

The above-described IMiD-responsive IKZF3 degron was also utilized to engineer an exemplary IMiD-responsive “OFF”-switch in a CAR-T cell system. As shown in FIG. 13 an OFF-switch CAR construct was designed that presents an engineered element capable of causing intracellular degradation of the CAR in a precise, input-responsive manner. The input-responsive element is referred to as an “engineered degron,” and the input to which the degron responds in certain aspects of the instant disclosure is an IMiD (e.g., pomalidomide). In the absence of IMiD administration, the OFF-switch CAR construct comprising the engineered degron is active (thereby recognizing tumor cell antigen and propagating signaling to produce cytokines, cytotoxic granules, T cell proliferation, etc.—see FIG. 13 at right). In contrast, when IMiD is administered to a T cell containing the CAR construct comprising the engineered degron, degradation of the CAR construct is triggered, and T cell activation is thereby terminated. The viability of IMiD-responsive IKZF3 degron-mediated degradation of a polypeptide was first tested in the context of IKZF3 degron inclusion within a GFP-tagged CD28 fusion protein. As shown in FIG. 7, when cell surface and total protein expression were assessed after 20h treatment with or without the IMiD pomalidomide, a CD28-degron-GFP fusion polypeptide (where the degron was an ImiD-responsive IKZF3 polypeptide comprising amino acids 130-189 of IKZF3) showed degradation-responsiveness to the IMiD pomalidomide, within cells expressing wild-type CRBN. Deletion of native CRBN in such cells demonstrated that such degron-mediated degradation was CRBN-dependent (as no pomalidomide-responsive degradation was observed in cells deleted for the native CRBN locus). In particular, the CD28-degron-GFP construct was observed to be internalized upon treatment with pomalidomide in a CRBN-dependent manner (FIG. 7 middle panel), while a dramatic degradation effect (as assessed by reduced normalized GFP signal) was observed at 10 μM pomalidomide for 293T cells expressing the CD28-degron-GFP construct (FIG. 7 right panel). Such pomalidomide-responsive degradation was rescued via disruption of CRBN in these cells—in particular, as shown in the histograms of the right panel of FIG. 7, a biallelic disruption of CRBN exon 5, CRBNΔe5, when integrated into these cells, resulted in no significant decline in normalized GFP levels in the presence of 10 μM pomalidomide, as contrasted with the highly pomalidomide-responsive degradation observed for 293T cells expressing wild-type CRBN.

Consistent with the above results observed for the CD28-degron-GFP construct, the IKZF3 degron (comprising amino acids 130-189 of IKZF3) also functioned as a highly pomalidomide-responsive OFF-switch when integrated into the context of a CAR construct (FIG. 8). As shown in FIG. 8, an ideal drug-OFF-switch would function by dramatically decreasing signal (e.g., CAR T signal) when an IMiD (e.g., pomalidomide) is administered, yet would revert to previously observed levels of activity/signal following removal of the IMiD. Indicative of such function, the IKZF3 degron mediated particularly robust pomalidomide-responsive degradation of CAR-like polypeptide constructs comprising transmembrane domains (TMDs), as compared to a cytosolic, untethered form of the IKZF3 degron (see lower panel of FIG. 8). Specifically, a cytosolic, untethered form of the IKZF3 degron exhibited an EC50 of 0.4 nM pomalidomide, whereas a CD28-degron construct and a CD28-CD3ζ-degron construct exhibited EC50 values of 24 nM and 8 nM pomalidomide, respectively.

### Example 5: Identification of a ZFP91/IKZF3 Hybrid Degron as an Apparently Enhanced Degron

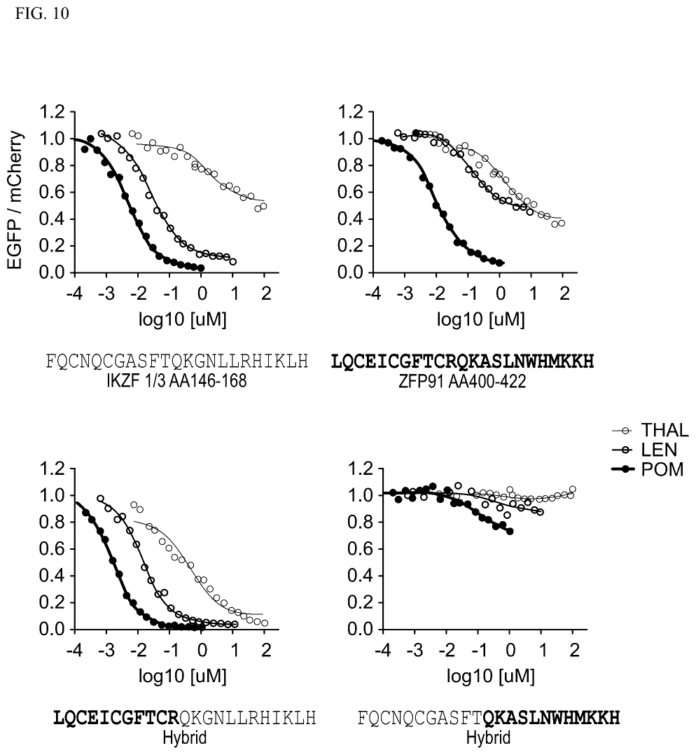

A hybrid sequence of two distinct CRBN substrates, IKZF3 and ZFP91, was constructed and examined for activity within an “OFF-switch” system of the instant disclosure. In such experiments, reporter constructs containing zinc finger sequences were expressed in HEK239T cells via lentiviral transduction, after which the cells were treated with a titration of (IMiD) drug concentrations for 20h. Flow cytometry was used to measure the EGFP/mCherry ratio normalized to the average of three DMSO treated controls. Data shown in FIG. 10 are representative of two experimental replicates (technical replicates=0, experimental replications=2). As shown in FIG. 10, a ZFP91/IKZF3 hybrid sequence (SEQ ID NO: 32) showed greater IMiD sensitivity than either the ZFP91 degron sequence alone or the IKZF3 degron sequence alone. Notably, the “reverse hybrid” configuration of IKZF3/ZFP91 showed minimal IMiD sensitivity/responsiveness, further underscoring that the ZFP91/IKZF3 hybrid sequence configuration was especially active/responsive.

### Example 6: Degron-Based Switches were Highly Responsive to Both IMiD Treatment and Withdrawal

To examine the kinetics of IMiD responsiveness of a degron-based system of the instant disclosure, an OFF-switch system as employed for FIG. 8 was examined. As for the FIG. 8 OFF-switch system, the CD28-CD3ζ-degron protein differed from the anti-CD19 CAR sequence published previously (PMID 19561539) in two ways. First, the FMC63 anti-CD19 scFv was exchanged for the CD28 Ig-like V-type extracellular domain. Second, the protein was fused in-frame at its C-terminus with the degron IKZF3aa130-189 that was shown to mediate lenalidomide-dependent degradation by CRL4^{CRBN} (PMID 24292625). Jurkat T cells were transduced with lentivirus encoding the three degron-eGFP fusion proteins (pSFFV-insert-linker-eGFP-IRES-mCherry), where mCherry expression served as an internal control for transgene expression.

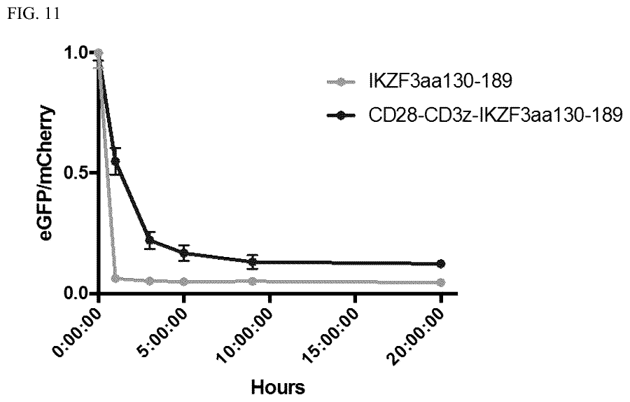

As shown in FIG. 11, transmembrane protein internalization and degradation of the exemplary OFF-switch CAR design occurred rapidly upon addition of pomalidomide. In particular, degradation of the presently exemplified degron-eGFP protein was measured at the specified times after addition of 1 μM pomalidomide (the experiment was performed in triplicate). The cytoplasmic IKZF3-derived degron was rapidly cleared, approaching 90% degradation by 1 hour after addition of pomalidomide. The CD28-CD3ζ-IKZF3aa130-189 polypeptide was degraded slightly less quickly, yet approached 80% degradation by 3 hours after drug addition. Thus, all polypeptides harboring the OFF-switch degron system were rapidly internalized and degraded in an IMiD-responsive manner.

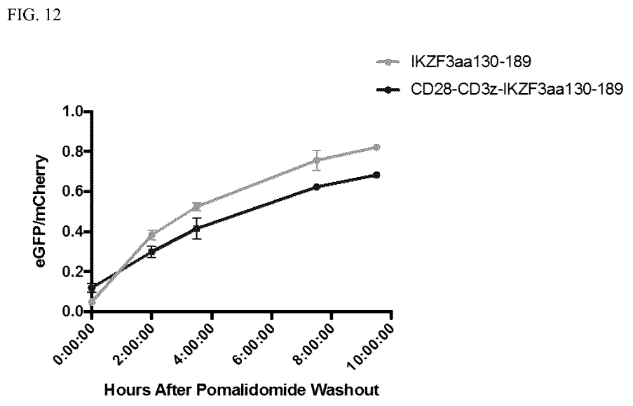

As shown in FIG. 12, re-synthesis of the exemplified transmembrane degron-tagged protein after washout of pomalidomide also occurred with kinetics that resembled those of the IKZF3 degron alone. In particular, cells with the degron-eGFP fusion proteins were cultured with 1 μM pomalidomide for 16 hours. Cells were then washed with media three times and resuspended for culture in media lacking pomalidomide, after which the return of degron-eGFP was compared to untreated cells at 2 h, 4h, 8 h and 10h time points (the experiment was performed in triplicate). After pomalidomide washout, resynthesis of both degron-tagged proteins (the tagged CD28-CD3ζ-IKZF3aa130-189 polypeptide or the tagged IKZF3aa130-189 degron) regenerated >50% of protein levels in less than 8 hours. The exemplified OFF-switch was therefore responsive to removal of IMiD.

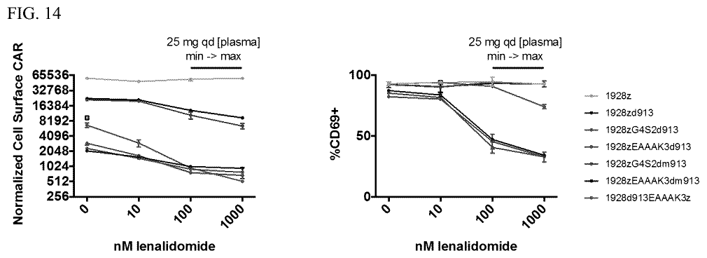

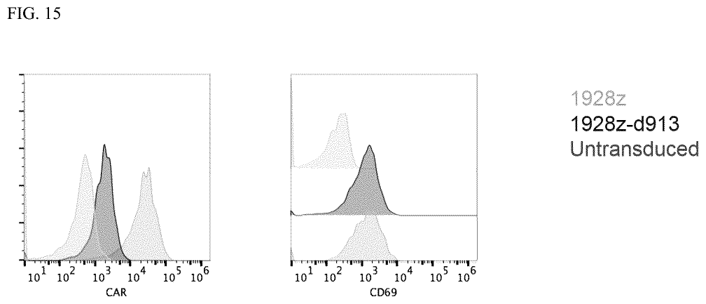

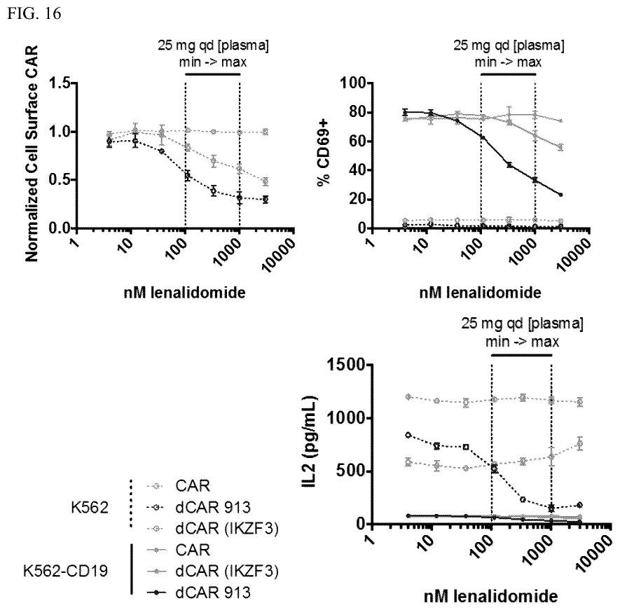

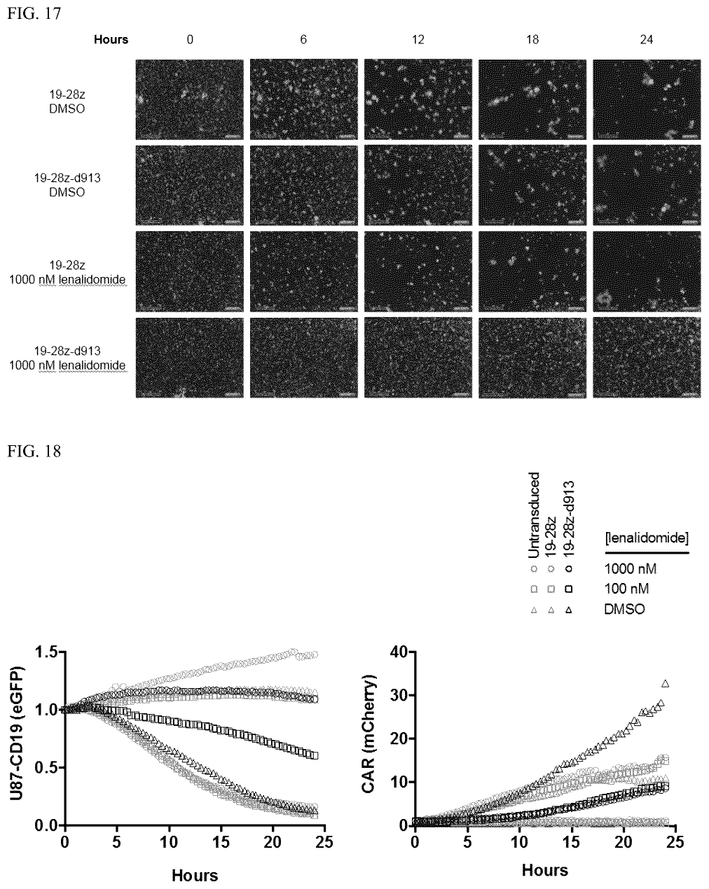

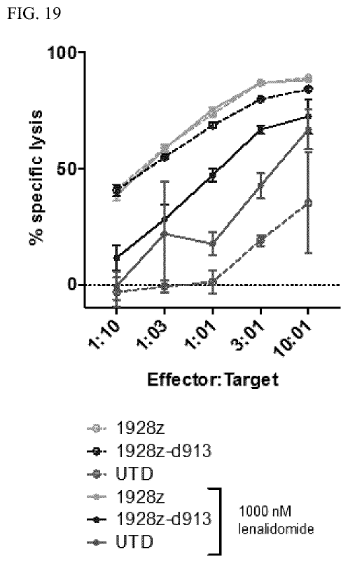

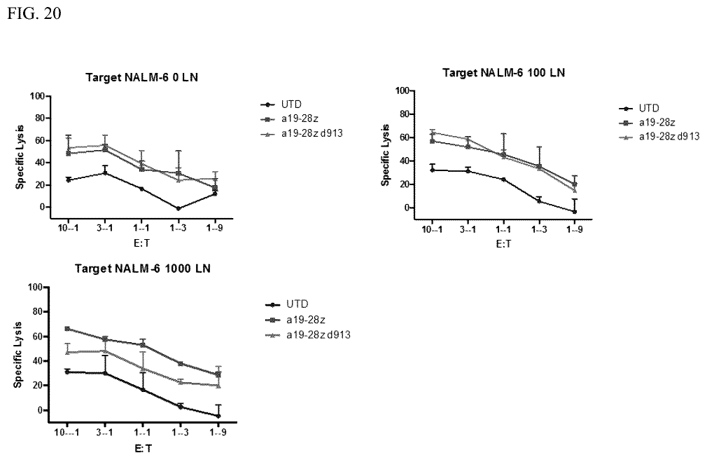

A highly effective IMiD-responsive OFF-switch degron format was identified, construction of which employed modification of the non-IKZF3 C2H2 zinc finger degron amino acids 400-410 with N-terminal and C-terminal additions of IKZF3 sequences (IKZF3 amino acids 130-145 were joined at the N-terminus, while IKZF3 amino acids 157-189 were joined at the C-terminus), thereby forming a “d913” hybrid degron sequence (a “K0” form of the d913 degron sequence was also produced, as described elsewhere herein). A d913 degron-tagged fusion protein was demonstrated to turn off the tumor cell killing of primary human T cells engineered to express degron-tagged CARs (FIGS. 13-20. More broadly, it was demonstrated that modification of any given non-IKZF3 C2H2 zinc finger degron sequence via addition of IKZF3 amino acids 130-145 to the N-terminus and IKZF3 amino acids 169-189 to the C-terminus, to generate an approximately 60 amino acid hybrid zinc finger could provide more sensitive/deep drug-induced protein degradation (noting that such longer hybrid degrons resulted in lower protein abundance).

### Example 7: Additional Functional, Drug-Inducible ON-Switch and OFF-Switch Formats

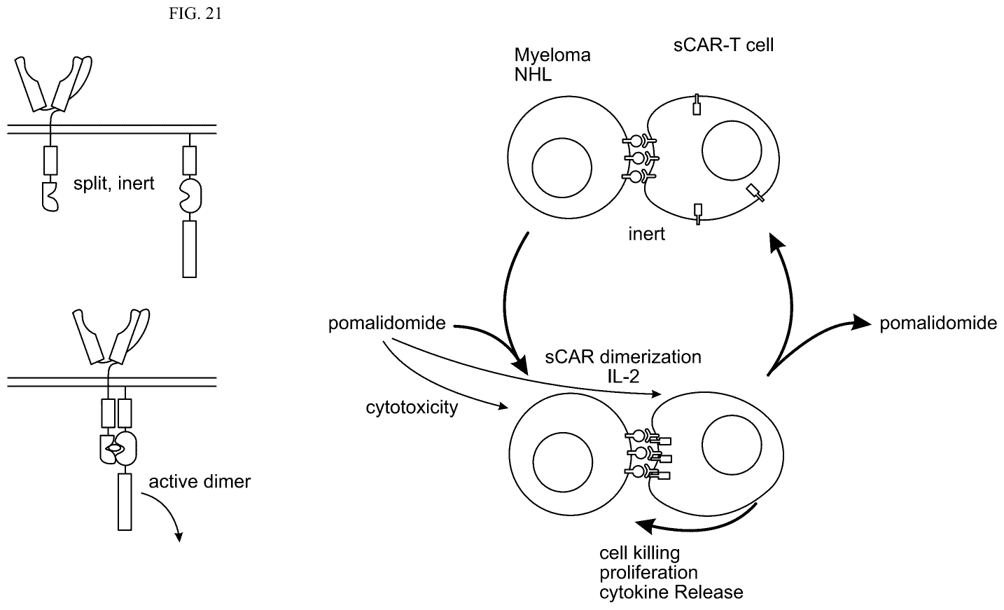

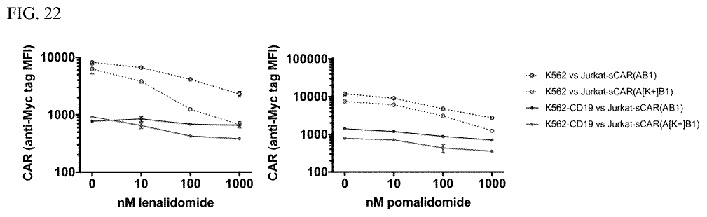

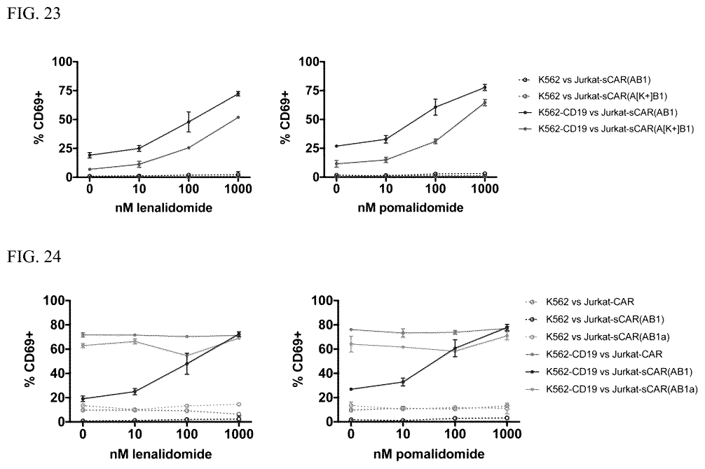

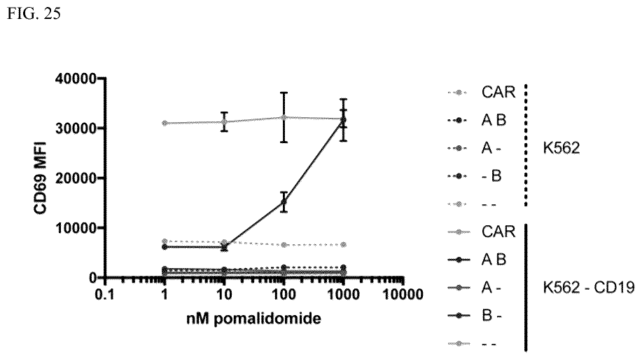

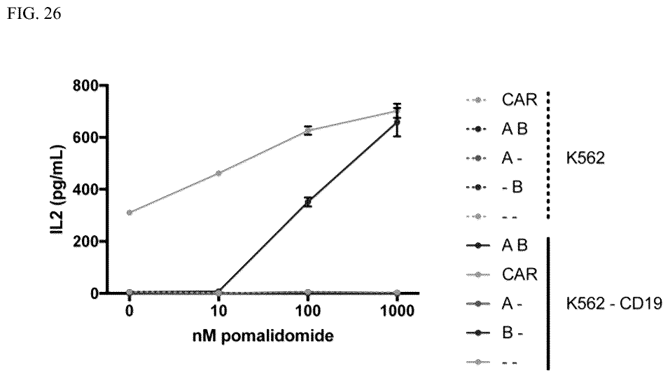

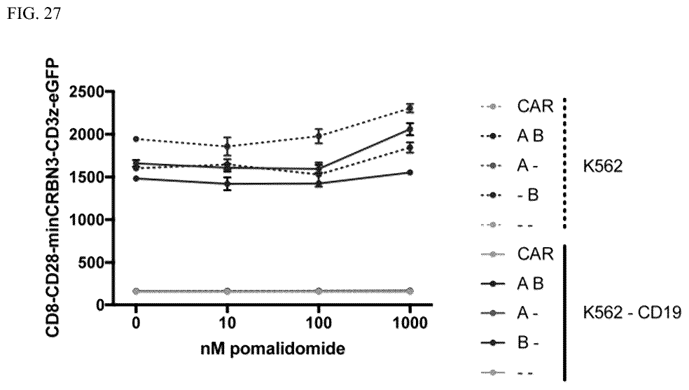

The IMiD-responsiveness of a split CAR ON-switch configuration was also further validated (including validating CAR-mediated signaling responsiveness to drug administration), as shown in FIGS. 21-27.

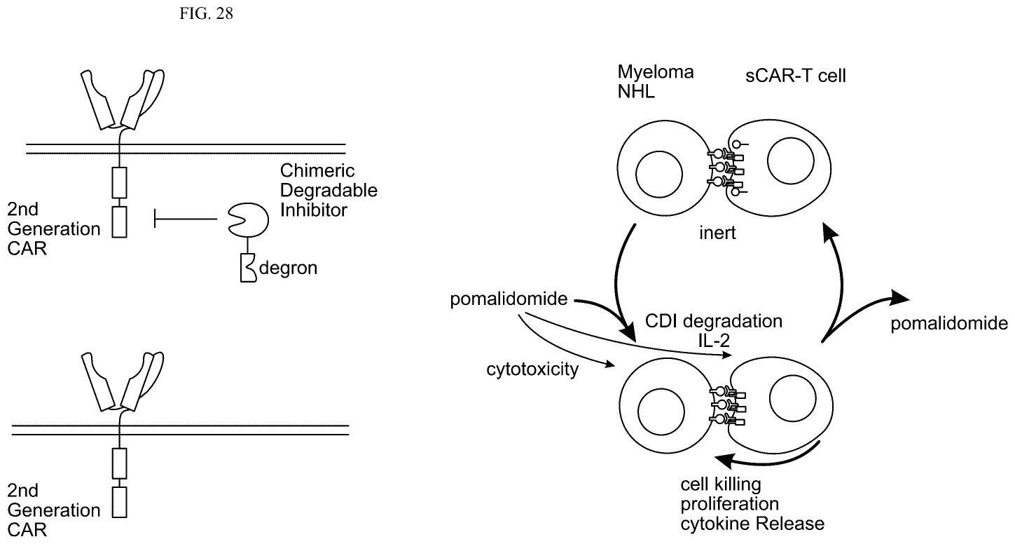

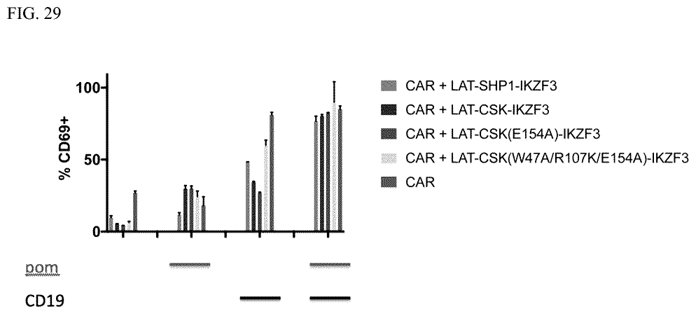

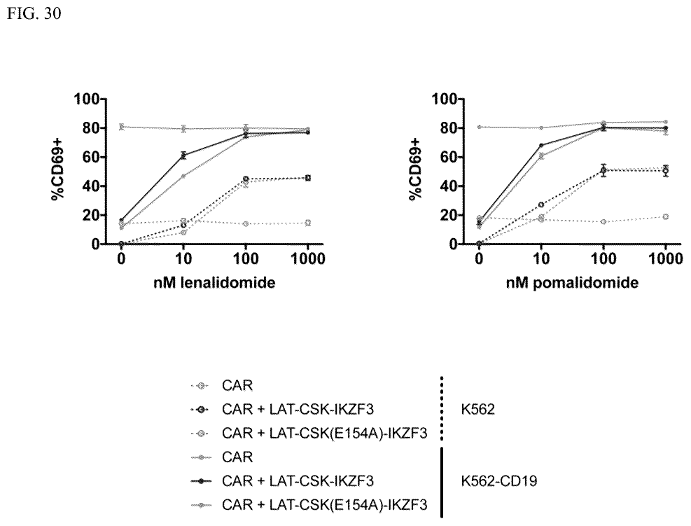

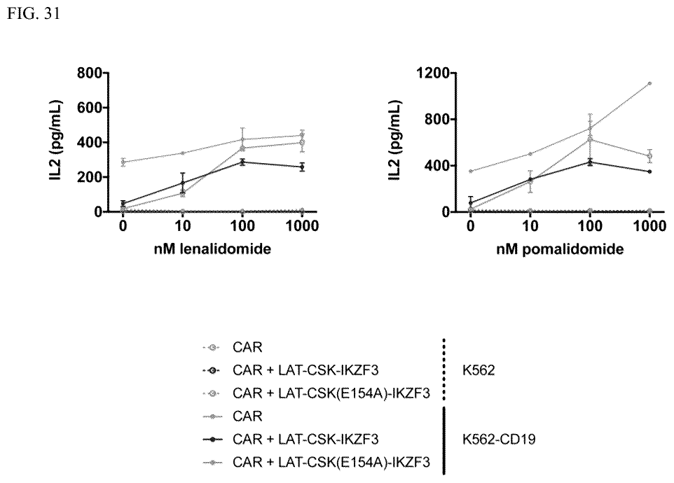

As shown in FIG. 28, a degron of the instant disclosure can be fused to an inhibitor of CAR, thereby producing a Chimeric Degradable Inhibitor (CDI), which functions as an ON-switch upon drug administration. The drug-responsiveness of such a CDI was validated, as shown in FIGS. 29-31.

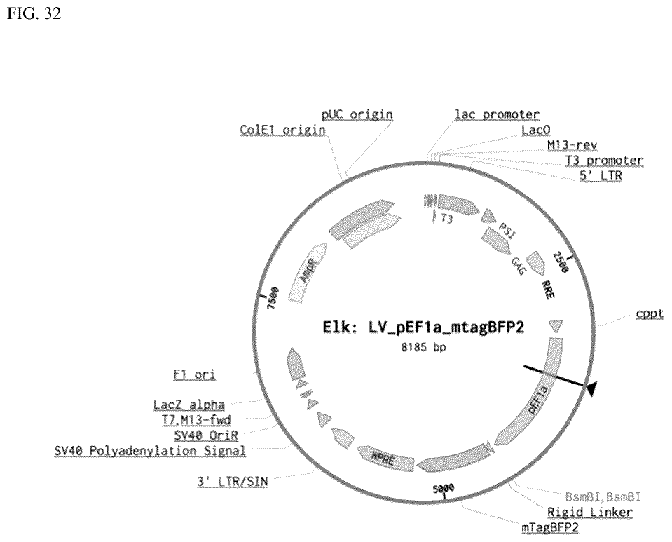

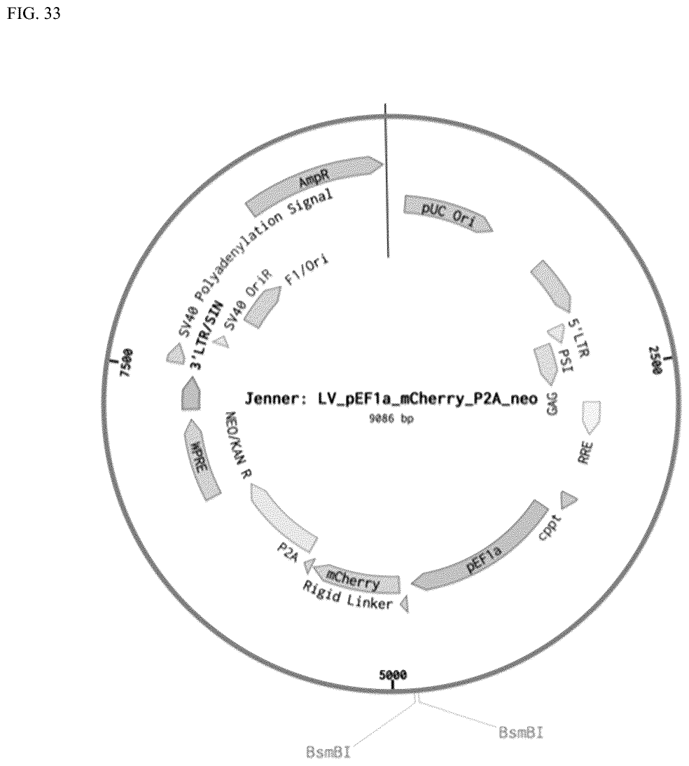

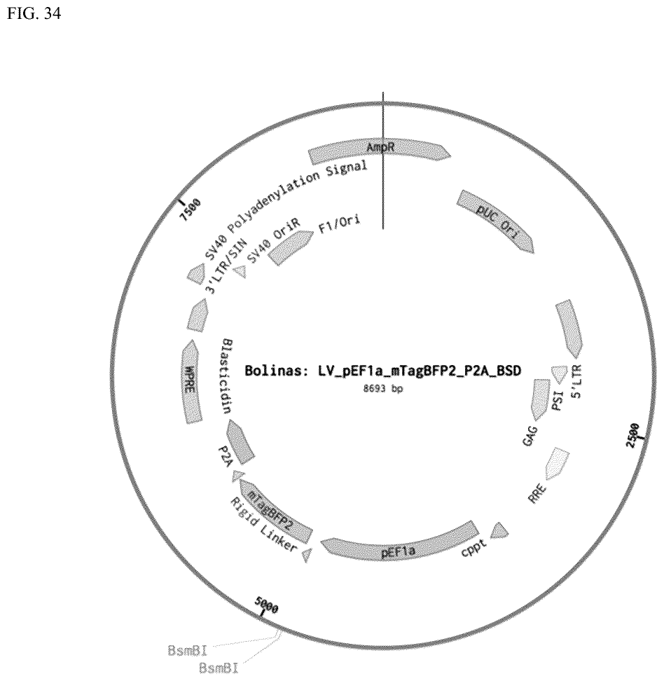

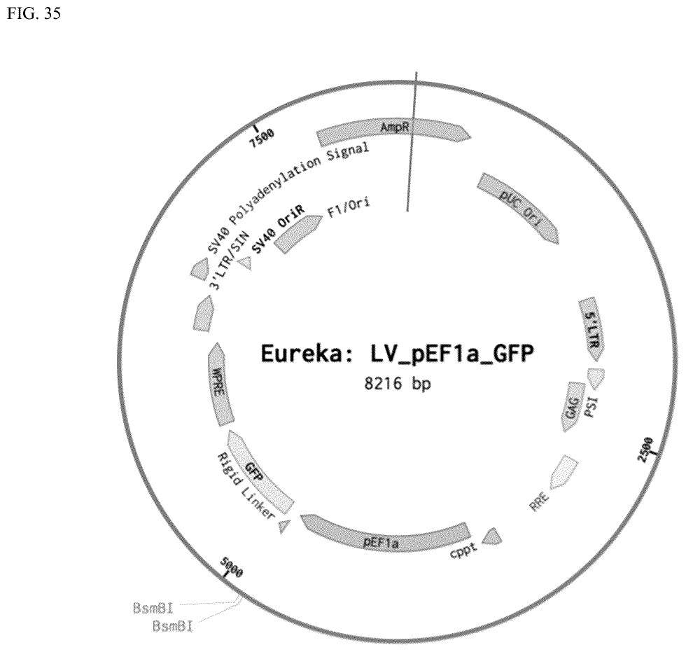

Additional nucleic acid vectors/constructs used in the above experiments are also depicted schematically in FIGS. 32-35.

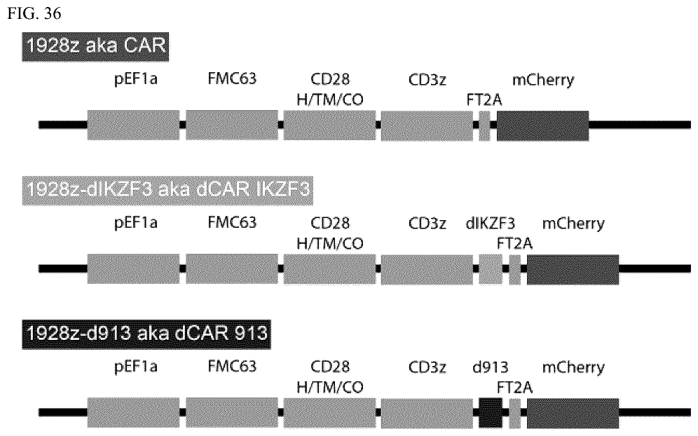

Further CAR degron constructs were made in the current study, including 1928z-dIKZF3 aka dCAR IKZF3 and 1928z-d913 aka dCAR 913 (FIG. 36).

As demonstrated above, a clinically applicable drug-inducible ON-switch peptide logic gate was generated by engineering heterodimerization domains that can regulate molecular assembly (i.e., split CAR design) to control the function of engineered proteins (see Examples 1-3 above).

In addition, a clinically applicable drug-inducible OFF-switch peptide tag that recruits the CRL4^{CRBN} E3 ubiquitin ligase to degrade engineered proteins was also generated and employed, as specifically exemplified in the context of CAR-T cells (see Examples 1 and 4 above).

Additional formats of both drug-inducible ON-switches and OFF-switches were also described and validated (see Example 7 above).

Each of these synthetic biology tools possesses clinical applications for enhancing the safety of CAR T cell therapy, as well as other engineered cell therapies.

All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

One skilled in the art would readily appreciate that the present disclosure is well adapted to carry out the objects and obtain the ends and advantages mentioned, as well as those inherent therein. The methods and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

In addition, where features or aspects of the disclosure are described in terms of Markush groups or other grouping of alternatives, those skilled in the art will recognize that the disclosure is also thereby described in terms of any individual member or subgroup of members of the Markush group or other group.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein, including the best mode known to the inventors for carrying out the disclosed invention. Variations of those embodiments may become apparent to those of ordinary skill in the art upon reading the foregoing description.

The disclosure illustratively described herein suitably can be practiced in the absence of any element or elements, limitation or limitations that are not specifically disclosed herein. Thus, for example, in each instance herein any of the terms “comprising”, “consisting essentially of′, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure provides preferred embodiments, optional features, modification and variation of the concepts herein disclosed may be resorted to by those skilled in the art, and that such modifications and variations are considered to be within the scope of this disclosure as defined by the description and the appended claims.

It will be readily apparent to one skilled in the art that varying substitutions and modifications can be made to the invention disclosed herein without departing from the scope and spirit of the invention. Thus, such additional embodiments are within the scope of the present disclosure and the following claims. The present disclosure teaches one skilled in the art to test various combinations and/or substitutions of chemical modifications described herein toward generating conjugates possessing improved contrast, diagnostic and/or imaging activity. Therefore, the specific embodiments described herein are not limiting and one skilled in the art can readily appreciate that specific combinations of the modifications described herein can be tested without undue experimentation toward identifying conjugates possessing improved contrast, diagnostic and/or imaging activity.

The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Those skilled in the art will recognize, or be able to ascertain using no more than routine experimentation, many equivalents to the specific embodiments of the disclosure described herein. Such equivalents are intended to be encompassed by the following claims.

We claim:

1. A method for treating a subject with a chimeric antigen receptor (CAR) cellular therapy, the method comprising administering to the subject a mammalian cell comprising a drug-responsive CAR comprising:
an extracellular antigen-binding domain,
a transmembrane domain (TMD),
a co-stimulatory domain,
a signaling domain and
a CRBN polypeptide substrate domain capable of binding CRBN in response to drug, thereby promoting ubiquitin pathway-mediated degradation of the drug-responsive CAR,
thereby treating a subject with a CAR cellular therapy.

2. The method of claim 1, wherein the mammalian cell overexpresses a CRBN polypeptide, optionally wherein the overexpressed CRBN polypeptide is targeted to the plasma membrane with a targeting sequence derived from LAT, PAG, LCK, FYN, LAX, CD2, CD3, CD4, CD5, CD7, CD8a, PD1, SRC, or LYN, optionally wherein the local concentration of the ubiquitin ligase CRL4^{CRBN} is increased at the plasma membrane, as compared to an appropriate control.

3. The method of claim 1, further comprising administering the drug.

4. The method of claim 1, further comprising identifying a CAR cellular therapy side effect in the subject.

5. The method of claim 4, further comprising administering the drug after the CAR cellular therapy side effect is identified in the subject.

6. The method of claim 1, wherein the drug is a small molecule drug.

7. The method of claim 1, wherein the drug is an FDA-approved drug.

8. The method of claim 1, wherein the drug can be administered to a human subject in a clinical setting.

9. The method of claim 1, wherein the drug is an IMiD.

10. The method of claim 1, wherein the drug is selected from the group consisting of thalidomide, lenalidomide and pomalidomide.

11. The method of claim 1, wherein the CRBN polypeptide substrate domain is selected from the group consisting of IKZF1, IKZF3, CK1α, ZFP91, GSPT1, MEIS2, GSS E4F1, ZN276, ZN517, ZN582, ZN653, ZN654, ZN692, ZN787, ZN827 or a fragment thereof that is capable of drug-inducible binding the CRBN polypeptide disrupted for or lacking a DDB1-interacting domain, or wherein the CRBN polypeptide substrate is a chimeric fusion product of native CRBN polypeptide sequences, optionally the IKZF3/ZFP91/IKZF3 polypeptide SEQ ID NO: 95.

12. The method of claim 1, wherein the CRBN polypeptide substrate domain comprises a hybrid fusion polypeptide comprised of ten or more residues of a non-IKZF3 C2H2 zinc finger degron sequence flanked by an N-terminal IKZF3 degron sequence and a C-terminal IKZF3 degron sequence, optionally wherein the N-terminal IKZF3 degron sequence comprises (optionally is) amino acids 130-145 (SEQ ID NO: 97) of IKZF3 or a K0 from thereof (SEQ ID NO: 100) and/or wherein the C-terminal IKZF3 degron sequence comprises (optionally is) amino acids 169-189 (SEQ ID NO: 102) of IKZF3 or a K0 form thereof (SEQ ID NO: 103), optionally wherein the non-IKZF3 C2H2 zinc finger degron sequence is a ZFP91 sequence.

13. The method of claim 1, wherein the CRBN polypeptide substrate domain is SEQ ID NO: 5.

14. The method of claim 1, wherein the signaling domain is selected from the group consisting of a CD3ζ domain, a CD3 gamma domain, a CD3 delta domain, a CD3 epsilon domain, a FcR gamma domain, a FcR beta domain, a CD5 domain, a CD79a domain, a CD79b domain, a CD66d domain, a CD4 domain, a CD8 domain, a Dap10 domain and a Dap-12 domain.

15. The method of claim 1, wherein the co-stimulatory domain is selected from the group consisting of a CD28 co-stimulatory domain, a 4-1BB co-stimulatory domain, or additional co-stimulatory domains from CD27, OX40, CD30, CD40, ICOS, LFA-1, CD2, CD7, NKG2C, or B7-H3, optionally wherein the co-stimulatory domain comprises K→R residue substitutions at positions 182 and 204 of a CD28 co-stimulatory domain sequence, or wherein the co-stimulatory domain comprises K->R residue substitutions at positions 214, 218, 219, and 225 of a 4-1BB co-stimulatory domain sequence, or wherein the co-stimulatory domain comprises K->R residue substitutions as shown in any of SEQ ID NOs: 46-69.

16. The method of claim 1, wherein the extracellular antigen-binding domain comprises a scFv.

17. The method of claim 1, wherein the extracellular antigen-binding domain comprises an anti-CD19/BCMA scFv, a scFv targeting CD19, CD20, CD22, BCMA, CD138, CD38, SLAMF7, kappa light chain, lambda light chain, CD123, CD33, CD45, CD30, CD40, CD70, ErbB2, EGFR, EpCAM, EGFRvIII, mesothelin, ROR1, LeY, PSMA, PSCA, CD34, CD90, TIM3, CD99, CD3, CD4, CD8, CD52, or TCR recognizing WT1.

18. The method of claim 1, wherein the subject has or is at risk of developing cancer.

19. A drug-responsive chimeric antigen receptor (CAR) comprising:
an extracellular antigen-binding domain,
a transmembrane domain (TMD),
a co-stimulatory domain,
a signaling domain and
a CRBN polypeptide substrate domain capable of binding CRBN in response to drug, thereby promoting ubiquitin pathway-mediated degradation of the drug-responsive CAR.

20. The drug-responsive CAR of claim 19 further comprising the drug.

21. The drug-responsive CAR of claim 19, wherein the drug is a small molecule drug.

22. The drug-responsive CAR of claim 19, wherein the drug is an FDA-approved drug.

23. The drug-responsive CAR of claim 19, wherein the drug can be administered to a human subject in a clinical setting.

24. The drug-responsive CAR of claim 19, wherein the drug is an IMiD.

25. The drug-responsive CAR of claim 19, wherein the drug is selected from the group consisting of thalidomide, lenalidomide and pomalidomide.

26. The drug-responsive CAR of claim 19, wherein the CRBN polypeptide substrate domain is selected from the group consisting of IKZF1, IKZF3, CK1α, ZFP91, GSPT1, MEIS2, GSS, E4F1, ZN276, ZN517, ZN582, ZN653, ZN654, ZN692, ZN787, ZN827 or a fragment thereof that is capable of drug-inducible binding the CRBN polypeptide disrupted for or lacking a DDB1-interacting domain, or wherein the CRBN polypeptide substrate is a chimeric fusion product of native CRBN polypeptide sequences, optionally the IKZF3/ZFP91/IKZF3 polypeptide SEQ ID NO: 95.

27. The method of claim 19, wherein the CRBN polypeptide substrate domain comprises a hybrid fusion polypeptide comprised of ten or more residues of a non-IKZF3 C2H2 zinc finger degron sequence flanked by an N-terminal IKZF3 degron sequence and a C-terminal IKZF3 degron sequence, optionally wherein the N-terminal IKZF3 degron sequence comprises (optionally is) amino acids 130-145 (SEQ ID NO: 97) of IKZF3 or a K0 from thereof (SEQ ID NO: 100) and/or wherein the C-terminal IKZF3 degron sequence comprises (optionally is) amino acids 169-189 (SEQ ID NO: 102) of IKZF3 or a K0 form thereof (SEQ ID NO: 103), optionally wherein the non-IKZF3 C2H2 zinc finger degron sequence is a ZFP91 sequence.

28. The drug-responsive CAR of claim 19, wherein the CRBN polypeptide substrate domain is SEQ ID NO: 5.

29. The drug-responsive CAR of claim 19, wherein the signaling domain is selected from the group consisting of a CD3ζ domain, a CD3 gamma domain, a CD3 delta domain, a CD3 epsilon domain, a FcR gamma domain, a FcR beta domain, a CD5 domain, a CD79a domain, a CD79b domain, a CD66d domain, a CD4 domain, a CD8 domain, a Dap10 domain and a Dap-12 domain.

30. The drug-responsive CAR of claim 19, wherein the co-stimulatory domain is selected from the group consisting of a CD28 co-stimulatory domain, a 4-1BB co-stimulatory domain, or additional co-stimulatory domains from CD27, OX40, CD30, CD40, ICOS, LFA-1, CD2, CD7, NKG2C, or B7-H3, optionally wherein the co-stimulatory domain comprises K→R residue substitutions at positions 182 and 204 of a CD28 co-stimulatory domain sequence, or wherein the co-stimulatory domain comprises K->R residue substitutions at positions 214, 218, 219, and 225 of a 4-1BB co-stimulatory domain sequence, or wherein the co-stimulatory domain comprises K->R residue substitutions as shown in any of SEQ ID NOs: 46-69.

31. The drug-responsive CAR of claim 19, wherein the extracellular antigen-binding domain comprises a scFv.

32. The drug-responsive CAR of claim 19, wherein the extracellular antigen-binding domain comprises an anti-CD19/BCMA scFv, a scFv targeting CD19, CD20, CD22, BCMA, CD138, CD38, SLAMF7, kappa light chain, lambda light chain, CD123, CD33, CD45, CD30, CD40, CD70, ErbB2, EGFR, EpCAM, EGFRvIII, mesothelin, ROR1, LeY, PSMA, PSCA, CD34, CD90, TIM3, CD99, CD3, CD4, CD8, CD52, or TCR recognizing WT1.

33. A mammalian cell comprising the drug-responsive CAR of claim 19.

34. The mammalian cell of claim 33, wherein the mammalian cell is a T cell.

35. The mammalian cell of claim 33, wherein the cell is selected from the group consisting of a B cell, plasma cell, NK cell, NKT cell, innate lymphoid cell, macrophage, dendritic cell, monocyte, neutrophil, basophil, eosinophil, mast cell, hematopoietic progenitor cell, hematopoietic stem cell, other adult stem cell such as neural, cornea, muscle, skin, small intestine, colon, bone, mesenchyme, embryonic stem cell and an induced pluripotent stem cell.

36. A polypeptide comprising SEQ ID NO: 95.

37. A nucleic acid comprising a sequence selected from the group consisting of SEQ ID NOs: 84-88.

38. A method for treating a subject with a chimeric antigen receptor (CAR) cellular therapy, the method comprising administering to the subject a mammalian cell comprising a split chimeric antigen receptor (CAR) system suitable for clinical application comprising a drug-inducible heterodimer, wherein the split CAR system comprises a first polypeptide and a second polypeptide, wherein:
the first polypeptide comprises an extracellular antigen-binding domain, a transmembrane domain (TMD), a co-stimulatory domain and a first domain of a drug-inducible heterodimer; and
the second polypeptide comprises a second domain of the drug-inducible heterodimer and a signaling domain,
thereby treating a subject with a chimeric antigen receptor (CAR) cellular therapy.

39. The method of claim 38, wherein the first and second domains of the drug-inducible heterodimer bind one another in the presence of the drug.

40. The method of claim 39, wherein the drug-inducible heterodimer is an IMiD-inducible CRBN/CRBN polypeptide substrate heterodimer.

41. The method of claim 39, wherein the IMiD-inducible CRBN/CRBN polypeptide substrate heterodimer comprises a CRBN polypeptide disrupted for or lacking a DDB1-interacting domain, optionally wherein the CRBN polypeptide is selected from the group consisting of SEQ ID NOs: 1-4.

42. The method of claim 39, wherein the IMiD-inducible CRBN/CRBN polypeptide substrate heterodimer comprises a CRBN polypeptide disrupted for or lacking a DDB1-interacting domain further comprising a residue substitution at one or more of positions 371 and 388, optionally wherein the CRBN polypeptide disrupted for or lacking a DDB1-interacting domain comprises a residue substitution selected from the group consisting of I371A, I371G, V388A, and V388G.

43. The method of claim 39, wherein the IMiD-inducible CRBN/CRBN polypeptide substrate heterodimer comprises a CRBN polypeptide substrate selected from the group consisting of IKZF3, IKZF1, ZFP91, GSPT1, GSS, or a fragment thereof that is capable of drug-inducible binding to CRBN polypeptide, optionally wherein the CRBN polypeptide substrate is SEQ ID NO: 5.

44. The method of claim 39, wherein the IMiD-inducible CRBN/CRBN polypeptide substrate heterodimer comprises a CRBN polypeptide substrate comprising a substituted form of IKZF3 aa130-189 that comprises K→R residue substitutions at positions 137, 158, 166, 172 and 175 of the IKZF3 aa130-189 polypeptide sequence.

45. The method of claim 39, wherein the IMiD-inducible CRBN/CRBN polypeptide substrate heterodimer comprises a CRBN polypeptide substrate comprising a substituted form of IKZF3 aa130-189 that comprises a residue substitution at position 153, optionally wherein the residue substitution at position 153 is selected from the group consisting of A153I, A153M, A153T, A153N, A153Q, A153R, A153H, A153K, A153D, A153E and A153C.

46. The method of claim 38 further comprising administering the drug to the subject.

47. The method of claim 46, wherein the CAR cellular therapy is induced upon administration of the drug to the subject.

48. The method of claim 38, wherein the drug is an FDA-approved drug, optionally an FDA-approved small molecule drug.

49. The method of claim 38, wherein the drug is a thalidomide analog immunomodulatory drug (IMiD).

50. The method of claim 38, wherein the drug is selected from the group consisting of thalidomide, lenalidomide and pomalidomide.

51. The method of claim 38, wherein the signaling domain is selected from the group consisting of a CD3ζ domain, a CD3 gamma domain, a CD3 delta domain, a CD3 epsilon domain, a FcR gamma domain, a FcR beta domain, a CD5 domain, a CD79a domain, a CD79b domain, a CD66d domain, a CD4 domain, a CD8 domain, a Dap10 domain and a Dap-12 domain.

52. The method of claim 38, wherein the second polypeptide comprising the signaling domain further comprises one or more domains selected from the group consisting of a transmembrane domain (TMD) and a co-stimulatory domain.

53. The method of claim 38, wherein the co-stimulatory domain is selected from the group consisting of a CD28 co-stimulatory domain, a 4-1BB co-stimulatory domain, or additional co-stimulatory domains from CD27, OX40, CD30, CD40, ICOS, LFA-1, CD2, CD7, NKG2C, or B7-H3, optionally wherein the co-stimulatory domain comprises K→R residue substitutions at positions 182 and 204 of a CD28 co-stimulatory domain sequence, or wherein the co-stimulatory domain comprises K->R residue substitutions at positions 214, 218, 219, and 225 of a 4-1BB co-stimulatory domain sequence, or wherein the co-stimulatory domain comprises K->R residue substitutions as shown in any of SEQ ID NOs: 46-69.

54. The method of claim 38, wherein the extracellular antigen-binding domain comprises a scFv.

55. The method of claim 38, wherein the extracellular antigen-binding domain comprises an anti-CD19/BCMA scFv or a scFv targeting CD19, CD20, CD22, BCMA, CD138, CD38, SLAMF7, kappa light chain, lambda light chain, CD123, CD33, CD45, CD30, CD40, CD70, ErbB2, EGFR, EpCAM, EGFRvIII, mesothelin, ROR1, LeY, PSMA, PSCA, CD34, CD90, TIM3, CD99, CD3, CD4, CD8, CD52 or TCR recognizing WT1.

56. The method of claim 38, further comprising identifying a CAR cellular therapy side effect in the subject.

57. The method of claim 56, further comprising halting administration of the drug after the CAR cellular therapy side effect is identified in the subject.

58. The method of claim 38, wherein the heterodimer is constitutively paired in the absence of the drug.

59. The method of claim 58, wherein the heterodimer is capable of being destabilized by administration of a small molecule.

60. The method of claim 59, wherein the drug-destabilized heterodimer is selected from the group consisting of an IMiD-destabilized CRBN/CRBN polypeptide substrate heterodimer, optionally wherein the heterodimer is CRBN/MEIS2, a MDM2/P53 polypeptide heterodimer inhibited by RG7112, a VHL/HIF-1α or VHL/HIF-2α polypeptide heterodimer inhibited by V_{H}298, or a cIAP/SMAC heterodimer inhibited by birinapant.

61. A method for treating a subject with a cellular therapy, the method comprising administering to the subject a mammalian cell comprising a drug-inducible heterodimer composition comprising:
(i) a first polypeptide having an N-terminus and a C-terminus and comprising a CRBN polypeptide disrupted for or lacking a DDB1-interacting domain and
(ii) a second polypeptide having an N-terminus and a C-terminus and comprising a CRBN polypeptide substrate,
wherein the CRBN polypeptide and the CRBN polypeptide substrate associate with one another upon administration of the drug,
thereby treating the subject.

62. The method of claim 61, wherein the cellular therapy is a CAR T cellular therapy.

63. The method of claim 61, wherein the drug is a small molecule.

64. The method of claim 61, wherein the drug is an FDA-approved drug.

65. The method of claim 61, wherein the drug can be administered to a human subject in a clinical setting.

66. The method of claim 61, wherein the drug is an IMiD.

67. The method of claim 61, wherein the drug is selected from the group consisting of thalidomide, lenalidomide and pomalidomide.

68. The method of claim 61, wherein the CRBN polypeptide disrupted for or lacking a DDB1-interacting domain is selected from the group consisting of SEQ ID NOs: 1-4.

69. The method of claim 61, wherein the CRBN polypeptide substrate is selected from the group consisting of IKZF1, IKZF3, CK1α, ZFP91, GSPT1, MEIS2, GSS E4F1, ZN276, ZN517, ZN582, ZN653, ZN654, ZN692, ZN787, ZN827 or a fragment thereof that is capable of drug-inducible binding the CRBN polypeptide disrupted for or lacking a DDB1-interacting domain, or wherein the CRBN polypeptide substrate is a chimeric fusion product of native CRBN polypeptide substrate sequences, optionally the ZFP91/IKZF3 polypeptide SEQ ID NO: 32.

70. The method of claim 61, wherein the CRBN polypeptide substrate is IKZF3 or a fragment thereof that is capable of drug-inducible binding of the CRBN polypeptide disrupted for or lacking a DDB1-interacting domain.

71. The method of claim 61, wherein the CRBN polypeptide substrate is SEQ ID NO: 5.

72. The method of claim 61, wherein the first and second polypeptides form a system selected from the group consisting of a drug-gated split chimeric antigen receptor (CAR) system, a drug-gated heterodimeric cytokine receptor, including class I cytokine receptors, class II cytokine receptors, TNF receptors, TL-1 receptors, tyrosine kinase receptors, and chemokine receptors, drug-gated heterodimeric TGF-beta receptors, drug-gated split genome editing proteins such as CAS9, drug-gated split transcription factors, optionally wherein a first component (“component A”) encodes a DNA binding motif and a second component (“component B”) encodes an effector motif such as transactivation, repression, or recruitment of an epigenetic reader, writer, or eraser protein.

73. The method of claim 61, wherein the drug-inducible heterodimer incorporates any of the components recited in claim 35 (receptors, kinases, transcription factors, epigenetic modifiers, genome editing proteins), wherein the second component serves as a tether to a particular subcellular localization, such that the drug-dependent heterodimerization serves as a location-based gain-, loss-, or change-of function switch.

74. The method of claim 61, wherein the first or second polypeptide comprises one or more domains selected from the group consisting of an extracellular antigen-binding domain, a transmembrane domain (TMD) and a co-stimulatory domain.

75. The method of claim 74, wherein the extracellular binding domain comprises a scFv.

76. The method of claim 74, wherein the extracellular binding domain comprises a scFv targeting CD19, CD20, CD22, BCMA, CD138, CD38, SLAMF7, kappa light chain, lambda light chain, CD123, CD33, CD45, CD30, CD40, CD70, ErbB2, EGFR, EpCAM, EGFRvIII, mesothelin, ROR1, LeY, PSMA, PSCA, CD34, CD90, TIM3, CD99, CD3, CD4, CD8, CD52, or TCR recognizing WT1.

77. The method of claim 61, wherein the first or second polypeptide comprises a signaling domain.

78. The method of claim 77, wherein the signaling domain is selected from the group consisting of a CD3ζ domain, a CD3 gamma domain, a CD3 delta domain, a CD3 epsilon domain, a FcR gamma domain, a FcR beta domain, a CD5 domain, a CD79a domain, a CD79b domain, a CD66d domain, a CD4 domain, a CD8 domain, a Dap10 domain and a Dap-12 domain.

79. The method of claim 77, wherein the first or second polypeptide comprising the signaling domain further comprises one or more domains selected from the group consisting of a transmembrane domain (TMD) and a co-stimulatory domain.

80. The method of claim 74, wherein the co-stimulatory domain is selected from the group consisting of a CD28 co-stimulatory domain, a 4-1BB co-stimulatory domain, or additional co-stimulatory domains from CD27, OX40, CD30, CD40, ICOS, LFA-1, CD2, CD7, NKG2C, or B7-H3.

81. The method of claim 74, wherein the co-stimulatory domain comprises K→R residue substitutions at positions 182 and 204 of a CD28 (Homo sapiens CD28 isoform 1 Uniprot identifier P10747-1) co-stimulatory domain sequence.

82. The method of claim 74, wherein the CRBN polypeptide substrate comprises a substituted form of IKZF3 aa130-189 that comprises a residue substitution at position 153, optionally wherein the residue substitution at position 153 is selected from the group consisting of A153I, A153M, A153T, A153N, A153Q, A153R, A153H, A153K, A153D, A153E and A153C.

83. The method of claim 61, wherein the CRBN polypeptide disrupted for or lacking a DDB1-interacting domain, such as the minCRBN variants 1-4 described in SEQ ID NOs: 1-4, additionally comprises a residue substitution at one or more of positions 371 and 388, optionally wherein the CRBN polypeptide disrupted for or lacking a DDB1-interacting domain comprises a residue substitution selected from the group consisting of I371A, I371G, V388A and V388G.

84. The method of claim 61, wherein the CRBN polypeptide substrate comprises a substituted form of IKZF3 aa130-189 that comprises K→R residue substitutions at positions 137, 158, 166, 172 and 175 of the IKZF3 aa130-189 polypeptide sequence.

85. The method of claim 61, wherein the subject has or is at risk of developing cancer.

86. The method of claim 61, wherein the cellular therapy is administered in a therapeutically effective amount.

87. The method of claim 61, further comprising identifying a cellular therapy side effect in the subject.

88. The method of claim 87, further comprising halting administration of the drug after the cellular therapy side effect is identified in the subject.

89. A drug-inducible heterodimer composition comprising:
(i) a first polypeptide having an N-terminus and a C-terminus and comprising a CRBN polypeptide disrupted for or lacking a DDB1-interacting domain and
(ii) a second polypeptide having an N-terminus and a C-terminus and comprising a CRBN polypeptide substrate,
wherein the CRBN polypeptide and the CRBN polypeptide substrate associate upon administration of the drug.

90. The drug-inducible heterodimer composition of claim 89 further comprising the drug.

91. The drug-inducible heterodimer composition of claim 89, wherein the drug is a small molecule.

92. The drug-inducible heterodimer composition of claim 89, wherein the drug is an FDA-approved drug.

93. The drug-inducible heterodimer composition of claim 89, wherein the drug can be administered to a human subject in a clinical setting.

94. The drug-inducible heterodimer composition of claim 89, wherein the drug is an IMiD.

95. The drug-inducible heterodimer composition of claim 89, wherein the drug is selected from the group consisting of thalidomide, lenalidomide and pomalidomide.

96. The drug-inducible heterodimer composition of claim 89, wherein the CRBN polypeptide disrupted for or lacking a DDB1-interacting domain is selected from the group consisting of SEQ ID NOs: 1-4.

97. The drug-inducible heterodimer composition of claim 89, wherein the CRBN polypeptide substrate is selected from the group consisting of IKZF1, IKZF3, CK1α, ZFP91, GSPT1, MEIS2, GSS E4F1, ZN276, ZN517, ZN582, ZN653, ZN654, ZN692, ZN787, ZN827 or a fragment thereof that is capable of drug-inducible binding the CRBN polypeptide disrupted for or lacking a DDB1-interacting domain, or wherein the CRBN polypeptide substrate is a chimeric fusion product of native CRBN polypeptide sequences, optionally the ZFP91/IKZF3 polypeptide SEQ ID NO: 32.

98. The drug-inducible heterodimer composition of claim 89, wherein the CRBN polypeptide substrate is IKZF3 or a fragment thereof that is capable of drug-inducible binding of the CRBN polypeptide disrupted for or lacking a DDB1-interacting domain.

99. The drug-inducible heterodimer composition of claim 89, wherein the CRBN polypeptide substrate is SEQ ID NO: 5.

100. The drug-inducible heterodimer composition of claim 89, wherein the first and second polypeptides form a system selected from the group consisting of a drug-gated split chimeric antigen receptor (CAR) system, a drug-gated heterodimeric cytokine receptor, including class I cytokine receptors, class II cytokine receptors, TNF receptors, IL-1 receptors, tyrosine kinase receptors, and chemokine receptors, drug-gated heterodimeric TGF-beta receptors, drug-gated split genome editing proteins such as CAS9, drug-gated split transcription factors, for example wherein component A encodes a DNA binding motif and component B encodes an effector motif such as transactivation, repression, or recruitment of an epigenetic reader, writer, or eraser protein, optionally wherein the drug-inducible heterodimer incorporates any of the preceding components (receptors, kinases, transcription factors, epigenetic modifiers, genome editing proteins), wherein the second component serves as a tether to a particular subcellular localization, such that the drug-dependent heterodimerization serves as a location-based gain-, loss-, or change-of function switch.

101. The drug-inducible heterodimer composition of claim 89, wherein the first or second polypeptide comprises one or more domains selected from the group consisting of an extracellular antigen-binding domain, a transmembrane domain (TMD) and a co-stimulatory domain.

102. The drug-inducible heterodimer composition of claim 101, wherein the extracellular antigen-binding domain comprises a scFv.

103. The drug-inducible heterodimer composition of claim 101, wherein the extracellular antigen-binding domain comprises a scFv targeting CD19, CD20, CD22, BCMA, CD138, CD38, SLAMF7, kappa light chain, lambda light chain, CD123, CD33, CD45, CD30, CD40, CD70, ErbB2, EGFR, EpCAM, EGFRvIII, mesothelin, ROR1, LeY, PSMA, PSCA, CD34, CD90, TIM3, CD99, CD3, CD4, CD8, CD52, or TCR recognizing WT1.

104. The drug-inducible heterodimer composition of claim 89, wherein the first or second polypeptide comprises a signaling domain.

105. The drug-inducible heterodimer composition of claim 104, wherein the signaling domain is selected from the group consisting of a CD3ζ domain, a CD3 gamma domain, a CD3 delta domain, a CD3 epsilon domain, a FcR gamma domain, a FcR beta domain, a CD5 domain, a CD79a domain, a CD79b domain, a CD66d domain, a CD4 domain, a CD8 domain, a Dap10 domain and a Dap-12 domain.

106. The drug-inducible heterodimer composition of claim 104, wherein the first or second polypeptide comprising the signaling domain further comprises one or more domains selected from the group consisting of a transmembrane domain (TMD) and a co-stimulatory domain.

107. The drug-inducible heterodimer composition of claim 101, wherein the co-stimulatory domain is selected from the group consisting of a CD28 co-stimulatory domain, a 4-1BB co-stimulatory domain, or additional co-stimulatory domains from CD27, OX40, CD30, CD40, ICOS, LFA-1, CD2, CD7, NKG2C, or B7-H3.

108. The drug-inducible heterodimer composition of claim 101, wherein the co-stimulatory domain comprises K→R residue substitutions at positions 182 and 204 of a CD28 (Homo sapiens CD28 isoform 1 Uniprot identifier P10747-1) co-stimulatory domain sequence.

109. The drug-inducible heterodimer composition of claim 101, wherein the CRBN polypeptide substrate comprises a substituted form of IKZF3 aa130-189 that comprises a residue substitution at position 153, optionally wherein the residue substitution at position 153 is selected from the group consisting of A153I, A153M, A153T, A153N, A153Q, A153R, A153H, A153K, A153D, A153E and A153C.

110. The drug-inducible heterodimer composition of claim 89, wherein the CRBN polypeptide disrupted for or lacking a DDB1-interacting domain, such as the minCRBN variants 1-4 described in SEQ ID NOs: 1-4, additionally comprises a residue substitution at one or more of positions 371 and 388, optionally wherein the CRBN polypeptide disrupted for or lacking a DDB1-interacting domain comprises a residue substitution selected from the group consisting of I371A, I371G, V388A and V388G.

111. The drug-inducible heterodimer composition of claim 89, wherein the CRBN polypeptide substrate comprises a substituted form of IKZF3 aa130-189 that comprises K→R residue substitutions at positions 137, 158, 166, 172 and 175 of the IKZF3 aa130-189 polypeptide sequence.

112. A mammalian cell comprising the drug-inducible heterodimer composition of claim 89.

113. The mammalian cell of claim 112, wherein the mammalian cell is a T cell, optionally a B cell, plasma cell, NK cell, NKT cell, innate lymphoid cell, macrophage, dendritic cell, monocyte, neutrophil, basophil, eosinophil, mast cell, hematopoietic progenitor cell, hematopoietic stem cell, other adult stem cell such as neural, cornea, muscle, skin, small intestine, colon, bone, mesenchyme, embryonic stem cell, or induced pluripotent stem cell.

114. The mammalian cell of claim 112, wherein the mammalian cell comprises a genomic disruption of native CRBN, optionally a biallelic disruption of native CRBN.

115. The mammalian cell of claim 112, wherein the mammalian cell comprises a genomic disruption of CRBN exon 5, optionally a CRBNΔe5 disruption.

116. A split chimeric antigen receptor (CAR) system suitable for clinical application comprising a drug-inducible heterodimer, wherein the split CAR system comprises a first polypeptide and a second polypeptide, wherein:
the first polypeptide comprises an extracellular antigen-binding domain, a transmembrane domain (TMD), a co-stimulatory domain and a first domain of a drug-inducible heterodimer; and
the second polypeptide comprises a second domain of the drug-inducible heterodimer and a signaling domain,
wherein the first and second domains of the drug-inducible heterodimer bind one another in the presence of the drug.

117. The split CAR system of claim 116, wherein the drug-inducible heterodimer is an IMiD-inducible CRBN/CRBN polypeptide substrate heterodimer.

118. The split CAR system of claim 116, wherein the heterodimer is constitutively paired.

119. The split CAR system of claim 118, wherein the heterodimer is capable of being destabilized with the addition of a small molecule, optionally wherein the drug-destabilized heterodimer is selected from the group consisting of an IMiD-destabilized CRBN/CRBN polypeptide substrate heterodimer such as CRBN/MEIS2, a MDM2/P53 polypeptide heterodimer inhibited by RG7112, a VHL/HIF-1α or VHL/HIF-2α polypeptide heterodimer inhibited by V_{H}298, a cIAP/SMAC heterodimer inhibited by birinapant.

120. The split CAR system of claim 117, wherein the IMiD-inducible CRBN/CRBN polypeptide substrate heterodimer comprises a CRBN polypeptide disrupted for or lacking a DDB1-interacting domain, optionally wherein the CRBN polypeptide is selected from the group consisting of SEQ ID NOs: 1-4.

121. The split CAR system of claim 117, wherein the IMiD-inducible CRBN/CRBN polypeptide substrate heterodimer comprises a CRBN polypeptide disrupted for or lacking a DDB1-interacting domain further comprising a residue substitution at one or more of positions 371 and 388, optionally wherein the CRBN polypeptide disrupted for or lacking a DDB1-interacting domain comprises a residue substitution selected from the group consisting of I371A, I371G, V388A, and V388G.

122. The split CAR system of claim 117, wherein the IMiD-inducible CRBN/CRBN polypeptide substrate heterodimer comprises a CRBN polypeptide substrate selected from the group consisting of IKZF3, IKZF1, ZFP91, GSPT1, GSS, or a fragment thereof that is capable of drug-inducible binding to CRBN polypeptide, optionally wherein the CRBN polypeptide substrate is SEQ ID NO: 5.

123. The split CAR system of claim 117, wherein the IMiD-inducible CRBN/CRBN polypeptide substrate heterodimer comprises a CRBN polypeptide substrate comprising a substituted form of IKZF3 aa130-189 that comprises K→R residue substitutions at positions 137, 158, 166, 172 and 175 of the IKZF3 aa130-189 polypeptide sequence.

124. The split CAR system of claim 117, wherein the IMiD-inducible CRBN/CRBN polypeptide substrate heterodimer comprises a CRBN polypeptide substrate comprising a substituted form of IKZF3 aa130-189 that comprises a residue substitution at position 153, optionally wherein the residue substitution at position 153 is selected from the group consisting of A153I, A153M, A153T, A153N, A153Q, A153R, A153H, A153K, A153D, A153E and A153C.

125. The split CAR system of claim 116 further comprising the drug.

126. The split CAR system of claim 116, wherein the drug is an FDA-approved drug, optionally an FDA-approved small molecule drug.

127. The split CAR system of claim 116, wherein the drug is an IMiD.

128. The split CAR system of claim 116, wherein the drug is selected from the group consisting of thalidomide, lenalidomide and pomalidomide.

129. The split CAR system of claim 116, wherein the signaling domain is selected from the group consisting of a CD3ζ domain, a CD3 gamma domain, a CD3 delta domain, a CD3 epsilon domain, a FcR gamma domain, a FcR beta domain, a CD5 domain, a CD79a domain, a CD79b domain, a CD66d domain, a CD4 domain, a CD8 domain, a Dap10 domain and a Dap-12 domain.

130. The split CAR system of claim 116, wherein the second polypeptide comprising the signaling domain further comprises one or more domains selected from the group consisting of a transmembrane domain (TMD) and a co-stimulatory domain.

131. The split CAR system of claim 116, wherein the co-stimulatory domain is selected from the group consisting of a CD28 co-stimulatory domain, a 4-1BB co-stimulatory domain, or additional co-stimulatory domains from CD27, OX40, CD30, CD40, ICOS, LFA-1, CD2, CD7, NKG2C, or B7-H3, optionally wherein the co-stimulatory domain comprises K→R residue substitutions at positions 182 and 204 of a CD28 co-stimulatory domain sequence, or wherein the co-stimulatory domain comprises K->R residue substitutions at positions 214, 218, 219, and 225 of a 4-1BB co-stimulatory domain sequence, or wherein the co-stimulatory domain comprises K->R residue substitutions as shown in any of SEQ ID NOs: 46-69.

132. The split CAR system of claim 116, wherein the extracellular antigen-binding domain comprises a scFv.

133. The split CAR system of claim 116, wherein the extracellular antigen-binding domain comprises an anti-CD19/BCMA scFv, a scFv targeting CD19, CD20, CD22, BCMA, CD138, CD38, SLAMF7, kappa light chain, lambda light chain, CD123, CD33, CD45, CD30, CD40, CD70, ErbB2, EGFR, EpCAM, EGFRvIII, mesothelin, ROR1, LeY, PSMA, PSCA, CD34, CD90, TIM3, CD99, CD3, CD4, CD8, CD52, or TCR recognizing WT1.

134. A mammalian cell comprising the split CAR system of claim 116.

135. The mammalian cell of claim 134, wherein the mammalian cell is a T cell.

136. The mammalian cell of claim 134, wherein the cell is selected from the group consisting of a B cell, plasma cell, NK cell, NKT cell, innate lymphoid cell, macrophage, dendritic cell, monocyte, neutrophil, basophil, eosinophil, mast cell, hematopoietic progenitor cell, hematopoietic stem cell, other adult stem cell such as neural, cornea, muscle, skin, small intestine, colon, bone, mesenchyme, embryonic stem cell and an induced pluripotent stem cell.

137. The mammalian cell of claim 134, wherein the mammalian cell comprises a genomic disruption of CRBN, optionally a biallelic disruption of CRBN.

138. The mammalian cell of claim 134, wherein the mammalian cell comprises a genomic disruption of CRBN exon 5, optionally a CRBNΔe5 disruption.

139. A CRBN polypeptide disrupted for or lacking a DDB1-interacting domain and comprising one or more domains selected from the group consisting of a CRBN thalidomide binding domain (TBD), a CRBN LLP1-C domain, a CRBN LLP1-N domain and a CRBN N-terminal domain, wherein the CRBN polypeptide is not SEQ ID NO: 1 or SEQ ID NO: 4.

140. A polypeptide comprising SEQ ID NO: 3.

141. A polypeptide comprising SEQ ID NO: 2 in the absence of any other CRBN sequence.

142. A drug-responsive polypeptide comprising:
an inhibitor of CAR signaling and
a CRBN polypeptide substrate domain capable of binding CRBN in response to drug, thereby promoting ubiquitin pathway-mediated degradation of the drug-responsive polypeptide and activating CAR signaling.

143. The drug-responsive polypeptide of claim 142, wherein the inhibitor of CAR signaling is a proximal, pan-CAR/TCR signal transduction inhibitor, optionally wherein the inhibitor of CAR signaling is Carboxy-terminal Src Kinase (CSK).

144. The drug-responsive polypeptide of claim 142, wherein the inhibitor of CAR signaling selectively abrogates a CAR signal transduction pathway and/or a CAR effector function, optionally wherein the inhibitor of CAR signaling selectively abrogates a pathway or function selected from the group consisting of Ras signaling, PKC, calcium-dependent signaling, NF-kappaB, NFAT, actin and cytoskeletal responses, cytokine secretion, cell proliferation, degranulation, and tumor cell killing, differentiation, or exhaustion.

145. The drug-responsive polypeptide of claim 142, wherein the inhibitor of CAR signaling is a ubiquitin ligase involved in TCR/CAR signal transduction, optionally wherein the inhibitor of CAR signaling is selected from the group consisting of c-CBL, CBL-B, ITCH, RNF125, RNF128 and WWP2.

146. The drug-responsive polypeptide of claim 142, wherein the inhibitor of CAR signaling is a TCR/CAR negative regulatory enzyme, optionally wherein the inhibitor of CAR signaling is selected from the group consisting of SHP1, SHP2, SHIP1, SHIP2, CD45, CSK, CD148, PTPN22, DGKalpha, DGKzeta, DRAK2, HPK1, HPK1, STS1, STS2 and SLAT.

147. The drug-responsive polypeptide of claim 142, wherein the inhibitor of CAR signaling is a TCR/CAR negative regulatory scaffold/adapter protein, optionally wherein the inhibitor of CAR signaling is selected from the group consisting of PAG, LIME, NTAL, LAX31, SIT, GAB2, GRAP, ALX, SLAP, SLAP2, DOK1 and DOK2.

148. The drug-responsive polypeptide of claim 142, wherein the inhibitor of CAR signaling is a dominant negative version of an activating TCR signaling component, optionally wherein the inhibitor of CAR signaling is selected from the group consisting of ZAP70, LCK, FYN, NCK, VAV1, SLP76, ITK, ADAP, GADS, PLCgamma1, LAT, p85, SOS, GRB2, NFAT, p50, p65, AP1, RAP1, CRKII, C3G, WAVE2, ARP2/3, ABL, ADAP, RIAM and SKAP55.

149. The drug-responsive polypeptide of claim 142, wherein the inhibitor of CAR signaling comprises the cytoplasmic tail of a TCR/CAR negative co-regulatory receptor, optionally wherein the inhibitor of CAR signaling comprises the cytoplasmic tail of a TCR/CAR negative co-regulatory receptor selected from the group consisting of CD5, PD1, CTLA4, BTLA, LAG3, B7-H1, B7-1, CD160, TIM3, 2B4 and TIGIT.

150. The drug-responsive polypeptide of claim 142, wherein the inhibitor of CAR signaling comprises a plasma membrane-targeting sequence, optionally wherein the plasma membrane-targeting sequence is derived from a sequence selected from the group consisting of LAT, PAG, LCK, FYN, LAX, CD2, CD3, CD4, CD5, CD7, CD8a, PD1, SRC, and LYN.

151. The drug-responsive polypeptide of claim 142, wherein the drug-responsive polypeptide is cytosolic.

152. The drug-responsive polypeptide of claim 142, wherein the drug-responsive polypeptide comprises a membrane tether and/or transmembrane domain.

153. The drug-responsive polypeptide of claim 142 further comprising the drug.

154. The drug-responsive polypeptide of claim 142, wherein the drug is a small molecule drug.

155. The drug-responsive polypeptide of claim 142, wherein the drug is an FDA-approved drug.

156. The drug-responsive polypeptide of claim 142, wherein the drug can be administered to a human subject in a clinical setting.

157. The drug-responsive polypeptide of claim 142, wherein the drug is an IMiD.

158. The drug-responsive polypeptide of claim 142, wherein the drug is selected from the group consisting of thalidomide, lenalidomide and pomalidomide.

159. The drug-responsive polypeptide of claim 142, wherein the CRBN polypeptide substrate domain is selected from the group consisting of IKZF1, IKZF3, CK1α, ZFP91, GSPT1, MEIS2, GSS, E4F1, ZN276, ZN517, ZN582, ZN653, ZN654, ZN692, ZN787, ZN827 or a fragment thereof that is capable of drug-inducible binding the CRBN polypeptide disrupted for or lacking a DDB1-interacting domain, or wherein the CRBN polypeptide substrate is a chimeric fusion product of native CRBN polypeptide sequences, optionally the ZFP91/IKZF3 polypeptide SEQ ID NO: 32.

160. The drug-responsive polypeptide of claim 142, wherein the CRBN polypeptide substrate domain is SEQ ID NO: 5.

161. A mammalian cell comprising the drug-responsive polypeptide of claim 142.

162. The mammalian cell of claim 161, wherein the mammalian cell is a T cell.

163. The mammalian cell of claim 161, wherein the cell is selected from the group consisting of a B cell, plasma cell, NK cell, NKT cell, innate lymphoid cell, macrophage, dendritic cell, monocyte, neutrophil, basophil, eosinophil, mast cell, hematopoietic progenitor cell, hematopoietic stem cell, other adult stem cell such as neural, cornea, muscle, skin, small intestine, colon, bone, mesenchyme, embryonic stem cell and an induced pluripotent stem cell.

164. A method for treating a subject with a chimeric antigen receptor (CAR) cellular therapy, the method comprising administering to the subject a mammalian cell comprising a CAR and a drug-responsive polypeptide comprising:
an inhibitor of CAR signaling and
a CRBN polypeptide substrate domain capable of binding CRBN in response to drug, thereby promoting ubiquitin pathway-mediated degradation of the drug-responsive polypeptide and activating CAR signaling,
thereby treating a subject with a CAR cellular therapy.

165. The method of claim 164, further comprising administering the drug.

166. The method of claim 164, wherein the drug is a small molecule drug.

167. The method of claim 164, wherein the drug is an FDA-approved drug.

168. The method of claim 164, wherein the drug can be administered to a human subject in a clinical setting.

169. The method of claim 164, wherein the drug is an IMiD.

170. The method of claim 164, wherein the drug is selected from the group consisting of thalidomide, lenalidomide and pomalidomide.


SEQUENCE LISTING (141 sequences)

SEQ ID NO: 1 - protein, 104 aa, synthetic construct
  REGION 1..104: note = Synthetic
SLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCL

SEQ ID NO: 2 - protein, 125 aa, synthetic construct
  REGION 1..125: note = Synthetic
CTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCL

SEQ ID NO: 3 - protein, 386 aa, synthetic construct
  REGION 1..386: note = Synthetic
AGEGDQQDAAHNMGNHLPLLPESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCL

SEQ ID NO: 4 - protein, 367 aa, synthetic construct
  REGION 1..367: note = Synthetic
MAGEGDQQDAAHNMGNHLPLLPESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCL

SEQ ID NO: 5 - protein, 60 aa, synthetic construct
  REGION 1..60: note = Synthetic
FNVLMVHKRSHTGERPFQCNQCGASFTQKGNLLRHIKLHTGEKPFKCHLCNYACQRRDAL

SEQ ID NO: 6 - protein, 442 aa, Homo sapiens [sequence omitted]

SEQ ID NO: 7 - protein, 386 aa, synthetic construct
  REGION 1..386: note = Synthetic
AGEGDQQDAAHNMGNHLPLLPESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLAGRPSTEHSWFPGYAWTVAQCKICASHIGWKFTATKKDMSPQKFWGLTRSALLPTIPDTEDEISPDKVILCL

SEQ ID NO: 8 - protein, 386 aa, synthetic construct
  REGION 1..386: note = Synthetic
AGEGDQQDAAHNMGNHLPLLPESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLGGRPSTEHSWFPGYAWTVAQCKICASHIGWKFTATKKDMSPQKFWGLTRSALLPTIPDTEDEISPDKVILCL

SEQ ID NO: 9 - protein, 386 aa, synthetic construct
  REGION 1..386: note = Synthetic
AGEGDQQDAAHNMGNHLPLLPESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTAAQCKICASHIGWKFTATKKDMSPQKFWGLTRSALLPTIPDTEDEISPDKVILCL

SEQ ID NO: 10 - protein, 386 aa, synthetic construct
  REGION 1..386: note = Synthetic
AGEGDQQDAAHNMGNHLPLLPESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTGAQCKICASHIGWKFTATKKDMSPQKFWGLTRSALLPTIPDTEDEISPDKVILCL

SEQ ID NO: 11 - protein, 477 aa, Homo sapiens
MDADEGQDMSQVSGKESPPVSDTPDEGDEPMPIPEDLSTTSGGQQSSKSDRVVASNVKVETQSDEENGRACEMNGEECAEDLRMLDASGEKMNGSHRDQGSSALSGVGGIRLPNGKLKCDICGIICIGPNVLMVHKRSHTGERPFQCNQCGASFTQKGNLLRHIKLHSGEKPFKCHLCNYACRRRDALTGHLRTHSVIKEETNHSEMAEDLCKIGSERSLVLDRLASNVAKRKSSMPQKFLGDKGLSDTPYDSSASYEKENEMMKSHVMDQAINNAINYLGAESLRPLVQTPPGGSEVVPVISPMYQLHKPLAEGTPRSNHSAQDSAVENLLLLSKAKLVPSEREASPSNSCQDSTDTESNNEEQRSGLIYLTNHIAPHARNGLSLKEEHRAYDLLRAASENSQDALRVVSTSGEQMKVYKCEHCRVLFLDHVMYTIHMGCHGFRDPFECNMCGYHSQDRYEFSSHITRGEHRFHMS

SEQ ID NO: 12 - protein, 262 aa, Homo sapiens
MGSERALVLDRLASNVAKRKSSMPQKFIGEKRHCFDVNYNSSYMYEKESELIQTRMMDQAINNAISYLGAEALRPLVQTPPAPTSEMVPVISSMYPIALTRAEMSNGAPQELEKKSIHLPEKSVPSERGLSPNNSGHDSTDTDSNHEERQNHIYQQNHMVLSRARNGMPLLKEVPRSYELLKPPPICPRDSVKVINKEGEVMDVYRCDHCRVLFLDYVMFTIHMGCHGFRDPFECNMCGYRSHDRYEFSSHIARGEHRALLK

SEQ ID NO: 13 - protein, 337 aa, Homo sapiens
MASSSGSKAEFIVGGKYKLVRKIGSGSFGDIYLAINITNGEEVAVKLESQKARHPQLLYESKLYKILQGGVGIPHIRWYGQEKDYNVLVMDLLGPSLEDLFNFCSRRFTMKTVLMLADQMISRIEYVHTKNFIHRDIKPDNFLMGIGRHCNKLFLIDFGLAKKYRDNRTRQHIPYREDKNLTGTARYASINAHLGIEQSRRDDMESLGYVLMYFNRTSLPWQGLKAATKKQKYEKISEKKMSTPVEVLCKGFPAEFAMYLNYCRGLRFEEAPDYMYLRQLFRILFRTLNHQYDYTFDWTMLKQKAAQQAASSSGQGQQAQTPTGKQTDKTKSNMKGF

SEQ ID NO: 14 - protein, 570 aa, Homo sapiens
MPGETEEPRPPEQQDQEGGEAAKAAPEEPQQRPPEAVAAAPAGTTSSRVLRGGRDRGRAAAAAAAAAVSRRRKAEYPRRRRSSPSARPPDVPGQQPQAAKSPSPVQGKKSPRLLCIEKVTTDKDPKEEKEEEDDSALPQEVSIAASRPSRGWRSSRTSVSRHRDTENTRSSRSKTGSLQLICKSEPNTDQLDYDVGEEHQSPGGISSEEEEEEEEEMLISEEEIPFKDDPRDETYKPHLERETPKPRRKSGKVKEEKEKKEIKVEVEVEVKEEENEIREDEEPPRKRGRRRKDDKSPRLPKRRKKPPIQYVRCEMEGCGTVLAHPRYLQHHIKYQHLLKKKYVCPHPSCGRLFRLQKQLLRHAKHHTDQRDYICEYCARAFKSSHNLAVHRMIHTGEKPLQCEICGFTCRQKASLNWHMKKHDADSFYQFSCNICGKKFEKKDSVVAHKAKSHPEVLIAEALAANAGALITSTDILGTNPESLTQPSDGQGLPLLPEPLGNSTSGECLLLEAEGMSKSYCSGTERVSLMADGKIFVGSGSSGGTEGLVMNSDILGATTEVLIEDSDSAGP

SEQ ID NO: 15 - protein, 633 aa, Homo sapiens
MDPGSGGGGGGGGSSSGSSSSDSAPDCWDQADMEAPGPGPCGGGGSLAAAAEAQRENLSAAFSRQLNVNAKPFVPNVHAAEFVPSFLRCPAAPPPPAGGAANNHGAGSGAGGRAAPVESSQEEQSLCEGSNSAVSMELSEPIENGETEMSPEESWEHKEEISEAEPGGGSLGDGRPPEESAHEMMEEEEEIPKPKSVVAPPGAPKKEHVNVVFIGHVDAGKSTIGGQIMYLTGMVDKRTLEKYEREAKEKNRETWYLSWALDTNQEERDKGKTVEVGRAYFETEKKHFTILDAPGHKSFVPNMIGGASQADLAVLVISARKGEFETGFEKGGQTREHAMLAKTAGVKHLIVLINKMDDPTVNWSNERYEECKEKLVPFLKKVGFNPKKDIHFMPCSGLTGANLKEQSDFCPWYIGLPFIPYLDNLPNFNRSVDGPIRLPIVDKYKDMGTVVLGKLESGSICKGQQLVMMPNKHNVEVLGILSDDVETDTVAPGENLKIRLKGIEEEEILPGFILCDPNNLCHSGRTFDAQIVIIEHKSIICPGYNAVLHIHTCIEEVEITALICLVDKKSGEKSKTRPRFVKQDQVCIARLRTAGTICLETFKDFPQMGRFTLRDEGKTIAIGKVLKLVPEKD

SEQ ID NO: 16 - protein, 474 aa, Homo sapiens
MATNWGSLLQDKQQLEELARQAVDRALAEGVLLRTSQEPTSSEVVSYAPFTLFPSLVPSALLEQAYAVQMDFNLLVDAVSQNAAFLEQTLSSTIKQDDFTARLFDIHKQVLKEGIAQTVFLGLNRSDYMFQRSADGSPALKQIEINTISASFGGLASRTPAVHRHVLSVLSKTKEAGKILSNNPSKGLALGIAKAWELYGSPNALVLLIAQEKERNIFDQRAIENELLARNIHVIRRTFEDISEKGSLDQDRRLFVDGQEIAVVYFRDGYMPRQYSLQNWEARLLLERSHAAKCPDIATQLAGTKKVQQELSRPGMLEMLLPGQPEAVARLRATFAGLYSLDVGEEGDQAIAEALAAPSRFVLKPQREGGGNNLYGEEMVQALKQLKDSEERASYILMEKIEPEPFENCLLRPGSPARVVQCISELGIFGVYVRQEKTLVMNKHVGHLLRTKAIEHADGGVAAGVAVLDNPYPV

SEQ ID NO: 17 - protein, 401 aa, Homo sapiens
MAQRYDELPHYGGMDGVGVPASMYGDPHAPRPIPPVHHLNHGPPLHATQHYGAHAPHPNVMPASMGSAVNDALKRDKDAIYGHPLFPLLALVFEKCELATCTPREPGVAGGDVCSSDSFNEDIAVFAKQVRAEKPLFSSNPELDNLMIQAIQVLRFHLLELEKVHELCDNFCHRYISCLKGKMPIDLVIDERDGSSKSDHEELSGSSTNLADHNPSSWRDHDDATSTHSAGTPGPSSGGHASQSGDNSSEQGDGLDNSVASPGTGDDDDPDKDKKRQKKRGIFPKVATNIMRAWLFQHLTHPYPSEEQKKQLAQDTGLTILQVNNWFINARRRIVQPMIDQSNRAGFLLDPSVSQGAAYSPEGQPMGSFVLDGQQHMGIRPAGPMSGMGMNMGMDGQWHYM

SEQ ID NO: 18 - protein, 524 aa, Homo sapiens
MPLVHMASSPAVDVSCRRREKRRQLDARRSKCRIRLGGHMEQWCLLKERLGFSLHSQLAKFLLDRYTSSGCVLCAGPEPLPPKGLQYLVLLSHAHSRECSLVPGLRGPGGQDGGLVWECSAGHTFSWGPSLSPTPSEAPKPASLPHTTRRSWCSEATSGQELADLESEHDERTQEARLPRRVGPPPETFPPPGEEEGEEEEDNDEDEEEMLSDASLWTYSSSPDDSEPDAPRLLPSPVTCTPKEGETPPAPAALSSPLAVPALSASSLSSRAPPPAEVRVQPQLSRTPQAAQQTEALASTGSQAQSAPTPAWDEDTAQIGPKRIRKAAKRELMPCDFPGCGRIFSNRQYLNHHKKYQHIHQKSFSCPEPACGKSFNFKKHLKEHMKLHSDTRDYICEFCARSFRTSSNLVIHRRIHTGEKPLQCEICGFTCRQKASLNWHQRKHAETVAALRFPCEFCGKRFEKPDSVAAHRSKSHPALLLAPQESPSGPLEPCPSISAPGPLGSSEGSRPSASPQAPTLLPQQ

SEQ ID NO: 19 - protein, 23 aa, synthetic construct
  REGION 1..23: note = Synthetic
FQCNQCGASFTQKGNLLRHIKLH

SEQ ID NO: 20 - protein, 23 aa, synthetic construct
  REGION 1..23: note = Synthetic
LQCEICGFTCRQKASLNWHMKKH

SEQ ID NO: 21 - protein, 23 aa, synthetic construct
  REGION 1..23: note = Synthetic
FQCNQCGASFTQKGNLLRHIKLH

SEQ ID NO: 22 - protein, 60 aa, synthetic construct
  REGION 1..60: note = Synthetic
PNVLMVHKRSHTGERPFQCNQCGASFTQKGNLLRHIKLHSGEKPFKCHLCNYACRRRDAL

SEQ ID NO: 23 - protein, 23 aa, synthetic construct
  REGION 1..23: note = Synthetic
HECKLCGASFRTKGSLIRHHRRH

SEQ ID NO: 24 - protein, 23 aa, synthetic construct
  REGION 1..23: note = Synthetic
LQCEVCGFQCRQRASLKYHMTKH

SEQ ID NO: 25 - protein, 23 aa, synthetic construct
  REGION 1..23: note = Synthetic
YRCRACGRACSRLSTLIQHQKVH

SEQ ID NO: 26 - protein, 23 aa, synthetic construct
  REGION 1..23: note = Synthetic
YQCKVCGRAFKRVSHLTVHYRIH

SEQ ID NO: 27 - protein, 23 aa, synthetic construct
  REGION 1..23: note = Synthetic
LQCEICGYQCRQRASLNWHMKKH

SEQ ID NO: 28 - protein, 23 aa, synthetic construct
  REGION 1..23: note = Synthetic
FACVICGRKFRNRGLMQKHLKNH

SEQ ID NO: 29 - protein, 23 aa, synthetic construct
  REGION 1..23: note = Synthetic
LQCEICGFTCRQKASLNWHQRKH

SEQ ID NO: 30 - protein, 23 aa, synthetic construct
  REGION 1..23: note = Synthetic
FVCPRCGRGFSQPKSLARHLRLH

SEQ ID NO: 31 - protein, 23 aa, synthetic construct
  REGION 1..23: note = Synthetic
FQCPICGLVIKRKSYWKRHMVIH

SEQ ID NO: 32 - protein, 23 aa, synthetic construct
  REGION 1..23: note = Synthetic
LQCEICGFTCRQKGNLLRHIKLH

SEQ ID NO: 33 - protein, 23 aa, synthetic construct
  REGION 1..23: note = Synthetic
FQCNQCGASFTQKASLNWHMKKH

SEQ ID NO: 34 - protein, 60 aa, synthetic construct
  REGION 1..60: note = Synthetic
FNVLMVHKRSHTGERPLQCEICGFTCRQKGNLLRHIKLHTGEKPFKCHLCNYACQRRDAL

SEQ ID NO: 35 - protein, 60 aa, synthetic construct
  REGION 1..60: note = Synthetic
FNVLMVHKRSHTGERPFQCNQCGISFTQKGNLLRHIKLHTGEKPFKCHLCNYACQRRDAL

SEQ ID NO: 36 - protein, 60 aa, synthetic construct
  REGION 1..60: note = Synthetic
FNVLMVHKRSHTGERPFQCNQCGMSFTQKGNLLRHIKLHTGEKPFKCHLCNYACQRRDAL

SEQ ID NO: 37 - protein, 60 aa, synthetic construct
  REGION 1..60: note = Synthetic
FNVLMVHKRSHTGERPFQCNQCGTSFTQKGNLLRHIKLHTGEKPFKCHLCNYACQRRDAL

SEQ ID NO: 38 - protein, 60 aa, synthetic construct
  REGION 1..60: note = Synthetic
FNVLMVHKRSHTGERPFQCNQCGNSFTQKGNLLRHIKLHTGEKPFKCHLCNYACQRRDAL

SEQ ID NO: 39 - protein, 60 aa, synthetic construct
  REGION 1..60: note = Synthetic
FNVLMVHKRSHTGERPFQCNQCGQSFTQKGNLLRHIKLHTGEKPFKCHLCNYACQRRDAL

SEQ ID NO: 40 - protein, 60 aa, synthetic construct
  REGION 1..60: note = Synthetic
FNVLMVHKRSHTGERPFQCNQCGRSFTQKGNLLRHIKLHTGEKPFKCHLCNYACQRRDAL

SEQ ID NO: 41 - protein, 60 aa, synthetic construct
  REGION 1..60: note = Synthetic
FNVLMVHKRSHTGERPFQCNQCGHSFTQKGNLLRHIKLHTGEKPFKCHLCNYACQRRDAL

SEQ ID NO: 42 - protein, 60 aa, synthetic construct
  REGION 1..60: note = Synthetic
FNVLMVHKRSHTGERPFQCNQCGKSFTQKGNLLRHIKLHTGEKPFKCHLCNYACQRRDAL

SEQ ID NO: 43 - protein, 60 aa, synthetic construct
  REGION 1..60: note = Synthetic
FNVLMVHKRSHTGERPFQCNQCGDSFTQKGNLLRHIKLHTGEKPFKCHLCNYACQRRDAL

SEQ ID NO: 44 - protein, 60 aa, synthetic construct
  REGION 1..60: note = Synthetic
FNVLMVHKRSHTGERPFQCNQCGESFTQKGNLLRHIKLHTGEKPFKCHLCNYACQRRDAL

SEQ ID NO: 45 - protein, 60 aa, synthetic construct
  REGION 1..60: note = Synthetic
FNVLMVHKRSHTGERPFQCNQCGCSFTQKGNLLRHIKLHTGEKPFKCHLCNYACQRRDAL

SEQ ID NO: 46 - protein, 60 aa, synthetic construct
  REGION 1..60: note = Synthetic
FNVLMVHRRSHTGERPFQCNQCGASFTQRGNLLRHIRLHTGERPFRCHLCNYACQRRDAL

SEQ ID NO: 47 - protein, 23 aa, synthetic construct
  REGION 1..23: note = Synthetic
FQCNQCGASFTQRGNLLRHIRLH

SEQ ID NO: 48 - protein, 23 aa, synthetic construct
  REGION 1..23: note = Synthetic
LQCEICGFTCRQRASLNWHMRRH

SEQ ID NO: 49 - protein, 23 aa, synthetic construct
  REGION 1..23: note = Synthetic
FQCNQCGASFTQRGNLLRHIRLH

SEQ ID NO: 50 - protein, 60 aa, synthetic construct
  REGION 1..60: note = Synthetic
PNVLMVHRRSHTGERPFQCNQCGASFTQRGNLLRHIRLHSGERPFRCHLCNYACRRRDAL

SEQ ID NO: 51 - protein, 23 aa, synthetic construct
  REGION 1..23: note = Synthetic
HECRLCGASFRTRGSLIRHHRRH

SEQ ID NO: 52 - protein, 23 aa, synthetic construct
  REGION 1..23: note = Synthetic
LQCEVCGFQCRQRASLRYHMTRH

SEQ ID NO: 53 - protein, 23 aa, synthetic construct
  REGION 1..23: note = Synthetic
YRCRACGRACSRLSTLIQHQRVH

SEQ ID NO: 54 - protein, 23 aa, synthetic construct
  REGION 1..23: note = Synthetic
YQCRVCGRAFRRVSHLTVHYRIH

SEQ ID NO: 55 - protein, 23 aa, synthetic construct
  REGION 1..23: note = Synthetic
LQCEICGYQCRQRASLNWHMRRH

SEQ ID NO: 56 - protein, 23 aa, synthetic construct
  REGION 1..23: note = Synthetic
FACVICGRRFRNRGLMQRHLRNH

SEQ ID NO: 57 - protein, 23 aa, synthetic construct
  REGION 1..23: note = Synthetic
LQCEICGFTCRQRASLNWHQRRH

SEQ ID NO: 58 - protein, 23 aa, synthetic construct
  REGION 1..23: note = Synthetic
FVCPRCGRGFSQPRSLARHLRLH

SEQ ID NO: 59 - protein, 23 aa, synthetic construct
  REGION 1..23: note = Synthetic
FQCPICGLVIRRRSYWRRHMVIH

SEQ ID NO: 60 - protein, 23 aa, synthetic construct
  REGION 1..23: note = Synthetic
LQCEICGFTCRQRGNLLRHIRLH

SEQ ID NO: 61 - protein, 23 aa, synthetic construct
  REGION 1..23: note = Synthetic
FQCNQCGASFTQRASLNWHMRRH

SEQ ID NO: 62 - protein, 60 aa, synthetic construct
  REGION 1..60: note = Synthetic
FNVLMVHRRSHTGERPLQCEICGFTCRQRGNLLRHIRLHTGERPFRCHLCNYACQRRDAL

SEQ ID NO: 63 - protein, 477 aa, Homo sapiens
MDADEGQDMSQVSGRESPPVSDTPDEGDEPMPIPEDLSTTSGGQQSSRSDRVVASNVRVETQSDEENGRACEMNGEECAEDLRMLDASGERMNGSHRDQGSSALSGVGGIRLPNGRLRCDICGIICIGPNVLMVHRRSHTGERPFQCNQCGASFTQRGNLLRHIRLHSGERPFRCHLCNYACRRRDALTGHLRTHSVIREETNHSEMAEDLCRIGSERSLVLDRLASNVARRRSSMPQRFLGDRGLSDTPYDSSASYERENEMMRSHVMDQAINNAINYLGAESLRPLVQTPPGGSEVVPVISPMYQLHRPLAEGTPRSNHSAQDSAVENLLLLSRARLVPSEREASPSNSCQDSTDTESNNEEQRSGLIYLTNHIAPHARNGLSLREEHRAYDLLRAASENSQDALRVVSTSGEQMRVYRCEHCRVLFLDHVMYTIHMGCHGFRDPFECNMCGYHSQDRYEFSSHITRGEHRFHMS

SEQ ID NO: 64 - protein, 262 aa, Homo sapiens
MGSERALVLDRLASNVARRRSSMPQRFIGERRHCFDVNYNSSYMYERESELIQTRMMDQAINNAISYLGAEALRPLVQTPPAPTSEMVPVISSMYPIALTRAEMSNGAPQELERRSIHLPERSVPSERGLSPNNSGHDSTDTDSNHEERQNHIYQQNHMVLSRARNGMPLLREVPRSYELLRPPPICPRDSVRVINREGEVMDVYRCDHCRVLFLDYVMFTIHMGCHGFRDPFECNMCGYRSHDRYEFSSHIARGEHRALLR

SEQ ID NO: 65 - protein, 365 aa, Homo sapiens
MASSSGSRAEFIVGGRYRLVRRIGSGSFGDIYLAINITNGEEVAVRLESQRARHPQLLYESRLYRILQGGVGIPHIRWYGQERDYNVLVMDLLGPSLEDLFNFCSRRFTMRTVLMLADQMISRIEYVHTRNFIHRDIRPDNFLMGIGRHCNRCLESPVGRRRRSMTVSTSQDPSFSGLNQLFLIDFGLARRYRDNRTRQHIPYREDRNLTGTARYASINAHLGIEQSRRDDMESLGYVLMYFNRTSLPWQGLRAATRRQRYERISERRMSTPVEVLCRGFPAEFAMYLNYCRGLRFEEAPDYMYLRQLFRILFRTLNHQYDYTFDWTMLRQRAAQQAASSSGQGQQAQTPTGRQTDRTRSNMRGF

SEQ ID NO: 66 - protein, 570 aa, Homo sapiens
MPGETEEPRPPEQQDQEGGEAARAAPEEPQQRPPEAVAAAPAGTTSSRVLRGGRDRGRAAAAAAAAAVSRRRRAEYPRRRRSSPSARPPDVPGQQPQAARSPSPVQGRRSPRLLCIERVTTDRDPREEREEEDDSALPQEVSIAASRPSRGWRSSRTSVSRHRDTENTRSSRSRTGSLQLICRSEPNTDQLDYDVGEEHQSPGGISSEEEEEEEEEMLISEEEIPFRDDPRDETYRPHLERETPRPRRRSGRVREERERREIRVEVEVEVREEENEIREDEEPPRRRGRRRRDDRSPRLPRRRRRPPIQYVRCEMEGCGTVLAHPRYLQHHIRYQHLLRRRYVCPHPSCGRLFRLQRQLLRHARHHTDQRDYICEYCARAFRSSHNLAVHRMIHTGERPLQCEICGFTCRQRASLNWHMRRHDADSFYQFSCNICGRRFERRDSVVAHRARSHPEVLIAEALAANAGALITSTDILGTNPESLTQPSDGQGLPLLPEPLGNSTSGECLLLEAEGMSRSYCSGTERVSLMADGRIFVGSGSSGGTEGLVMNSDILGATTEVLIEDSDSAGP

SEQ ID NO: 67 - protein, 633 aa, Homo sapiens
MDPGSGGGGGGGGSSSGSSSSDSAPDCWDQADMEAPGPGPCGGGGSLAAAAEAQRENLSAAFSRQLNVNARPFVPNVHAAEFVPSFLRCPAAPPPPAGGAANNHGAGSGAGGRAAPVESSQEEQSLCEGSNSAVSMELSEPIENGETEMSPEESWEHREEISEAEPGGGSLGDGRPPEESAHEMMEEEEEIPRPRSVVAPPGAPRREHVNVVFIGHVDAGRSTIGGQIMYLTGMVDRRTLERYEREARERNRETWYLSWALDTNQEERDRGRTVEVGRAYFETERRHFTILDAPGHRSFVPNMIGGASQADLAVLVISARRGEFETGFERGGQTREHAMLARTAGVRHLIVLINRMDDPTVNWSNERYEECRERLVPFLRRVGFNPRRDIHFMPCSGLTGANLREQSDFCPWYIGLPFIPYLDNLPNFNRSVDGPIRLPIVDRYRDMGTVVLGRLESGSICRGQQLVMMPNRHNVEVLGILSDDVETDTVAPGENLRIRLRGIEEEEILPGFILCDPNNLCHSGRTFDAQIVIIEHRSIICPGYNAVLHIHTCIEEVEITALICLVDRRSGERSRTRPRFVRQDQVCIARLRTAGTICLETFRDFPQMGRFTLRDEGRTIAIGRVLRLVPERD

SEQ ID NO: 68 - protein, 474 aa, Homo sapiens
MATNWGSLLQDRQQLEELARQAVDRALAEGVLLRTSQEPTSSEVVSYAPFTLFPSLVPSALLEQAYAVQMDFNLLVDAVSQNAAFLEQTLSSTIRQDDFTARLFDIHRQVLREGIAQTVFLGLNRSDYMFQRSADGSPALRQIEINTISASFGGLASRTPAVHRHVLSVLSRTREAGRILSNNPSRGLALGIARAWELYGSPNALVLLIAQERERNIFDQRAIENELLARNIHVIRRTFEDISERGSLDQDRRLFVDGQEIAVVYFRDGYMPRQYSLQNWEARLLLERSHAARCPDIATQLAGTRRVQQELSRPGMLEMLLPGQPEAVARLRATFAGLYSLDVGEEGDQAIAEALAAPSRFVLRPQREGGGNNLYGEEMVQALRQLRDSEERASYILMERIEPEPFENCLLRPGSPARVVQCISELGIFGVYVRQERTLVMNRHVGHLLRTRAIEHADGGVAAGVAVLDNPYPV

SEQ ID NO: 69 - protein, 401 aa, Homo sapiens
MAQRYDELPHYGGMDGVGVPASMYGDPHAPRPIPPVHHLNHGPPLHATQHYGAHAPHPNVMPASMGSAVNDALRRDRDAIYGHPLFPLLALVFERCELATCTPREPGVAGGDVCSSDSFNEDIAVFARQVRAERPLFSSNPELDNLMIQAIQVLRFHLLELERVHELCDNFCHRYISCLRGRMPIDLVIDERDGSSRSDHEELSGSSTNLADHNPSSWRDHDDATSTHSAGTPGPSSGGHASQSGDNSSEQGDGLDNSVASPGTGDDDDPDRDRRRQRRRGIFPRVATNIMRAWLFQHLTHPYPSEEQRRQLAQDTGLTILQVNNWFINARRRIVQPMIDQSNRAGFLLDPSVSQGAAYSPEGQPMGSFVLDGQQHMGIRPAGPMSGMGMNMGMDGQWHYM

SEQ ID NO: 70 - DNA, 10568 bp, synthetic construct
  misc_feature 1..10568: note = Synthetic
  source 1..10568: mol_type = other DNA
cgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccctattccagcacatatgagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcggccggtcgaatcaagcttatcgataccgtcgactccggaatagccaccatggagacggacgtctcagctgaagctgctgcaaaggaagctgcagctaaggaggctgcagctaaggctgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaatgcatgagtaactgaggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccctggaattcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaactagtaagcttggcgtaactagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttgggctcgagggggcccgggtgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgccgtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagc

SEQ ID NO: 71 - DNA, 5051 bp, synthetic construct
  misc_feature 1..5051: note = Synthetic
  source 1..5051: mol_type = other DNA
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctggtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtattaatacgactcactatagggctagcgatcgccatggaataagtaaggaatccacatggcacaggttatcaacacgtttgacggggttgcggattatcttcagacatatcataagctacctgataattacattacaaaatcagaagcacaagccctcggctgggtggcatcaaaagggaaccttgcagacgtcgctccggggaaaagcatcggcggagacatcttctcaaacagggaaggcaaactcccgggcaaaagcggacgaacatggcgtgaagcggatattaactatacatcaggcttcagaaattcagaccggattctttactcaagcgactggctgatttacaaaacaacggaccattatcagacctttacaaaaatcagataatgtttaatgaccccgtgtcgagctctcgagccaaccactgaggatctgtactttcagagcgataacgatggatccgaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtctactctggagatttccggttaatagaattctagagtcgacctgcaggcatgcaagctgatccggctgctaacaaagcccgaaaggaagctgagttggctgctgccaccgctgagcaataactagcataaccccttggggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggtttttttaagcaagtaaaacctctacaaatgtggtaaaatcgaattctaatggatcctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgagcttcaaaagaattgccagctggggcgccctctggtaaggttgggaagccctgcaaagtaaactggatggctttcttgccgccaaggatctgatggcgcaggggatcaagatctgatcaagagacaggatgacggtcgtttcgcatgcttgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgcactcggatggaagccggtcttgtcgatcaggatgatctggacgaagagcatcaggggctcgcgccagccgaactgttcgccaggctcaaggcgcgtatgccggatggtgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaaccggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatttcaagaagatcctttgatcttttctacggggtctgacgctcagtggaacgaaaactcacgttaagggattttggtcatgagattatcaaaaaggatcttcacctagatccttttatagtccggaaatacaggaacgcacgctggatggcccttcgctgggatggtgaaaccatgaaaaatggcagcttcagtggattaagtgggggtaatgtggcctgtaccctctggttgcataggtattcatacggttaaaatttatcaggcgcgattgcggcagtttttcgggtggtttgttgccatttttacctgtctgctgccgtgatcgcgctgaacgcgttttagcggtgcgtacaattaagggattatggtaaatccacttactgtctgccctcgtagccatcgagataaaccgcagtactccggccacgatgcgtccggcgtagaggatcgagatct

SEQ ID NO: 72 - DNA, 5119 bp, synthetic construct
  misc_feature 1..5119: note = Synthetic
  source 1..5119: mol_type = other DNA
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctggtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtattaatacgactcactatagggctagcgatcgccatggaataagtaaggaatccacatggcacaggttatcaacacgtttgacggggttgcggattatcttcagacatatcataagctacctgataattacattacaaaatcagaagcacaagccctcggctgggtggcatcaaaagggaaccttgcagacgtcgctccggggaaaagcatcggcggagacatcttctcaaacagggaaggcaaactcccgggcaaaagcggacgaacatggcgtgaagcggatattaactatacatcaggcttcagaaattcagaccggattctttactcaagcgactggctgatttacaaaacaacggaccattatcagacctttacaaaaatcagataatgtttaatgaccccgtgtcgagctctcggctcgagcggcgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaggccgcgactctagagtcgacctgcaggcatgcaagctgatccggctgctaacaaagcccgaaaggaagctgagttggctgctgccaccgctgagcaataactagcataaccccttggggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggtttttttaagcaagtaaaacctctacaaatgtggtaaaatcgaattttaacaaaatattaacgcttacaatttcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttaattaactgttgacaattaatcatcggcatagtatatcggcatagtataatacgacaaggtgaggaactaaacccaggaggcagatcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatcctctttgcgcttgcgttttcccttgtccagatagcccagtagctgacattcatccggggtcagcaccgtttctgcggactggctttctacccggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatttcaagaagatcctttgatcttttctacggggtctgacgctcagtggaacgaaaactcacgttaagggattttggtcatgagattatcaaaaaggatcttcacctagatccttttatagtccggaaatacaggaacgcacgctggatggcccttcgctgggatggtgaaaccatgaaaaatggcagcttcagtggattaagtgggggtaatgtggcctgtaccctctggttgcataggtattcatacggttaaaatttatcaggcgcgattgcggcagtttttcgggtggtttgttgccatttttacctgtctgctgccgtgatcgcgctgaacgcgttttagcggtgcgtacaattaagggattatggtaaatccacttactgtctgccctcgtagccatcgagataaaccgcagtactccggccacgatgcgtccggcgtagaggatcgagatct

SEQ ID NO: 73 - protein, 439 aa, synthetic construct
  REGION 1..439: note = Synthetic
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSSGFNVLMVHKRSHTGERPFQCNQCGASFTQKGNLLRHIKLHTGEKPFKCHLCNYACQRRDAL

SEQ ID NO: 74 - protein, 439 aa, synthetic construct
  REGION 1..439: note = Synthetic
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSRRSRLLHSDYMNMTPRRPGPTRRHYQPYAPPRDFAAYRSSGFNVLMVHRRSHTGERPFQCNQCGASFTQRGNLLRHIRLHTGERPFRCHLCNYACQRRDAL

SEQ ID NO: 75 - protein, 522 aa, synthetic construct
  REGION 1..522: note = Synthetic
MALPVTALLLPLALLLHAARPTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSGGGGSAGEGDQQDAAHNMGNHLPLLPESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCL

SEQ ID NO: 76 - protein, 453 aa, synthetic construct
  REGION 1..453: note = Synthetic
MGCIKSKRKDNLNDDGVDMKTRSKRSRLLHSDYMNMTPRRPGPTRKHYQPYAPPRDFAAYRSGGGGSAGEGDQQDAAHNMGNHLPLLPESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCL

SEQ ID NO: 77 - protein, 555 aa, synthetic construct
  REGION 1..555: note = Synthetic
MALPVTALLLPLALLLHAARPERRAEVPTAHPSPSPRPAGQFQTLVVGVVGGLLGSLVLLVWVLAVICSRAARGTIGARRTGQPLKEDPSAVPVFSVDFGELDFQWREKTPEPPVPCVPEQTEFATIVFPSGMGTSSPARRGSADGPRSAQPLRPEDGHCSWPLGGGGSAGEGDQQDAAHNMGNHLPLLPESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCL

SEQ ID NO: 78 - protein, 635 aa, synthetic construct
  REGION 1..635: note = Synthetic
MALPVTALLLPLALLLHAARPTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSGGGGSAGEGDQQDAAHNMGNHLPLLPESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCLSLGFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 79 - protein, 566 aa, synthetic construct
  REGION 1..566: note = Synthetic
MGCIKSKRKDNLNDDGVDMKTRSKRSRLLHSDYMNMTPRRPGPTRKHYQPYAPPRDFAAYRSGGGGSAGEGDQQDAAHNMGNHLPLLPESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCLSLGFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 80 - protein, 668 aa, synthetic construct
  REGION 1..668: note = Synthetic
MALPVTALLLPLALLLHAARPERRAEVPTAHPSPSPRPAGQFQTLVVGVVGGLLGSLVLLVWVLAVICSRAARGTIGARRTGQPLKEDPSAVPVFSVDFGELDFQWREKTPEPPVPCVPEQTEFATIVFPSGMGTSSPARRGSADGPRSAQPLRPEDGHCSWPLGGGGSAGEGDQQDAAHNMGNHLPLLPESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCLSLGFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 81 - protein, 561 aa, synthetic construct
  REGION 1..561: note = Synthetic
MLLLVTSLLLCELPHPAFLLIPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGFNVLMVHKRSHTGERPFQCNQCGASFTQKGNLLRHIKLHTGEKPFKCHLCNYACQRRDAL

SEQ ID NO: 82 - protein, 282 aa, synthetic construct
  REGION 1..282: note = Synthetic
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSSGFNVLMVHKRSHTGERPFQCNQCGASFTQKGNLLRHIKLHTGEKPFKCHLCNYACQRRDAL

SEQ ID NO: 83 - protein, 392 aa, synthetic construct
  REGION 1..392: note = Synthetic
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRSGFNVLMVHKRSHTGERPFQCNQCGASFTQKGNLLRHIKLHTGEKPFKCHLCNYACQRRDAL

SEQ ID NO: 84 - DNA, 1635 bp, synthetic construct
  misc_feature 1..1635: note = Synthetic
  source 1..1635: mol_type = other DNA
atggaggaggccatcctggtcccctgcgtgctggggctcctgctgctgcccatcctggccatgttgatggcactgtgtgtgcactgccacagactgccaatgtcagcaatacaggccgcctggccatccggtacagaatgtattgccaagtacaacttccacggcactgccgagcaggacctgcccttctgcaaaggggacgtgctcaccattgtggccgtcaccaaggaccccaactggtacaaagccaaaaacaaggtgggccgtgagggcatcatcccagccaactacgtccagaagcgggagggcgtgaaggcgggtaccaaactcagcctcatgccttggttccacggcaagatcacacgggagcaggctgagcggcttctgtacccgccggagacaggcctgttcctggtgcgggagagcaccaactaccccggagactacacgctgtgcgtgagctgcgacggcaaggtggagcactaccgcatcatgtaccatgccagcaagctcagcatcgacgaggaggtgtactttgagaacctcatgcagctggtggagcactacacctcagacgcagatggactctgtacgcgcctcattaaaccaaaggtcatggagggcacagtggcggcccaggatgagttctaccgcagcggctgggccctgaacatgaaggagctgaagctgctgcagaccatcgggaagggggagttcggggacgtgatgctgggcgattaccgagggaacaaagtcgccgtcaagtgcattaagaacgacgccactgcccaggccttcctggctgaagcctcagtcatgacgcaactgcggcatagcaacctggtgcagctcctgggcgtgatcgtggaggagaagggcgggctctacatcgtcactgagtacatggccaaggggagccttgtggactacctgcggtctaggggtcggtcagtgctgggcggagactgtctcctcaagttctcgctagatgtctgcgaggccatggaatacctggagggcaacaatttcgtgcatcgagacctggctgcccgcaatgtgctggtgtctgaggacaacgtggccaaggtcagcgactttggtctcaccaaggaggcgtccagcacccaggacacgggcaagctgccagtcaagtggacagcccctgaggccctgagagagaagaaattctccactaagtctgacgtgtggagtttcggaatccttctctgggaaatctactcctttgggcgagtgccttatccaagaattcccctgaaggacgtcgtccctcgggtggagaagggctacaagatggatgcccccgacggctgcccgcccgcagtctatgaagtcatgaagaactgctggcacctggacgccgccatgcggccctccttcctacagctccgagagcagcttgagcacatcaaaacccacgagctgcacctgtccggattcaatgtcttaatggttcataagcgaagccatactggtgaacgcccattccagtgtaatcagtgtggggcatcttttactcagaaaggtaacctcctccgccacattaaactgcacacaggggaaaaaccttttaagtgtcacctctgcaactatgcatgccaaagaagagatgcgctc

SEQ ID NO: 85 - DNA, 1635 bp, synthetic construct
  misc_feature 1..1635: note = Synthetic
  source 1..1635: mol_type = other DNA
atggaggaggccatcctggtcccctgcgtgctggggctcctgctgctgcccatcctggccatgttgatggcactgtgtgtgcactgccacagactgccaatgtcagcaatacaggccgcctggccatccggtacagaatgtattgccaagtacaacttccacggcactgccgagcaggacctgcccttctgcaaaggggacgtgctcaccattgtggccgtcaccaaggaccccaactggtacaaagccaaaaacaaggtgggccgtgagggcatcatcccagccaactacgtccagaagcgggagggcgtgaaggcgggtaccaaactcagcctcatgccttggttccacggcaagatcacacgggagcaggctgagcggcttctgtacccgccggagacaggcctgttcctggtgcgggagagcaccaactaccccggagactacacgctgtgcgtgagctgcgacggcaaggtggagcactaccgcatcatgtaccatgccagcaagctcagcatcgacgaggaggtgtactttgagaacctcatgcagctggtggcgcactacacctcagacgcagatggactctgtacgcgcctcattaaaccaaaggtcatggagggcacagtggcggcccaggatgagttctaccgcagcggctgggccctgaacatgaaggagctgaagctgctgcagaccatcgggaagggggagttcggggacgtgatgctgggcgattaccgagggaacaaagtcgccgtcaagtgcattaagaacgacgccactgcccaggccttcctggctgaagcctcagtcatgacgcaactgcggcatagcaacctggtgcagctcctgggcgtgatcgtggaggagaagggcgggctctacatcgtcactgagtacatggccaaggggagccttgtggactacctgcggtctaggggtcggtcagtgctgggcggagactgtctcctcaagttctcgctagatgtctgcgaggccatggaatacctggagggcaacaatttcgtgcatcgagacctggctgcccgcaatgtgctggtgtctgaggacaacgtggccaaggtcagcgactttggtctcaccaaggaggcgtccagcacccaggacacgggcaagctgccagtcaagtggacagcccctgaggccctgagagagaagaaattctccactaagtctgacgtgtggagtttcggaatccttctctgggaaatctactcctttgggcgagtgccttatccaagaattcccctgaaggacgtcgtccctcgggtggagaagggctacaagatggatgcccccgacggctgcccgcccgcagtctatgaagtcatgaagaactgctggcacctggacgccgccatgcggccctccttcctacagctccgagagcagcttgagcacatcaaaacccacgagctgcacctgtccggattcaatgtcttaatggttcataagcgaagccatactggtgaacgcccattccagtgtaatcagtgtggggcatcttttactcagaaaggtaacctcctccgccacattaaactgcacacaggggaaaaaccttttaagtgtcacctctgcaactatgcatgccaaagaagagatgcgctc

SEQ ID NO: 86 - DNA, 1635 bp, synthetic construct
  misc_feature 1..1635: note = Synthetic
  source 1..1635: mol_type = other DNA
atggaggaggccatcctggtcccctgcgtgctggggctcctgctgctgcccatcctggccatgttgatggcactgtgtgtgcactgccacagactgccaatgtcagcaatacaggccgcctggccatccggtacagaatgtattgccaagtacaacttccacggcactgccgagcaggacctgcccttctgcaaaggggacgtgctcaccattgtggccgtcaccaaggaccccaacgcgtacaaagccaaaaacaaggtgggccgtgagggcatcatcccagccaactacgtccagaagcgggagggcgtgaaggcgggtaccaaactcagcctcatgccttggttccacggcaagatcacacgggagcaggctgagcggcttctgtacccgccggagacaggcctgttcctggtgaaggagagcaccaactaccccggagactacacgctgtgcgtgagctgcgacggcaaggtggagcactaccgcatcatgtaccatgccagcaagctcagcatcgacgaggaggtgtactttgagaacctcatgcagctggtggcgcactacacctcagacgcagatggactctgtacgcgcctcattaaaccaaaggtcatggagggcacagtggcggcccaggatgagttctaccgcagcggctgggccctgaacatgaaggagctgaagctgctgcagaccatcgggaagggggagttcggggacgtgatgctgggcgattaccgagggaacaaagtcgccgtcaagtgcattaagaacgacgccactgcccaggccttcctggctgaagcctcagtcatgacgcaactgcggcatagcaacctggtgcagctcctgggcgtgatcgtggaggagaagggcgggctctacatcgtcactgagtacatggccaaggggagccttgtggactacctgcggtctaggggtcggtcagtgctgggcggagactgtctcctcaagttctcgctagatgtctgcgaggccatggaatacctggagggcaacaatttcgtgcatcgagacctggctgcccgcaatgtgctggtgtctgaggacaacgtggccaaggtcagcgactttggtctcaccaaggaggcgtccagcacccaggacacgggcaagctgccagtcaagtggacagcccctgaggccctgagagagaagaaattctccactaagtctgacgtgtggagtttcggaatccttctctgggaaatctactcctttgggcgagtgccttatccaagaattcccctgaaggacgtcgtccctcgggtggagaagggctacaagatggatgcccccgacggctgcccgcccgcagtctatgaagtcatgaagaactgctggcacctggacgccgccatgcggccctccttcctacagctccgagagcagcttgagcacatcaaaacccacgagctgcacctgtccggattcaatgtcttaatggttcataagcgaagccatactggtgaacgcccattccagtgtaatcagtgtggggcatcttttactcagaaaggtaacctcctccgccacattaaactgcacacaggggaaaaaccttttaagtgtcacctctgcaactatgcatgccaaagaagagatgcgctc

SEQ ID NO: 87 - DNA, 1464 bp, synthetic construct
  misc_feature 1..1464: note = Synthetic
  source 1..1464: mol_type = other DNA
atggaggaggccatcctggtcccctgcgtgctggggctcctgctgctgcccatcctggccatgttgatggcactgtgtgtgcactgccacagactgccatcaggcgcctttgtctacctgcggcagccgtactatgccacgagggtgaatgcggctgacattgagaaccgagtgttggaactgaacaagaagcaggagtccgaggatacagccaaggctggcttctgggaggagtttgagagtttgcagaagcaggaggtgaagaacttgcaccagcgtctggaagggcagcggccagagaacaagggcaagaaccgctacaagaacattctcccctttgaccacagccgagtgatcctgcagggacgggacagtaacatccccgggtccgactacatcaatgccaactacatcaagaaccagctgctaggccctgatgagaacgctaagacctacatcgccagccagggctgtctggaggccacggtcaatgacttctggcagatggcgtggcaggagaacagccgtgtcatcgtcatgaccacccgagaggtggagaaaggccggaacaaatgcgtcccatactggcccgaggtgggcatgcagcgtgcttatgggccctactctgtgaccaactgcggggagcatgacacaaccgaatacaaactccgtaccttacaggtctccccgctggacaatggagacctgattcgggagatctggcattaccagtacctgagctggcccgaccatggggtccccagtgagcctgggggtgtcctcagcttcctggaccagatcaaccagcggcaggaaagtctgcctcacgcagggcccatcatcgtgcactgcagcgccggcatcggccgcacaggcaccatcattgtcatcgacatgctcatggagaacatctccaccaagggcctggactgtgacattgacatccagaagaccatccagatggtgcgggcgcagcgctcgggcatggtgcagacggaggcgcagtacaagttcatctacgtggccatcgcccagttcattgaaaccactaagaagaagctggaggtcctgcagtcgcagaagggccaggagtcggagtacgggaacatcacctatcccccagccatgaagaatgcccatgccaaggcctcccgcacctcgtccaaacacaaggaggatgtgtatgagaacctgcacactaagaacaagagggaggagaaagtgaagaagcagcggtcagcagacaaggagaagagcaagggttccctcaagaggaagtccggattcaatgtcttaatggttcataagcgaagccatactggtgaacgcccattccagtgtaatcagtgtggggcatcttttactcagaaaggtaacctcctccgccacattaaactgcacacaggggaaaaaccttttaagtgtcacctctgcaactatgcatgccaaagaagagatgcgctc

SEQ ID NO: 88 - DNA, 1122 bp, synthetic construct
  misc_feature 1..1122: note = Synthetic
  source 1..1122: mol_type = other DNA
atggcgctcccagtcactgccctgcttttgcccctggcacttcttcttcacgctgccagacccacaacgaccccagctccacgcccgccgactcccgcgccaactatagccagtcagcccctgtcactgcggccggaggcgtgtcgccctgcagcggggggagccgtccacacacgaggtcttgacttcgcctgtgacatctatatctgggcgcctctggccggtacatgcggcgtgttgttgcttagcctcgtgataacactctattgcaggagtaagaggagcaggctcctgcacagtgactacatgaacatgactcctagaaggcctggacccacccgcaagcattaccagccctatgccccaccacgcgacttcgcagcctatcgctccggagggggtggttcttgtacttccctttgctgtaaacaatgtcaagaaacagaaataacaaccaaaaatgaaatattcagtttatccttatgtgggccgatggcagcttatgtgaatcctcatggatatgtgcatgagacacttactgtgtataaggcttgcaacttgaatctgataggccggccttctacagaacacagctggtttcctgggtatgcctggactgttgcccagtgtaagatctgtgcaagccatattggatggaagtttacggccaccaaaaaagacatgtcacctcaaaaattttggggcttaacgcgatctgctctgttgcccacgatcccagacactgaagatgaaataagtccagacaaagtaatactttgcttgtctctcggcttcagtcgatcagcagatgctccagcgtaccagcaaggccagaaccaactttataatgaactgaatttgggccgacgcgaggaatacgacgttcttgacaagcggaggggccgcgatccagaaatgggggggaaaccgcaacgaagaaagaatccccaggaggggctctacaatgaacttcaaaaggataaaatggcagaggcctatagcgaaatcggtatgaagggggagcgaaggcgaggtaaggggcatgatgggttgtatcaaggcctgtccacggcgaccaaggatacctatgacgctcttcacatgcaagcattgcctcctaga

SEQ ID NO: 89 - DNA, 8216 bp, synthetic construct
  misc_feature 1..8216: note = Synthetic
  source 1..8216: mol_type = other DNA
ttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccctattccagcacatatgaggcttggcgtaactagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttgggctcgagggggcccgggtgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaataccgtcgactccggaatagccaccatggagacggacgtctcagctgaagctgctgcaaaggaagctgcagctaaggaggctgcagctaaggctgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaatgcatgagtaactgaggatccagggacagcagagatacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgctt

SEQ ID NO: 90 - DNA, 8185 bp, synthetic construct
  misc_feature 1..8185: note = Synthetic
  source 1..8185: mol_type = other DNA
ctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccctattccagcacatatgaggcttggcgtaactagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttgggctcgagggggcccgggtgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctaccggtcgccaccatggagacggacgtctcagctgaagctgctgcaaaggaagctgcagctaaggaggctgcagctaaggctatggtgtctaagggcgaagagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagaccctgtaccccgctgacggcggcctggaaggcagaaacgacatggccctgaagctcgtgggcgggagccatctgatcgcaaacgccaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaacagggacagcagagatacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 91 - DNA, 8693 bp, synthetic construct
  misc_feature 1..8693: note = Synthetic
  source 1..8693: mol_type = other DNA
ttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccctattccagcacatatgaggcttggcgtaactagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttgggctcgagggggcccgggtgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaataccgtcgactccggaatagccaccatggagacggacgtctcagctgaagctgctgcaaaggaagctgcagctaaggaggctgcagctaaggctatggtgtctaagggcgaagagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagaccctgtaccccgctgacggcggcctggaaggcagaaacgacatggccctgaagctcgtgggcgggagccatctgatcgcaaacgccaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaatggactcagagtttgggtaggaagcggagctactaacttcagcctgctgaagcaggctggagatgtggaggagaaccctggacctatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaaatgcatgagtaactgaggatccagggacagcagagatacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgctt

SEQ ID NO: 92 - DNA, 9086 bp, synthetic construct
  misc_feature 1..9086: note = Synthetic
  source 1..9086: mol_type = other DNA
ttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccctattccagcacatatgaggcttggcgtaactagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttgggctcgagggggcccgggtgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaataccgtcgactccggaatagccaccatggagacggacgtctcagctgaagctgctgcaaaggaagctgcagctaaggaggctgcagctaaggctatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggactcagagtttgggtaggaagcggagctactaacttcagcctgctgaagcaggctggagatgtggaggagaaccctggacctatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggccgaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaatgcatgagtaactgaggatccagggacagcagagatacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgctt

SEQ ID NO: 93 - protein, 10 aa, synthetic construct
  REGION 1..10: note = Synthetic
GGGGSGGGGS

SEQ ID NO: 94 - protein, 17 aa, synthetic construct
  REGION 1..17: note = Synthetic
AEAAAKEAAAKEAAAKA

SEQ ID NO: 95 - protein, 60 aa, synthetic construct; identical to SEQ ID NO: 34
  REGION 1..60: note = Synthetic

SEQ ID NO: 96 - protein, 60 aa, synthetic construct; identical to SEQ ID NO: 62
  REGION 1..60: note = Synthetic

SEQ ID NO: 97 - protein, 16 aa, synthetic construct
  REGION 1..16: note = Synthetic
FNVLMVHKRSHTGERP

SEQ ID NO: 98 - protein, 11 aa, synthetic construct
  REGION 1..11: note = Synthetic
LQCEICGFTCR

SEQ ID NO: 99 - protein, 33 aa, synthetic construct
  REGION 1..33: note = Synthetic
QKGNLLRHIKLHTGEKPFKCHLCNYACQRRDAL

SEQ ID NO: 100 - protein, 16 aa, synthetic construct
  REGION 1..16: note = Synthetic
FNVLMVHRRSHTGERP

SEQ ID NO: 101 - protein, 33 aa, synthetic construct
  REGION 1..33: note = Synthetic
QRGNLLRHIRLHTGERPFRCHLCNYACQRRDAL

SEQ ID NO: 102 - protein, 21 aa, synthetic construct
  REGION 1..21: note = Synthetic
TGEKPFKCHLCNYACQRRDAL

SEQ ID NO: 103 - protein, 21 aa, synthetic construct
  REGION 1..21: note = Synthetic
TGERPFRCHLCNYACQRRDAL

SEQ ID NO: 104 - protein, 23 aa, synthetic construct
  REGION 1..23: note = Synthetic
HECKLCGASFRTKGSLIRHHRRH

SEQ ID NO: 105 - protein, 23 aa, synthetic construct
  REGION 1..23: note = Synthetic
LQCEVCGFQCRQRASLKYHMTKH

SEQ ID NO: 106 - protein, 23 aa, synthetic construct
  REGION 1..23: note = Synthetic
YRCRACGRACSRLSTLIQHQKVH

SEQ ID NO: 107 - protein, 23 aa, synthetic construct
  REGION 1..23: note = Synthetic
YQCKVCGRAFKRVSHLTVHYRIH

SEQ ID NO: 108 - protein, 23 aa, synthetic construct
  REGION 1..23: note = Synthetic
LQCEICGYQCRQRASLNWHMKKH

SEQ ID NO: 109 - protein, 23 aa, synthetic construct
  REGION 1..23: note = Synthetic
FACVICGRKFRNRGLMQKHLKNH

SEQ ID NO: 110 - protein, 23 aa, synthetic construct
  REGION 1..23: note = Synthetic
LQCEICGFTCRQKASLNWHQRKH

SEQ ID NO: 111 - protein, 23 aa, synthetic construct
  REGION 1..23: note = Synthetic
FVCPRCGRGFSQPKSLARHLRLH

SEQ ID NO: 112 - protein, 23 aa, synthetic construct
  REGION 1..23: note = Synthetic
FQCPICGLVIKRKSYWKRHMVIH

SEQ ID NO: 113 - protein, 23 aa, synthetic construct
  REGION 1..23: note = Synthetic
LQCEICGFTCRQKASLNWHMKKH

SEQ ID NO: 114 - protein, 74 aa, synthetic construct
  REGION 1..74: note = Synthetic
AGSGGSGGSGGSPVPSTPPTNSSSTPPTPSPSPVPSTPPTNSSSTPPTPSPSPVPSTPPTNSSSTPPTPSPSAS

SEQ ID NO: 115 - protein, 74 aa, synthetic construct
  REGION 1..74: note = Synthetic
AGSGGSGGSGGSPVPSTPPTPSPSTPPTPSPSPVPSTPPTNSSSTPPTPSPSPVPSTPPTPSPSTPPTPSPSAS

SEQ ID NO: 116 - protein, 66 aa, synthetic construct
  REGION 1..66: note = Synthetic
AGSGGSGGSGGSPVPSTPPTPSPSTPPTPSPSGGSGNSSGSGGSPVPSTPPTPSPSTPPTPSPSAS

SEQ ID NO: 117 - protein, 74 aa, synthetic construct
  REGION 1..74: note = Synthetic
AGSGGSGGSGGSPVPSTPPTPSPSTPPTPSPSPVPSTPPTPSPSTPPTPSPSPVPSTPPTPSPSTPPTPSPSAS

SEQ ID NO: 118 - protein, 152 aa, synthetic construct
  REGION 1..152: note = Synthetic
AGSGGSGGSGGSPVPSTPPTPSPSTPPTPSPSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDPVPSTPPTPSPSTPPTPSPSAS

SEQ ID NO: 119 - protein, 144 aa, synthetic construct
  REGION 1..144: note = Synthetic
AGSGGSGGSGGSPVPSTPPTPSPSTPPTPSPSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDGGSGGSGGSGGSAS

SEQ ID NO: 120 - protein, 242 aa, synthetic construct
  REGION 1..242: note = Synthetic
AGPVPSTPPTPSPSTPPTPSPSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDGGSGGSGGSGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDGGSGGSGGSGAS

SEQ ID NO: 121 - protein, 262 aa, synthetic construct
  REGION 1..262: note = Synthetic
AGSGGSGGSGGSPVPSTPPTPSPSTPPTPSPSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDGGSGGSGGSGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDPVPSTPPTPSPSTPPTPSPSAS

SEQ ID NO: 122 - protein, 121 aa, synthetic construct
  REGION 1..121: note = Synthetic
AGSGGSGGSGGSPVPSTPPTPSPSTPPTPSPSQIFVKTLTGKTITLEVEPSDTIENVKAKIQDKEGIPPDQQRLIFAGKQLEDGRTLSDYNIQKESTLHLVLRLRGGGGSGGSGGSGGSAS

SEQ ID NO: 123 - protein, 326 aa, synthetic construct
  REGION 1..326: note = Synthetic
AGSGGSGGSGGSPVPSTPPTPSPSTPPTPSPSDGRYSLTYIYTGLSKHVEDVPAFQALGSLNDLQFFRYNSKDRKSQPMGLWRQVEGMEDWKQDSQLQKAREDIFMETLKDIVEYYNDSNGSHVLQGRFGCEIENNRSSGAFWKYYYDGKDYIEFNKEIPAWVPFDPAAQITKQKWEAEPVYVQRAKAYLEEECPATLRKYLKYSKNILDRQDPPSVVVTSHQAPGEKKKLKCLAYDFYPGKIDVHWTRAGEVQEPELRGDVLHNGNGTYQSWVVVAVPPQDTAPYSCHVQHSSLAQPLVVPWEASPVPSTPPTPSPSTPPTPSAS

SEQ ID NO: 124 - protein, 606 aa, synthetic construct
  REGION 1..606: note = Synthetic
AGSGGSGGSGGSGGSGGSGGSGGSDGRYSLTYIYTGLSKHVEDVPAFQALGSLNDLQFFRYNSKDRKSQPMGLWRQVEGMEDWKQDSQLQKAREDIFMETLKDIVEYYNDSNGSHVLQGRFGCEIENNRSSGAFWKYYYDGKDYIEFNKEIPAWVPFDPAAQITKQKWEAEPVYVQRAKAYLEEECPATLRKYLKYSKNILDRQDPPSVVVTSHQAPGEKKKLKCLAYDFYPGKIDVHWTRAGEVQEPELRGDVLHNGNGTYQSWVVVAVPPQDTAPYSCHVQHSSLAQPLVVPWEASGGSGGSGGSGGSDGRYSLTYIYTGLSKHVEDVPAFQALGSLNDLQFFRYNSKDRKSQPMGLWRQVEGMEDWKQDSQLQKAREDIFMETLKDIVEYYNDSNGSHVLQGRFGCEIENNRSSGAFWKYYYDGKDYIEFNKEIPAWVPFDPAAQITKQKWEAEPVYVQRAKAYLEEECPATLRKYLKYSKNILDRQDPPSVVVTSHQAPGEKKKLKCLAYDFYPGKIDVHWTRAGEVQEPELRGDVLHNGNGTYQSWVVVAVPPQDTAPYSCHVQHSSLAQPLVVPWEASPVPSTPPTPSPSTPPTPSPSAS

SEQ ID NO: 125 - protein, 46 aa, synthetic construct
  REGION 1..46: note = Synthetic
AGSGNSSGSGGSGGSGNSSGSGGSPVPSTPPTPSPSTPPTPSPSAS

SEQ ID NO: 126 - protein, 141 aa, synthetic construct
  REGION 1..141: note = Synthetic
KLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQRSAGGGGSGGGGSGGGGAS

SEQ ID NO: 127 - protein, 245 aa, synthetic construct
  REGION 1..245: note = Synthetic
KLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGAS

SEQ ID NO: 128 - protein, 431 aa, synthetic construct
  REGION 1..431: note = Synthetic
KLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNAIEYYQKALELDPNNAEAWYNLGNAYYKQGDYQKAIEYYQKALELDPNNAEAWYNLGNAYYKQGDYQKAIEDYQKALELDPNNLQRSAGGGGSGGGGSGGGGAS

SEQ ID NO: 129 - protein, 451 aa, synthetic construct
  REGION 1..451: note = Synthetic
KLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSAGGGGSGGGGSGGGGAS

SEQ ID NO: 130 - protein, 7 aa, synthetic construct
  REGION 1..7: note = Synthetic
GGGGSAS

SEQ ID NO: 131 - protein, 12 aa, synthetic construct
  REGION 1..12: note = Synthetic
GGGGSGGGGSAS

SEQ ID NO: 132 - protein, 17 aa, synthetic construct
  REGION 1..17: note = Synthetic
GGGGSGGGGSGGGGSAS

SEQ ID NO: 133 - protein, 27 aa, synthetic construct
  REGION 1..27: note = Synthetic
GGGGSGGGGSGGGGSGGGGSGGGGSAS

SEQ ID NO: 134 - protein, 32 aa, synthetic construct
  REGION 1..32: note = Synthetic
GGGGSGGGGSGGGGSGGGGSGGGGSGGGGSAS

SEQ ID NO: 135 - protein, 37 aa, synthetic construct
  REGION 1..37: note = Synthetic
AGGGSGGGGSGGGGSGGGGSGGGGSGGGGSGGGGSAS

SEQ ID NO: 136 - protein, 42 aa, synthetic construct
  REGION 1..42: note = Synthetic
AGGGSGGGGSGGGGSGGGGSGGGGSGGGGSGGGGSGGGGSAS

SEQ ID NO: 137 - protein, 47 aa, synthetic construct
  REGION 1..47: note = Synthetic
AGGGSGGGGSGGGGSGGGGSGGGGSGGGGSGGGGSGGGGSGGGGSAS

SEQ ID NO: 138 - protein, 561 aa, synthetic construct
  REGION 1..561: note = Synthetic
MLLLVTSLLLCELPHPAFLLIPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGFNVLMVHKRSHTGERPLQCEICGFTCRQKGNLLRHIKLHTGEKPFKCHLCNYACQRRDAL

SEQ ID NO: 139 - protein, 449 aa, synthetic construct
  REGION 1..449: note = Synthetic
MLLLVTSLLLCELPHPAFLLIPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSRRSRLLHSDYMNMTPRRPGPTRRHYQPYAPPRDFAAYRSSGFNVLMVHRRSHTGERPLQCEICGFTCRQRGNLLRHIRLHTGERPFRCHLCNYACQRRDAL

SEQ ID NO: 140 - DNA, 9899 bp, synthetic construct
  misc_feature 1..9899: note = Synthetic
  source 1..9899: mol_type = other DNA
ttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccctattccagcacatatgaggcttggcgtaactagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttgggctcgagggggcccgggtgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaataccgtcgactccggaatagccaccatgcttctcctggtgacaagccttctgctctgtgagttaccacacccagcattcctcctgatcccagagcagaaactcatctcagaagaggatctggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcacagtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactcctagaaggcctggacccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctccggattcaatgtcttaatggttcataagcgaagccatactggtgaacgcccattgcagtgcgaaatatgcggctttacctgccgccagaaaggtaacctcctccgccacattaaactgcacacaggggaaaaaccttttaagtgtcacctctgcaactatgcatgccaaagaagagatgcgctccgcaaaagacgcggatccggcgagggtagaggcagtctcctcacatgtggcgatgtggaagaaaacccaggccccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaatgcatgagtaactgaggatccagggacagcagagatacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgctt

SEQ ID NO: 141 - DNA, 9563 bp, synthetic construct
  misc_feature 1..9563: note = Synthetic
  source 1..9563: mol_type = other DNA
ttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccctattccagcacatatgaggcttggcgtaactagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttgggctcgagggggcccgggtgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaataccgtcgactccggaatagccaccatgcttctcctggtgacaagccttctgctctgtgagttaccacacccagcattcctcctgatcccagagcagaaactcatctcagaagaggatctggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcacagtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtcggaggagcaggctcctgcacagtgactacatgaacatgactcctagaaggcctggacccacccgccggcattaccagccctatgccccaccacgcgacttcgcagcctatcgctcctccggattcaatgtcttaatggttcatcggcgaagccatactggtgaacgcccattgcagtgcgaaatatgcggctttacctgccgccagcgcggtaacctcctccgccacattcgtctgcacacaggggaacggccttttcggtgtcacctctgcaactatgcatgccaaagaagagatgcgctcagaaggagacgcggatccggcgagggtagaggcagtctcctcacatgtggcgatgtggaagaaaacccaggccccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaatgcatgagtaactgaggatccagggacagcagagatacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgctt